SEQUENCE LISTING (614 sequences)

SEQ ID NO: 1 - DNA, 18001 bp, H. Sapiens
gaagtcatcagcaatgcaactgttcacatggaggatactccctgcttgaggggtcagacaggcctgctgggcaacccaggaggcttggatgaccgtctaccccagtgtttttgggatggaaagttccacattctgagaaccctcagtccctgggcaacctggggtggttagtcaccacagcttgtggcttgggcccatgacagcaggtagaaatgacgtggactgccgccagccgggcacagtggctcacgcctgtaatcccagcactttgggaggctgaggcatgtggatcacttgaggtcaggagttcgaaaccagcctggtcaacacggtgaaaccccatctctgctaaaaaaaaaaaatatatatatataaattagccaggcatggtgacgtgcacctgtggtcccagctactcaggaggctgaggcacaagaatcacttgaacccgggaggtggaggttgcagtgagattgcaccagtgcactctccagcctggcaacagagcaagactctgtctcaaacaaacaaaacaaaacaaacaaaaagacgtaagatgtggaccgctggagaatgggggtgctgcctgcagtcaaaacggagtgggggtgcccagctcagggccagaatgatcctattcccggcacttctcagtgaggctctgtggctcacctaagaaaccagcctcccttgcaggcaacggcctagctggcctggtctggaggctctcttcaaatatttacatccacacccaagatacagtcttgagatttgactcgcatgattgctatgggacaagttttcatctgcagtttaaatctgtttcccaacttacattaggggtttggaattctagatcgtatttgaagtgttggtgccacacacaccttaacacctgcacgctggcaacaaaaccgtccgctctgcagcacagctggggtcacctgacctttctcctgtcccccccacttgagctcagtggctgggcagcaggggatgcatggccactggccggccaggtgcagctctcagctggggtgttcagaggacgcctgtgtcctcccctcccccatccctctgtcacccttggaggcagagaactttgcccgtcagtcccatggggaatgtcaacaggcaggggcagcactgcagagatttcatcatggtctcccaggccctcaggctcctctgccttctgcttgggcttcagggctgcctggctgcaggtgcgtccggggaggttttctccataaacttggtggaagggcagtgggcaaatccaggagccagcccgggcttcccaaaccccgcccttgctccggacacccccatccaccaggagggttttctggcggctcctgttcaatttctttccttctagaaaccagcatccaggcacaggaggggaggcccttcttggtggcccaggctttggcgggattatttttcaaagaactttaggagtgggtggtgctttcctggcccccatgggcccctgcctgtgaggtcggacaagcgcagggagtctggggcctctcagagtgcaggaagtgcgcacagggtgctcccaggctggggagcacaggtaggggacggtgcgtgggggatggcgcctggggcatgggggatggggtgtgggaaacggcatgtggggcgtaggggatggggtgtggaggatcgggggtggggatggcgtgtggggtgtgggggatgggccgtgggggggtggggcctgggaaacagcatgtggggcatggggtgtgggggtgaggtgtgggaaagtgtgtggggtgtgggggatggggcatggaaagggcgtgtggggtgcaggggatggggcatggaggtgtgggggatggggtgtgtggggtgtcggggatggggcatgtggggtgtgggggatggggcatggaaagggcgtgtggggtgcagaggatggggcatgggggggtggggatggcgagtggggctggggcctgggaatggtgagtggggcatggggatggcgagtagggggtgtggcgtgaggatggctagtggggcgtggggatggcgtgtggggatggcgagtggggggtgggctgtgagggacagtgcctgggatgtggggctgcagccctagctcacagcatggccttatgaccccggccaccttcctgccccaggcggggtcgctaaggcctcaggaggagaaacacgggacatgccgtggaagccggggcctcacagaggtgagcagggactgccactggttttgtcctggggcccagtgggggccaacatcacctccttcccctcccatggcaaagagccagcccgcggggtggctactgcagtgccccccaaggagggtgttccctgctcgagaggaagtgaccgctccagcttggccttccctgggactggggtgcaggcgattttatcttctttgctccattctgttccttccagataatcgtgtgttcttcatcaggttttcctcagttcttgagagcttttctgatgcaaatctgctttcaccccagggcggtcaccggctctgctcacaccagcctccaagggtgtgggtgtcccgggagtgtgggtgtcccgggggcgtgggtgtcccaggagtgtgggtgtcccgggggcgtgggtgtcccgggagtgtgggtgtcccgggggcgtgggtgtcccgggagtgtgggtgtcccggaggcgagggtgtcccgggagtgtgggtgtcccgggggagtgggtgtcccgggagtgtgggtgtcccggaggcgagggtgtcccgggagtgtgggtgtcccgggggcgtgggtgtcccgggagtgtgggtgtcccgggggagtgggtgtcccgggagtgtgggtgtcccggaggcgagggtgtcccgggagtgtgggtgtcccgggggagtgggtgtcccgggagtgtgggtgtcccggaggcgagggtgtcccgggagtgtgggtgtcccgggggcgtgggtgtcccgggagcgtgggtgtcccgggggcgtgggtgtcccgggagtgtgggtgtcccgggggcgtgggtgtcccgggagtgtgggtgtcccgggggcgtgggtgtcccgggagtgtgggtgtcccgggagtgtgggtgttccggaggcgagggtgtcccgggagtgtgcgtgtcccgggggcgtgggtgtcccgggggcgtgggtgtcccgggggcgtgggtgttccggaggcgagggtatcccagaagtgtgagtgtcccaggggcgtgggtgtcccgggggtgtgggtgtcccgggggcgtgggtgtcccgggagtgtgggtgttccggaggtgagggtgtcccgggagtgtgggtgttccggaggcgagggtgtcccgggagtgtgggtgtcccgggggcgtgggtgtcccgggagtgtgggtgttccggaggtgagggtgtcccgggagtgtgggtgttccggaggcgagggtgtcccgggagtgtgggtgtcccaggggcgtgggtgtcccgggagtgtgggtgttccggaggcgagggtgtcccgggagtgtgggtgttccggaggcgagggtgtcccgggagtgtgggtgtcccgggggcgtgggtgtcccgggggttgtgggtgtcccgggagtgtgggtgttccggaggcgagggtgtcccgggagtgtgggtgttccggaggcgagggtgtcccgggagtgtgggtgtcccgggggtgtgggtgtcccgggggtgtgggtgtcccgggagtgtgggtgtcccgggggagtgggtgtcccgggagtgtgggtgttccggaggcgagggtgtcccaggagcgtgggtgtcccggaggcgagggtgtcccgggagcgtgggtgtcccgggggcgtgggtgtcccgggagtgtgggtgtcccgggggagtgggtgtcccgggagtgtgggtgtcccggaggcgagggtgtcccaggagtgtgggtgtcccgggggcgtgggtgtcccgggagtgtgggtgttccagaggcgagggtatcccagaagtgtgagtgtcccgggggtgtgggtgtcccgggggtcgtgggtgtcccgggagtgtgggtgttccagaggcgagggtgtcccgggagtgtgggtgtcccaggggtgtgggtgtcccgggggcgtgggtgtcccgggagtgtgggtgtcccgggggagtgggtgtcccgggagtgtgggtgttccggaggcgagggtgtcccgggagtgtgggtgttccggaggcgagggtgtcccgggagcgtgggtgtcccgggggcgtgggtgtcccgggagcgtgggtgtcccaggggtgtgggtgtcccgggggcgtgggtgtcccgggagtgtgggtgtcccgggggagtggatgtcccgggagtgtgggtgttccggaggcgagggtgtcccgggagtgtgggtgttccggaggcgagggtgtcccgggagtgtgggtgtcccgggggcgtgggtgtcccgggagtgtgggtgtcccgggggcgtgggtatcccagaagtgtgagtgtcccaggggcgtgggtgtccggggggcgtgggtgtcccgggggtgtgggtgtcccgggggtcgtgggtgtcccgggagcgtgggtgtcggggactgcagggacatgggcctcccctcccactcctgccgcccagggcacctcctgtgaggactcggagtccgtgagttcccacctccttgagcccgattctttggtgtccccgcctgcatcctcagcctccttccaaaccagaccagttctctaggggcgtcgacgtgtgaaactgattttaaagaaaacaggcagtggcctttctctcggccccacgtggcccagtagcgctcaccttccgtcccttcttccgcgctcagtaaccaatttaggccgctcctgcagaactcgggctcctgcccaccggcccacagcgtccacctgaggcctcgtcctcccagcaaaggtcgtccctccggaacgcgcctcctgcggcctctccagagcccctcccgcgcgtcctctcagccccgctcgcctcctcccggggcctccctctcccgcctgcccccaggcccgtctcccctcgcgggctgaggcaggttcgggcagcacggccgccccggggcgggggtcactctccaccaccgcgtggtgcccacagctcacggcgctcccgggtgacggtcccctcggctgtagggcgtcctgaagagcggcctgctcggagctgagcgcacggggttgcctcgccctgggcgtctctggccctcaccagccccgtcttcccatgggcaaaacggcggtcctgtttgtccacaagtaaccgtcggggttacggaggggccaggagctgcggcggggggctgtgctctcaggaccggccccaggaggatccgcgcgaggtctggagctctcaggggtcgcgggggacagaggggccccaagcggaggcggggaaggcggcagaagcccaggaccgccaagagctggcgaggaagcccggggctcgctgtcgggggagccgggcaggggccgcgcctcggaccaggacggaggcctggggaaggcggatctggccgccggagacgcggtgcgggtggagacgagggatttggatttccgcgggcggctgtacggatttccacgcgcggttcacgtgggccccagggggttgcccggcacccggggccgcgccgccttctcctcgccggcatcgacccgcagcctcacgtttacgcggcggcgcccgcagcccccttcggcccggcttccgcgcgtgcccccgagcgcgccctcgggatcagcccccggaagcagagaggccaggccgggaaggatgggcgacgggggtggctgacccgggagcacggcagggaggacacccagccaggcccgcgagcagcgccgctcccctcctccaggacgggcgggaacctgcgatgcccccgccgcgtgggccgtggggcggtctccgaggcactgggcggggcacgcggtgggcgcttcacggaactcgcatttcccagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacggtgagcccagcctcggggcgccccgcgccgcggacactgcaggcggcggtgaaccaggccgcgtggggccgcctgcgtctctttggctgcggctgtgggcggcgaacacgcagcggcgcccgcgcggcgctttctgcgggggtcgctttccgcccggggtgactccgctttcctgggcgatgccccccacccccaggcacgcgctctccccgtgcggccgcaccgcgcatgccggttttcacatcagaaaatacgatttgcaaagcacacttagggtgtcccccttaacttcccaagggagtccccccagtccccgaagggtccagggcagcctgcgcatcgcagacgcgcgcggctcgcagaagggacgtggtgagaagctggcccacagcatgccaccagcggcacctcctcagggcacgtgtcggggagaaacaacacttagggacctgggactttctccagctcacgctcacgggtcacctcacactccaagatcacctcaaagaggacacctcacacagggcacacttcacactcacaggtcacctcacactcacaggacacctcacactcacagggcacacttcacactcacgggtcacctcacactccaagatcacctcaaagaggacacctcacacagggcacacttcacactcacaggtcacctcacactcacaggacacctcacactcacagggcacacttcacactcacgggtcacctcacactccaagatcacctcaaagaggacacctcacacagggcacacttcacactcacgggtcacctcacactcacaggacacctcacacaagacacctcacacggggcacacttcacactcacaggtcacctcacacccacaggacacctcacacagggcacacttcacactcacgggtcacctcacactcacaggacacctcacacaagacacctcacacggggcacacttcacactcacaggtcacctcacacccacaggacacctcacacagggcacacttcacactcacgggtcacctcacactcacaggacacctcacactcagggcgcacttcacactcacgggtcacctcacacccacaggacacctcacagaggtcacctcacacaggacacctcacactcagggtgcacttcaaacccacaggtcatttcacctcacactcacaggacacctcacacaagataccacacggggcacacttcacactcacaggtcacctcacactcacaggacacctcacagaggtcacctcacacggggcacacttcacactcacaggtcacctcacacccacaggacacctcacagaggtcacctcacacccacaggacacctcacacaggacacctcacagaggtcacctcacacccacaggacacctcacactcataggtcacctcagtcttacaggacaactcacactcacaggtcaccttactctcacaggacacctcacactcacaggtcaccttactctcacaggacacctcactctcacaggacacctcacacagggcacacttcactcccacaggtcaccatacctcacacagatcacctcatactcacagatcacttcattctcacaggatacctcacactcagggcacacttcacactcacaggtcacacctcacacagatcatctcattctcacaggacacctccctctcacaggtcacctcacactcacaggacacctcacagaggtcacctcacacccacaggacacctcacagaggtcacctcacacggggcacacttcacactcaggtcacctcacacccacaggacacctcacagaggtcacctcacacccacaggacaactcacagaggtcacctcacacaggacacctcacaaaggtcacctcacacccacaggacacctcacactcataggtcacctcagtcttacaggacaactcacactcacaggtcaccttactctcacaggacacctcacactcacaggtcaccttactctcacaggacacctcacacagggcacacttcactcccacaggtcaccatacctcacacagatcacctcatactcacagatcacttcattctcacaggatacctcacactcagggcacacttcacactcacaggtcacacctcacacagatcatctcattctcacaggacacctccctctcacaggtcaccttacactcatctcacactcacaggtcgccacacctcacactcacaggatgcctcacactcacagaaccacatctcatatgcacaagacacctcacactcaggacacctcatgctcaaagaagcctcacactcacaggaggtccagctgtctgaggcaaaggctaacatgaccctttccagacaaattgaggatggtcatgcctagcatttttatacacctagttttgaaagcatttctcatctgttgtattctcacagcaccccgtgagtttaagttcaggtggccaacagtttcttcagcaatcacttttttctgtggagtgcttttgctgtttgtggaatattttgcatctgctactgcaccctctccccgtatgtgtggccaccctgtcagaggtggagctgtggctcagagcctgtgtacctcgtcccaggtccacagctcagcgacagaagagtcagggttgaacctcgggtgttctgacttgggagcaggaaatgtgtggtcacccatagttccagatgtcctggggaggggccaagattagaagaaacctacctcagctccagaggaaagtctggcttcctgagcccaccccgccagacccaggtccaagtcccccaaccccagttcatggtgtgtccagtgcttaccgttgggtgctctggtgaaggtgcatctcacgaggcttgctctcttgttccttcagaagctgttctggatttcttacagtggtgagtggatgatcaccaccagtcctgcctgcaacccttctcagcttactgacaccagcccactccacagatggggaccagtgtgcctcaagtccatgccagaatgggggctcctgcaaggaccagctccagtcctatatctgcttctgcctccctgccttcgagggccggaactgtgagacgcgtaaggccccactttgggtcccatatttgcagagggccctggggagctggtggaggtggcctggccaaccgggctgcagggtgcacaacctggtggggtgtgtaggccgggcattcagggctcagccccagttggaaattggtctaggtgaccttgaaatcccttccagtctgaggtctttgacagggacccaaggttctgattatcagactcagtggccccttgggctcccggccctgggcaattctcagccctcgagatggcccagctgagagtccctgtgtccctgtcccacttccacatcccaccacgcaggaccgcttggtaaacttccccttctctactttccattacaaaggtttgaggggtttgttttttttttaaccatctgaatattaaattatcacaaagtttgaggcccccaacctcccttgggttcagtaattcactagaaggactcatagaatccactgaagtggatacactcacaggtaccgtttattacagcaaaggatgcaggcttaagtctgcagagggaccaggcacaagcttccccttgtcctctccctgtggggtcatgtggacagtccttaattctcccagaatgacgtgtgacgagacgtgggaagtactgccaacttgggaagctctacgagccccggtgtccagaggttttatcagggctcaatcacatagacccagctgaccacccgcatggctgacctcagtctcagcccctccagaggctacgccgatagtgcggcccaaggccccaccatacatcacattgtcagctagaccatccagcatggctcaaggcccaggtaaacaccaacattccctcaggcaagaccttccaagggcttagcggtcatttcccaggagccaaggcaaaggctaccctttctctggcacagcagttcatccttgaccacccaagaccacattcttacactgaatgagctctcctgtgcagcagccattttcttctctaagcagaagagagcccagcaagctggaggaggctgaagagagaggcttcctgctggtcatctgggtccagaatgcctggagatctctgctcagccctggtgcccagcagccctggtgtgcatcctgcagggcaggccttcccgccggagtcctggacttgctcagggccactccccttgcccatgtcaaccaaagtcaggctgccggttctgcttcttctgtctgagcccatgaccagtgctgggactaactgtccccaggcgggctcacggtggtacgaggccagcttggagaactgtctcagctctctggtcctctcgtcagttgggtctctgattggaaagtcccttggacacttttaccatccccattggactttcactttcccccaggctcccatcagctgctcggaagagtggtcaccctggaggccactgcccaccagccaggcaccccccaaatgcaaccgcagccagcactgccagccactggcaaggctgttcagacatgtggctcctctgatccacgccttgtcctttggatcagtccacggagcaggtggtgccaagctcaggctctgtcacccacagctcagtgccaccttccaggcagaacaccactgctgacccagggcatggccaccccgggggctggctctcgctgacccccagaagcccctctcagggtgtccccttcctgtccccagacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacaggtgaccaggcttcatgtcccagtcccagatgacaccagtccctgtcccactacggattatcttactggacaaaagacgggtgggagtggcttcacatctactgagcactaactatgcactgaccaattgtgaggtgggatctgggcaccaagggtggcacaggccagagcgacagtgactaggatgggcaccctgggggcaatccctgaatggcctcaggccccctgccaattctaggcagaccaggggagccaagcaaggcactatctcacgtccaactcccactcgcaggacctccgccagggttcatgaatctacttcggcacagccaatgtctgtactgactgctgcccactctgcattccaaaactcgtaaaggctcctgggaaaatgggatgtttctccaaaccagcctggaacgaatgggctgcacttccaaaagcagggacaccccacacccactgtgtctcaaagaggcggacgtgcccaccctggccacacagcctgggactcagcctgccacctcctcgggcttcctttctggcccaagaccttgattgaagcagatcaaaactaagcatgggatcaaaacaacacagtttgattcatctttaggtagaatttcattcaccttctactaaagtcaaacaacacatcttctccctgaaaagtgagcagagggcggttttaagacgtaagccctctgtttcctccaaaaccagccctgaccattgtctcctcagccagccacttcttcaagggcctctcatggccgggccccaccagtcaggcccagccgaggccctgccttccaccacccctgggccctgggagctcctgctcctgggggcctcccatagcctcggcctcaaggcctctcagaggatgggtgtttctgaatctttcctagtggcacgttcatccctcacaaatctctgcatctttctgacttttgttttacacagttgaatatccatgtggaaaaatacctattctagaaaaaagaaatgccagcaaaccccaaggccgaattgtggggggcaaggtgtgccccaaaggggagtgtccatggcaggtaaggcttcccctggcttcaggattccaagccctgagggtcttgaagccttttgaatgtgaacaacagctctggaagggaaaatgggcaggtcagccccaagcccaccaggctccaagtcagcacacctagcacctccagctcgcggcacccccatgcttttagtggggcaaggaaggagaaaagaaaacgacactcactgagggtctaccctgtgcagagaaccctgcgagatgccccatccgagttgtcacgtcgtcctcacggttactctttgaggtgggatctttgcctgatctttgcaaaatcaggagcattggatcaaagctatgtgaagatcctgtgaggtgaacagtgaaatctcacagcgacatttgtattcttgggccgtgcccaagagcacgtctcggctagagaggggcacagcctcccagagccaggtctgagcagctttgcctgggagggatctgcaaagaccccaggatttcagaaagaaattgtgcaatgccagaggttccttggcatgcccgggagggcgagtcatcagagaaacaatgacagcaatgtgacttccacacctcctgtccccccgcccaggtcctgttgttggtgaatggagctcagttgtgtggggggaccctgatcaacaccatctgggtggtctccgcggcccactgtttcgacaaaatcaagaactggaggaacctgatcgcggtgctgggtgggtaccactctcccctgtccgaccgcggtgctgggtgggtgccactcttccctgtccgaccgcggtgctgggtgggtgccactctcccctgtccgaccgcggtgctgggtgggtgccactctcccctgtccgaccgcggtgctgggtgggtgccactctccgctgtccgaccgcggtgctgggtgggtaccactctcccctgtctgaccgcagctctcaagtgtctcaggggctgtggctctgggcttcgtgctgtcacttccacagacagacagacatccccaaaaggggagcaaccatgctgggcacgactgctgtggccaccgtgctctcagccactttcccatgcccaaataaaacgataaaagactgggggcttctgcccatcctgcctcacttgaccaagagcccagaagaggatgcgacacccagggcctcatgggaccaccggctggcaggggttctgctcactgggtttatgggtgagacgagcactcccaggagggccactgggccgggaagaactgtggagaatcggggcacgccctgtcctcccagctgccagggcacagcatcccttccccacctcaacacccagaccccagattcaccccagttcacttgtccccacacgagccacaggctgccacctggggcaggctggccccaccttggggttagatgcaggtccccttgccccagaaggagactgcagcccctgcagacctagaaatggccacagcccatccccatgcaccagggggtgaggtggcaggtggtggaaagggcctgaggggggcttcttccttccaggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacacccatattagtgtccatggagtggccgtggcagagacgtccagccggacagaccagctgacccgccaagcccagcatggttagtgtcaggacctctgctgaagatgcttgctgaccctggccagaccccggttcctaatgccccctaaacgggacgggagccagtggcgggccctgatccaggtcagagctggctctgctttctcttttgtccgagtgaccatgcctcagtttcctcatgtgtaaaacaggagcccaccgtgatgcttatggtgggatgagatcagcatggatggaacaaggccctggaagggcccatgccatggtcatcgacagcaaagccactctgcagacagatgcttcagtgaattggtagaaaattctgcaaccagaatgcccggggctcctgagggcctaagcccagcccagggttctggaagccactctgacttcttgggagtggaagttggcaggactcttcctgggaagaagcggagggtggggatgagaggacagttcaggagcccacccagacccacaggaggaaactaggggagtcatgcggggtcctggtggagcgccagcctcccttcctgccaatgggaaatgcaggcgcccacctcatggtgctgccggaggagggggcccgggactccccagaggcttcgctgaagggcctgggcgcccccaaaggctacatgtttcatatgggacgtgccacctgccacggctcagctccagctttctgtgagtggcgagatagaatacggggaggccactggccatgggcctgggacagggtgggatgaggcggcaggcttgggccaccaaagccagcatcgccacccagcattgatgacaaagactgcgtgtctgccatgagcatcctgctgttggtgcacacaccgcattggtctctccatacaaacatgcctagaggcgatgtcagagggtggagaccaggagaggcaggagtcagacatctggtgccaccaggaaggcccttctcagaggaccaggctgtgcgtggtgcccgccgtgggaggccagcctggcgttggcatccagcatcatcagtttgtgcagtcgggtggggctcagtgagtgcctcctgtgtgccaggcacaatgacgcacaatgtgtgcacaccaggctcatgtgcaggtggctgcgagacagggcgacccatcaaggcagatgcaccatgaggcagtggccagtgctgtgggtgttaggggcattgctccccggccactacggcatagcaggcagtgatcgccacactggccaagctttagaccatttattccagagaccccagaggcaaaaagcccggctgcacctcccagtgactcccacagccattgagcagagacactcaggaccttgtgatgggaggtttctgcactggagaacgagcccagaagccctctcagcctcggaacagtgtggccagtggtgggcaggtcaggaggggcttcagacacagcctgtccctccagatggtcacgggaaggtcactccccacagaagtacgttttggggccatgcgggcacagaaggtttgggggtgggtggggcaggtgccagcctggcctgtgggaggccatggtgcagatgccaagccccccccgtgacatgagaccacctgataccacccagagagtggctgtgagcggaagggcccgcccagaaacaagcagggccttggggcagaagtcctgggctcagatcccacgctcactgccagcggcctcggctcaggcttctgcgctctctaaacttagttttctcttctggaaaaatgatggggaaaatgatatttgtatgtgaggactgagagttaaatgtaaacatctggaaactacaaaatgagcacgaaatgatgtttttattcttagaacagaaagtccccacacccgcggccctggtgactgatgaggatgaggttctgcggggcctctctggccgcccagctctgcctggggaaggtggggcca

SEQ ID NO: 2 - DNA, 3075 bp, H. Sapiens
agtcccatggggaatgtcaacaggcaggggcagcactgcagagatttcatcatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacaaaaaaaaaaaaaaaaa

SEQ ID NO: 3 - DNA, 564 bp, H. Sapiens
  Synthetic Oligonucleotide
atcatggtctcccaggccctcaggctcctctgccttctgcttgggcttcagggctgcctggctgcaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgtggtgagtggatgatcaccaccagtcctgcctgcaacccttctcagcttactgacaccagcccactccacagatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaata

SEQ ID NO: 4 - DNA, 20 bp, Artificial Sequence
  Synthetic Oligonucleotide
agtcctgggtcatcagccgg

SEQ ID NO: 5 - DNA, 20 bp, Artificial Sequence
  Synthetic Oligonucleotide
gagaccctggtgtacacccc

SEQ ID NO: 6 - DNA, 20 bp, Artificial Sequence
  Synthetic Oligonucleotide
cctgcagccaggcagccctg

SEQ ID NO: 7 - DNA, 20 bp, Artificial Sequence
  Synthetic Oligonucleotide
tcctgaggccttagcgaccc

SEQ ID NO: 8 - DNA, 20 bp, Artificial Sequence
  Synthetic Oligonucleotide
gaggccccggcttccacggc

SEQ ID NO: 9 - DNA, 20 bp, Artificial Sequence
  Synthetic Oligonucleotide
tgttgacattccccatggga

SEQ ID NO: 10 - DNA, 20 bp, Artificial Sequence
  Synthetic Oligonucleotide
ccatgatgaaatctctgcag

SEQ ID NO: 11 - DNA, 20 bp, Artificial Sequence
  Synthetic Oligonucleotide
cctgggagaccatgatgaaa

SEQ ID NO: 12 - DNA, 20 bp, Artificial Sequence
  Synthetic Oligonucleotide
tgaagcccaagcagaaggca

SEQ ID NO: 13 - DNA, 20 bp, Artificial Sequence
  Synthetic Oligonucleotide
cagccctgaagcccaagcag

SEQ ID NO: 14 - DNA, 20 bp, Artificial Sequence
  Synthetic Oligonucleotide
ctgcagccaggcagccctga

SEQ ID NO: 15 - DNA, 20 bp, Artificial Sequence
  Synthetic Oligonucleotide
taagaaatccagaacagctt

SEQ ID NO: 16 - DNA, 20 bp, Artificial Sequence
  Synthetic Oligonucleotide
ttgaggcacactggtcccca

SEQ ID NO: 17 - DNA, 20 bp, Artificial Sequence
  Synthetic Oligonucleotide
ctggtccttgcaggagcccc

SEQ ID NO: 18 - DNA, 20 bp, Artificial Sequence
  Synthetic Oligonucleotide
tggagctggtccttgcagga

SEQ ID NO: 19 - DNA, 20 bp, Artificial Sequence
  Synthetic Oligonucleotide
tataggactggagctggtcc

SEQ ID NO: 20 - DNA, 20 bp, Artificial Sequence
  Synthetic Oligonucleotide
agaagcagatataggactgg

SEQ ID NO: 21 - DNA, 20 bp, Artificial Sequence
  Synthetic Oligonucleotide
agggaggcagaagcagatat

SEQ ID NO: 22 - DNA, 20 bp, Artificial Sequence
  Synthetic Oligonucleotide
tctcacagttccggccctcg

SEQ ID NO: 23 - DNA, 20 bp, Artificial Sequence
  Synthetic Oligonucleotide
agatcagctggtcatccttg

SEQ ID NO: 24 - DNA, 20 bp, Artificial Sequence
  Synthetic Oligonucleotide
tgctcacagccgccgttctc

SEQ ID NO: 25 - DNA, 20 bp, Artificial Sequence
  Synthetic Oligonucleotide
tgcagtactgctcacagccg

SEQ ID NO: 26 - DNA, 20 bp, Artificial Sequence
  Synthetic Oligonucleotide
gacaccccgtctgccagcag

SEQ ID NO: 27 - DNA, 20 bp, Artificial Sequence
  Synthetic Oligonucleotide
tgcaggacaccccgtctgcc

SEQ ID NO: 28 - DNA, 20 bp, Artificial Sequence
  Synthetic Oligonucleotide
tccacatggatattcaactg

SEQ ID NO: 29 - DNA, 20 bp, Artificial Sequence
  Synthetic Oligonucleotide
gtatttttccacatggatat

SEQ ID NO: 30 - DNA, 20 bp, Artificial Sequence
  Synthetic Oligonucleotide
agaataggtatttttccaca

SEQ ID NO: 31 - DNA, 20 bp, Artificial Sequence
  Synthetic Oligonucleotide
tccattcaccaacaacagga

SEQ ID NO: 32 - DNA, 20 bp, Artificial Sequence
  Synthetic Oligonucleotide
gagaccacccagatggtgtt

SEQ ID NO: 33 - DNA, 20 bp, Artificial Sequence
  Synthetic Oligonucleotide
ttgtcgaaacagtgggccgc

SEQ ID NO: 34 - DNA, 20 bp, Artificial Sequence
  Synthetic Oligonucleotide
tcttgattttgtcgaaacag

SEQ ID NO: 35 - DNA, 20 bp, Artificial Sequence
  Synthetic Oligonucleotide
tgatgacctgcgccacccgc

SEQ ID NO: 36 - DNA, 20 bp, Artificial Sequence
  Synthetic Oligonucleotide
tcagtgaggaccacgggctg

SEQ ID NO: 37 - DNA, 20 bp, Artificial Sequence
  Synthetic Oligonucleotide
cacatggtcagtgaggacca

SEQ ID NO: 38 - DNA, 20 bp, Artificial Sequence
  Synthetic Oligonucleotide
gggcaccacatggtcagtga

SEQ ID NO: 39 - DNA, 20 bp, Artificial Sequence
  Synthetic Oligonucleotide
tccgttcgggcaggcagagg

SEQ ID NO: 40 - DNA, 20 bp, Artificial Sequence
  Synthetic Oligonucleotide
gcaggcagtcctgggtcatc

SEQ ID NO: 41 - DNA, 20 bp, Artificial Sequence
  Synthetic Oligonucleotide
gtgactgctgcaggcagtcc

SEQ ID NO: 42 - DNA, 20 bp, Artificial Sequence
  Synthetic Oligonucleotide
tggccccagctgacgatgcc

SEQ ID NO: 43 - DNA, 20 bp, Artificial Sequence
  Synthetic Oligonucleotide
gcagccctggccccagctga

SEQ ID NO: 44 - DNA, 20 bp, Artificial Sequence
  Synthetic Oligonucleotide
gtactgggagaccctggtgt

SEQ ID NO: 45 - DNA, 20 bp, Artificial Sequence
  Synthetic Oligonucleotide
gccactcgatgtactgggag

SEQ ID NO: 46 - DNA, 20 bp, Artificial Sequence
  Synthetic Oligonucleotide
tttgcagccactcgatgtac

SEQ ID NO: 47 - DNA, 20 bp, Artificial Sequence
  Synthetic Oligonucleotide
gcatgagcttttgcagccac

SEQ ID NO: 48 - DNA, 20 bp, Artificial Sequence
  Synthetic Oligonucleotide
cctgaggccagcagatcacg

SEQ ID NO: 49 - DNA, 20 bp, Artificial Sequence
  Synthetic Oligonucleotide
cagcagcctgaggccagcag

SEQ ID NO: 50 - DNA, 20 bp, Artificial Sequence
  Synthetic Oligonucleotide
cacacatggagtcagcatcg

SEQ ID NO: 51 - DNA, 20 bp, Artificial Sequence
  Synthetic Oligonucleotide
gaggacagcacacatggagt

SEQ ID NO: 52 - DNA, 20 bp, Artificial Sequence
  Synthetic Oligonucleotide
gagagctaaacaaccgcctt

SEQ ID NO: 53 - DNA, 20 bp, Artificial Sequence
  Synthetic Oligonucleotide
ggtgatgcttctgaattgtc

SEQ ID NO: 54 - DNA, 20 bp, Artificial Sequence
  Synthetic Oligonucleotide
gcagccgtttattgtgaagc

SEQ ID NO: 55 - DNA, 20 bp, Artificial Sequence
  Synthetic Oligonucleotide
gcatctcagaggatgagcac

SEQ ID NO: 56 - DNA, 20 bp, Artificial Sequence
  Synthetic Oligonucleotide
gagggttcatttcagtgatg

SEQ ID NO: 57 - DNA, 20 bp, Artificial Sequence
  Synthetic Oligonucleotide
ccatgtgagggttcatttca

SEQ ID NO: 58 - DNA, 20 bp, Artificial Sequence
  Synthetic Oligonucleotide
agcctcaaacatctatcaaa

SEQ ID NO: 59 - DNA, 20 bp, Artificial Sequence
  Synthetic Oligonucleotide
tgggagctacagcctcaaac

SEQ ID NO: 60 - DNA, 20 bp, Artificial Sequence
  Synthetic Oligonucleotide
aatatcattgacaagggctg

SEQ ID NO: 61 - DNA, 20 bp, Artificial Sequence
  Synthetic Oligonucleotide
ccactgcagccagggcctgg

SEQ ID NO: 62 - DNA, 20 bp, Artificial Sequence
  Synthetic Oligonucleotide
agagtgcagcttgccaggtc

SEQ ID NO: 63 - DNA, 20 bp, Artificial Sequence
  Synthetic Oligonucleotide
cagcaagagtgcagcttgcc

SEQ ID NO: 64 - DNA, 20 bp, Artificial Sequence
  Synthetic Oligonucleotide
gggactcagcaagagtgcag

SEQ ID NO: 65 - DNA, 20 bp, Artificial Sequence
  Synthetic Oligonucleotide
gcttgccggagtctgagtgg

SEQ ID NO: 66 - DNA, 20 bp, Artificial Sequence
  Synthetic Oligonucleotide
gatggcatcgagtccactct

SEQ ID NO: 67 - DNA, 20 bp, Artificial Sequence
  Synthetic Oligonucleotide
gctctgaagtagatgatgcc

SEQ ID NO: 68 - DNA, 20 bp, Artificial Sequence
  Synthetic Oligonucleotide
tttacaagagcagggtgcct

SEQ ID NO: 69 - DNA, 20 bp, Artificial Sequence
  Synthetic Oligonucleotide
ttgcaggcaggactggtggt

SEQ ID NO: 70 - DNA, 20 bp, Artificial Sequence
  Synthetic Oligonucleotide
cagggcgaggcaaccccgtg

SEQ ID NO: 71 - DNA, 20 bp, Artificial Sequence
  Synthetic Oligonucleotide
gttacgaagactgggaaatg

SEQ ID NO: 72 - DNA, 20 bp, Artificial Sequence
  Synthetic Oligonucleotide
tccccaggacatctggaact

SEQ ID NO: 73 - DNA, 20 bp, Artificial Sequence
  Synthetic Oligonucleotide
gtggccatgccctgggtcag

SEQ ID NO: 74 - DNA, 20 bp, Artificial Sequence
  Synthetic Oligonucleotide
gaagccttacctgccatgga

SEQ ID NO: 75 - DNA, 20 bp, Artificial Sequence
  Synthetic Oligonucleotide
tagaccctcagtgagtgtcg

SEQ ID NO: 76 - DNA, 20 bp, Artificial Sequence
  Synthetic Oligonucleotide
gcagaggagcctgagggcct

SEQ ID NO: 77 - DNA, 20 bp, Artificial Sequence
  Synthetic Oligonucleotide
ccaggcagccctgaagccca

SEQ ID NO: 78 - DNA, 20 bp, Artificial Sequence
  Synthetic Oligonucleotide
cagggagcccggccgcagct

SEQ ID NO: 79 - DNA, 20 bp, Artificial Sequence
  Synthetic Oligonucleotide
catggacttgaggcacactg

SEQ ID NO: 80 - DNA, 20 bp, Artificial Sequence
  Synthetic Oligonucleotide
ccccattctggcatggactt

SEQ ID NO: 81 - DNA, 20 bp, Artificial Sequence
  Synthetic Oligonucleotide
tcgaaggcagggaggcagaa

SEQ ID NO: 82 - DNA, 20 bp, Artificial Sequence
  Synthetic Oligonucleotide
gttccggccctcgaaggcag

SEQ ID NO: 83 - DNA, 20 bp, Artificial Sequence
  Synthetic Oligonucleotide
cacacagatcagctggtcat

SEQ ID NO: 84 - DNA, 20 bp, Artificial Sequence
  Synthetic Oligonucleotide
cgtgtggtcactgcagtact

SEQ ID NO: 85 - DNA, 20 bp, Artificial Sequence
  Synthetic Oligonucleotide
gcttggtgcccgtgtggtca

SEQ ID NO: 86 - DNA, 20 bp, Artificial Sequence
  Synthetic Oligonucleotide
ccagcagagagtacccctcg

SEQ ID NO: 87 - DNA, 20 bp, Artificial Sequence
  Synthetic Oligonucleotide
gggtgtgcaggacaccccgt

SEQ ID NO: 88 - DNA, 20 bp, Artificial Sequence
  Synthetic Oligonucleotide
ccccacaattcggccttggg

SEQ ID NO: 89 - DNA, 20 bp, Artificial Sequence
  Synthetic Oligonucleotide
tggtgttgatcagggtcccc

SEQ ID NO: 90 - DNA, 20 bp, Artificial Sequence
  Synthetic Oligonucleotide
cacccagatggtgttgatca

SEQ ID NO: 91 - DNA, 20 bp, Artificial Sequence
  Synthetic Oligonucleotide
ccgcggagaccacccagatg

SEQ ID NO: 92 - DNA, 20 bp, Artificial Sequence
  Synthetic Oligonucleotide
gaaacagtgggccgcggaga

SEQ ID NO: 93 - DNA, 20 bp, Artificial Sequence
  Synthetic Oligonucleotide
ccagttcttgattttgtcga

SEQ ID NO: 94 - DNA, 20 bp, Artificial Sequence
  Synthetic Oligonucleotide
accacgggctggtgcaggcg

SEQ ID NO: 95 - DNA, 20 bp, Artificial Sequence
  Synthetic Oligonucleotide
aatgagaagcgcacgaaggc

SEQ ID NO: 96 - DNA, 20 bp, Artificial Sequence
  Synthetic Oligonucleotide
ccccagccgctgaccaatga

SEQ ID NO: 97 - DNA, 20 bp, Artificial Sequence
  Synthetic Oligonucleotide
ccatccgagtagccggcaca

SEQ ID NO: 98 - DNA, 20 bp, Artificial Sequence
  Synthetic Oligonucleotide
agtccttgctgccatccgag

SEQ ID NO: 99 - DNA, 20 bp, Artificial Sequence
  Synthetic Oligonucleotide
ccccttgcaggagtccttgc

SEQ ID NO: 100 - DNA, 20 bp, Artificial Sequence
  Synthetic Oligonucleotide
cccggtagtgggtggcatgt

SEQ ID NO: 101 - DNA, 20 bp, Artificial Sequence
  Synthetic Oligonucleotide
gatgcccgtcaggtaccacg

SEQ ID NO: 102 - DNA, 20 bp, Artificial Sequence
  Synthetic Oligonucleotide
ccagctgacgatgcccgtca

SEQ ID NO: 103 - DNA, 20 bp, Artificial Sequence
  Synthetic Oligonucleotide
gttgcgcagccctggcccca

SEQ ID NO: 104 - DNA, 20 bp, Artificial Sequence
  Synthetic Oligonucleotide
gtggcccacggttgcgcagc

SEQ ID NO: 105 - DNA, 20 bp, Artificial Sequence
  Synthetic Oligonucleotide
acaccccaaagtggcccacg

SEQ ID NO: 106 - DNA, 20 bp, Artificial Sequence
  Synthetic Oligonucleotide
cctggtgtacaccccaaagt

SEQ ID NO: 107 - DNA, 20 bp, Artificial Sequence
  Synthetic Oligonucleotide
tcgatgtactgggagaccct

SEQ ID NO: 108 - DNA, 20 bp, Artificial Sequence
  Synthetic Oligonucleotide
tgagcgcatgagcttttgca

SEQ ID NO: 109 - DNA, 20 bp, Artificial Sequence
  Synthetic Oligonucleotide
ccacaggccagggctgctgg

SEQ ID NO: 110 - DNA, 20 bp, Artificial Sequence
  Synthetic Oligonucleotide
tcgacgcagccttggctttc

SEQ ID NO: 111 - DNA, 20 bp, Artificial Sequence
  Synthetic Oligonucleotide
caggacagttcgacgcagcc

SEQ ID NO: 112 - DNA, 20 bp, Artificial Sequence
  Synthetic Oligonucleotide
gatttggtgccaggacagtt

SEQ ID NO: 113 - DNA, 20 bp, Artificial Sequence
  Synthetic Oligonucleotide
gaatatatgggatttggtgc

SEQ ID NO: 114 - DNA, 20 bp, Artificial Sequence
  Synthetic Oligonucleotide
ccaggacaaccttggcactc

SEQ ID NO: 115 - DNA, 20 bp, Artificial Sequence
  Synthetic Oligonucleotide
aggtaaggaggctcagctgg

SEQ ID NO: 116 - DNA, 20 bp, Artificial Sequence
  Synthetic Oligonucleotide
cttggctgaagggaggtaag

SEQ ID NO: 117 - DNA, 20 bp, Artificial Sequence
  Synthetic Oligonucleotide
gcagagcagcagcctgaggc

SEQ ID NO: 118 - DNA, 20 bp, Artificial Sequence
  Synthetic Oligonucleotide
caatgaaggcagagcagcag

SEQ ID NO: 119 - DNA, 20 bp, Artificial Sequence
  Synthetic Oligonucleotide
cttcagaggacagcacacat

SEQ ID NO: 120 - DNA, 20 bp, Artificial Sequence
  Synthetic Oligonucleotide
gaaccagaaaagtgagagct

SEQ ID NO: 121 - DNA, 20 bp, Artificial Sequence
  Synthetic Oligonucleotide
tgataatggataagaaccag

SEQ ID NO: 122 - DNA, 20 bp, Artificial Sequence
  Synthetic Oligonucleotide
ctgaagtgaagatgataatg

SEQ ID NO: 123 - DNA, 20 bp, Artificial Sequence
  Synthetic Oligonucleotide
atgcatggtgatgcttctga

SEQ ID NO: 124 - DNA, 20 bp, Artificial Sequence
  Synthetic Oligonucleotide
ggcattcgccaccatgcatg

SEQ ID NO: 125 - DNA, 20 bp, Artificial Sequence
  Synthetic Oligonucleotide
gaagggagaaatacatttgg

SEQ ID NO: 126 - DNA, 20 bp, Artificial Sequence
  Synthetic Oligonucleotide
cacccagcgaagggagaaat

SEQ ID NO: 127 - DNA, 20 bp, Artificial Sequence
  Synthetic Oligonucleotide
tgcagcccggcacccagcga

SEQ ID NO: 128 - DNA, 20 bp, Artificial Sequence
  Synthetic Oligonucleotide
tgtgaagctgggaagcaggt

SEQ ID NO: 129 - DNA, 20 bp, Artificial Sequence
  Synthetic Oligonucleotide
gagacgcagccgtttattgt

SEQ ID NO: 130 - DNA, 20 bp, Artificial Sequence
  Synthetic Oligonucleotide
cacaggtgtgcggaggagac

SEQ ID NO: 131 - DNA, 20 bp, Artificial Sequence
  Synthetic Oligonucleotide
gcaggcaccacaggtgtgcg

SEQ ID NO: 132 - DNA, 20 bp, Artificial Sequence
  Synthetic Oligonucleotide
ccagtgggtggcaggcacca

SEQ ID NO: 133 - DNA, 20 bp, Artificial Sequence
  Synthetic Oligonucleotide
aatcatgggcaacccagtgg

SEQ ID NO: 134 - DNA, 20 bp, Artificial Sequence
  Synthetic Oligonucleotide
tccaaaaatgaatcatgggc

SEQ ID NO: 135 - DNA, 20 bp, Artificial Sequence
  Synthetic Oligonucleotide
aaagagcatctcagaggatg

SEQ ID NO: 136 - DNA, 20 bp, Artificial Sequence
  Synthetic Oligonucleotide
ttgtgaaagaaaagagcatc

SEQ ID NO: 137 - DNA, 20 bp, Artificial Sequence
  Synthetic Oligonucleotide
cagtgatgttgaaaattgtg

SEQ ID NO: 138 - DNA, 20 bp, Artificial Sequence
  Synthetic Oligonucleotide
agcttccatgtgagggttca

SEQ ID NO: 139 - DNA, 20 bp, Artificial Sequence
  Synthetic Oligonucleotide
aacagcttttgtttttaaaa

SEQ ID NO: 140 - DNA, 20 bp, Artificial Sequence
  Synthetic Oligonucleotide
ggatcctgggagctacagcc

SEQ ID NO: 141 - DNA, 20 bp, Artificial Sequence
  Synthetic Oligonucleotide
acatccaattccacaggatc

SEQ ID NO: 142 - DNA, 20 bp, Artificial Sequence
  Synthetic Oligonucleotide
cagggagagaacatccaatt

SEQ ID NO: 143 - DNA, 20 bp, Artificial Sequence
  Synthetic Oligonucleotide
tgtgaaatatcattgacaag

SEQ ID NO: 144 - DNA, 20 bp, Artificial Sequence
  Synthetic Oligonucleotide
aacttgcatttagtgatgcg

SEQ ID NO: 145 - DNA, 20 bp, Artificial Sequence
  Synthetic Oligonucleotide
ggcctgggaacttgcattta

SEQ ID NO: 146 - DNA, 20 bp, Artificial Sequence
  Synthetic Oligonucleotide
gccgtgcttgccggagtctg

SEQ ID NO: 147 - DNA, 20 bp, Artificial Sequence
  Synthetic Oligonucleotide
gcagggatggcatcgagtcc

SEQ ID NO: 148 - DNA, 20 bp, Artificial Sequence
  Synthetic Oligonucleotide
gtgcccaggacggccctgca

SEQ ID NO: 149 - DNA, 20 bp, Artificial Sequence
  Synthetic Oligonucleotide
aagtagatgatgcctgagtg

SEQ ID NO: 150 - DNA, 20 bp, Artificial Sequence
  Synthetic Oligonucleotide
ttggaagcagcccacggctg

SEQ ID NO: 151 - DNA, 20 bp, Artificial Sequence
  Synthetic Oligonucleotide
agagcagggtgccttggaag

SEQ ID NO: 152 - DNA, 20 bp, Artificial Sequence
  Synthetic Oligonucleotide
cacactggtccccatcactg

SEQ ID NO: 153 - DNA, 20 bp, Artificial Sequence
  Synthetic Oligonucleotide
aggacctgccatggacactc

SEQ ID NO: 154 - DNA, 20 bp, Artificial Sequence
  Synthetic Oligonucleotide
acaacaggacctgccatgga

SEQ ID NO: 155 - DNA, 20 bp, Artificial Sequence
  Synthetic Oligonucleotide
tcaccaacaacaggacctgc

SEQ ID NO: 156 - DNA, 20 bp, Artificial Sequence
  Synthetic Oligonucleotide
gcccagcaccgcgatcaggt

SEQ ID NO: 157 - DNA, 20 bp, Artificial Sequence
  Synthetic Oligonucleotide
cgaagactgcagccaggcag

SEQ ID NO: 158 - DNA, 20 bp, Artificial Sequence
  Synthetic Oligonucleotide
tcctgggttacgaagactgc

SEQ ID NO: 159 - DNA, 20 bp, Artificial Sequence
  Synthetic Oligonucleotide
tcctgcagccaggcagccct

SEQ ID NO: 160 - DNA, 13001 bp, Mus musculus
gtagagtcctcgaggctaatacacagagcaaaacagacccagagaagacacagacccagagcaagacagggatccagagcaagatacagatccagagcaagacacagatccagagcaagacacaaatccagagcaagacacagatccagagcaagacagatccagaggaaataaaagtgatactgacctttctgattcagatcagctctctagttcttaaacttcaagttactggtggcattgccactggtccccaaccttgcagggtgtcagaacccacaggcatcatcaaactctgtgattaggactgctccaaagattctgcttccccatggagtgagggagcctgcatctgtgtaggtgtcatggatgatgtgagtgtgaaggtgtagcgtgcatgtatacaagggtgtgcataggctgtgtgtggagtgtgtgcataggtgtaagaatggtatgcaactgtgcaaagacagtgtgcatgtgtacaagtccataaggaagtgtgagcataatgtatgtgtgtgtccccaggtaattctgagcccactgtcacttacgggtctctcatgtgtgtcacatgtacaagggtgaggctgacatgtagcagcatgctgagggttccagtcctgttctgcagactgtgggggagcccttggggagctggctggggatgaggcttagtgcttcctatcccagagaaggggcaaatggtccctgagcacatacaaggaatatccacataagaaggactcccggatcccagaagaagcctgtttatctttcctgagaaggtaagtggggaggagtccaaaagacggacagacagtgaccccatgctatccctccatgtgtctgcactatgtatgtacagtgctaatggtacccatggtacaggtgtgaaaagaagaccaagcaaacaaggcccctgcaggtccaaatgggatccttgacgcctgagacgcaggagccagccaaggcacgtgccacagggaggtcgccatctgctgaagaactttagccagagcccaagcaccaggcgggctcagcatctgtcctctgaagcaactccaattgttctgctgctccagaaagctaaatacacagttcctggaactcaatagggaagagaatcctgagaccaggtccagcctcacataagagagctgggatgaatggctgccatctcctctcatgtccctggagttaccctgtcacgagagcattggattttactgggtgaaatttgacctctatcccagaagaagcaactggggtcccaagtgtgaagggaccccaggagtcaaaggtgcccctggggtacaccatgctggcaaagattaaagtccctgactagagagggagaggtactgtagtgtcccacaaatgaggtctgactcccaaaagagtggtccagccttgtactgccccctccctgaaaagaacattttcctcagagggtccaactgaagcccacagtgtagtctaggaggctgcatgaccataaaccgagtgtccttgggctagatccagtttttacacagaaagtttcacgtgctgagaaaccttgttgatctcaggcaacccagtgtccacaactccacctggagcctgggccagaggacacaaagggacaagtcatgggccacctgccgatagctgtttccacagtgtgggaagaggaggggcagggcagcccttgagagccattgacggctgcacagcccaggtctgccctacaggtcttttccaggtcataagcccataccctgtgctttaacaatgtcatcactgtgaatatggacatccatcggtgggtgagctctgttcacacctccggtctgagcccacattgcctgccttagctttcctctttccctggatgtacaagtgccagagacataagtggcagcctccaaccggcctccatgcttccagccaatgctccagctttctccacccctctcccctcccccctgacctttgccctcccggcagcatctggacagtcaccagccagaagccacagtctcatcatggttccacaggcgcatgggctgctgcttctctgctttctgctccagctccagggacctctagggactgcaggtatatatgtctgtctgtccggggagggtttctctgtgcatggagttgctgctctttgcagaggcagctgggacatcaacaggaaccccctcatgagcagcacacttgatgtgtgtccctgacaaaagcagatagcatctggatgcttgccacccggaaataggcagctcttcacaggggtgggatctttggctgtcgtcctccagagacgttttagaagtagggtgctttcctgccctctatggacacagacacttgcctgtgaaattagagcctgggggtcactcagtacagagctctgtggcaaggggtcagatgtgtcagaacagccacagggcaactgcactagagggctcgctcactgccttggctctacatccacatccacagcccctcccaccaagtcccccaccacatacatacatgtgtgcctctaaggccttgggggagaaagctcgtagttatgaatcacaagttgcagtcttacagaaagagatgctgccaggaacattcccatgcatagtgacatgggtggtcgagccagtactgtcccccttggaagcccatggcacatagggattgcagtggattctcctgtggaactgagtgacaacaggagcaatggctttcccaagttctctgcagtggtatggaagaccttggggtcaggtttgctttggatctccaggagattggctgaagtaaatttaccttctcctaagggcccaccccttacacaatcacttgctgtagaagtgtatgtgtcccagtggctggtggcatatggatccttctctgcctggtgaaggacattgattggtccagttggacagtgtctccatcctctctaggaacaccagagagtgttcctcggcccttcctcttccctctctctgtcccttcccaaaatggtttatctgacttccactcttgtccttccaagggaccctcctctggggtgtaccaacgtcctcctggtaagcagagccacctttcaaccccacctagacccagctcctcccagtaaggccctctgagcctcctctgcatagggaaagcctgtctctgttagccctcctcctgaatacattggatgctattgggtcttccataactgccttccttcaaggctcttcttcccatctcagttacacgtcttcccgtccacagatatcccaggcacacagtttatgtgacacagttgtcatatgaggactttgagggcctcaacaccatccttggtttccacccctgactacctttaggtgatttaaacatctgcaccccatctccacacccactcctgggattcagacccaaatctttgcttttccaaagccaagtgtggaacactgtcttctagtttctgtcgaggtcactgacattcaaggtcgtgtggtagaggctgcatctgaaacacttccaatgtagaaaatcacaggactcagaaccccaggtgaggcagctgtggccatggtacccagggctaatgctatgttatggtaagatctgaagccccagggctatgacttgagattcttcgattgactccctggacacaggatagaggacacagatgggagaagagagggagatttaaatatatgtttgtgcacacattttatgagtacccaagaaaggggttgcacaatgtgtgattaactgtctctttgtggcttggtgtcacatgtaaacctcgtgttcactggcctcacaggagaccacaccatttggcaatctcgttactaccgatgtgtcatttcgtgaccttttgtaagctctccaagcatcttctcattataaattcctaagagagatataggagaaatgccaaatcacatggatatggtaaactttcatctcatctaataaatatgtcaccatatacttctacataatactgtgccatgttctctgtcaccatcaatgaaagacacacccatttttcttaaagatatatttatttaatgtatatgagtacaccgtagctgtcttcagacacacctgaagagggcatcagatcccattacagatggttgtgaaccacaatgtggttgctgggaattgaactcaggacctctggaagagcaatcagtgctcttaaccgctgagccatctctccagcacaacacatccaattttaaattcagctgtgcctggtctgtgtcattccctgtgaccttcagcccacagaagatggtaagccattgacggaaacaacctatgcaccttccgttccttgaggtgaacaggaactctttggtccccaccaaagagtggaatggaaaaagattaatgtggatcagttgttcccatcactgcgggagatgggtgtttctgccactgagagatctggtgtgggtggttttcatgggctgacccagtgtggtttccttcactcactggaatgatccagtgtggctgtttccattcctgatgggaccaagagtgggtggcttctgagcagcttgagggaagctgccaggagcacatgagaccttctgtttctcagttttcataacccaggaggaagcacatggtgtcctacacaggcaaaggcgtgccaactcactcctggaggagctttggcccggctctctggagagagagtgcaatgaggaacagtgctcctttgaggaggcccgggagatcttcaagagccctgagaggaccgtgagtgccccatctcgtgcctgctcttgtgtaacacactgagactggaaggaagtgagctggggattgtgagggtcacacctatgtctctggatctgtctccccctcactgcggaggctgccactcagtactcatagatggacaggaaccctgacacacacaggtttctctctcaaccatgccacaagcagttttacaagaaatacaactcataaactacactggctgtgtctcctattttcccagaggggaatcccttattccttacgggctgcccatgaagtaaacctgagagggaaaattcacctcaagaaggcaggacacacacacacacacacacacacacacacacacacacacacgcaggcctcactggaaacgtgtgaggaacaccaaggcttcctctgccacacttggccatgtgcacagatttgcctccactcctgagttactcttttttatgtttgtttgtttgtttgtggtttgttgtttttttttttttttgagacagggttttgctatgtagcccaggctgacctccaattcttcatcttcattgcctcccaattattggaattataagtatgtgctaccccgcctggccctgtggtttcattttgtaaaagatacatatttatacatcactgagcatgttcctgtctggttagacatctcactctggagggttttcccagcaagggtgtgtcaggataagacaaggctttaagacggggtccactgtgtttcattttgtcatcatctctggaaaaaatagtcagtcacagcttcaggggcggttgtgaggggctggttgttcgttctccaagggcttttcttctcaccagccttggctcatatcacacctcacactcacaggaggctcccctcacactcataggaggctcccctcacactcacaggaggatcacctcacacttacaggaagctcacctcacactcataggaggatcaccttgcactcacaggaagctcacctcacactcacaggaagctcacctcacactcgcagaacacctctctctctctcaaaagagacctcacattcgcagaagtcctcacacaggaatttcacatgtactggagaatttacacccacagaacacctcacagaggaaccctcattcccacaggatagctcacaaacccaaagaatgttccacacaggaaattacaccgcacacttctctcacaagtgccctcacactcactcagagcaactcacacagggagcctggagaagctgctttcctcactgactggagcaaccacctgacagaaataactggaggaaagacaggtttattctggttggtcgttcaggagtcagggaaggcgtgacagtcacagcgcatccagtcaggaggtagagagtaagggatgctggtgttgagctcactatctcctctttattcagaccagaactcccatgggatgacacctcccacatttagaggtccagcccagaactggagaaagcaggatcagggtcagaactctggtgcgctcacttgggaaaggcaattgtgtggcgacttaaatatctctagaaagggcgtagagaaacatcaacctcagataatccctcctcgtctccagaggaacgctatcttcccaagccccacacagatgcccaggtcaggagccccagccatagctcctgagctccctccgtggacactaagtgctacttctcatgaccgtgcctcacatggcttcttcttccctctcagaagcagttctggattgtttacagtggtgagtaggtgaaagccatctgtgcccctcccccacaccccttctttgcagcacactaatgtcagctcacccacagatggggaccagtgtgcctcgaatccatgtcagaacggaggtacctgccaggatcatctcaagtcttacgtctgcttctgcctcctagactttgagggtcggaactgtgagaaaagtgagaccaacttcttgcctccgtttgcaatggatggagtcgggagggaagggcctgggtgactggttccagagaataacagcctcatcgagtaaggagagggtgtccaggccagcctcggcccctgaaggaaattgatctgggagacattaagtatttcttttaaacccatggcctttgatttatgagtctaagattctgggtctcagactcgctgtctgtcccttttgactccttgccctgagaattctgctgaactggagagatttaggggagggagggtccatctctgtccccaccaggtaagtcagctaggtaaacttcttctcctctgctggtttaaaggtataaagtatttattcatctttgaaatatcaattattgttatgcttggagaccaccaaactgctctgggctcaacggttcactactggactccatagagttcactgacagcagagatacacatgagtattttattacaccaacgtttgccatggccagaagcaggctttctgttgttctcttctgtgagctcacatagacagctcagttcttctagaatgcatgtaaagtgctgtgcagtatcaccaatagaaagtcacttaaccccgggcctaggtgtgagagacctgtctccagcccctccggtcccaaatcccaccatcaatcaaacctttggttagccatctggtatggcccaaggccccagacaagtggcagtattccagttgggggatgcccacgaggtcacctcttcagacaggacaaaagtgtcctttctgcagaccaaggactccttattgcccaagtgggttcttcctggtatcagctgcctccggttctcatcagaaagaggccagagagagtaaagggggaaggggagaagggctcactaacagtcactgatccatggaggcctgtaaggtttggtgatcagtgactgtgaatgacaggacagtcctctatactaaaatgaactgaccaccctcgcctgcaaatggccagtgctggagccaagggcctagatggctcacatggtcatgagggttgccccaagagtaacttctccctttgacatgtccgtgggccagttccccattctattgagaacatttcctttccttggaaatgtttgcttaccacctccagaacccttcacatcttctcattagagccttggaaggaagatgtcatcacacaccagcctagcaccctccaagatttctcagacttgtgactcccaaatccagctctccatcccgccagatcagtccatagggcaggtgacgcaagctcagccttaatagcccacagctacatgccatattccaaggggacaagagcaaccaccaccatggcccgtctgccgaccttgcagagacctctcaggttgtcttctccttttctcaggcaagaatgagcagctgatctgtgcaaatgaaaatggtgactgtgaccagtactgcagggaccatgtagggaccaagcgtacctgtagctgtcatgaggactacacgctacagccagatgaggtgtcctgcaaaccaaaaggtaatagactgtggggactggagctgtcttaggctctgaagaggcagtcacctgtgtgggcctgtggcaagttatcagctccatgatgtgtaacaaggtcagacctctgcttatcaagtacctggtagatgctgactgacttgaggtgaaagggtagagacttgaaggacacctgacctgagagctgttagtctggaaccctgggccagcatcctgcactgcctcaagtatcctgaccattgaagccggccaggagtggatgaggaggtcctacaccaactcttatgtcccacttagagtctctgagacataatcgggagtcacataggcacacctggcatctgtatccactgctgcagttaacaacataagatgacctattattattattattattattattattattattatatgcatgtgggcagtattacagatgatgacagtcctgaagggatcagagaatgtcagctcccctgggcactgacgttacaggcagttgtgagctgcttgttgtgggtgctgggatccactcttgccccctgctctgaaaacatatctccacaagaagactattcgagctctgcttccgaaggcggaggcttccccggaagaggatggggctcagtctgtctcatgaagctggaaagttggaagcccacagctcagtgttctttcttacccgaagccttcgcagtgggtggcgcccgagtctgccttaaggaccctgtcttcctgtgctggtggcccatgcttacacagatcgtcactttcttcatagcgaaccagaacgacacgttctccctgtgaggctctggcagaccccagctctagaaagcaggtcctcagctgtccccacaccgttgtctgcctcctctcctagccgctgcacaaggccctccctgcccactctagaagtaagccccaagttcctctgtcctgaggcctctcactgcccccagcaggagccatggcagcagccaactgcttctcatcccacagtcaccttcgcctctgcaccctgctctctctgacttctgtttcatacagttgagtacccgtgtgggagaatacctgttgtagaaaaaagaaactccagcagccgccaaggccgcattgtgggaggcaacgtgtgccccaaaggggagtgtccatggcaggtaatgctctccgtccagggaaaacctctctctccagctccggctcccctgacattagggtttctactctggggtatggaagttttgagttgggttacatttatacagaagagacatgggccagcctccatccccctatcccctcccccacactcttgtccagagaaagggaaagaaagcaggtattggggcccatcccatgaaaacccatgccaggcccacagttgccttgcacaagtcacttgaagagctgtagttgcacctcatcatccatgcaccccacccttctgcacccctgttgtctaccagagccagcagatctagggctgctctaccctgctgggggaatccccaaagggatggtgggcaggaattgtgtcaagtcagctgcttccttgggggcaaggagaccataaaagtagcatggctctctttgtctgtgtgcccccgcccaggctgtgctgaaaatcaatgggttattgctgtgtggggccgtcctgctggacgccagatggatagtgaccgcagcccactgcttcgataatatccgctactggggaaacatcacagtggtgatgggtaggtatggcccatccctggcctctggcctgagggcattttcagattaagattggctcagaggctgagagccaatggcctgacaatgtctctaaccaggtgatatcttcaagagtggcactggagatgacccacttgtcctctgggcccccctcctgtcagagaataggaaactggggggctactctgtccaccttgccctactcagattgggtgtcaaggaccccattcactcctgcaggctctgcccattcacaaccttccctccagaactgcagtggaccctgggccaggcttctcctgagaccctggcaaggagtcaaatcctcagacaccagaggctgaactgaccacagccctatccacatactctaggttagagaagcaagacaaacaagagctctgtgtggggacatggccacacatggtaggatacgtgccatgagacatggtaggatatgtgccatgaggcatggtaggatatgtgccatgaggcatggtaggatatgtgccatgaggcatggtaggatatgtgccatgaggcatggtaggatatgtgccatgaggcatggtaggatacgtgccatgaggcatggtaggatatgtgccatgagtggcgtggtaggatatgtgccatgagtggcgtggtgtcctcccaggtgaacatgacttcagtgagaaggatggggatgagcaagtacgacgggtgacacaggtcatcatgcccgacaagtacatccgcggcaagatcaaccacgacattgccctgctccgccttcaccggcctgtgaccttcactgactacgtggtgcccctgtgtctgcctgaaaagtccttctccgagaacaccctagccagaatccgcttctcaagggtcagtggctggggccagctactggaccgtggtgccacagccctggaactcatgtccatcgaggtgccccggctgatgacccaggactgtctggagcacgccaagcacagctctaacacccccaaaatcacagagaacatgttctgcgctggctacatggatggtaccaaggacgcctgcaagggtgacagcggtggcccacatgccacgcactaccatggcacatggtatctgacaggtgtggtcagctggggggagggctgtgcagctattggtcacattggggtgtacaccagggtctcccagtacatagactggctggtcagacacatggactccaagctccaggttggggttttccgactcccactactgtagctccttggatagcccaacccgtcccaagaaggaagctacggcctgtgaagctgttctatggactttcctgctattcttgtgtaagggaagagaatgagataaagagagagtgaagaaagcagagggggaggtaaatgagagaggctgggaaaggggaaacagaaagcagggccgggggaagagtctaagttagagactcacaaagaaactcaagaggggctgggcagtgcagtcacagtcaggcagctgaggggcagggtgtccctgagggaggcgaggctcaggccttgctcccgtctccccgtagctgcctcctgtctgcatgcattcggtctgcagtactacacagtaggtatgcacatgagcacgtaggacacgtgaatgtgccgcatgcatgtgcgtgcctgtgtgtccatcattggcactgttgctcacttgtgcttcctgtgagcaccctgtcttggtttcaattaaatgagaaacatggtctccacgtgtagggtcatgcttccgggttgcctaagggagtgtttctgaagtacagtgtgcacccctgccatggactgtttccccctggcagggaccggctgatttcagcacaccttggggatacctgctgttaccctgggttgtccttaagtccctgagtggagcctctgatgtcaagtcacatacacatcggcggggctgctcttcctgggaaaaggaaggggaaggtggactagagtcaaagagtaagctgagagggcttgatcaagtgcgtcatggccttagtttccccaatcactagtaggtaggcaggcatccatctcacagagccaatctgagttgccaccaagggcccttgggaggccttactgagaatctagctccctgaagactgacgtgctcagtatgggttgtgcttattctccagtcagcatgtcctttctgggggtggggccatagagaggggaggcgacaaagggaggaccccctcaagccagctttagtgtttgtccctagtcccagaccacaggtctgctgtgagtacctcacagactcagtgtaggctcagtgacacagtacacgggcatcatcaggcagggcagcgatcaggcaccagactcctgccaacagccaccagagcaccgcgcacatttcccaagttctgttatgtctgccacagcctaggaaatgacagtgacttccctcctggtgagatgccagcatcagcctcaacatctgatgtcacgggtgtgtgtgtttgtcaggtacccacagcacgccaaggccctctcagcactggtaacagtgataagccaagtgtcatcaccgtactctctggctacgtgcatgcagccacagtcatgagccattgcctaaccaaactccagtacagtgagcatgagaagcctattgctgctcactaattagcaggcatggtgcgcatgctccgcagactcaaaaatagctctgccagatgaagctttgaaggaggaagggctcagcatgtagaaggtggagtcagggtgagcactgcctcctcacaggccaaagttagggagttttgttgttgttgctgctgggactgctcctgcagttctctagggacaccggagcatgggcctgtagccccaattacatgtacacacacactctcagaagagtcaaatggccacagcatgtgagaggagctgtttccacatctatagctctcagatggct

SEQ ID NO: 161 - DNA, 20 bp, Artificial Sequence
  Synthetic Oligonucleotide
ccatagaacagcttcacagg

SEQ ID NO: 162 - DNA, 15001 bp, Macaca mulatta
  misc_feature (3049)...(4102): n = A,T,C or G
tcaaaacagagtggggctgcccagctcagggccagaatgatcctattcccagcacttctcagtcaggctctgtggctcaactaagaaaccggcctcccttgcagacaatggcctagctggcctggtctcctggaggctctcttcaaatatttacatccacacccaagatatgctctccagaattgactcgcattattgctatgggccaagttttcatctgcagtttaaatctgtttcccaacctacgttcctatgtcctaggggtttggaattctagatcgtatttgaagtgttggtgtcacacacacaccttaacacctgcacgctggcaacaaaaccatccgctttgcagcacaactggggccgcctgacctttctcctgtcctccctgcttgagctcagcagctgggcagcaggggatgcatggccactggccggccaggtgcagctctcagctggggtgttcagaggacgcctctgtccccccctcccccatccctctgtcgcccttggaggcagagaactttgcccgccagtcccatgcggaatgtcaacaggcagaggcagcgctgcagagatttcatcatggtctctcgagccctcgggctcctctgccttctgcttgggcttcagggctgtctggctgcaggtgcgtccggggagattttccccataaacttggtggaagggcagtgggcaaatccaggagccgacccgggcttcccaaaccgtccttgctctggacacccccattcaccaggagggttttctggtggctcctgttcaattgttttccttccagaaaccagcatccaggcacaggaggggaggcccttcttagtagcccaggctttggtgggattatttttcaaagaactttaggagtgggtggtgctttcttggcccccatgggcccctgcctgttaggttggacaagcacagggagtcgggggcctctcagagtatgggaggtgctcacaggctgctcccaggctggggaggacaagtgtgtgggggatggtgcctggggcatgggggatggggtgtggaggatgggggttggggatggcatgtggggtgtggaggatgggccatgagggggtgggtcctgggaaacggtatgtggggtatgagggatggggcgtggggtgcgggaggggggtgtgggaaagtgtgtggggtgtgggggatgggatgtgggaagtggcatgtggagtgcaaggaatggggcatggaggtgttgagcatggggtgtgtcgggtgtgtggggtgtgggggagggggaatggaaagggtgtgtggtgtgtgggggatggggtgaggggatggcgtgggaggtggggcatggggatggcaggtgtggcgtggggatggcgagtagggggtggggcgtggggatggtgactgtggggtggggatggcgagtggggctggggcctgggaatggtgagtggggtggggatggcgagtacagggtgtggcatggggatggcgaatggggcatgaggatggcgtgtggggatggcgagcaggggggtgggctgtgagggacagtgcctgagatgtggggctgcagccccagctcacacatggccttatgaccccagccaccttcctgccccaggcggggtcgctgaggcctcaggaggagaaaacacaggacctgctgtggaagccagggcctcacagaggtgagcagggactgccactggtttagtcccggggcccagtgggggccaacatcacctccttggcctcccatggcaaggagccagcccgcggggtggctactgcactgccccccaaggagggtgttccctgctcaagaggaagtgaccgctccagttcagccttccctgggactggggtgcaggtgaccttatcttctttgttaaatcctgttccttccagacaatcctgtgttattcatcaggtttgcctcagttcttgagagcttttctgatgcaaatctgctttcatcccagggcggtaggggctcagctcacgccagcctccaggggtgtgggtgtcctagaagtgtgggtgtcccgggggcgtgggtgtccctggagtgtgggtgtcctgggggcatgggtgtcccagagcgtgggtgtccctggagtgtgggtgtcccaggggcgtgggtgtcccggaggcatgggtgtcccggggcgtgggtgtcccggggcgtgggtgtcccaggggcgtgggtgtcccggaagtgtgggtgtcccggggcgtgggtggcttgggggcatgggtgtcccgggggcgtgggtggcttgggggcgtgggtgtcccgggggtgtgggtgtcccgggagcgggtgtcccgggagtgtgagtgtcctgggggtgtgggtgtcccgggagtgtgagtgtcccaggggcctggatgtcgggggactgcagggacacccttcccactcctgctgcccggggcacctcccctgaggactccgcctccaagagctcccacctcctggattctttggtgacccccgcctgcatcctcagcctccttccaaaccagaccggttctctagggacgtggacgtgtgaaactgattttaaaggaaacagacggtggcgtttctctgggccccacgtggcccagtagcgcccaccttccgtcccttcttccgcgctcagtaaccgatttaggccgctcctgcagaactcgggctcctgcccacctaccacctgcgtccacctgaggcctcgtcctcccagcaaaggtcgtccctcccgaacgcgcctcctgcggcctctccagagcccctcccgcgcgtcctctcggcctcctcccgggcctccctctcccgcctgccccacggcccggccagtctcccctcgcgggctgaggcgggttcaggcagcgcggccgccccgggggtcactcctcgtccaccaccgcgtggtgcccacagctcacagctcccgggagacggtcccctcagctgcagggcgtcctgaagaacggcctgctcagagctgagcgcacgggcttgcctcgccctgggcgcccttggccctcgccgaccccgttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccctaccaggcacacgctctccctacgcggccactccgcgcatgccggttttcacatcagaaaatacgatttgaaaagcacacttagggtgtcccccttaacttcctaagggaggccccccaatcccataaggatccggggcagtctgcgcatcacggatgcgcggctcacagaagggacgtggtgagaagctggcctgggggagctgctcgcggcccacaccatgccaccagcggcacctccgcagggcacatgtcggggagaaaaaacatgtggggacctggggctttctccacctcacactcacgggtcacctcacacaggacacctcacactcagggtgcacttcaaactcacaggtcattacacctcacacaggacgcctcacacaagacacctcacatggtgcacttcacactcacaggtcacctcacattcgacacctcacactgagcacacttcacactcgggacacctcacactcagggtgcacttcaaactcacaggtcattacacctcacacaggacgcctcacacaagacacctcacacagggcacacttcacactcacgggtcacctcacattcgacacctaacacagatcacctcactcagaggacacctcacactgggcacacttcacactcaggacacctcacactcagggtgcacttcagactcacaggtcatgacacctcacacagatcacctcactcttataggacacttcacactcacagatcacctcactctcacaggacacttcggacaggacacacttcacacaggccacctcaggttatgtcaaactcacaggtcccctcacacagtcacctcacacagtgtacacttcacactcacaggtcccctcacacaggtcacctcacacagtcacctcacacagtgtacacttcacactcacaggtcccctcacacaggacacctcacacagtcacctcacacagtgcacacttcacacaggtcccctcacacagtcacctcacacacagggcacacttcacactcacaggtcccctcacacaggtcacctcacacaagatgcacttcacactcatgggtcccatcacacacaggacacctcacactcgtcacctcacacatgttacatcaaactctcaggtcccctcacacagtcacctcacacacagggcacacttcacattcgcaggtcacctcacaccaggcccacgactctcacaggtcaccacacctcacacacatcagctcacatagatcatctcaccctcacaggacatccatcacactcacaggtcacgtcacactcatacctcacccacaggtcacctcacacaaatcaccttgctcacacagatcacctcacacgcagggcacacttcacactcacaggttccctcacacaggacacctcacactcacagatcacctcaacaccgatcacctcacacaggggacacttcattctcacaggtcacctcacataggacatctcacacacaggtcacctcactcacagatcacttcacacagggcacacttcactcacagatcacacctcacctcccgctcacagatcacctcactctcacagggcacctcactctcacaggacacctcatacagggcacacttcactcccacaggtcaccatacctcacacagatcagatcacttcattctcacaggatacctcacactcagggcacacttcacactcacaggtcacctcacacaaggcacccttcacacaggtcaccacacctcacacagatcatctcactctcacaggacccctcacactcagattatctcacactcaggtcaccacacctcacactcttaggatgtctcacgcaggatgcctcacagtcacagagaaccacatctcatatgcacaagacacttcacattcacaggacacctcatgctcacaggaagcctcacactcacaggaagtccagctgtctgagacaaaggctaacatgaccctttccgggcaaattgaggatggtcatgcctagcatttttatccacctagttttcaaagcatttctcatctgttgtattctcacagcaccctgtgagtttaagtttaggtggccaacagtttcttcagcaatcacttttttctgtggagtgcttttgctgtttgtggaagattttgcatctgctactgcaccctctcccggtgtcagccggtgtgtgtggccaccctgtcagagatggagctgtggctcaaagcctgtgtacctcatcccaggtccacagctcagcgacagaagagtcagggctgaacctcgggtgttctgacctgggagcaggaaatgtgtggtcacccatagtttcagaagtcctggggaggggccaagattggaagaaatctacctcagctctgcaggaaagtctggcttcctgagcccaccccgccaggcccaggtccaagttccccaaccccagctcgtggtttgtccagtgctcaccgttgggtgcactggtgaaggtgctcacgaggctttctcttttgttccctcagaagctgttctggatttcttacagtggtgagtagatgatcgccaccaatcctgcctgcaacccttctcctcagcgtactgacgccagcccattccacagatggggaccagtgtgcctcaaatccgtgccagaatgggggctcctgcaaggaccagctccagtcctatatctgcttctgcctcccttccttcgagggccggaactgtgagaagagtgaggccccactttgggtcccatatttgcagagggcctggccaaccgggttgcagggtgcacaacctggtggggtgtgtggaccgggcattctgagctcagccccagttggaatttggtctaggtgaccttgaagtcccttctagtctgaggtctttgacagggacccaaggttctaattctcagactcagtggccccttgggctcccggccctgggcaattctcagccctcgagatggcccagctgagagtccctgtgtccctgtcccacttccacgtcccaccaggcagcaccgcttggtaaacttccccttctctactttccattacaaaggtttgaggtgttttttgttttgtttgtttgtttttggttttgttttgttttgtttaccatctgaatattaaattattgcaaagtttgaggcccccaacttcccttaggttcagtaattcactagaaggactcatagaacccactgaagtggatacactcacagttaccatttattacagcaaaggaagctgacttaagtctgcagaggaaccgggcacaaacttcccattgtcccctccctgtggggtcatgtggacacttctcccagaaagacgtgtgatgagacgtgggaagtactgccaacttgggaagctctatgagccccggtgtccagaggttttatcagggctcaatcacacagacccagctgaccacccacacggctgacctcagtctcagcccctccagaggccaagccaatagtgtggcccgaggccctgccatcatcacattgtcagctagaccatccagcatggcccaaggtccgggtaaacaccaacattccctcagggcttagcgatcacttcccaggaaatgtgtggtcacccttccaagggcttagcgatcacttcccaggaaatgtgtggtcacccttccaagggcttagcgatcacttcccaggaaatgtgtggtcacccttccaagggcttagtgatcacttcccaggagccaaggcaaaggctaccctttccctgggaacagcagctcatccttgaccacccaaggtggttcattctcacactgaacgagctctccggcacagcagccactttcttctctaagtagaagagagcccagcaaggtggggcaggctgaagagagaggcttcctgctggtcatctgggtccagaatgcctggggatctctgctcagccctggtgcccagcagccctggtgtgcatcctgcagggcaggccttcccgccggagtcctggacttactcagggccactgcccttgcccacatcaatcaaagtcgggctgccggttctgctgcttctgtctgagcccatggccagtgctgggactgactgtccctaggcgggctcgcggtggcatgaggccagcttggagaactgtctcagcgctctggtcctctcgtcagttgagtctctgattggaagtcccttggatacttttaccatccctacgggactttcactttcccccaggctcccctcagcttcccatcagctgctcggaagagtggtcaccctggaggccactgcccaccagccaggcacccccccaaatgcaactgcagccagcgctgcccccgactggcaaggctgttcagacgtgactcctctgatccaggccttgtcctttggatcagtccacggagcaggcggtgccaagctcaggctctgtcgcccacagctcagtgccccttccaggcagaacgccgctgctgacttagggcatggcatcccccggggctggctctcactgacccaaagaggcccctctcagggtatccccttcctgtccgcagacaaggatgaccagctgatctgcgtgaacgagaacggcggctgtgagcagtactgcagtgaccacgcgggtgccaagcgctcctgttggtgccacgaggggtactcgctgctggcagacggggtgtcctgcatgcccacaggtgaccaggcttcatgtcccagtcccagatgacaccagtccctgtcccactacggattctcttactggacaaaagacgggtgggggtggcttcacatctgagcaccaaccatgcgctgaccaaccgtgaggcaggatctgggcaccaagggtggcacaggccagagcgacagtgactaggatgggcaccctgggggcagtccctgaatggcctcaggccccctacccatgctaggcagaccaggggagccaagcaaggctctatctcacgtccaactcccactcgcaggacctccgctggggttcgtgaatctaccttggcacaggcagtgtctgtactgactgctgcccgctctgaattccaaaacttgtaaaggctcctgggaaaatgggatgtttctccaaaccagcctggaacaaatgggctgcacttccaaaggcagggacaccccacgcccactgtgtctcgaagaggtggacgtgcccaccctggccacacagcctgggactcagcccaccacctcctcaggttttctttctggcccacgaccttgattggagcagatcaaaactaagcgtgggatcaaaacaacagagttgtttgtgacgttgattcatctttaggtagaatttcattcaccttttactaaagtcaagcaacacattttccccctgaaaagtgagcagagggcaatattaagacgtaagccctccatctcctccaaaaccagccctgaccattgtctcctcagccagccacttccgcaagggcctctcatggcccagccccaccagtcaggcccagccccaccagtcaggcccagccgaggccctgctttccaccatccctgggccctggcagctcctgctcctgggggcctcccatagcctcggcctcaaggcctctcagaggatgggtgtttctgaatctttcctagtggctcgttcatccttcacaaatttctgcatctttctgacttttgttttacacagttgaatatccatgtggaaaaatacctattctggaaaaaagaaatgccagcaaaccccaaggccgaattgtcgggggcagggtgtgccccaaaggggagtgtccatggcaggtaaggcttcccttggcttcaggattctaagccctgagggtcttggagccttttgaatgtgagctgaacaacagttctggaagggaaaatgggcaggtcagccccaaggccaccaggctccaagtcagcccacctagaacctctagctcgctgcacccccatgctttcagtggggcaaggaaggagaaaagaaggcgacactcgctgagggtctaccctgtgcagagaaccctgcgagatgcccctcccgagttgtcacgtcgtcctcactgttactctttgaggtgggatctttgcctgatctttgcaaaatcaggagcattggatcaaagctatgtgaagatcccgtgaggtgaacagtgaaatctcacagcgacgtttgtattgttgggctgtgcccaagagcacgtctcggctagagaggggcgcagcctcccagagccaggtctgagcagctttgcctgggagggatctgcaaagaccccaggatttcagaaacaaattgtgcaatgccagaggtcccttggcgtgcccgggagggcgagtcatcagagaaacaatgacagtaatgtgacttccatgcctcctgtccccccgcccaggtcctgttgttggtgaatggagctcagctgtgtggagggaccctgataaacaccatctgggtggtctctgcggcccactgtttcgacaaaatcaagagctggaggaacttgaccgcggtgctgggtaggtgccgctctcccctgtgtgaccgcggtgctgggtaggtgccgctctcccctgtgtgaccgcggtgctgggtaggtaccactctcctctgaccgcgttgctgggtgggtactgctctcccatctgactgctgtgctggttacgcgccgttctcccgtctgacgatggtgctgagtaggcgccactctcccctgtctgaccacggctctcaagtgtctcaggggccgcagctctgggcttcgtgctgtcacttccacagacagacagacatctccaaaaggggagcaactgtgctaggcatgactgctgtggccaccgtcctctcagccactttcccatgcccaaataaaatggtaaaagacaggggttctgcccatcctgcctcacctggccaagagcccataggaggatgcaacttccagggcttcatgggaccactgggtggcagggactgtgctcactgggtttacaggtgagatgaacattcccaggagggcacttggctgggaagaactgtggagaatcagggcacaccctgcccccccagctgccaggtcgcagcaccccttccccacctcaacgcccaggccccagattcaccccagttcacacgtccccatgtgagccacaggctgccacctgcggcaggctggccaggtcaccttggggttggatgcaggcccccctcaccccaaaaggagactgcagcccctgcagacctagaaatggccacagcccgtccccatgcaccagggggccaggcagcaggtagtgggatgggcctgagcaaggctccctccttccaggcgagcacgacctcagcgagcacgaaggggatgagcagagccggcgggtggcgcaggtcatcatccccagcacgtatgtcctgggcgccaccaaccacgacatcgcgctgctccgcctgcagcagcccgtggtcctcactgaccatgtggtgcccctctgcctgcccgaacggacgttctccgagaggacgctggccttcgtgcgcttctcgttggtcagcggctggggtcagctgctggaccgtggtgccacagccctggagctcatggccctcaacgtgccccggctgatgacccaggactgcctgcagcagtcacagaaggcagaagcctccccgaatatcacggagtacatgttctgtgccggctactcggacggcagcagggactcctgcaagggggacagtggaggcccacacgccacccgctaccggggcacgtggtacctgacaggcatcgtcagctggggccagggctgcgcggccgtgggccacttcggggtgtacaccagggtctcccagtacatcgagtggctgcaaaagctcatgcactcagagccacgcccaggcgtcctcctgcgagccccatttccctagcctagcagccctgccccctggagagaaagccaaggctgtgtagaactgttctggcacaaaatcccatcgattcttctgcagttcatggggtagaggagggcatgggagggagggagaggtggggagggagacagagacagaaacagagagacaaagagacagggagagactgagggagaggttctgaggacatggagagactcaaagagactccaagattcaaagagcctaatagagacacagagaaggaatcgaaaagatgagatgcagaggcagacaggcgctggacagaggggcaggggaatgctgcggttgtcctggaggcagacagcccagctgagcctccttatctctcttcagccaagcccacctgcccgtgatctgctggcctcaggctgctgttctgccttcattgctggagacactagaggcatgtacacacgtggatgcatacacacacaccaatgcacacacacagagatatgcacacacacggatgcacacacagagggtcacacagagatatgcaaacacactgacacacacatacagagatatgcacatacacagatgcatatacacagatatgcgcacacacggatgcgtgcacaccacaccaatgcacacacacactaatgcacccacacggatgcagagagatatgcacacaccgatgtgcacatacacagatatgcacacacatggatgagtgcacacacaccaatgtacacacacagatatgcacacacggatgcacacacaccgatgctgactccatgtgtgctgtcctccaaaggcggttgtttagctctcacttttctcgttcttatccattatcatcttcatttcagacaattcagaagcatcaccatgcatgttggcaaatgccccaaactctcccccaaatgtgccgggctgcacaggccgttccccaccggcttcccaacttcacaataaatggctgcatctcctccgcacgcctgtggggcctgccacccaccgtgtagcctgtgattcattttcagagcctccagtgctcatcctctgagatgcttttttctttcacagttttcagcatcactgaaatgaaccctcacatggcagctgttctttttaaaaacaaaagctctttgatagatgtttgaggctgtagctcccaggaccctgtggaattggttgttctctccctgccacagcccttgtcaatgatatttcgcagagaccctgggagcacctgcttgagaatcagggacataccactaaatgcaggttcccaggccctggctgcagtgggaggacctggcaagctgcactcttgctgagtccccagggtggtgggggaagaatgagaaacacatgagcagagaaatggggaggtgacagacactgcccgcactcagactccagcaagcatggctcagagagcggactcaacgccatccctgtagggccgtcctgggcaccagtggcgctcacagcagcaaggcaggcaccagcaacccacctcgggggcactcaggcaacatctactttagagcagacagggtccgtgaactacagctgagggctgcttctaagccacccggctcttgtaaataaagttttatgggaacacacccacgttagtgtccatggagtggccgtgacagagatgtctagccagacagaccagctgacctgccaagcccagcatgattagtgtcaggacctctgccgaagatgctggctgaccctggccagaccccagttcctaatgcccccacacagggacgggggccagtggcgggccctgatcaggtcagagctggctctgctttctcttttgtccgagtgactggggagtcatgcggggtcctggtggggtgccagcctcccttcttgccaatgggaaatgcaggcacccacctcacggtgctgctgaaggagggggcccgggactctccagaaactttgctgaagggcctgggcaccctcgaaggctacatttcttatgggacgtgccacctgccatggctcagctccagctttctgtgagtggcgagatagaatacagggaggccactggccatgggcctgcgacagggtggggcgaggcagcaggctcgggcctccaaagccagcatcaccacccagcgttgatgaaaaagactgcatgtctgccatgagcatcctgctgctggtgcacacaccacattggtctctccatacaaacgtgcctagaggcgatgtcggagtgtggagaccacgagaggcaggagtcagacatctggtgccaccaggaaggcccctctcagaggactgggctgtgcgtggtgcccaccgtgggaggctaccctggcgttggcacccagtgccatcagtttgtgtagtcgggtggggcccagtgagcacctcctgtgtgccaggcacaatgacgcacaatgtgtgcacaccaggcccaggtgcaggtggctgcgagacgggcaacacatcaaggcagacacaccgtgaggcagtggccagcactgtgggttttaggggcgttgctccggccactacggcatagcaggtagtgattgccacactggccaagttttagaccatttattccagggaccccagaagcaaaaatcctggctgcacctcccggtgactcccacagccattgagtggagacgctcagggacctggtgacaggaggtttctgtgctggacaatgagcccagaagccctctcagccttggaacagtgtggccagtggtgggcaggtcaggaggggtttcagacagagcctgtccctccagatggtcaggggagggctactccccacagaagtacatgttgggaccatgtgggcacagaaggtttgggggtgggtggggcaggtaccagcctggcctgtgggagaccgtggtgcagatgccaagcccccccgtgacatcagaccacctgacaccacccagagaatggc

SEQ ID NO: 163 - DNA, 3068 bp, Macaca mulatta
  misc_feature (182)...(342): n = A,T,C or G
agtcccatgcggaatgtcaacaggcagaggcagcgctgcagagatttcatcatggtctctcgagccctcgggctcctctgccttctgcttgggcttcagggctgtctggctgcaggcggggtcgctgaggcctcaggaggagaaacacaggacctgctgtggaagccagggcctcacagagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagctgttctggatttcttacagtgatggggaccagtgtgcctcaaatccgtgccagaatgggggctcctgcaaggaccagctccagtcctatatctgcttctgcctcccttccttcgagggccggaactgtgagaagaacaaggatgaccagctgatctgcgtgaacgagaacggcggctgtgagcagtactgcagtgaccacgcgggtgccaagcgctcctgttggtgccacgaggggtactcgctgctggcagacggggtgtcctgcatgcccacagttgaatatccatgtggaaaaatacctattctggaaaaaagaaatgccagcaaaccccaaggccgaattgtcgggggcagggtgtgccccaaaggggagtgtccatggcaggtcctgttgttggtgaatggagctcagctgtgtggagggaccctgataaacaccatctgggtggtctctgcggcccactgtttcgacaaaatcaagagctggaggaacttgaccgcggtgctgggcgagcacgacctcagcgagcacgaaggggatgagcagagccggcgggtggcgcaggtcatcatccccagcacgtatgtcctgggcgccaccaaccacgacatcgcgctgctccgcctgcagcagcccgtggtcctcactgaccatgtggtgcccctctgcctgcccgaacggacgttctccgagaggacgctggccttcgtgcgcttctcgttggtcagcggctggggtcagctgctggaccgtggtgccacagccctggagctcatggccctcaacgtgccccggctgatgacccaggactgcctgcagcagtcacagaaggcagaagcctccccgaatatcacggagtacatgttctgtgccggctactcggacggcagcagggactcctgcaagggggacagtggaggcccacacgccacccgctaccggggcacgtggtacctgacaggcatcgtcagctggggccagggctgcgcggccgtgggccacttcggggtgtacaccagggtctcccagtacatcgagtggctgcaaaagctcatgcactcagagccacgcccaggcgtcctcctgcgagccccatttccctagcctagcagccctgccccctggagagaaagccaaggctgtgtagaactgttctggcacaaaatcccatcgattcttctgcagttcatggggtagaggagggcatgggagggagggagaggtggggagggagacagagacagaaacagagagacaaagagacagggagagactgagggagaggttctgaggacatggagagactcaaagagactccaagattcaaagagcctaatagagacacagagaaggaatcgaaaagatgagatgcagaggcagacaggcgctggacagaggggcaggggaatgctgcggttgtcctggaggcagacagcccagctgagcctccttatctctcttcagccaagcccacctgcccgtgatctgctggcctcaggctgctgttctgccttcattgctggagacactagaggcatgtacacacgtggatgcatacacacacaccaatgcacacacacagagatatgcacacacacggatgcacacacagagggtcacacagagatatgcaaacacactgacacacacatacagagatatgcacatacacagatgcatatacacagatatgcgcacacacggatgcgtgcacaccacaccaatgcacacacacactaatgcacccacacggatgcagagagatatgcacacaccgatgtgcacatacacagatatgcacacacatggatgagtgcacacacaccaatgtacacacacagatatgcacacacggatgcacacacaccgatgctgactccatgtgtgctgtcctccaaaggcggttgtttagctctcacttttctcgttcttatccattatcatcttcatttcagacaattcagaagcatcaccatgcatgttggcaaatgccccaaactctcccccaaatgtgccgggctgcacaggccgttccccaccggcttcccaacttcacaataaatggctgcatctcctccgcacgcctgtggggcctgccacccaccgtgtagcctgtgattcattttcagagcctccagtgctcatcctctgagatgcttttttctttcacagttttcagcatcactgaaatgaaccctcacatggcagctgttctttttaaaaacaaaagctctttgatagatgtttgaggctgtagctcccaggaccctgtggaattggttgttctctccctgccacagcccttgtcaatgatatttcgcagagaccctgggagcacctgcttgagaatcagggacataccactaaatgcaggttcccaggccctggctgcagtgggaggacctggcaagctgcactcttgctgagtccccagggtggtgggggaagaatgagaaacacatgagcagagaaatggggaggtgacagacactgcccgcactcagactccagcaagcatggctcagagagcggactcaacgccatccctgtagggccgtcctgggcaccagtggcgctcacagcagcaaggcaggcaccagcaacccacctcgggggcactcaggcaacatctactttagagcagacagggtccgtgaactacagctgagggctgcttctaagccacccggctcttgtaaataaagttttatgggaacaca

SEQ ID NO: 164 - DNA, 20 bp, Artificial Sequence
  Primer
gggaccctgatcaacaccat

SEQ ID NO: 165 - DNA, 24 bp, Artificial Sequence
  Primer
ccagttcttgattttgtcgaaaca

SEQ ID NO: 166 - DNA, 18 bp, Artificial Sequence
  Probe
tgggtggtctccgcggcc

SEQ ID NO: 167 - DNA, 3141 bp, H. Sapiens
agtcccatggggaatgtcaacaggcaggggcagcactgcagagatttcatcatggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacaaaaaaaaaaaaaaaaa

SEQ ID NO: 168 - DNA, 20 bp, Artificial Sequence
  Synthetic Oligonucleotide
ggatcattctggccctgagc

SEQ ID NO: 169 - DNA, 20 bp, Artificial Sequence
  Synthetic Oligonucleotide
tcttgggtgtggatgtaaat

SEQ ID NO: 170 - DNA, 20 bp, Artificial Sequence
  Synthetic Oligonucleotide
cagatttaaactgcagatga

SEQ ID NO: 171 - DNA, 20 bp, Artificial Sequence
  Synthetic Oligonucleotide
tctagaattccaaaccccta

SEQ ID NO: 172 - DNA, 20 bp, Artificial Sequence
  Synthetic Oligonucleotide
caacacttcaaatacgatct

SEQ ID NO: 173 - DNA, 20 bp, Artificial Sequence
  Synthetic Oligonucleotide
cgtgcaggtgttaaggtgtg

SEQ ID NO: 174 - DNA, 20 bp, Artificial Sequence
  Synthetic Oligonucleotide
ggccagtggccatgcatccc

SEQ ID NO: 175 - DNA, 20 bp, Artificial Sequence
  Synthetic Oligonucleotide
gagagctgcacctggccggc

SEQ ID NO: 176 - DNA, 20 bp, Artificial Sequence
  Synthetic Oligonucleotide
ctgaacaccccagctgagag

SEQ ID NO: 177 - DNA, 20 bp, Artificial Sequence
  Synthetic Oligonucleotide
gagaccatgatgaaatctct

SEQ ID NO: 178 - DNA, 20 bp, Artificial Sequence
  Synthetic Oligonucleotide
aagcagaaggcagaggagcc

SEQ ID NO: 179 - DNA, 20 bp, Artificial Sequence
  Synthetic Oligonucleotide
agcccaagcagaaggcagag

SEQ ID NO: 180 - DNA, 20 bp, Artificial Sequence
  Synthetic Oligonucleotide
ccctgaagcccaagcagaag

SEQ ID NO: 181 - DNA, 20 bp, Artificial Sequence
  Synthetic Oligonucleotide
ctgcccttccaccaagttta

SEQ ID NO: 182 - DNA, 20 bp, Artificial Sequence
  Synthetic Oligonucleotide
gttctttgaaaaataatccc

SEQ ID NO: 183 - DNA, 20 bp, Artificial Sequence
  Synthetic Oligonucleotide
gtgtttctcctcctgaggcc

SEQ ID NO: 184 - DNA, 20 bp, Artificial Sequence
  Synthetic Oligonucleotide
tagccaccccgcgggctggc

SEQ ID NO: 185 - DNA, 20 bp, Artificial Sequence
  Synthetic Oligonucleotide
tcagaaaagctctcaagaac

SEQ ID NO: 186 - DNA, 20 bp, Artificial Sequence
  Synthetic Oligonucleotide
gcagatttgcatcagaaaag

SEQ ID NO: 187 - DNA, 20 bp, Artificial Sequence
  Synthetic Oligonucleotide
ctttaaaatcagtttcacac

SEQ ID NO: 188 - DNA, 20 bp, Artificial Sequence
  Synthetic Oligonucleotide
ggttactgagcgcggaagaa

SEQ ID NO: 189 - DNA, 20 bp, Artificial Sequence
  Synthetic Oligonucleotide
cgagttctgcaggagcggcc

SEQ ID NO: 190 - DNA, 20 bp, Artificial Sequence
  Synthetic Oligonucleotide
aggagcccgagttctgcagg

SEQ ID NO: 191 - DNA, 20 bp, Artificial Sequence
  Synthetic Oligonucleotide
gacgaggcctcaggtggacg

SEQ ID NO: 192 - DNA, 20 bp, Artificial Sequence
  Synthetic Oligonucleotide
ttgctgggaggacgaggcct

SEQ ID NO: 193 - DNA, 20 bp, Artificial Sequence
  Synthetic Oligonucleotide
gacgacctttgctgggagga

SEQ ID NO: 194 - DNA, 20 bp, Artificial Sequence
  Synthetic Oligonucleotide
acgccgtgggcttcctcctg

SEQ ID NO: 195 - DNA, 20 bp, Artificial Sequence
  Synthetic Oligonucleotide
gcagctcctccaggaacgcg

SEQ ID NO: 196 - DNA, 20 bp, Artificial Sequence
  Synthetic Oligonucleotide
agggagcccggccgcagctc

SEQ ID NO: 197 - DNA, 20 bp, Artificial Sequence
  Synthetic Oligonucleotide
agcactgctcctccttgcac

SEQ ID NO: 198 - DNA, 20 bp, Artificial Sequence
  Synthetic Oligonucleotide
ctgatgtgaaaaccggcatg

SEQ ID NO: 199 - DNA, 20 bp, Artificial Sequence
  Synthetic Oligonucleotide
gtattttctgatgtgaaaac

SEQ ID NO: 200 - DNA, 20 bp, Artificial Sequence
  Synthetic Oligonucleotide
taggcatgaccatcctcaat

SEQ ID NO: 201 - DNA, 20 bp, Artificial Sequence
  Synthetic Oligonucleotide
gtgagaatacaacagatgag

SEQ ID NO: 202 - DNA, 20 bp, Artificial Sequence
  Synthetic Oligonucleotide
gggtgcagtagcagatgcaa

SEQ ID NO: 203 - DNA, 20 bp, Artificial Sequence
  Synthetic Oligonucleotide
gggtgaccacacatttcctg

SEQ ID NO: 204 - DNA, 20 bp, Artificial Sequence
  Synthetic Oligonucleotide
ctgtaagaaatccagaacag

SEQ ID NO: 205 - DNA, 20 bp, Artificial Sequence
  Synthetic Oligonucleotide
gagaagggttgcaggcagga

SEQ ID NO: 206 - DNA, 20 bp, Artificial Sequence
  Synthetic Oligonucleotide
caggagcccccattctggca

SEQ ID NO: 207 - DNA, 20 bp, Artificial Sequence
  Synthetic Oligonucleotide
gtccttgcaggagcccccat

SEQ ID NO: 208 - DNA, 20 bp, Artificial Sequence
  Synthetic Oligonucleotide
ggactggagctggtccttgc

SEQ ID NO: 209 - DNA, 20 bp, Artificial Sequence
  Synthetic Oligonucleotide
agatataggactggagctgg

SEQ ID NO: 210 - DNA, 20 bp, Artificial Sequence
  Synthetic Oligonucleotide
agcagatataggactggagc

SEQ ID NO: 211 - DNA, 20 bp, Artificial Sequence
  Synthetic Oligonucleotide
gaggcagaagcagatatagg

SEQ ID NO: 212 - DNA, 20 bp, Artificial Sequence
  Synthetic Oligonucleotide
acagttccggccctcgaagg

SEQ ID NO: 213 - DNA, 20 bp, Artificial Sequence
  Synthetic Oligonucleotide
aaatatgggacccaaagtgg

SEQ ID NO: 214 - DNA, 20 bp, Artificial Sequence
  Synthetic Oligonucleotide
ccctctgcaaatatgggacc

SEQ ID NO: 215 - DNA, 20 bp, Artificial Sequence
  Synthetic Oligonucleotide
caccccaccaggttgtgcac

SEQ ID NO: 216 - DNA, 20 bp, Artificial Sequence
  Synthetic Oligonucleotide
ggctgagaattgcccagggc

SEQ ID NO: 217 - DNA, 20 bp, Artificial Sequence
  Synthetic Oligonucleotide
tctcgagggctgagaattgc

SEQ ID NO: 218 - DNA, 20 bp, Artificial Sequence
  Synthetic Oligonucleotide
taatttaatattcagatggt

SEQ ID NO: 219 - DNA, 20 bp, Artificial Sequence
  Synthetic Oligonucleotide
tatgagtccttctagtgaat

SEQ ID NO: 220 - DNA, 20 bp, Artificial Sequence
  Synthetic Oligonucleotide
tgtccacatgaccccacagg

SEQ ID NO: 221 - DNA, 20 bp, Artificial Sequence
  Synthetic Oligonucleotide
gagcttcccaagttggcagt

SEQ ID NO: 222 - DNA, 20 bp, Artificial Sequence
  Synthetic Oligonucleotide
tgataaaacctctggacacc

SEQ ID NO: 223 - DNA, 20 bp, Artificial Sequence
  Synthetic Oligonucleotide
gggctgagactgaggtcagc

SEQ ID NO: 224 - DNA, 20 bp, Artificial Sequence
  Synthetic Oligonucleotide
agggtagcctttgccttggc

SEQ ID NO: 225 - DNA, 20 bp, Artificial Sequence
  Synthetic Oligonucleotide
agatgaccagcaggaagcct

SEQ ID NO: 226 - DNA, 20 bp, Artificial Sequence
  Synthetic Oligonucleotide
ggacccagatgaccagcagg

SEQ ID NO: 227 - DNA, 20 bp, Artificial Sequence
  Synthetic Oligonucleotide
gcattctggacccagatgac

SEQ ID NO: 228 - DNA, 20 bp, Artificial Sequence
  Synthetic Oligonucleotide
atgcacaccagggctgctgg

SEQ ID NO: 229 - DNA, 20 bp, Artificial Sequence
  Synthetic Oligonucleotide
gcaggatgcacaccagggct

SEQ ID NO: 230 - DNA, 20 bp, Artificial Sequence
  Synthetic Oligonucleotide
cgggaaggcctgccctgcag

SEQ ID NO: 231 - DNA, 20 bp, Artificial Sequence
  Synthetic Oligonucleotide
tgaccactcttccgagcagc

SEQ ID NO: 232 - DNA, 20 bp, Artificial Sequence
  Synthetic Oligonucleotide
cgtggactgatccaaaggac

SEQ ID NO: 233 - DNA, 20 bp, Artificial Sequence
  Synthetic Oligonucleotide
gacagagcctgagcttggca

SEQ ID NO: 234 - DNA, 20 bp, Artificial Sequence
  Synthetic Oligonucleotide
tactgctcacagccgccgtt

SEQ ID NO: 235 - DNA, 20 bp, Artificial Sequence
  Synthetic Oligonucleotide
ggtcactgcagtactgctca

SEQ ID NO: 236 - DNA, 20 bp, Artificial Sequence
  Synthetic Oligonucleotide
ggactggtgtcatctgggac

SEQ ID NO: 237 - DNA, 20 bp, Artificial Sequence
  Synthetic Oligonucleotide
ccacccttggtgcccagatc

SEQ ID NO: 238 - DNA, 20 bp, Artificial Sequence
  Synthetic Oligonucleotide
cagggtgcccatcctagtca

SEQ ID NO: 239 - DNA, 20 bp, Artificial Sequence
  Synthetic Oligonucleotide
tcctgcgagtgggagttgga

SEQ ID NO: 240 - DNA, 20 bp, Artificial Sequence
  Synthetic Oligonucleotide
atcccattttcccaggagcc

SEQ ID NO: 241 - DNA, 20 bp, Artificial Sequence
  Synthetic Oligonucleotide
agaaacatcccattttccca

SEQ ID NO: 242 - DNA, 20 bp, Artificial Sequence
  Synthetic Oligonucleotide
ccaggctggtttggagaaac

SEQ ID NO: 243 - DNA, 20 bp, Artificial Sequence
  Synthetic Oligonucleotide
atgaaattctacctaaagat

SEQ ID NO: 244 - DNA, 20 bp, Artificial Sequence
  Synthetic Oligonucleotide
aggtgaatgaaattctacct

SEQ ID NO: 245 - DNA, 20 bp, Artificial Sequence
  Synthetic Oligonucleotide
gacaatggtcagggctggtt

SEQ ID NO: 246 - DNA, 20 bp, Artificial Sequence
  Synthetic Oligonucleotide
tggctggctgaggagacaat

SEQ ID NO: 247 - DNA, 20 bp, Artificial Sequence
  Synthetic Oligonucleotide
cagaaacacccatcctctga

SEQ ID NO: 248 - DNA, 20 bp, Artificial Sequence
  Synthetic Oligonucleotide
tttttccacatggatattca

SEQ ID NO: 249 - DNA, 20 bp, Artificial Sequence
  Synthetic Oligonucleotide
taggtatttttccacatgga

SEQ ID NO: 250 - DNA, 20 bp, Artificial Sequence
  Synthetic Oligonucleotide
ggtttgctggcatttctttt

SEQ ID NO: 251 - DNA, 20 bp, Artificial Sequence
  Synthetic Oligonucleotide
gccatggacactcccctttg

SEQ ID NO: 252 - DNA, 20 bp, Artificial Sequence
  Synthetic Oligonucleotide
tctgcacagggtagaccctc

SEQ ID NO: 253 - DNA, 20 bp, Artificial Sequence
  Synthetic Oligonucleotide
ggttctctgcacagggtaga

SEQ ID NO: 254 - DNA, 20 bp, Artificial Sequence
  Synthetic Oligonucleotide
aaagatcccacctcaaagag

SEQ ID NO: 255 - DNA, 20 bp, Artificial Sequence
  Synthetic Oligonucleotide
aaagatcaggcaaagatccc

SEQ ID NO: 256 - DNA, 20 bp, Artificial Sequence
  Synthetic Oligonucleotide
atagctttgatccaatgctc

SEQ ID NO: 257 - DNA, 20 bp, Artificial Sequence
  Synthetic Oligonucleotide
tcccaggcaaagctgctcag

SEQ ID NO: 258 - DNA, 20 bp, Artificial Sequence
  Synthetic Oligonucleotide
gattttgtcgaaacagtggg

SEQ ID NO: 259 - DNA, 20 bp, Artificial Sequence
  Synthetic Oligonucleotide
tgacagcacgaagcccagag

SEQ ID NO: 260 - DNA, 20 bp, Artificial Sequence
  Synthetic Oligonucleotide
gccatttctaggtctgcagg

SEQ ID NO: 261 - DNA, 20 bp, Artificial Sequence
  Synthetic Oligonucleotide
cgccggctctgctcatcccc

SEQ ID NO: 262 - DNA, 20 bp, Artificial Sequence
  Synthetic Oligonucleotide
ctgcgccacccgccggctct

SEQ ID NO: 263 - DNA, 20 bp, Artificial Sequence
  Synthetic Oligonucleotide
ggatgatgacctgcgccacc

SEQ ID NO: 264 - DNA, 20 bp, Artificial Sequence
  Synthetic Oligonucleotide
ggcggagcagcgcgatgtcg

SEQ ID NO: 265 - DNA, 20 bp, Artificial Sequence
  Synthetic Oligonucleotide
tggtcagtgaggaccacggg

SEQ ID NO: 266 - DNA, 20 bp, Artificial Sequence
  Synthetic Oligonucleotide
caccacatggtcagtgagga

SEQ ID NO: 267 - DNA, 20 bp, Artificial Sequence
  Synthetic Oligonucleotide
gaagcgcacgaaggccagcg

SEQ ID NO: 268 - DNA, 20 bp, Artificial Sequence
  Synthetic Oligonucleotide
ggcagtcctgggtcatcagc

SEQ ID NO: 269 - DNA, 20 bp, Artificial Sequence
  Synthetic Oligonucleotide
tgctgcaggcagtcctgggt

SEQ ID NO: 270 - DNA, 20 bp, Artificial Sequence
  Synthetic Oligonucleotide
aacatgtactccgtgatatt

SEQ ID NO: 271 - DNA, 20 bp, Artificial Sequence
  Synthetic Oligonucleotide
cactgtcccccttgcaggag

SEQ ID NO: 272 - DNA, 20 bp, Artificial Sequence
  Synthetic Oligonucleotide
tgtgggcctccactgtcccc

SEQ ID NO: 273 - DNA, 20 bp, Artificial Sequence
  Synthetic Oligonucleotide
ccctggccccagctgacgat

SEQ ID NO: 274 - DNA, 20 bp, Artificial Sequence
  Synthetic Oligonucleotide
tgggagaccctggtgtacac

SEQ ID NO: 275 - DNA, 20 bp, Artificial Sequence
  Synthetic Oligonucleotide
gcagccactcgatgtactgg

SEQ ID NO: 276 - DNA, 20 bp, Artificial Sequence
  Synthetic Oligonucleotide
gcttttgcagccactcgatg

SEQ ID NO: 277 - DNA, 20 bp, Artificial Sequence
  Synthetic Oligonucleotide
tgagcttttgcagccactcg

SEQ ID NO: 278 - DNA, 20 bp, Artificial Sequence
  Synthetic Oligonucleotide
cagccttggctttctctcca

SEQ ID NO: 279 - DNA, 20 bp, Artificial Sequence
  Synthetic Oligonucleotide
ccctgcccctctgtccagcg

SEQ ID NO: 280 - DNA, 20 bp, Artificial Sequence
  Synthetic Oligonucleotide
tgtctgcctccaggacaacc

SEQ ID NO: 281 - DNA, 20 bp, Artificial Sequence
  Synthetic Oligonucleotide
ctcagctgggctgtctgcct

SEQ ID NO: 282 - DNA, 20 bp, Artificial Sequence
  Synthetic Oligonucleotide
gcaggtgggcttggctgaag

SEQ ID NO: 283 - DNA, 20 bp, Artificial Sequence
  Synthetic Oligonucleotide
cagcctgaggccagcagatc

SEQ ID NO: 284 - DNA, 20 bp, Artificial Sequence
  Synthetic Oligonucleotide
gtctccagcaatgaaggcag

SEQ ID NO: 285 - DNA, 20 bp, Artificial Sequence
  Synthetic Oligonucleotide
gacagcacacatggagtcag

SEQ ID NO: 286 - DNA, 20 bp, Artificial Sequence
  Synthetic Oligonucleotide
aagtgagagctaaacaaccg

SEQ ID NO: 287 - DNA, 20 bp, Artificial Sequence
  Synthetic Oligonucleotide
agatgataatggataagaac

SEQ ID NO: 288 - DNA, 20 bp, Artificial Sequence
  Synthetic Oligonucleotide
catggtgatgcttctgaatt

SEQ ID NO: 289 - DNA, 20 bp, Artificial Sequence
  Synthetic Oligonucleotide
tgtgagggttcatttcagtg

SEQ ID NO: 290 - DNA, 20 bp, Artificial Sequence
  Synthetic Oligonucleotide
tacagcctcaaacatctatc

SEQ ID NO: 291 - DNA, 20 bp, Artificial Sequence
  Synthetic Oligonucleotide
gagctacagcctcaaacatc

SEQ ID NO: 292 - DNA, 20 bp, Artificial Sequence
  Synthetic Oligonucleotide
attgacaagggctgtggcag

SEQ ID NO: 293 - DNA, 20 bp, Artificial Sequence
  Synthetic Oligonucleotide
gcaggtgctcccagggtctc

SEQ ID NO: 294 - DNA, 20 bp, Artificial Sequence
  Synthetic Oligonucleotide
caggtcctcccactgcagcc

SEQ ID NO: 295 - DNA, 20 bp, Artificial Sequence
  Synthetic Oligonucleotide
gtgcagcttgccaggtcctc

SEQ ID NO: 296 - DNA, 20 bp, Artificial Sequence
  Synthetic Oligonucleotide
actcagcaagagtgcagctt

SEQ ID NO: 297 - DNA, 20 bp, Artificial Sequence
  Synthetic Oligonucleotide
taaaactttatttacaagag

SEQ ID NO: 298 - DNA, 20 bp, Artificial Sequence
  Synthetic Oligonucleotide
ggtgtgttcccataaaactt

SEQ ID NO: 299 - DNA, 20 bp, Artificial Sequence
  Synthetic Oligonucleotide
aaagcagagccagctctgac

SEQ ID NO: 300 - DNA, 20 bp, Artificial Sequence
  Synthetic Oligonucleotide
gcctgcatttcccattggca

SEQ ID NO: 301 - DNA, 20 bp, Artificial Sequence
  Synthetic Oligonucleotide
gccactcacagaaagctgga

SEQ ID NO: 302 - DNA, 20 bp, Artificial Sequence
  Synthetic Oligonucleotide
caggatgctcatggcagaca

SEQ ID NO: 303 - DNA, 20 bp, Artificial Sequence
  Synthetic Oligonucleotide
gtttgtatggagagaccaat

SEQ ID NO: 304 - DNA, 20 bp, Artificial Sequence
  Synthetic Oligonucleotide
tggtggcaccagatgtctga

SEQ ID NO: 305 - DNA, 20 bp, Artificial Sequence
  Synthetic Oligonucleotide
cattgtgcctggcacacagg

SEQ ID NO: 306 - DNA, 20 bp, Artificial Sequence
  Synthetic Oligonucleotide
gcctggtgtgcacacattgt

SEQ ID NO: 307 - DNA, 20 bp, Artificial Sequence
  Synthetic Oligonucleotide
ctgggttacgaagactgcag

SEQ ID NO: 308 - DNA, 20 bp, Artificial Sequence
  Synthetic Oligonucleotide
agcgaccccgcctgcagcca

SEQ ID NO: 309 - DNA, 20 bp, Artificial Sequence
  Synthetic Oligonucleotide
agaaatccagaacagcttcg

SEQ ID NO: 310 - DNA, 20 bp, Artificial Sequence
  Synthetic Oligonucleotide
cccatcactgtaagaaatcc

SEQ ID NO: 311 - DNA, 20 bp, Artificial Sequence
  Synthetic Oligonucleotide
actggtccccatcactgtaa

SEQ ID NO: 312 - DNA, 20 bp, Artificial Sequence
  Synthetic Oligonucleotide
aggcacactggtccccatca

SEQ ID NO: 313 - DNA, 20 bp, Artificial Sequence
  Synthetic Oligonucleotide
catggatattcaactgtggg

SEQ ID NO: 314 - DNA, 20 bp, Artificial Sequence
  Synthetic Oligonucleotide
ccaacaacaggacctgccat

SEQ ID NO: 315 - DNA, 20 bp, Artificial Sequence
  Synthetic Oligonucleotide
cgtgctcgcccagcaccgcg

SEQ ID NO: 316 - DNA, 20 bp, Artificial Sequence
  Synthetic Oligonucleotide
ggaagaagggacggaaggtg

SEQ ID NO: 317 - DNA, 20 bp, Artificial Sequence
  Synthetic Oligonucleotide
cggaagaagggacggaaggt

SEQ ID NO: 318 - DNA, 20 bp, Artificial Sequence
  Synthetic Oligonucleotide
gcggaagaagggacggaagg

SEQ ID NO: 319 - DNA, 20 bp, Artificial Sequence
  Synthetic Oligonucleotide
cgcggaagaagggacggaag

SEQ ID NO: 320 - DNA, 20 bp, Artificial Sequence
  Synthetic Oligonucleotide
gcgcggaagaagggacggaa

SEQ ID NO: 321 - DNA, 20 bp, Artificial Sequence
  Synthetic Oligonucleotide
agcgcggaagaagggacgga

SEQ ID NO: 322 - DNA, 20 bp, Artificial Sequence
  Synthetic Oligonucleotide
gagcgcggaagaagggacgg

SEQ ID NO: 323 - DNA, 20 bp, Artificial Sequence
  Synthetic Oligonucleotide
tgagcgcggaagaagggacg

SEQ ID NO: 324 - DNA, 20 bp, Artificial Sequence
  Synthetic Oligonucleotide
ctgagcgcggaagaagggac

SEQ ID NO: 325 - DNA, 20 bp, Artificial Sequence
  Synthetic Oligonucleotide
actgagcgcggaagaaggga

SEQ ID NO: 326 - DNA, 20 bp, Artificial Sequence
  Synthetic Oligonucleotide
tactgagcgcggaagaaggg

SEQ ID NO: 327 - DNA, 20 bp, Artificial Sequence
  Synthetic Oligonucleotide
ttactgagcgcggaagaagg

SEQ ID NO: 328 - DNA, 20 bp, Artificial Sequence
  Synthetic Oligonucleotide
gttactgagcgcggaagaag

SEQ ID NO: 329 - DNA, 20 bp, Artificial Sequence
  Synthetic Oligonucleotide
tctgcaggagcggcctaaat

SEQ ID NO: 330 - DNA, 20 bp, Artificial Sequence
  Synthetic Oligonucleotide
ttctgcaggagcggcctaaa

SEQ ID NO: 331 - DNA, 20 bp, Artificial Sequence
  Synthetic Oligonucleotide
gttctgcaggagcggcctaa

SEQ ID NO: 332 - DNA, 20 bp, Artificial Sequence
  Synthetic Oligonucleotide
agttctgcaggagcggccta

SEQ ID NO: 333 - DNA, 20 bp, Artificial Sequence
  Synthetic Oligonucleotide
gagttctgcaggagcggcct

SEQ ID NO: 334 - DNA, 20 bp, Artificial Sequence
  Synthetic Oligonucleotide
ccgagttctgcaggagcggc

SEQ ID NO: 335 - DNA, 20 bp, Artificial Sequence
  Synthetic Oligonucleotide
cccgagttctgcaggagcgg

SEQ ID NO: 336 - DNA, 20 bp, Artificial Sequence
  Synthetic Oligonucleotide
gcccgagttctgcaggagcg

SEQ ID NO: 337 - DNA, 20 bp, Artificial Sequence
  Synthetic Oligonucleotide
agcccgagttctgcaggagc

SEQ ID NO: 338 - DNA, 20 bp, Artificial Sequence
  Synthetic Oligonucleotide
gagcccgagttctgcaggag

SEQ ID NO: 339 - DNA, 20 bp, Artificial Sequence
  Synthetic Oligonucleotide
ggagcccgagttctgcagga

SEQ ID NO: 340 - DNA, 20 bp, Artificial Sequence
  Synthetic Oligonucleotide
tcagggctggttttggagga

SEQ ID NO: 341 - DNA, 20 bp, Artificial Sequence
  Synthetic Oligonucleotide
gtcagggctggttttggagg

SEQ ID NO: 342 - DNA, 20 bp, Artificial Sequence
  Synthetic Oligonucleotide
ggtcagggctggttttggag

SEQ ID NO: 343 - DNA, 20 bp, Artificial Sequence
  Synthetic Oligonucleotide
tggtcagggctggttttgga

SEQ ID NO: 344 - DNA, 20 bp, Artificial Sequence
  Synthetic Oligonucleotide
atggtcagggctggttttgg

SEQ ID NO: 345 - DNA, 20 bp, Artificial Sequence
  Synthetic Oligonucleotide
aatggtcagggctggttttg

SEQ ID NO: 346 - DNA, 20 bp, Artificial Sequence
  Synthetic Oligonucleotide
caatggtcagggctggtttt

SEQ ID NO: 347 - DNA, 20 bp, Artificial Sequence
  Synthetic Oligonucleotide
acaatggtcagggctggttt

SEQ ID NO: 348 - DNA, 20 bp, Artificial Sequence
  Synthetic Oligonucleotide
agacaatggtcagggctggt

SEQ ID NO: 349 - DNA, 20 bp, Artificial Sequence
  Synthetic Oligonucleotide
gagacaatggtcagggctgg

SEQ ID NO: 350 - DNA, 20 bp, Artificial Sequence
  Synthetic Oligonucleotide
ggagacaatggtcagggctg

SEQ ID NO: 351 - DNA, 20 bp, Artificial Sequence
  Synthetic Oligonucleotide
aggagacaatggtcagggct

SEQ ID NO: 352 - DNA, 20 bp, Artificial Sequence
  Synthetic Oligonucleotide
gaggagacaatggtcagggc

SEQ ID NO: 353 - DNA, 20 bp, Artificial Sequence
  Synthetic Oligonucleotide
tgaggagacaatggtcaggg

SEQ ID NO: 354 - DNA, 20 bp, Artificial Sequence
  Synthetic Oligonucleotide
ctgaggagacaatggtcagg

SEQ ID NO: 355 - DNA, 20 bp, Artificial Sequence
  Synthetic Oligonucleotide
gctgaggagacaatggtcag

SEQ ID NO: 356 - DNA, 20 bp, Artificial Sequence
  Synthetic Oligonucleotide
ggctgaggagacaatggtca

SEQ ID NO: 357 - DNA, 20 bp, Artificial Sequence
  Synthetic Oligonucleotide
tggctgaggagacaatggtc

SEQ ID NO: 358 - DNA, 20 bp, Artificial Sequence
  Synthetic Oligonucleotide
ctggctgaggagacaatggt

SEQ ID NO: 359 - DNA, 20 bp, Artificial Sequence
  Synthetic Oligonucleotide
gctggctgaggagacaatgg

SEQ ID NO: 360 - DNA, 20 bp, Artificial Sequence
  Synthetic Oligonucleotide
ccgccggctctgctcatccc

SEQ ID NO: 361 - DNA, 20 bp, Artificial Sequence
  Synthetic Oligonucleotide
cccgccggctctgctcatcc

SEQ ID NO: 362 - DNA, 20 bp, Artificial Sequence
  Synthetic Oligonucleotide
acccgccggctctgctcatc

SEQ ID NO: 363 - DNA, 20 bp, Artificial Sequence
  Synthetic Oligonucleotide
cacccgccggctctgctcat

SEQ ID NO: 364 - DNA, 20 bp, Artificial Sequence
  Synthetic Oligonucleotide
ccacccgccggctctgctca

SEQ ID NO: 365 - DNA, 20 bp, Artificial Sequence
  Synthetic Oligonucleotide
gccacccgccggctctgctc

SEQ ID NO: 366 - DNA, 20 bp, Artificial Sequence
  Synthetic Oligonucleotide
cgccacccgccggctctgct

SEQ ID NO: 367 - DNA, 20 bp, Artificial Sequence
  Synthetic Oligonucleotide
gcgccacccgccggctctgc

SEQ ID NO: 368 - DNA, 20 bp, Artificial Sequence
  Synthetic Oligonucleotide
tgcgccacccgccggctctg

SEQ ID NO: 369 - DNA, 20 bp, Artificial Sequence
  Synthetic Oligonucleotide
gcctgaggccagcagatcac

SEQ ID NO: 370 - DNA, 20 bp, Artificial Sequence
  Synthetic Oligonucleotide
agcctgaggccagcagatca

SEQ ID NO: 371 - DNA, 20 bp, Artificial Sequence
  Synthetic Oligonucleotide
gcagcctgaggccagcagat

SEQ ID NO: 372 - DNA, 20 bp, Artificial Sequence
  Synthetic Oligonucleotide
agcagcctgaggccagcaga

SEQ ID NO: 373 - DNA, 20 bp, Artificial Sequence
  Synthetic Oligonucleotide
gcacacatggagtcagcatc

SEQ ID NO: 374 - DNA, 20 bp, Artificial Sequence
  Synthetic Oligonucleotide
agcacacatggagtcagcat

SEQ ID NO: 375 - DNA, 20 bp, Artificial Sequence
  Synthetic Oligonucleotide
cagcacacatggagtcagca

SEQ ID NO: 376 - DNA, 20 bp, Artificial Sequence
  Synthetic Oligonucleotide
acagcacacatggagtcagc

SEQ ID NO: 377 - DNA, 20 bp, Artificial Sequence
  Synthetic Oligonucleotide
gcatggtgatgcttctgaat

SEQ ID NO: 378 - DNA, 20 bp, Artificial Sequence
  Synthetic Oligonucleotide
tgcatggtgatgcttctgaa

SEQ ID NO: 379 - DNA, 20 bp, Artificial Sequence
  Synthetic Oligonucleotide
catgcatggtgatgcttctg

SEQ ID NO: 380 - DNA, 14 bp, Artificial Sequence
  Synthetic Oligonucleotide
ccctgagctgggca

SEQ ID NO: 381 - DNA, 14 bp, Artificial Sequence
  Synthetic Oligonucleotide
gagccacagagcct

SEQ ID NO: 382 - DNA, 14 bp, Artificial Sequence
  Synthetic Oligonucleotide
tcttgggtgtggat

SEQ ID NO: 383 - DNA, 14 bp, Artificial Sequence
  Synthetic Oligonucleotide
cagatgaaaacttg

SEQ ID NO: 384 - DNA, 14 bp, Artificial Sequence
  Synthetic Oligonucleotide
caggtgttaaggtg

SEQ ID NO: 385 - DNA, 14 bp, Artificial Sequence
  Synthetic Oligonucleotide
aggagaaaggtcag

SEQ ID NO: 386 - DNA, 14 bp, Artificial Sequence
  Synthetic Oligonucleotide
tgagagctgcacct

SEQ ID NO: 387 - DNA, 14 bp, Artificial Sequence
  Synthetic Oligonucleotide
caaagttctctgcc

SEQ ID NO: 388 - DNA, 14 bp, Artificial Sequence
  Synthetic Oligonucleotide
tgaaatctctgcag

SEQ ID NO: 389 - DNA, 14 bp, Artificial Sequence
  Synthetic Oligonucleotide
catgatgaaatctc

SEQ ID NO: 390 - DNA, 14 bp, Artificial Sequence
  Synthetic Oligonucleotide
gagaccatgatgaa

SEQ ID NO: 391 - DNA, 14 bp, Artificial Sequence
  Synthetic Oligonucleotide
tgaagcccaagcag

SEQ ID NO: 392 - DNA, 14 bp, Artificial Sequence
  Synthetic Oligonucleotide
agccctgaagccca

SEQ ID NO: 393 - DNA, 14 bp, Artificial Sequence
  Synthetic Oligonucleotide
gcacctgcagccag

SEQ ID NO: 394 - DNA, 14 bp, Artificial Sequence
  Synthetic Oligonucleotide
caccaagtttatgg

SEQ ID NO: 395 - DNA, 14 bp, Artificial Sequence
  Synthetic Oligonucleotide
gcctggatgctggt

SEQ ID NO: 396 - DNA, 14 bp, Artificial Sequence
  Synthetic Oligonucleotide
ttctttgaaaaata

SEQ ID NO: 397 - DNA, 14 bp, Artificial Sequence
  Synthetic Oligonucleotide
ctctgagaggcccc

SEQ ID NO: 398 - DNA, 14 bp, Artificial Sequence
  Synthetic Oligonucleotide
gtcataaggccatg

SEQ ID NO: 399 - DNA, 14 bp, Artificial Sequence
  Synthetic Oligonucleotide
gcagtccctgctca

SEQ ID NO: 400 - DNA, 14 bp, Artificial Sequence
  Synthetic Oligonucleotide
cagggaacaccctc

SEQ ID NO: 401 - DNA, 14 bp, Artificial Sequence
  Synthetic Oligonucleotide
gtcccagggaaggc

SEQ ID NO: 402 - DNA, 14 bp, Artificial Sequence
  Synthetic Oligonucleotide
tctggaaggaacag

SEQ ID NO: 403 - DNA, 14 bp, Artificial Sequence
  Synthetic Oligonucleotide
ctgaggatgcaggc

SEQ ID NO: 404 - DNA, 14 bp, Artificial Sequence
  Synthetic Oligonucleotide
gctactgggccacg

SEQ ID NO: 405 - DNA, 14 bp, Artificial Sequence
  Synthetic Oligonucleotide
tgagcgcggaagaa

SEQ ID NO: 406 - DNA, 14 bp, Artificial Sequence
  Synthetic Oligonucleotide
ggagggacgacctt

SEQ ID NO: 407 - DNA, 14 bp, Artificial Sequence
  Synthetic Oligonucleotide
accacgcggtggtg

SEQ ID NO: 408 - DNA, 14 bp, Artificial Sequence
  Synthetic Oligonucleotide
cgtgcgctcagctc

SEQ ID NO: 409 - DNA, 14 bp, Artificial Sequence
  Synthetic Oligonucleotide
aaaccggcatgcgc

SEQ ID NO: 410 - DNA, 14 bp, Artificial Sequence
  Synthetic Oligonucleotide
cctaagtgtgcttt

SEQ ID NO: 411 - DNA, 14 bp, Artificial Sequence
  Synthetic Oligonucleotide
agcttctcaccacg

SEQ ID NO: 412 - DNA, 14 bp, Artificial Sequence
  Synthetic Oligonucleotide
gagtgtgaggcttc

SEQ ID NO: 413 - DNA, 14 bp, Artificial Sequence
  Synthetic Oligonucleotide
catgttagcctttg

SEQ ID NO: 414 - DNA, 14 bp, Artificial Sequence
  Synthetic Oligonucleotide
atgaccatcctcaa

SEQ ID NO: 415 - DNA, 14 bp, Artificial Sequence
  Synthetic Oligonucleotide
atgagaaatgcttt

SEQ ID NO: 416 - DNA, 14 bp, Artificial Sequence
  Synthetic Oligonucleotide
tgctgaagaaactg

SEQ ID NO: 417 - DNA, 14 bp, Artificial Sequence
  Synthetic Oligonucleotide
tgcagtagcagatg

SEQ ID NO: 418 - DNA, 14 bp, Artificial Sequence
  Synthetic Oligonucleotide
gaggtacacaggct

SEQ ID NO: 419 - DNA, 14 bp, Artificial Sequence
  Synthetic Oligonucleotide
acacccgaggttca

SEQ ID NO: 420 - DNA, 14 bp, Artificial Sequence
  Synthetic Oligonucleotide
tgaccacacatttc

SEQ ID NO: 421 - DNA, 14 bp, Artificial Sequence
  Synthetic Oligonucleotide
aatcttggcccctc

SEQ ID NO: 422 - DNA, 14 bp, Artificial Sequence
  Synthetic Oligonucleotide
agccagactttcct

SEQ ID NO: 423 - DNA, 14 bp, Artificial Sequence
  Synthetic Oligonucleotide
tccagaacagcttc

SEQ ID NO: 424 - DNA, 14 bp, Artificial Sequence
  Synthetic Oligonucleotide
tgtaagaaatccag

SEQ ID NO: 425 - DNA, 14 bp, Artificial Sequence
  Synthetic Oligonucleotide
ctggtccccatctg

SEQ ID NO: 426 - DNA, 14 bp, Artificial Sequence
  Synthetic Oligonucleotide
acactggtccccat

SEQ ID NO: 427 - DNA, 14 bp, Artificial Sequence
  Synthetic Oligonucleotide
gaggcacactggtc

SEQ ID NO: 428 - DNA, 14 bp, Artificial Sequence
  Synthetic Oligonucleotide
cttgcaggagcccc

SEQ ID NO: 429 - DNA, 14 bp, Artificial Sequence
  Synthetic Oligonucleotide
tggtccttgcagga

SEQ ID NO: 430 - DNA, 14 bp, Artificial Sequence
  Synthetic Oligonucleotide
ggagctggtccttg

SEQ ID NO: 431 - DNA, 14 bp, Artificial Sequence
  Synthetic Oligonucleotide
taggactggagctg

SEQ ID NO: 432 - DNA, 14 bp, Artificial Sequence
  Synthetic Oligonucleotide
agatataggactgg

SEQ ID NO: 433 - DNA, 14 bp, Artificial Sequence
  Synthetic Oligonucleotide
gaagcagatatagg

SEQ ID NO: 434 - DNA, 14 bp, Artificial Sequence
  Synthetic Oligonucleotide
aggcagaagcagat

SEQ ID NO: 435 - DNA, 14 bp, Artificial Sequence
  Synthetic Oligonucleotide
ccggccctcgaagg

SEQ ID NO: 436 - DNA, 14 bp, Artificial Sequence
  Synthetic Oligonucleotide
acagttccggccct

SEQ ID NO: 437 - DNA, 14 bp, Artificial Sequence
  Synthetic Oligonucleotide
ggacccaaagtggg

SEQ ID NO: 438 - DNA, 14 bp, Artificial Sequence
  Synthetic Oligonucleotide
cggttggccaggcc

SEQ ID NO: 439 - DNA, 14 bp, Artificial Sequence
  Synthetic Oligonucleotide
gtcacctagaccaa

SEQ ID NO: 440 - DNA, 14 bp, Artificial Sequence
  Synthetic Oligonucleotide
gagaattgcccagg

SEQ ID NO: 441 - DNA, 14 bp, Artificial Sequence
  Synthetic Oligonucleotide
cagggactctcagc

SEQ ID NO: 442 - DNA, 14 bp, Artificial Sequence
  Synthetic Oligonucleotide
tgaattactgaacc

SEQ ID NO: 443 - DNA, 14 bp, Artificial Sequence
  Synthetic Oligonucleotide
tgaccccacaggga

SEQ ID NO: 444 - DNA, 14 bp, Artificial Sequence
  Synthetic Oligonucleotide
cagtacttcccacg

SEQ ID NO: 445 - DNA, 14 bp, Artificial Sequence
  Synthetic Oligonucleotide
ctctggacaccggg

SEQ ID NO: 446 - DNA, 14 bp, Artificial Sequence
  Synthetic Oligonucleotide
ctgacaatgtgatg

SEQ ID NO: 447 - DNA, 14 bp, Artificial Sequence
  Synthetic Oligonucleotide
ctgagggaatgttg

SEQ ID NO: 448 - DNA, 14 bp, Artificial Sequence
  Synthetic Oligonucleotide
gtggtcaaggatga

SEQ ID NO: 449 - DNA, 14 bp, Artificial Sequence
  Synthetic Oligonucleotide
gctgagcagagatc

SEQ ID NO: 450 - DNA, 14 bp, Artificial Sequence
  Synthetic Oligonucleotide
ggatgcacaccagg

SEQ ID NO: 451 - DNA, 14 bp, Artificial Sequence
  Synthetic Oligonucleotide
tgacgagaggacca

SEQ ID NO: 452 - DNA, 14 bp, Artificial Sequence
  Synthetic Oligonucleotide
ctcttccgagcagc

SEQ ID NO: 453 - DNA, 14 bp, Artificial Sequence
  Synthetic Oligonucleotide
tccaaaggacaagg

SEQ ID NO: 454 - DNA, 14 bp, Artificial Sequence
  Synthetic Oligonucleotide
ctgagcttggcacc

SEQ ID NO: 455 - DNA, 14 bp, Artificial Sequence
  Synthetic Oligonucleotide
gtcatccttgtctg

SEQ ID NO: 456 - DNA, 14 bp, Artificial Sequence
  Synthetic Oligonucleotide
ctggtcatccttgt

SEQ ID NO: 457 - DNA, 14 bp, Artificial Sequence
  Synthetic Oligonucleotide
atcagctggtcatc

SEQ ID NO: 458 - DNA, 14 bp, Artificial Sequence
  Synthetic Oligonucleotide
gccgttctcgttca

SEQ ID NO: 459 - DNA, 14 bp, Artificial Sequence
  Synthetic Oligonucleotide
acagccgccgttct

SEQ ID NO: 460 - DNA, 14 bp, Artificial Sequence
  Synthetic Oligonucleotide
actgctcacagccg

SEQ ID NO: 461 - DNA, 14 bp, Artificial Sequence
  Synthetic Oligonucleotide
gcagtactgctcac

SEQ ID NO: 462 - DNA, 14 bp, Artificial Sequence
  Synthetic Oligonucleotide
tcactgcagtactg

SEQ ID NO: 463 - DNA, 14 bp, Artificial Sequence
  Synthetic Oligonucleotide
tacccctcgtggca

SEQ ID NO: 464 - DNA, 14 bp, Artificial Sequence
  Synthetic Oligonucleotide
ccgtctgccagcag

SEQ ID NO: 465 - DNA, 14 bp, Artificial Sequence
  Synthetic Oligonucleotide
ggacaccccgtctg

SEQ ID NO: 466 - DNA, 14 bp, Artificial Sequence
  Synthetic Oligonucleotide
tttgtccagtaaga

SEQ ID NO: 467 - DNA, 14 bp, Artificial Sequence
  Synthetic Oligonucleotide
catcctagtcactg

SEQ ID NO: 468 - DNA, 14 bp, Artificial Sequence
  Synthetic Oligonucleotide
agtcagtacagaca

SEQ ID NO: 469 - DNA, 14 bp, Artificial Sequence
  Synthetic Oligonucleotide
ccaggagcctttac

SEQ ID NO: 470 - DNA, 14 bp, Artificial Sequence
  Synthetic Oligonucleotide
tgagtcccaggctg

SEQ ID NO: 471 - DNA, 14 bp, Artificial Sequence
  Synthetic Oligonucleotide
cctaaagatgaatc

SEQ ID NO: 472 - DNA, 14 bp, Artificial Sequence
  Synthetic Oligonucleotide
gggacactcacact

SEQ ID NO: 473 - DNA, 14 bp, Artificial Sequence
  Synthetic Oligonucleotide
ctggttttggagga

SEQ ID NO: 474 - DNA, 14 bp, Artificial Sequence
  Synthetic Oligonucleotide
aagtggctggctga

SEQ ID NO: 475 - DNA, 14 bp, Artificial Sequence
  Synthetic Oligonucleotide
tcagaaagatgcag

SEQ ID NO: 476 - DNA, 14 bp, Artificial Sequence
  Synthetic Oligonucleotide
tggatattcaactg

SEQ ID NO: 477 - DNA, 14 bp, Artificial Sequence
  Synthetic Oligonucleotide
ccacatggatattc

SEQ ID NO: 478 - DNA, 14 bp, Artificial Sequence
  Synthetic Oligonucleotide
tttttccacatgga

SEQ ID NO: 479 - DNA, 14 bp, Artificial Sequence
  Synthetic Oligonucleotide
aggtatttttccac

SEQ ID NO: 480 - DNA, 14 bp, Artificial Sequence
  Synthetic Oligonucleotide
ggtttgctggcatt

SEQ ID NO: 481 - DNA, 14 bp, Artificial Sequence
  Synthetic Oligonucleotide
aattcggccttggg

SEQ ID NO: 482 - DNA, 14 bp, Artificial Sequence
  Synthetic Oligonucleotide
cactcccctttggg

SEQ ID NO: 483 - DNA, 14 bp, Artificial Sequence
  Synthetic Oligonucleotide
tgccatggacactc

SEQ ID NO: 484 - DNA, 14 bp, Artificial Sequence
  Synthetic Oligonucleotide
gacctgcccatttt

SEQ ID NO: 485 - DNA, 14 bp, Artificial Sequence
  Synthetic Oligonucleotide
gggtagaccctcag

SEQ ID NO: 486 - DNA, 14 bp, Artificial Sequence
  Synthetic Oligonucleotide
tgattttgcaaaga

SEQ ID NO: 487 - DNA, 14 bp, Artificial Sequence
  Synthetic Oligonucleotide
gatcttcacatagc

SEQ ID NO: 488 - DNA, 14 bp, Artificial Sequence
  Synthetic Oligonucleotide
tgctcagacctggc

SEQ ID NO: 489 - DNA, 14 bp, Artificial Sequence
  Synthetic Oligonucleotide
tcaccaacaacagg

SEQ ID NO: 490 - DNA, 14 bp, Artificial Sequence
  Synthetic Oligonucleotide
acccagatggtgtt

SEQ ID NO: 491 - DNA, 14 bp, Artificial Sequence
  Synthetic Oligonucleotide
agaccacccagatg

SEQ ID NO: 492 - DNA, 14 bp, Artificial Sequence
  Synthetic Oligonucleotide
aaacagtgggccgc

SEQ ID NO: 493 - DNA, 14 bp, Artificial Sequence
  Synthetic Oligonucleotide
tgtcgaaacagtgg

SEQ ID NO: 494 - DNA, 14 bp, Artificial Sequence
  Synthetic Oligonucleotide
gattttgtcgaaac

SEQ ID NO: 495 - DNA, 14 bp, Artificial Sequence
  Synthetic Oligonucleotide
agacacttgagagc

SEQ ID NO: 496 - DNA, 14 bp, Artificial Sequence
  Synthetic Oligonucleotide
tgacagcacgaagc

SEQ ID NO: 497 - DNA, 14 bp, Artificial Sequence
  Synthetic Oligonucleotide
ttatttgggcatgg

SEQ ID NO: 498 - DNA, 14 bp, Artificial Sequence
  Synthetic Oligonucleotide
ctaggtctgcaggg

SEQ ID NO: 499 - DNA, 14 bp, Artificial Sequence
  Synthetic Oligonucleotide
ctcgcctggaagga

SEQ ID NO: 500 - DNA, 14 bp, Artificial Sequence
  Synthetic Oligonucleotide
aggtcgtgctcgcc

SEQ ID NO: 501 - DNA, 14 bp, Artificial Sequence
  Synthetic Oligonucleotide
cgctgaggtcgtgc

SEQ ID NO: 502 - DNA, 14 bp, Artificial Sequence
  Synthetic Oligonucleotide
gtgctcgctgaggt

SEQ ID NO: 503 - DNA, 14 bp, Artificial Sequence
  Synthetic Oligonucleotide
atgacctgcgccac

SEQ ID NO: 504 - DNA, 14 bp, Artificial Sequence
  Synthetic Oligonucleotide
ggatgatgacctgc

SEQ ID NO: 505 - DNA, 14 bp, Artificial Sequence
  Synthetic Oligonucleotide
atgtcgtggttggt

SEQ ID NO: 506 - DNA, 14 bp, Artificial Sequence
  Synthetic Oligonucleotide
agcagcgcgatgtc

SEQ ID NO: 507 - DNA, 14 bp, Artificial Sequence
  Synthetic Oligonucleotide
aggaccacgggctg

SEQ ID NO: 508 - DNA, 14 bp, Artificial Sequence
  Synthetic Oligonucleotide
cagtgaggaccacg

SEQ ID NO: 509 - DNA, 14 bp, Artificial Sequence
  Synthetic Oligonucleotide
atggtcagtgagga

SEQ ID NO: 510 - DNA, 14 bp, Artificial Sequence
  Synthetic Oligonucleotide
accacatggtcagt

SEQ ID NO: 511 - DNA, 14 bp, Artificial Sequence
  Synthetic Oligonucleotide
ttcgggcaggcaga

SEQ ID NO: 512 - DNA, 14 bp, Artificial Sequence
  Synthetic Oligonucleotide
gtccgttcgggcag

SEQ ID NO: 513 - DNA, 14 bp, Artificial Sequence
  Synthetic Oligonucleotide
cagccgctgaccaa

SEQ ID NO: 514 - DNA, 14 bp, Artificial Sequence
  Synthetic Oligonucleotide
cggtccagcagctg

SEQ ID NO: 515 - DNA, 14 bp, Artificial Sequence
  Synthetic Oligonucleotide
ggtcatcagccggg

SEQ ID NO: 516 - DNA, 14 bp, Artificial Sequence
  Synthetic Oligonucleotide
gggtcatcagccgg

SEQ ID NO: 517 - DNA, 14 bp, Artificial Sequence
  Synthetic Oligonucleotide
tgggtcatcagccg

SEQ ID NO: 518 - DNA, 14 bp, Artificial Sequence
  Synthetic Oligonucleotide
ctgggtcatcagcc

SEQ ID NO: 519 - DNA, 14 bp, Artificial Sequence
  Synthetic Oligonucleotide
cctgggtcatcagc

SEQ ID NO: 520 - DNA, 14 bp, Artificial Sequence
  Synthetic Oligonucleotide
tcctgggtcatcag

SEQ ID NO: 521 - DNA, 14 bp, Artificial Sequence
  Synthetic Oligonucleotide
gtcctgggtcatca

SEQ ID NO: 522 - DNA, 14 bp, Artificial Sequence
  Synthetic Oligonucleotide
agtcctgggtcatc

SEQ ID NO: 523 - DNA, 14 bp, Artificial Sequence
  Synthetic Oligonucleotide
cagtcctgggtcat

SEQ ID NO: 524 - DNA, 14 bp, Artificial Sequence
  Synthetic Oligonucleotide
ggcagtcctgggtc

SEQ ID NO: 525 - DNA, 14 bp, Artificial Sequence
  Synthetic Oligonucleotide
ctgcaggcagtcct

SEQ ID NO: 526 - DNA, 14 bp, Artificial Sequence
  Synthetic Oligonucleotide
gactgctgcaggca

SEQ ID NO: 527 - DNA, 14 bp, Artificial Sequence
  Synthetic Oligonucleotide
cagaacatgtactc

SEQ ID NO: 528 - DNA, 14 bp, Artificial Sequence
  Synthetic Oligonucleotide
agtagccggcacag

SEQ ID NO: 529 - DNA, 14 bp, Artificial Sequence
  Synthetic Oligonucleotide
ccttgcaggagtcc

SEQ ID NO: 530 - DNA, 14 bp, Artificial Sequence
  Synthetic Oligonucleotide
gtggcatgtgggcc

SEQ ID NO: 531 - DNA, 14 bp, Artificial Sequence
  Synthetic Oligonucleotide
gccccagctgacga

SEQ ID NO: 532 - DNA, 14 bp, Artificial Sequence
  Synthetic Oligonucleotide
ctggtgtacacccc

SEQ ID NO: 533 - DNA, 14 bp, Artificial Sequence
  Synthetic Oligonucleotide
cctggtgtacaccc

SEQ ID NO: 534 - DNA, 14 bp, Artificial Sequence
  Synthetic Oligonucleotide
ccctggtgtacacc

SEQ ID NO: 535 - DNA, 14 bp, Artificial Sequence
  Synthetic Oligonucleotide
accctggtgtacac

SEQ ID NO: 536 - DNA, 14 bp, Artificial Sequence
  Synthetic Oligonucleotide
gaccctggtgtaca

SEQ ID NO: 537 - DNA, 14 bp, Artificial Sequence
  Synthetic Oligonucleotide
agaccctggtgtac

SEQ ID NO: 538 - DNA, 14 bp, Artificial Sequence
  Synthetic Oligonucleotide
gagaccctggtgta

SEQ ID NO: 539 - DNA, 14 bp, Artificial Sequence
  Synthetic Oligonucleotide
ggagaccctggtgt

SEQ ID NO: 540 - DNA, 14 bp, Artificial Sequence
  Synthetic Oligonucleotide
gggagaccctggtg

SEQ ID NO: 541 - DNA, 14 bp, Artificial Sequence
  Synthetic Oligonucleotide
tgggagaccctggt

SEQ ID NO: 542 - DNA, 14 bp, Artificial Sequence
  Synthetic Oligonucleotide
ctgggagaccctgg

SEQ ID NO: 543 - DNA, 14 bp, Artificial Sequence
  Synthetic Oligonucleotide
actgggagaccctg

SEQ ID NO: 544 - DNA, 14 bp, Artificial Sequence
  Synthetic Oligonucleotide
tactgggagaccct

SEQ ID NO: 545 - DNA, 14 bp, Artificial Sequence
  Synthetic Oligonucleotide
gtactgggagaccc

SEQ ID NO: 546 - DNA, 14 bp, Artificial Sequence
  Synthetic Oligonucleotide
tgtactgggagacc

SEQ ID NO: 547 - DNA, 14 bp, Artificial Sequence
  Synthetic Oligonucleotide
atgtactgggagac

SEQ ID NO: 548 - DNA, 14 bp, Artificial Sequence
  Synthetic Oligonucleotide
actcgatgtactgg

SEQ ID NO: 549 - DNA, 14 bp, Artificial Sequence
  Synthetic Oligonucleotide
cagccactcgatgt

SEQ ID NO: 550 - DNA, 14 bp, Artificial Sequence
  Synthetic Oligonucleotide
ttttgcagccactc

SEQ ID NO: 551 - DNA, 14 bp, Artificial Sequence
  Synthetic Oligonucleotide
tgagcttttgcagc

SEQ ID NO: 552 - DNA, 14 bp, Artificial Sequence
  Synthetic Oligonucleotide
gctcgcaggaggac

SEQ ID NO: 553 - DNA, 14 bp, Artificial Sequence
  Synthetic Oligonucleotide
cagccttggctttc

SEQ ID NO: 554 - DNA, 14 bp, Artificial Sequence
  Synthetic Oligonucleotide
aggctcagctgggc

SEQ ID NO: 555 - DNA, 14 bp, Artificial Sequence
  Synthetic Oligonucleotide
taaggaggctcagc

SEQ ID NO: 556 - DNA, 14 bp, Artificial Sequence
  Synthetic Oligonucleotide
tgggcttggctgaa

SEQ ID NO: 557 - DNA, 14 bp, Artificial Sequence
  Synthetic Oligonucleotide
gccagcagatcacg

SEQ ID NO: 558 - DNA, 14 bp, Artificial Sequence
  Synthetic Oligonucleotide
ctgaggccagcaga

SEQ ID NO: 559 - DNA, 14 bp, Artificial Sequence
  Synthetic Oligonucleotide
gcagcctgaggcca

SEQ ID NO: 560 - DNA, 14 bp, Artificial Sequence
  Synthetic Oligonucleotide
gcaatgaaggcaga

SEQ ID NO: 561 - DNA, 14 bp, Artificial Sequence
  Synthetic Oligonucleotide
tggagtcagcatcg

SEQ ID NO: 562 - DNA, 14 bp, Artificial Sequence
  Synthetic Oligonucleotide
acacatggagtcag

SEQ ID NO: 563 - DNA, 14 bp, Artificial Sequence
  Synthetic Oligonucleotide
acagcacacatgga

SEQ ID NO: 564 - DNA, 14 bp, Artificial Sequence
  Synthetic Oligonucleotide
taaacaaccgcctt

SEQ ID NO: 565 - DNA, 14 bp, Artificial Sequence
  Synthetic Oligonucleotide
gtgagagctaaaca

SEQ ID NO: 566 - DNA, 14 bp, Artificial Sequence
  Synthetic Oligonucleotide
gaaaagtgagagct

SEQ ID NO: 567 - DNA, 14 bp, Artificial Sequence
  Synthetic Oligonucleotide
ctgaattgtctgaa

SEQ ID NO: 568 - DNA, 14 bp, Artificial Sequence
  Synthetic Oligonucleotide
atgcttctgaattg

SEQ ID NO: 569 - DNA, 14 bp, Artificial Sequence
  Synthetic Oligonucleotide
tggtgatgcttctg

SEQ ID NO: 570 - DNA, 14 bp, Artificial Sequence
  Synthetic Oligonucleotide
atgcatggtgatgc

SEQ ID NO: 571 - DNA, 14 bp, Artificial Sequence
  Synthetic Oligonucleotide
gtgcagcccggcac

SEQ ID NO: 572 - DNA, 14 bp, Artificial Sequence
  Synthetic Oligonucleotide
cagaggatgagcac

SEQ ID NO: 573 - DNA, 14 bp, Artificial Sequence
  Synthetic Oligonucleotide
catctcagaggatg

SEQ ID NO: 574 - DNA, 14 bp, Artificial Sequence
  Synthetic Oligonucleotide
tcatttcagtgatg

SEQ ID NO: 575 - DNA, 14 bp, Artificial Sequence
  Synthetic Oligonucleotide
agggttcatttcag

SEQ ID NO: 576 - DNA, 14 bp, Artificial Sequence
  Synthetic Oligonucleotide
atgtgagggttcat

SEQ ID NO: 577 - DNA, 14 bp, Artificial Sequence
  Synthetic Oligonucleotide
gcctcaaacatcta

SEQ ID NO: 578 - DNA, 14 bp, Artificial Sequence
  Synthetic Oligonucleotide
ctacagcctcaaac

SEQ ID NO: 579 - DNA, 14 bp, Artificial Sequence
  Synthetic Oligonucleotide
gggagctacagcct

SEQ ID NO: 580 - DNA, 14 bp, Artificial Sequence
  Synthetic Oligonucleotide
attgacaagggctg

SEQ ID NO: 581 - DNA, 14 bp, Artificial Sequence
  Synthetic Oligonucleotide
atatcattgacaag

SEQ ID NO: 582 - DNA, 14 bp, Artificial Sequence
  Synthetic Oligonucleotide
tcccagggtctctg

SEQ ID NO: 583 - DNA, 14 bp, Artificial Sequence
  Synthetic Oligonucleotide
cagccagggcctgg

SEQ ID NO: 584 - DNA, 14 bp, Artificial Sequence
  Synthetic Oligonucleotide
cactgcagccaggg

SEQ ID NO: 585 - DNA, 14 bp, Artificial Sequence
  Synthetic Oligonucleotide
cttgccaggtcctc

SEQ ID NO: 586 - DNA, 14 bp, Artificial Sequence
  Synthetic Oligonucleotide
tgcagcttgccagg

SEQ ID NO: 587 - DNA, 14 bp, Artificial Sequence
  Synthetic Oligonucleotide
aagagtgcagcttg

SEQ ID NO: 588 - DNA, 14 bp, Artificial Sequence
  Synthetic Oligonucleotide
tcagcaagagtgca

SEQ ID NO: 589 - DNA, 14 bp, Artificial Sequence
  Synthetic Oligonucleotide
gggactcagcaaga

SEQ ID NO: 590 - DNA, 14 bp, Artificial Sequence
  Synthetic Oligonucleotide
tgcccaggacggcc

SEQ ID NO: 591 - DNA, 14 bp, Artificial Sequence
  Synthetic Oligonucleotide
tgcctgagtgcccc

SEQ ID NO: 592 - DNA, 14 bp, Artificial Sequence
  Synthetic Oligonucleotide
tccatggacactaa

SEQ ID NO: 593 - DNA, 14 bp, Artificial Sequence
  Synthetic Oligonucleotide
ggtcagctggtctg

SEQ ID NO: 594 - DNA, 14 bp, Artificial Sequence
  Synthetic Oligonucleotide
gggcccgccactgg

SEQ ID NO: 595 - DNA, 14 bp, Artificial Sequence
  Synthetic Oligonucleotide
ctcggacaaaagag

SEQ ID NO: 596 - DNA, 14 bp, Artificial Sequence
  Synthetic Oligonucleotide
atttcccattggca

SEQ ID NO: 597 - DNA, 14 bp, Artificial Sequence
  Synthetic Oligonucleotide
gctggagctgagcc

SEQ ID NO: 598 - DNA, 14 bp, Artificial Sequence
  Synthetic Oligonucleotide
tggccagtggcctc

SEQ ID NO: 599 - DNA, 14 bp, Artificial Sequence
  Synthetic Oligonucleotide
gctcatggcagaca

SEQ ID NO: 600 - DNA, 14 bp, Artificial Sequence
  Synthetic Oligonucleotide
tggagagaccaatg

SEQ ID NO: 601 - DNA, 14 bp, Artificial Sequence
  Synthetic Oligonucleotide
tggtgtgcacacat

SEQ ID NO: 602 - DNA, 14 bp, Artificial Sequence
  Synthetic Oligonucleotide
tggccactgcctca

SEQ ID NO: 603 - DNA, 14 bp, Artificial Sequence
  Synthetic Oligonucleotide
tgccgtagtggccg

SEQ ID NO: 604 - DNA, 14 bp, Artificial Sequence
  Synthetic Oligonucleotide
cttggccagtgtgg

SEQ ID NO: 605 - DNA, 14 bp, Artificial Sequence
  Synthetic Oligonucleotide
acaggccaggctgg

SEQ ID NO: 606 - DNA, 14 bp, Artificial Sequence
  Synthetic Oligonucleotide
aggtgacccgtgag

SEQ ID NO: 607 - DNA, 14 bp, Artificial Sequence
  Synthetic Oligonucleotide
cctgtgagtgtgag

SEQ ID NO: 608 - DNA, 14 bp, Artificial Sequence
  Synthetic Oligonucleotide
ggtccccatcactg

SEQ ID NO: 609 - DNA, 14 bp, Artificial Sequence
  Synthetic Oligonucleotide
ggacctgccatgga

SEQ ID NO: 610 - DNA, 14 bp, Artificial Sequence
  Synthetic Oligonucleotide
caacaggacctgcc

SEQ ID NO: 611 - DNA, 14 bp, Artificial Sequence
  Synthetic Oligonucleotide
gtgctcgcccagca

SEQ ID NO: 612 - DNA, 23 bp, Artificial Sequence
  Primer
aatgaggaacagtgctcctttga

SEQ ID NO: 613 - DNA, 25 bp, Artificial Sequence
  Primer
tgtaaacaatccagaactgcttggt

SEQ ID NO: 614 - DNA, 22 bp, Artificial Sequence
  Probe
cccgggagatcttcaagagccc

(19) United States
(12) Patent Application Publication
(10) Pub. No.: US 2012/0214862 A1
(43) Pub. Date: Aug. 23, 2012

(54) MODULATION OF FACTOR 7 EXPRESSION

(75) Inventors: Susan M. Freier, San Diego, CA (US); Brett P. Monia, Encinitas, CA (US); Hong Zhang, Fremont, CA (US); Jeffrey R. Crosby, Carlsbad, CA (US); Chenguang Zhao, San Diego, CA (US)

(73) Assignee: ISIS Pharmaceuticals, Inc., Carlsbad, CA (US)

(21) Appl. No.: 13/384,327

(22) PCT Filed: Jul. 15, 2010
(86) PCT No.: PCT/US10/42187

§ 371 Date: May 2, 2012

Related U.S. Application Data
(60) Provisional application No. 61/226,253, filed on Jul. 16, 2009.

(51) Int. Cl. A61K 31/712

(52) U.S. Cl. 514/44 A

(57) ABSTRACT

Disclosed herein are antisense compounds and methods for decreasing Factor 7 and treating or preventing thromboembolic complications in an individual in need thereof. Examples of disease conditions that can be ameliorated with the administration of antisense compounds targeted to Factor 7 include thrombosis, embolism, and thromboembolism, such as, deep vein thrombosis, pulmonary embolism, myocardial infarction, and stroke. Antisense compounds targeting Factor 7 can also be used as a prophylactic treatment to prevent individuals at risk for thrombosis and embolism.

### RELATED APPLICATIONS

This application claims the benefit of the priority date of U.S. Provisional Application No. 61/226,253 filed Jul. 16, 2009, which is hereby incorporated by reference in its entirety, including the Sequence Listing submitted therewith, where permitted.

### SEQUENCE LISTING

The present application is being filed along with a Sequence Listing in electronic format. The Sequence Listing is provided as a file entitled ISIS_{—}119VPC_SEQ.txt created Jul. 13, 2010, which is 177 KB in size. The information in the electronic format of the sequence listing is incorporated herein by reference in its entirety.

### FIELD OF THE INVENTION

Embodiments of the present invention provide methods, compounds, and compositions for reducing expression of Factor 7 mRNA and protein in an animal. Such methods, compounds, and compositions are useful to treat, prevent, or ameliorate thromboembolic complications.

### BACKGROUND OF THE INVENTION

The circulatory system requires mechanisms that prevent blood loss, as well as those that counteract inappropriate intravascular obstructions. Generally, coagulation comprises a cascade of reactions culminating in the conversion of soluble fibrinogen to an insoluble fibrin gel. The steps of the cascade involve the conversion of an inactive zymogen to an activated enzyme. The active enzyme then catalyzes the next step in the cascade.

### Coagulation Cascade

The coagulation cascade may be initiated through two branches, the tissue factor pathway (also “extrinsic pathway”), which is the primary pathway, and the contact activation pathway (also “intrinsic pathway”).

The tissue factor pathway is initiated by the cell surface receptor tissue factor (TF, also referred to as factor III), which is expressed constitutively by extravascular cells (pericytes, cardiomyocytes, smooth muscle cells, and keratinocytes) and expressed by vascular monocytes and endothelial cells upon induction by inflammatory cytokines or endotoxin. (Drake et al., Am J Pathol 1989, 134:1087-1097). TF is the high affinity cellular receptor for coagulation factor VIIa, a serine protease. In the absence of TF, VIIa has very low catalytic activity, and binding to TF is necessary to render VIIa functional through an allosteric mechanism. (Drake et al., Am J Pathol 1989, 134:1087-1097). The TF-VIIa complex activates factor X to Xa. Xa in turn associates with its co-factor factor Va into a prothrombinase complex which in turn activates prothrombin, (also known as factor II or factor 2) to thrombin (also known as factor IIa, or factor 2a). Thrombin activates platelets, converts fibrinogen to fibrin and promotes fibrin cross-linking by activating factor XIII, thus forming a stable plug at sites where TF is exposed on extravascular cells. In addition, thrombin reinforces the coagulation cascade response by activating factors V and VIII.

The contact activation pathway is triggered by activation of factor XII to XIIa. Factor XIIa converts XI to XIa, and XIa converts IX to IXa. IXa associates with its cofactor VIIIa to convert X to Xa. The two pathways converge at this point as factor Xa associates factor Va to activate prothrombin (factor II) to thrombin (factor IIa).

### Inhibition of Coagulation

At least three mechanisms keep the coagulation cascade in check, namely the action of activated protein C, antithrombin, and tissue factor pathway inhibitor. Activated protein C is a serine protease that degrades cofactors Va and VIIIa Protein C is activated by thrombin with thrombomodulin, and requires coenzyme Protein S to function. Antithrombin is a serine protease inhibitor (serpin) that inhibits serine proteases: thrombin, Xa, XIIa, XIa and IXa. Tissue factor pathway inhibitor inhibits the action of Xa and the TF-VIIa complex. (Schwartz A L et al., Trends Cardiovasc Med. 1997; 7:234-239.)

### Disease

Thrombosis is the pathological development of blood clots, and an embolism occurs when a blood clot migrates to another part of the body and interferes with organ function. Thromboembolism may cause conditions such as deep vein thrombosis, pulmonary embolism, myocardial infarction, and stroke. Significantly, thromboembolism is a major cause of morbidity affecting over 2 million Americans every year. (Adcock et al. American Journal of Clinical Pathology. 1997; 108:434-49). While most cases of thrombosis are due to acquired extrinsic problems, for example, surgery, cancer, immobility, some cases are due to a genetic predisposition, for example, antiphospholipid syndrome and the autosomal dominant condition, Factor V Leiden. (Bertina R M et al. Nature 1994; 369:64-67.)

### Treatment

The most commonly used anticoagulants, warfarin, heparin, and low molecular weight heparin (LMWH) all possess significant drawbacks.

Warfarin is typically used to treat patients suffering from atrial fibrillation. The drug interacts with vitamin K-dependent coagulation factors which include factors II, VII, IX and X. Anticoagulant proteins C and S are also inhibited by warfarin. Drug therapy using warfarin is further complicated by the fact that warfarin interacts with other medications, including drugs used to treat atrial fibrillation, such as amiodarone. Because therapy with warfarin is difficult to predict, patients must be carefully monitored in order to detect any signs of anomalous bleeding.

Heparin functions by activating antithrombin which inhibits both thrombin and factor X. (Bjork I, Lindahl U. Mol Cell Biochem. 1982 48: 161-182.) Treatment with heparin may cause an immunological reaction that makes platelets aggregate within blood vessels that can lead to thrombosis. This side effect is known as heparin-induced thrombocytopenia (HIT) and requires patient monitoring. Prolonged treatment with heparin may also lead to osteoporosis. LMWH can also inhibit Factor 2, but to a lesser degree than unfractioned heparin (UFH). LMWH has been implicated in the development of HIT.

Thus, current anticoagulant agents lack predictability and specificity and, therefore, require careful patient monitoring to prevent adverse side effects, such as bleeding complications. There are currently no anticoagulants which target only the intrinsic or extrinsic pathway.

### SUMMARY OF THE INVENTION

Provided herein are methods, compounds, and compositions for modulating expression of Factor 7 mRNA and protein. In certain embodiments, Factor 7 specific inhibitors modulate expression of Factor 7 mRNA and protein. In certain embodiments, Factor 7 specific inhibitors are nucleic acids, proteins, or small molecules.

In certain embodiments, modulation can occur in a cell or tissue. In certain embodiments, the cell or tissue is in an animal. In certain embodiments, the animal is a human. In certain embodiments, Factor 7 mRNA levels are reduced. In certain embodiments, Factor 7 protein levels are reduced. Such reduction can occur in a time-dependent manner or in a dose-dependent manner.

Also provided are methods, compounds, and compositions useful for preventing, treating, and ameliorating diseases, disorders, and conditions. In certain embodiments, such diseases, disorders, and conditions are thromboembolic complications. Such thromboembolic complications include the categories of thrombosis, embolism, and thromboembolism. In certain embodiments such thromboembolic complications include deep vein thrombosis, pulmonary embolism, myocardial infarction, and stroke.

Such diseases, disorders, and conditions can have one or more risk factors, causes, or outcomes in common. Certain risk factors and causes for development of a thromboembolic complication include immobility, surgery (particularly orthopedic surgery), malignancy, pregnancy, older age, use of oral contraceptives, atrial fibrillation, previous thromboembolic complication, chronic inflammatory disease, and inherited or acquired prothrombotic clotting disorders. Certain outcomes associated with development of a thromboembolic complication include decreased blood flow through an affected vessel, death of tissue, and death.

In certain embodiments, methods of treatment include administering a Factor 7 specific inhibitor to an individual in need thereof.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“Active pharmaceutical agent” means the substance or substances in a pharmaceutical composition that provide a therapeutic benefit when administered to an individual. For example, in certain embodiments an antisense oligonucleotide targeted to Factor 7 is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Amelioration” refers to a lessening of at least one indicator, sign, or symptom of an associated disease, disorder, or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antidote compound” refers to a compound capable decreasing the intensity or duration of any antisense activity.

“Antidote oligonucleotide” means an antidote compound comprising an oligonucleotide that is complementary to and capable of hybridizing with an antisense compound.

“Antidote protein” means an antidote compound comprising a peptide.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Coagulation factor” means any of factors I, II, III, IV, V, VII, VIII, IX, X, XI, XII, or XIII in the blood coagulation cascade. “Coagulation factor nucleic acid” means any nucleic acid encoding a coagulation factor. For example, in certain embodiments, a coagulation factor nucleic acid includes, without limitation, a DNA sequence encoding a coagulation factor (including genomic DNA comprising introns and exons), an RNA sequence transcribed from DNA encoding a coagulation factor, and an mRNA sequence encoding a coagulation factor. “Coagulation factor mRNA” means an mRNA encoding a coagulation factor protein.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, the diluent in an injected composition may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Factor 7 nucleic acid” or “Factor VII nucleic acid” means any nucleic acid encoding Factor 7. For example, in certain embodiments, a Factor 7 nucleic acid includes a DNA sequence encoding Factor 7, an RNA sequence transcribed from DNA encoding Factor 7 (including genomic DNA comprising introns and exons), and an mRNA sequence encoding Factor 7. “Factor 7 mRNA” means an mRNA encoding a Factor 7 protein.

“Factor 7 specific inhibitor” refers to any agent capable of specifically inhibiting the expression of Factor 7 mRNA and/or Factor 7 protein at the molecular level. For example, Factor 7 specific inhibitors include nucleic acids (including antisense compounds), peptides, antibodies, small molecules, and other agents capable of inhibiting the expression of Factor 7 mRNA and/or Factor 7 protein. In certain embodiments, by specifically modulating Factor 7 mRNA expression and/or Factor 7 protein expression, Factor 7 specific inhibitors may affect other components of the coagulation cascade including downstream components. Similarly, in certain embodiments, Factor 7 specific inhibitors may affect other molecular processes in an animal.

“Factor 7 specific inhibitor antidote” means a compound capable of decreasing the effect of a Factor 7 specific inhibitor. In certain embodiments, a Factor 7 specific inhibitor antidote is selected from a Factor 7 peptide; a Factor 7 antidote oligonucleotide, including a Factor 7 antidote compound complementary to a Factor 7 antisense compound; and any compound or protein that affects the intrinsic or extrinsic coagulation pathway.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap segment” and the external regions may be referred to as “wing segments.”

“Gap-widened” means a chimeric antisense compound having a gap segment of 12 or more contiguous 2′-deoxyribonucleosides positioned between and immediately adjacent to 5′ and 3′ wing segments having from one to six nucleosides.

“Hybridization” means the annealing of complementary nucleic acid molecules. In certain embodiments, complementary nucleic acid molecules include an antisense compound and a target nucleic acid.

“Identifying an animal at risk for thromboembolic complications” means identifying an animal having been diagnosed with a thromboembolic complication or identifying an animal predisposed to develop a thromboembolic complication. Individuals predisposed to develop a thromboembolic complication include those having one or more risk factors for thromboembolic complications including immobility, surgery (particularly orthopedic surgery), malignancy, pregnancy, older age, use of oral contraceptives, and inherited or acquired prothrombotic clotting disorders. Such identification may be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Internucleoside linkage” refers to the chemical bond between nucleosides.

“Linked nucleosides” means adjacent nucleosides which are bonded together.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising a modified internucleoside linkage, a modified sugar, or a modified nucleobase.

“Modified sugar” refers to a substitution or change from a natural sugar.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleotide” means a nucleoside having a phosphate group covalently linked to the sugar portion of the nucleoside.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition may comprise one or more active pharmaceutical agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Thromboembolic complication” means any disease, disorder, or condition involving an embolism caused by a thrombus. Examples of such diseases, disorders, and conditions include the categories of thrombosis, embolism, and thromboembolism. In certain embodiments, such disease disorders, and conditions include deep vein thrombosis, pulmonary embolism, myocardial infarction, and stroke.

“Treat” refers to administering a pharmaceutical composition to effect an alteration or improvement of a disease, disorder, or condition.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### CERTAIN EMBODIMENTS

Embodiments of the present invention provide methods, compounds, and compositions for decreasing Factor 7 mRNA and protein expression.

Embodiments of the present invention provide methods, compounds, and compositions for the treatment, prevention, or amelioration of diseases, disorders, and conditions associated with Factor 7 in an individual in need thereof. Also contemplated are methods and compounds for the preparation of a medicament for the treatment, prevention, or amelioration of a disease, disorder, or condition associated with Factor 7. Factor 7 associated diseases, disorders, and conditions include thromboembolic complications such as thrombosis, embolism, thromboembolism, deep vein thrombosis, pulmonary embolism, myocardial infarction, and stroke.

Embodiments of the present invention provide a Factor 7 specific inhibitor for use in treating, preventing, or ameliorating a Factor 7 associated disease. In certain embodiments, Factor 7 specific inhibitors are nucleic acids (including antisense compounds), peptides, antibodies, small molecules, and other agents capable of inhibiting the expression of Factor 7 mRNA and/or Factor 7 protein.

In certain embodiments of the present invention, Factor 7 specific inhibitors are peptides or proteins, such as, but not limited to, E-56 peptide (Ac-ALCDDPRVDRWYCQFVEG-NH₂) (Nature. 2000 Mar. 30; 404(6777):465-70) and peptide A-183 (EEWEVLCWTWETCER) (Biochemistry. 2001 Aug. 14; 40(32):9513-21).

In certain embodiments of the present invention, Factor 7 specific inhibitors are antibodies, such as, but not limited to 12D10 neutralizing monoclonal antibody (Thromb Haemost. 1995 February; 73(2):223-30); hVII-B101/B1, hVII-DC2/D4, and hVII-DC6/3D8 monoclonal antibodies (Thromb Haemost. 1998 January; 79(1):104-9); C6 monoclonal antibody (Biochemistry. 1996 Oct. 29; 35(43):13826-32); CLB-CAg A monoclonal antibody (1994) J. Biol. Chem. 269, 7150-7155); MC1476 and MC1839 monoclonal antibodies (J Clin Invest. 1985 September; 76(3):937-46); and anti-hFVII Ab, polyclonal antibody (J Surg Res. 2003 September; 114(1):37-41).

In certain embodiments of the present invention, Factor 7 specific inhibitors are small molecules, such as, but not limited to TGF-beta and nitric oxide (Biochem Biophys Res Commun. 2004 Aug. 27; 321(3):688-94), Nafamostat mesilate (Thromb Res. 1994 Apr. 15; 74(2):155-61), and 2-aryl substituted 4H-3,1-benzoxazin-4-ones (Bioorg Med. Chem. 2000 August; 8(8):2095-103).

Embodiments of the present invention provide a Factor 7 specific inhibitor, as described herein, for use in treating, preventing, or ameliorating thromboembolic complications such as thrombosis, embolism, thromboembolism, deep vein thrombosis, pulmonary embolism, myocardial infarction, and stroke.

Embodiments of the present invention provide the use of Factor 7 specific inhibitors as described herein in the manufacture of a medicament for treating, ameliorating, or preventing a thromboembolic complication such as thrombosis, embolism, thromboembolism, deep vein thrombosis, pulmonary embolism, myocardial infarction, and stroke.

Embodiments of the present invention provide a Factor 7 specific inhibitor as described herein for use in treating, preventing, or ameliorating a thromboembolic complication as described herein by combination therapy with an additional agent or therapy as described herein. Agents or therapies can be co-administered or administered concomitantly.

Embodiments of the present invention provide the use of a Factor 7 specific inhibitor as described herein in the manufacture of a medicament for treating, preventing, or ameliorating a thromboembolic complication as described herein by combination therapy with an additional agent or therapy as described herein. Agents or therapies can be co-administered or administered concomitantly.

Embodiments of the present invention provide the use of a Factor 7 specific inhibitor as described herein in the manufacture of a medicament for treating, preventing, or ameliorating a thromboembolic complication as described herein in a patient who is subsequently administered an additional agent or therapy as described herein.

Embodiments of the present invention provide a kit for treating, preventing, or ameliorating a thromboembolic complication as described herein wherein the kit comprises:

(i) a Factor 7 specific inhibitor as described herein; and alternatively

(ii) an additional agent or therapy as described herein.

A kit of the present invention may further include instructions for using the kit to treat, prevent, or ameliorate a thromboembolic complication as described herein by combination therapy as described herein.

Embodiments of the present invention provide antisense compounds targeted to a Factor 7 nucleic acid. In certain embodiments, the human Factor 7 nucleic acid is any of the sequences set forth in GENBANK Accession No. NT_{—}027140.6, truncated at 1255000 to 1273000 (incorporated herein as SEQ ID NO: 1), GENBANK Accession No. NM_{—}019616.2, (incorporated herein as SEQ ID NO: 2), GENBANK Accession No. DB184141.1 (incorporated herein as SEQ ID NO: 3), and GENBANK® Accession No. NM_{—}000131.3 (incorporated herein as SEQ ID NO: 167). In certain embodiments, the rhesus monkey Factor 7 nucleic acid is any of the sequences set forth in GENBANK Accession No NW_{—}001104507.1, truncated at nucleotides 691000 to 706000 (incorporated herein as SEQ ID NO: 162) and GENBANK Accession No. 3360_{—}061_B (incorporated herein as SEQ ID NO: 163). In certain embodiments, the murine Factor 7 nucleic acid is the sequence set forth in GENBANK Accession No. NT_{—}039455.6, truncated at nucleotides 10024000 to 10037000 (incorporated herein as SEQ ID NO: 160).

Embodiments of the present invention provide compounds comprising a modified oligonucleotide consisting of 12 to 30 linked nucleosides and having a nucleobase sequence comprising at least 12 contiguous nucleobases of a nucleobase sequence selected from among the nucleobase sequences recited in SEQ ID NOs: 4 to 159 and 168 to 611.

In certain embodiments, the compound consists of a single-stranded modified oligonucleotide.

In certain embodiments, the modified oligonucleotide consists of 20 linked nucleosides.

In certain embodiments, the nucleobase sequence of the modified oligonucleotide is 100% complementary to a nucleobase sequence of SEQ ID NO: 1, SEQ ID NO: 2, SEQ ID NO: 3, and SEQ ID NO: 167.

In certain embodiments, the compound has at least one modified internucleoside linkage. In certain embodiments, the internucleoside linkage is a phosphorothioate internucleoside linkage.

In certain embodiments, the compound has at least one nucleoside comprising a modified sugar. In certain embodiments, the at least one modified sugar is a bicyclic sugar. In certain embodiments, the at least one modified sugar comprises a 2′-O-methoxyethyl.

In certain embodiments, the compound has at least one nucleoside comprising a modified nucleobase. In certain embodiments, the modified nucleobase is a 5-methylcytosine.

In certain embodiments, the modified oligonucleotide of the compound comprises:

(i) a gap segment consisting of linked deoxynucleosides;

(ii) a 5′ wing segment consisting of linked nucleosides;

(iii) a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In certain embodiments, the modified oligonucleotide of the compound comprises:

(i) a gap segment consisting of ten linked deoxynucleosides;

(ii) a 5′ wing segment consisting of five linked nucleosides;

(iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the modified oligonucleotide of the compound comprises:

(i) a gap segment consisting of fourteen linked deoxynucleosides;

(ii) a 5′ wing segment consisting of three linked nucleosides;

(iii) a 3′ wing segment consisting of three linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the modified oligonucleotide of the compound comprises:

(i) a gap segment consisting of thirteen linked deoxynucleosides;

(ii) a 5′ wing segment consisting of two linked nucleosides;

(iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

Embodiments of the present invention provide a composition comprising a modified oligonucleotide consisting of 12 to 30 linked nucleosides and having a nucleobase sequence comprising at least 12 contiguous nucleobases of a nucleobase sequence selected from among the nucleobase sequences recited in SEQ ID NOs: 4 to 159 and 168 to 611 or a salt thereof and a pharmaceutically acceptable carrier or diluent.

Embodiments of the present invention provide methods comprising administering to an animal a compound comprising a modified oligonucleotide consisting of 12 to 30 linked nucleosides and having a nucleobase sequence comprising at least 8 contiguous nucleobases of a nucleobase sequence selected from among the nucleobase sequences recited in SEQ ID NOs: 4 to 159 and 168 to 611.

In certain embodiments, the animal is a human.

In certain embodiments, the administering prevents deep vein thrombosis or pulmonary embolism.

In certain embodiments, the compound is co-administered with any of the group selected from aspirin, clopidogrel, dipyridamole, heparin, lepirudin, ticlopidine, warfarin, apixaban, rivaroxaban, and lovenox.

In certain embodiments, the compound is co-administered with any Factor Xa inhibitor.

In certain embodiment, the Factor Xa inhibitor is any of Rivaroxaban, LY517717, YM150, apixaban, PRT054021, and DU-176b.

In certain embodiments, the compound is administered concomitantly with any of the group selected from aspirin, clopidogrel, dipyridamole, heparin, lepirudin, ticlopidine, warfarin, apixaban, rivaroxaban, and lovenox are administered concomitantly.

In certain embodiments, the administering is parenteral administration. In certain embodiments, the parenteral administration is any of subcutaneous or intravenous administration.

Embodiments of the present invention provide methods comprising identifying an animal at risk for developing thromboembolic complications and administering to the at risk animal a therapeutically effective amount of a compound comprising a modified oligonucleotide consisting of 12 to 30 linked nucleosides, wherein the modified oligonucleotide is complementary to a Factor 7 nucleic acid.

In certain embodiments, the thromboembolic complication is deep vein thrombosis, pulmonary embolism, or a combination thereof.

Embodiments of the present invention provide methods comprising identifying an animal having a clotting disorder by administering to the animal a therapeutically effective amount of a compound comprising a modified oligonucleotide consisting of 12 to 30 linked nucleosides, wherein the modified oligonucleotide is complementary to a Factor 7 nucleic acid.

In certain embodiments, the compound is co-administered with any of the group selected from aspirin, clopidogrel, dipyridamole, heparin, lepirudin, ticlopidine, warfarin, apixaban, rivaroxaban, and lovenox.

In certain embodiments, the compound is administered concomitantly with any of the group selected from aspirin, clopidogrel, dipyridamole, heparin, lepirudin, ticlopidine, warfarin, apixaban, rivaroxaban, and lovenox are administered concomitantly.

Embodiments of the present invention provide methods comprising reducing the risk for thromboembolic complications in an animal and administering to the animal a therapeutically effective amount of a compound comprising a modified oligonucleotide consisting of 12 to 30 linked nucleosides, wherein the modified oligonucleotide is complementary to a Factor 7 nucleic acid.

Embodiments of the present invention provide methods comprising treating a clotting disorder in an animal and administering to the animal a therapeutically effective amount of a compound comprising a modified oligonucleotide consisting of 12 to 30 linked nucleosides, wherein the modified oligonucleotide is complementary to a Factor 7 nucleic acid.

Embodiments of the present invention provide methods comprising inhibiting Factor 7 expression in an animal and administering to the animal a therapeutically effective amount of a compound comprising a modified oligonucleotide consisting of 12 to 30 linked nucleosides, wherein the modified oligonucleotide is complementary to a Factor 7 nucleic acid.

In certain embodiments, the Factor 7 inhibition in the animal is reversed by administering an antidote to the modified oligonucleotide.

In certain embodiments, the antidote is an oligonucleotide complementary to the modified oligonucleotide.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a Factor 7 nucleic acid is 12 to 30 subunits in length. In other words, antisense compounds are from 12 to 30 linked subunits. In other embodiments, the antisense compound is 8 to 80, 12 to 50, 15 to 30, 18 to 24, 19 to 22, or 20 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleotides.

In certain embodiments, a shortened or truncated antisense compound targeted to a Factor 7 nucleic acid has a single subunit deleted from the 5′ end (5′ truncation), or alternatively from the 3′ end (3′ truncation). A shortened or truncated antisense compound targeted to a Factor 7 nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a Factor 7 nucleic acid have chemically modified subunits arranged in patterns, or motifs, to confer to the antisense compounds properties such as enhanced the inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers of the present invention include, but are not limited to, for example 5-10-5, 4-8-4, 4-12-3, 4-12-4, 3-14-3, 2-13-5, 2-16-2, 1-18-1, 3-10-3, 2-10-2, 1-10-1 or 2-8-2.

In certain embodiments, the antisense compound as a “wingmer” motif, having a wing-gap or gap-wing configuration, i.e. an X-Y or Y-Z configuration as described above for the gapmer configuration. Thus, wingmer configurations of the present invention include, but are not limited to, for example 5-10, 8-4, 4-12, 12-4, 3-14, 16-2, 18-1, 10-3, 2-10, 1-10, 8-2, 2-13, or 5-13.

In certain embodiments, antisense compounds targeted to a Factor 7 nucleic acid possess a 5-10-5 gapmer motif.

In certain embodiments, antisense compounds targeted to a Factor 7 nucleic acid possess a 3-14-3 gapmer motif.

In certain embodiments, antisense compounds targeted to a Factor 7 nucleic acid possess a 2-13-5 gapmer motif.

In certain embodiments, an antisense compound targeted to a Factor 7 nucleic acid has a gap-widened motif.

In certain embodiments, a gap-widened antisense oligonucleotide targeted to a Factor 7 nucleic acid has a gap segment of fourteen 2′-deoxyribonucleotides positioned immediately adjacent to and between wing segments of three chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to a Factor 7 nucleic acid has a gap segment of thirteen 2′-deoxyribonucleotides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

Embodiments of the present invention provide antisense compounds targeted to a Factor 7 nucleic acid. In certain embodiments, the human Factor 7 nucleic acid is any of the sequences set forth in GENBANK Accession No. NT_{—}027140.6, truncated at 1255000 to 1273000 (incorporated herein as SEQ ID NO: 1), GENBANK Accession No. NM_{—}019616.2, (incorporated herein as SEQ ID NO: 2), GENBANK Accession No. DB184141.1 (incorporated herein as SEQ ID NO: 3), and GENBANK® Accession No. NM_{—}000131.3 (incorporated herein as SEQ ID NO: 167). In certain embodiments, the rhesus monkey Factor 7 nucleic acid is any of the sequences set forth in GENBANK Accession No NW_{—}001104507.1, truncated at nucleotides 691000 to 706000 (incorporated herein as SEQ ID NO: 162) and GENBANK Accession No. 3360_{—}061_B (incorporated herein as SEQ ID NO: 163). In certain embodiments, the murine Factor 7 nucleic acid is the sequence set forth in GENBANK Accession No. NT_{—}039455.6, truncated at nucleotides 10024000 to 10037000 (incorporated herein as SEQ ID NO: 160).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for Factor 7 can be obtained by accession number from sequence databases such as NCBI and such information is incorporated herein by reference. In certain embodiments, a target region may encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in Factor 7 mRNA levels are indicative of inhibition of Factor 7 expression. Reductions in levels of a Factor 7 protein are also indicative of inhibition of target mRNA expression. Further, phenotypic changes are indicative of inhibition of Factor 7 expression. For example, a prolonged aPTT time can be indicative of inhibition of Factor 7 expression. In another example, prolonged aPTT time in conjunction with a normal PT time can be indicative of inhibition of Factor 7 expression. In another example, a decreased quantity of Platelet Factor 4 (PF-4) can be indicative of inhibition of Factor 7 expression. In another example, reduced formation of thrombus or increased time for thrombus formation can be indicative of inhibition of Factor 7 expression.

### Hybridization

In some embodiments, hybridization occurs between an antisense compound disclosed herein and a Factor 7 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a Factor 7 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a Factor 7 nucleic acid).

Non-complementary nucleobases between an antisense compound and a Factor 7 nucleic acid may be tolerated provided that the antisense compound remains able to specifically hybridize to a target nucleic acid. Moreover, an antisense compound may hybridize over one or more segments of a Factor 7 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a Factor 7 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound may be fully complementary to a Factor 7 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a Factor 7 nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a Factor 7 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### MODIFICATIONS

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a Factor 7 nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity or some other beneficial biological property to the antisense compounds. In certain embodiments, nucleosides comprise a chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R1)(R)2 (R═H, C1-C12 alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH3 and 2′-O(CH2)2OCH3 substituent groups. The substituent at the 2′ position can also be selected from allyl, amino, azido, thio, O-allyl, O—C1-C10 alkyl, OCF3, O(CH2)2SCH3, O(CH2)2-O—N(Rm)(Rn), and O—CH2-C(═O)—N(Rm)(Rn), where each Rm and Rn is, independently, H or substituted or unsubstituted C1-C10 alkyl.

Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH2)-O-2′ (LNA); 4′-(CH2)-S-2′; 4′-(CH2)-O-2′ (LNA); 4′-(CH2)2-O-2′ (ENA); 4′-C(CH3)2-O-2′ (see PCT/US2008/068922); 4′-CH(CH3) [symbol] -O-2′ and 4′-C [symbol] H(CH2OCH3) [symbol] -O-2′ (see U.S. Pat. No. 7,399,845, issued on Jul. 15, 2008); 4′-CH2-N(OCH3)-2′ (see PCT/US2008/064591); 4′-CH2-O—N(CH3)-2′ (see published U.S. Patent Application US2004-0171570, published Sep. 2, 2004); 4′-CH2-N(R)—O-2′ (see U.S. Pat. No. 7,427,672, issued on Sep. 23, 2008); 4′-CH2-C(CH3)-2′ and 4′-CH2-C [symbol] (═CH2)-2′ (see PCT/US2008/066154); and wherein R is, independently, H, C1-C12 alkyl, or a protecting group. Each of the foregoing BNAs include various stereochemical sugar configurations including for example α-L-ribofuranose and β-D-ribofuranose (see PCT international application PCT/DK98/00393, published on Mar. 25, 1999 as WO 99/14226).

In certain embodiments, nucleosides are modified by replacement of the ribosyl ring with a sugar surrogate. Such modification includes without limitation, replacement of the ribosyl ring with a surrogate ring system (sometimes referred to as DNA analogs) such as a morpholino ring, a cyclohexenyl ring, a cyclohexyl ring or a tetrahydropyranyl ring such as one having one of the formula:

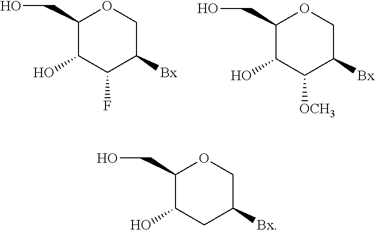

Many other bicyclo and tricyclo sugar surrogate ring systems are also know in the art that can be used to modify nucleosides for incorporation into antisense compounds (see for example review article: Leumann, Christian J.). Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds targeted to a Factor 7 nucleic acid comprise one or more nucleotides having modified sugar moieties. In certain embodiments, the modified sugar moiety is 2′-MOE. In certain embodiments, the 2′-MOE modified nucleotides are arranged in a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a Factor 7 nucleic acid comprise one or more modified nucleobases. In certain embodiments, gap-widened antisense oligonucleotides targeted to a Factor 7 nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides may be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compound targeted to a Factor 7 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a Factor 7 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of Factor 7 nucleic acids can be tested in vitro in a variety of cell types. Cell types used for such analyses are available from commercial vendors (e.g. American Type Culture Collection, Manassus, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE® (Invitrogen, Carlsbad, Calif.). Antisense oligonucleotide is mixed with LIPOFECTAMINE® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE®. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of a Factor 7 nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM® 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT, real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN® (Invitrogen, Inc. Carlsbad, Calif.). Cyclophilin A expression is quantified by real time PCR, by being run simultaneously with the target, multiplexing, or separately. Total RNA is quantified using RIBOGREEN® RNA quantification reagent (Invetrogen, Inc. Eugene, Oreg.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a Factor 7 nucleic acid. Methods for designing real-time PCR probes and primers are well known in the art, and may include the use of software such as PRIMER EXPRESS® Software (Applied Biosystems, Foster City, Calif.).

### Analysis of Protein Levels

Antisense inhibition of Factor 7 nucleic acids can be assessed by measuring Factor 7 protein levels. Protein levels of Factor 7 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of human and rat Factor 7 are commercially available.

### In Vivo Testing of Antisense Compounds

Antisense compounds, for example, antisense oligonucleotides, are tested in animals to assess their ability to inhibit expression of Factor 7 and produce phenotypic changes, such as, prolonged aPTT, prolonged aPTT time in conjunction with a normal PT, decreased quantity of Platelet Factor 4 (PF-4), and reduced formation of thrombus or increased time for thrombus formation. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from liver tissue and changes in Factor 7 nucleic acid expression are measured. Changes in Factor 7 protein levels are also measured using a thrombin generation assay. In addition, effects on clot times, e.g. PT and aPTT, are determined using plasma from treated animals.

### Certain Indications

In certain embodiments, the invention provides methods of treating an individual comprising administering one or more pharmaceutical compositions of the present invention. In certain embodiments, the individual has a thromboembolic complication. In certain embodiments, the individual is at risk for a blood clotting disorder, including, but not limited to, infarct, thrombosis, embolism, thromboembolism such as deep vein thrombosis, pulmonary embolism, myocardial infarction, and stroke. This includes individuals with an acquired problem, disease, or disorder that leads to a risk of thrombosis, for example, surgery, cancer, immobility, sepsis, atherosclerosis atrial fibrillation, as well as genetic predisposition, for example, antiphospholipid syndrome and the autosomal dominant condition, Factor V Leiden. In certain embodiments, the individual has been identified as in need of anti-coagulation therapy. Examples of such individuals include, but are not limited to, those undergoing major orthopedic surgery (e.g., hip/knee replacement or hip fracture surgery) and patients in need of chronic treatment, such as those suffering from arterial fibrillation to prevent stroke. In certain embodiments the invention provides methods for prophylactically reducing Factor 7 expression in an individual. Certain embodiments include treating an individual in need thereof by administering to an individual a therapeutically effective amount of an antisense compound targeted to a Factor 7 nucleic acid.

In one embodiment, administration of a therapeutically effective amount of an antisense compound targeted to a Factor 7 nucleic acid is accompanied by monitoring of Factor 7 levels in the serum of an individual, to determine an individual's response to administration of the antisense compound. An individual's response to administration of the antisense compound is used by a physician to determine the amount and duration of therapeutic intervention.

In certain embodiments, administration of an antisense compound targeted to a Factor 7 nucleic acid results in reduction of Factor 7 expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a Factor 7 nucleic acid results in a change in a measure of blood clotting as measured by a standard test, for example, but not limited to, activated partial thromboplastin time (aPTT) test, prothrombin time (PT) test, thrombin time (TCT), bleeding time, or D-dimer. In certain embodiments, administration of a Factor 7 antisense compound increases the measure by at least 15, 20, 25, 30, 35, 40, 45, 50, 55, 60, 65, 70, 75, 80, 85, 90, 95 or 99%, or a range defined by any two of these values. In some embodiments, administration of a Factor 7 antisense compound decreases the measure by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to Factor 7 are used for the preparation of a medicament for treating a patient suffering or susceptible to a thromboembolic complication.

### Certain Combination Therapies

In certain embodiments, one or more pharmaceutical compositions of the present invention are co-administered with one or more other pharmaceutical agents. In certain embodiments, such one or more other pharmaceutical agents are designed to treat the same disease, disorder, or condition as the one or more pharmaceutical compositions of the present invention. In certain embodiments, such one or more other pharmaceutical agents are designed to treat a different disease, disorder, or condition as the one or more pharmaceutical compositions of the present invention. In certain embodiments, such one or more other pharmaceutical agents are designed to treat an undesired side effect of one or more pharmaceutical compositions of the present invention. In certain embodiments, one or more pharmaceutical compositions of the present invention are co-administered with another pharmaceutical agent to treat an undesired effect of that other pharmaceutical agent. In certain embodiments, one or more pharmaceutical compositions of the present invention are co-administered with another pharmaceutical agent to produce a combinational effect. In certain embodiments, one or more pharmaceutical compositions of the present invention are co-administered with another pharmaceutical agent to produce a synergistic effect.

In certain embodiments, one or more pharmaceutical compositions of the present invention and one or more other pharmaceutical agents are administered at the same time. In certain embodiments, one or more pharmaceutical compositions of the present invention and one or more other pharmaceutical agents are administered at different times. In certain embodiments, one or more pharmaceutical compositions of the present invention and one or more other pharmaceutical agents are prepared together in a single formulation. In certain embodiments, one or more pharmaceutical compositions of the present invention and one or more other pharmaceutical agents are prepared separately.

In certain embodiments, pharmaceutical agents that may be co-administered with a pharmaceutical composition of the present invention include anticoagulant or antiplatelet agents. In certain embodiments, pharmaceutical agents that may be co-administered with a pharmaceutical composition of the present invention include NSAID/Cyclooxygenase inhibitors, such as, aspirin. In certain embodiments, pharmaceutical agents that may be co-administered with a pharmaceutical composition of the present invention include adenosine diphosphate (ADP) receptor inhibitors, such as, clopidogrel (Plavix) and ticlopidine (Ticlid). In certain embodiments, pharmaceutical agents that may be co-administered with a pharmaceutical composition of the present invention include phosphodiesterase inhibitors, such as, cilostazol (Pletal). In certain embodiments, pharmaceutical agents that may be co-administered with a pharmaceutical composition of the present invention include, glycoprotein IIB/IIIA inhibitors, such as, abciximab (ReoPro), eptifibatide (Integrilin), tirofiban (Aggrastat), and defibrotide. In certain embodiments, pharmaceutical agents that may be co-administered with a pharmaceutical composition of the present invention include, adenosine reuptake inhibitors, such as, to dipyridamole (Persantine). In certain embodiments, pharmaceutical agents that may be co-administered with a pharmaceutical composition of the present invention include, but are not limited to warfarin (and related coumarins), heparin, direct thrombin inhibitors (such as lepirudin, bivalirudin), apixaban, lovenox, and small molecular compounds that interfere directly with the enzymatic action of particular coagulation factors (e.g. rivaroxaban, which interferes with Factor Xa). In certain embodiments, pharmaceutical agents that may be co-administered with a Factor 7 specific inhibitor of the present invention include, but are not limited to, an additional Factor 7 inhibitor. In certain embodiments, the anticoagulant or antiplatelet agent is administered prior to administration of a pharmaceutical composition of the present invention. In certain embodiments, the anticoagulant or antiplatelet agent is administered following administration of a pharmaceutical composition of the present invention. In certain embodiments the anticoagulant or antiplatelet agent is administered at the same time as a pharmaceutical composition of the present invention. In certain embodiments the dose of a co-administered anticoagulant or antiplatelet agent is the same as the dose that would be administered if the anticoagulant or antiplatelet agent was administered alone. In certain embodiments the dose of a co-administered anticoagulant or antiplatelet agent is lower than the dose that would be administered if the anticoagulant or antiplatelet agent was administered alone. In certain embodiments the dose of a co-administered anticoagulant or antiplatelet agent is greater than the dose that would be administered if the anticoagulant or antiplatelet agent was administered alone.

In certain embodiments, the co-administration of a second compound enhances the anticoagulant effect of a first compound, such that co-administration of the compounds results in an anticoagulant effect that is greater than the effect of administering the first compound alone. In other embodiments, the co-administration results in anticoagulant effects that are additive of the effects of the compounds when administered alone. In certain embodiments, the co-administration results in anticoagulant effects that are supra-additive of the effects of the compounds when administered alone. In certain embodiments, the co-administration of a second compound increases antithrombotic activity without increased bleeding risk. In certain embodiments, the first compound is an antisense compound. In certain embodiments, the second compound is an antisense compound.

In certain embodiments, an antidote is administered anytime after the administration of a Factor 7 specific inhibitor. In certain embodiments, an antidote is administered anytime after the administration of an antisense oligonucleotide targeting Factor 7. In certain embodiments, the antidote is administered minutes, hours, days, weeks, or months after the administration of an antisense compound targeting Factor 7. In certain embodiments, the antidote is a complementary (e.g. the sense strand) to the antisense compound targeting Factor 7. In certain embodiments, the antidote is a Factor 7, Factor 7a, Factor 11, or Factor 11a protein. In certain embodiments, the Factor 7, Factor 7a, Factor 11, or Factor 11a protein is a human Factor 7, human Factor 7a, human Factor 11, or human Factor 11a protein. In certain embodiments, the Factor 7 protein is NovoSeven.

### Certain Co-Administered Antiplatelet Therapies

In certain embodiments, Factor 7 inhibitors are combined with antiplatelet therapies. In certain embodiments, administration of a Factor 7 inhibitor in combination with an antiplatelet therapy results in little to no appreciable or detectable increase in risk of bleeding as compared to antiplatelet therapy alone. In certain embodiments, the risk profile or risk indications are unchanged over anti-platelet therapy alone. In certain embodiments administration of a Factor 7 inhibitor in combination with Plavix (clopidogrel) results in increased antithrombotic activity without increased bleeding risk.

The combination of antiplatelet and anticoagulant therapy is used in clinical practice most frequently in patients diagnosed with, for example, thromboembolism, atrial fibrillation, a heart valve disorder, valvular heart disease, stroke, CAD, and in patients having a mechanical valve. The benefit of dual therapy relates to the probable additive effect of suppressing both platelet and coagulation factor activities. The risk of dual therapy is the potential for increased bleeding (Dowd, M. Plenary Sessions/Thrombosis Research 123 (2008)).

Prior combinations of antiplatelet and anticoagulant therapy have been shown to increase the risk of bleeding compared with anticoagulant or antiplatelet therapy alone. Such combinations include, FXa inhibitors (e.g., apixiban and rivaroxaban) with ADP receptor/P2Y12 inhibitors (Thienopyridines such as clopidogrel—also known as Plavix) and NSAIDs (e.g., aspirin and naproxen) (Kubitza, D. et al., Br. J. Clin. Pharmacol. 63:4 (2006); Wong, P. C. et al. Journal of Thrombosis and Haemostasis 6 (2008); FDA Advisory Committee Briefing Document for New Drug Application 22-406 (2009)). For example, Wong reports that addition of certain doses of apixaban to aspirin and to aspirin plus clopidogrel produced a significant increase in bleeding time compared with aspirin alone and asprin plus clopidogrel. Kubitza reports that the combination administration of rivaroxaban and naproxen significantly increased bleeding time over naproxen alone.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of Human Factor 7 mRNA in HepB3 Cells

Antisense oligonucleotides targeted to a Factor 7 nucleic acid were tested for their effects on Factor 7 mRNA in vitro. Cultured HepB3 cells at a density of 4,000 cells per well were transfected using lipofectin reagent with 50 nM antisense oligonucleotide. After a treatment period of approximately 24 hours, RNA was isolated from the cells and Factor 7 mRNA levels were measured by real-time RT-PCR, as described herein. Factor 7 mRNA levels were adjusted according to total RNA content as measured by RIBOGREEN®. Results are presented as percent inhibition of Factor 7, relative to untreated control cells.

The chimeric antisense oligonucleotides in Table 1 were designed as 5-10-5 MOE gapmers. The gapmers are 20 nucleotides in length, wherein the central gap segment is comprised of ten 2′-deoxynucleotides and is flanked on both sides (in the 5′ and 3′ directions) by wings comprising five nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. Each gapmer listed in Table 1 is targeted to human gene sequences, SEQ ID NO: 1 (nucleotides 1255000 to 1273000 of GENBANK Accession No. NT_{—}027140.6), SEQ ID NO: 2 (GENBANK Accession No. NM_{—}019616.2), or SEQ ID NO: 3 (GENBANK Accession No. DB184141.1). “Human Target start site” indicates the 5′-most nucleotide to which the gapmer is targeted in the specified human gene sequence. “Human Target stop site” indicates the 3′-most nucleotide to which the gapmer is targeted in the specified human gene sequence.

TABLE 1

Inhibition of human Factor 7 mRNA levels by chimeric antisense
oligonucleotides having 5-10-5 MOE wings and deoxy gap
targeted to SEQ ID NO: 1, SEQ ID NO: 2, and SEQ ID NO: 3

| | | | | | | |
|---|---|---|---|---|---|---|
| | Target | | | | | |
| | SEQ ID | Target | Target | | % | SEQ ID |
| Oligo ID | NO | Start Site | Stop Site | Sequence (5′ to 3′) | Inhibition | NO |

| | | | | | | |
|---|---|---|---|---|---|---|
| 403090 | 1 | 14017 | 14036 | AGTCCTGGGTCATCAGCCGG | 69 | 4 |
| 403093 | 1 | 14231 | 14250 | GAGACCCTGGTGTACACCCC | 75 | 5 |
| 407594 | 1 | 1208 | 1227 | CCTGCAGCCAGGCAGCCCTG | 87 | 6 |
| 407595 | 1 | 2169 | 2188 | TCCTGAGGCCTTAGCGACCC | 71 | 7 |
| 407596 | 1 | 2206 | 2225 | GAGGCCCCGGCTTCCACGGC | 61 | 8 |
| 407597 | 1 | 1114 | 1133 | TGTTGACATTCCCCATGGGA | 39 | 9 |
| 407598 | 1 | 1147 | 1166 | CCATGATGAAATCTCTGCAG | 72 | 10 |
| 407599 | 1 | 1156 | 1175 | CCTGGGAGACCATGATGAAA | 73 | 11 |
| 407600 | 1 | 1190 | 1209 | TGAAGCCCAAGCAGAAGGCA | 61 | 12 |
| 407601 | 1 | 1196 | 1215 | CAGCCCTGAAGCCCAAGCAG | 52 | 13 |
| 407602 | 1 | 1207 | 1226 | CTGCAGCCAGGCAGCCCTGA | 81 | 14 |
| 407603 | 1 | 9073 | 9092 | TAAGAAATCCAGAACAGCTT | 54 | 15 |
| 407605 | 1 | 9169 | 9188 | TTGAGGCACACTGGTCCCCA | 58 | 16 |
| 407606 | 1 | 9204 | 9223 | CTGGTCCTTGCAGGAGCCCC | 89 | 17 |
| 407607 | 1 | 9209 | 9228 | TGGAGCTGGTCCTTGCAGGA | 76 | 18 |
| 407608 | 1 | 9217 | 9236 | TATAGGACTGGAGCTGGTCC | 13 | 19 |
| 407609 | 1 | 9226 | 9245 | AGAAGCAGATATAGGACTGG | 35 | 20 |
| 407610 | 1 | 9234 | 9253 | AGGGAGGCAGAAGCAGATAT | 31 | 21 |
| 407611 | 1 | 9259 | 9278 | TCTCACAGTTCCGGCCCTCG | 82 | 22 |
| 407612 | 1 | 10982 | 11001 | AGATCAGCTGGTCATCCTTG | 82 | 23 |
| 407613 | 1 | 11010 | 11029 | TGCTCACAGCCGCCGTTCTC | 66 | 24 |
| 407614 | 1 | 11018 | 11037 | TGCAGTACTGCTCACAGCCG | 78 | 25 |
| 407615 | 1 | 11088 | 11107 | GACACCCCGTCTGCCAGCAG | 65 | 26 |
| 407616 | 1 | 11093 | 11112 | TGCAGGACACCCCGTCTGCC | 64 | 27 |
| 407617 | 1 | 12084 | 12103 | TCCACATGGATATTCAACTG | 67 | 28 |
| 407618 | 1 | 12091 | 12110 | GTATTTTTCCACATGGATAT | 42 | 29 |
| 407619 | 1 | 12098 | 12117 | AGAATAGGTATTTTTCCACA | 64 | 30 |
| 407623 | 1 | 12795 | 12814 | TCCATTCACCAACAACAGGA | 69 | 31 |
| 407624 | 1 | 12842 | 12861 | GAGACCACCCAGATGGTGTT | 73 | 32 |
| 407625 | 1 | 12863 | 12882 | TTGTCGAAACAGTGGGCCGC | 100 | 33 |
| 407626 | 1 | 12871 | 12890 | TCTTGATTTTGTCGAAACAG | 82 | 34 |
| 407628 | 1 | 13777 | 13796 | TGATGACCTGCGCCACCCGC | 38 | 35 |
| 407629 | 1 | 13856 | 13875 | TCAGTGAGGACCACGGGCTG | 34 | 36 |
| 407630 | 1 | 13863 | 13882 | CACATGGTCAGTGAGGACCA | 29 | 37 |
| 407631 | 1 | 13869 | 13888 | GGGCACCACATGGTCAGTGA | 66 | 38 |
| 407632 | 1 | 13888 | 13907 | TCCGTTCGGGCAGGCAGAGG | 46 | 39 |
| 407633 | 1 | 14023 | 14042 | GCAGGCAGTCCTGGGTCATC | 71 | 40 |
| 407634 | 1 | 14032 | 14051 | GTGACTGCTGCAGGCAGTCC | 24 | 41 |
| 407635 | 1 | 14186 | 14205 | TGGCCCCAGCTGACGATGCC | 64 | 42 |
| 407636 | 1 | 14193 | 14212 | GCAGCCCTGGCCCCAGCTGA | 75 | 43 |
| 407637 | 1 | 14238 | 14257 | GTACTGGGAGACCCTGGTGT | 71 | 44 |
| 407638 | 1 | 14248 | 14267 | GCCACTCGATGTACTGGGAG | 0 | 45 |
| 407639 | 1 | 14254 | 14273 | TTTGCAGCCACTCGATGTAC | 76 | 46 |
| 407640 | 1 | 14263 | 14282 | GCATGAGCTTTTGCAGCCAC | 79 | 47 |
| 407641 | 1 | 14707 | 14726 | CCTGAGGCCAGCAGATCACG | 94 | 48 |
| 407642 | 1 | 14713 | 14732 | CAGCAGCCTGAGGCCAGCAG | 78 | 49 |
| 407643 | 1 | 15098 | 15117 | CACACATGGAGTCAGCATCG | 84 | 50 |
| 407644 | 1 | 15106 | 15125 | GAGGACAGCACACATGGAGT | 66 | 51 |
| 407645 | 1 | 15128 | 15147 | GAGAGCTAAACAACCGCCTT | 65 | 52 |
| 407646 | 1 | 15185 | 15204 | GGTGATGCTTCTGAATTGTC | 81 | 53 |
| 407647 | 1 | 15300 | 15319 | GCAGCCGTTTATTGTGAAGC | 83 | 54 |
| 407648 | 1 | 15388 | 15407 | GCATCTCAGAGGATGAGCAC | 8 | 55 |
| 407649 | 1 | 15430 | 15449 | GAGGGTTCATTTCAGTGATG | 24 | 56 |
| 407650 | 1 | 15436 | 15455 | CCATGTGAGGGTTCATTTCA | 39 | 57 |
| 407651 | 1 | 15482 | 15501 | AGCCTCAAACATCTATCAAA | 32 | 58 |
| 407652 | 1 | 15492 | 15511 | TGGGAGCTACAGCCTCAAAC | 30 | 59 |
| 407653 | 1 | 15546 | 15565 | AATATCATTGACAAGGGCTG | 21 | 60 |
| 407654 | 1 | 15630 | 15649 | CCACTGCAGCCAGGGCCTGG | 34 | 61 |
| 407655 | 1 | 15653 | 15672 | AGAGTGCAGCTTGCCAGGTC | 28 | 62 |
| 407656 | 1 | 15658 | 15677 | CAGCAAGAGTGCAGCTTGCC | 43 | 63 |
| 407657 | 1 | 15664 | 15683 | GGGACTCAGCAAGAGTGCAG | 26 | 64 |
| 407658 | 1 | 15749 | 15768 | GCTTGCCGGAGTCTGAGTGG | 16 | 65 |
| 407659 | 1 | 15778 | 15797 | GATGGCATCGAGTCCACTCT | 55 | 66 |
| 407660 | 1 | 15905 | 15924 | GCTCTGAAGTAGATGATGCC | 16 | 67 |
| 407661 | 1 | 15963 | 15982 | TTTACAAGAGCAGGGTGCCT | 47 | 68 |
| 407663 | 1 | 9112 | 9131 | TTGCAGGCAGGACTGGTGGT | 44 | 69 |
| 407664 | 1 | 5219 | 5238 | CAGGGCGAGGCAACCCCGTG | 70 | 70 |
| 407665 | 1 | 6001 | 6020 | GTTACGAAGACTGGGAAATG | 29 | 71 |
| 407666 | 1 | 8877 | 8896 | TCCCCAGGACATCTGGAACT | 52 | 72 |
| 407667 | 1 | 10897 | 10916 | GTGGCCATGCCCTGGGTCAG | 71 | 73 |
| 407668 | 1 | 12187 | 12206 | GAAGCCTTACCTGCCATGGA | 54 | 74 |
| 407669 | 1 | 12387 | 12406 | TAGACCCTCAGTGAGTGTCG | 57 | 75 |
| 407886 | 1 | 1173 | 1192 | GCAGAGGAGCCTGAGGGCCT | 72 | 76 |
| 407887 | 1 | 1201 | 1220 | CCAGGCAGCCCTGAAGCCCA | 78 | 77 |
| 407890 | 1 | 6080 | 6099 | CAGGGAGCCCGGCCGCAGCT | 60 | 78 |
| 407891 | 1 | 9176 | 9195 | CATGGACTTGAGGCACACTG | 66 | 79 |
| 407892 | 1 | 9187 | 9206 | CCCCATTCTGGCATGGACTT | 68 | 80 |
| 407893 | 1 | 9242 | 9261 | TCGAAGGCAGGGAGGCAGAA | 33 | 81 |
| 407894 | 1 | 9252 | 9271 | GTTCCGGCCCTCGAAGGCAG | 77 | 82 |
| 407895 | 1 | 10987 | 11006 | CACACAGATCAGCTGGTCAT | 72 | 83 |
| 407896 | 1 | 11029 | 11048 | CGTGTGGTCACTGCAGTACT | 73 | 84 |
| 407897 | 1 | 11039 | 11058 | GCTTGGTGCCCGTGTGGTCA | 77 | 85 |
| 407898 | 1 | 11075 | 11094 | CCAGCAGAGAGTACCCCTCG | 49 | 86 |
| 407899 | 1 | 11098 | 11117 | GGGTGTGCAGGACACCCCGT | 42 | 87 |
| 407900 | 1 | 12141 | 12160 | CCCCACAATTCGGCCTTGGG | 91 | 88 |
| 407901 | 1 | 12829 | 12848 | TGGTGTTGATCAGGGTCCCC | 89 | 89 |
| 407902 | 1 | 12837 | 12856 | CACCCAGATGGTGTTGATCA | 86 | 90 |
| 407903 | 1 | 12847 | 12866 | CCGCGGAGACCACCCAGATG | 98 | 91 |
| 407904 | 1 | 12858 | 12877 | GAAACAGTGGGCCGCGGAGA | 83 | 92 |
| 407905 | 1 | 12876 | 12895 | CCAGTTCTTGATTTTGTCGA | 40 | 93 |
| 407906 | 1 | 13847 | 13866 | ACCACGGGCTGGTGCAGGCG | 4 | 94 |
| 407907 | 1 | 13928 | 13947 | AATGAGAAGCGCACGAAGGC | 35 | 95 |
| 407908 | 1 | 13943 | 13962 | CCCCAGCCGCTGACCAATGA | 47 | 96 |
| 407909 | 1 | 14093 | 14112 | CCATCCGAGTAGCCGGCACA | 77 | 97 |
| 407910 | 1 | 14104 | 14123 | AGTCCTTGCTGCCATCCGAG | 86 | 98 |
| 407911 | 1 | 14115 | 14134 | CCCCTTGCAGGAGTCCTTGC | 74 | 99 |
| 407912 | 1 | 14149 | 14168 | CCCGGTAGTGGGTGGCATGT | 46 | 100 |
| 407913 | 1 | 14172 | 14191 | GATGCCCGTCAGGTACCACG | 74 | 101 |
| 407914 | 1 | 14181 | 14200 | CCAGCTGACGATGCCCGTCA | 82 | 102 |
| 407915 | 1 | 14198 | 14217 | GTTGCGCAGCCCTGGCCCCA | 83 | 103 |
| 407916 | 1 | 14208 | 14227 | GTGGCCCACGGTTGCGCAGC | 53 | 104 |
| 407917 | 1 | 14218 | 14237 | ACACCCCAAAGTGGCCCACG | 70 | 105 |
| 407918 | 1 | 14226 | 14245 | CCTGGTGTACACCCCAAAGT | 72 | 106 |
| 407919 | 1 | 14243 | 14262 | TCGATGTACTGGGAGACCCT | 84 | 107 |
| 407920 | 1 | 14268 | 14287 | TGAGCGCATGAGCTTTTGCA | 65 | 108 |
| 407921 | 1 | 14331 | 14350 | CCACAGGCCAGGGCTGCTGG | 65 | 109 |
| 407922 | 1 | 14354 | 14373 | TCGACGCAGCCTTGGCTTTC | 82 | 110 |
| 407923 | 1 | 14363 | 14382 | CAGGACAGTTCGACGCAGCC | 76 | 111 |
| 407924 | 1 | 14373 | 14392 | GATTTGGTGCCAGGACAGTT | 68 | 112 |
| 407925 | 1 | 14383 | 14402 | GAATATATGGGATTTGGTGC | 64 | 113 |
| 407926 | 1 | 14633 | 14652 | CCAGGACAACCTTGGCACTC | 79 | 114 |
| 407927 | 1 | 14664 | 14683 | AGGTAAGGAGGCTCAGCTGG | 55 | 115 |
| 407928 | 1 | 14677 | 14696 | CTTGGCTGAAGGGAGGTAAG | 55 | 116 |
| 407929 | 1 | 14719 | 14738 | GCAGAGCAGCAGCCTGAGGC | 53 | 117 |
| 407930 | 1 | 14727 | 14746 | CAATGAAGGCAGAGCAGCAG | 70 | 118 |
| 407931 | 1 | 15111 | 15130 | CTTCAGAGGACAGCACACAT | 56 | 119 |
| 407932 | 1 | 15141 | 15160 | GAACCAGAAAAGTGAGAGCT | 55 | 120 |
| 407933 | 1 | 15154 | 15173 | TGATAATGGATAAGAACCAG | 58 | 121 |
| 407934 | 1 | 15166 | 15185 | CTGAAGTGAAGATGATAATG | 45 | 122 |
| 407935 | 1 | 15191 | 15210 | ATGCATGGTGATGCTTCTGA | 94 | 123 |
| 407936 | 1 | 15204 | 15223 | GGCATTCGCCACCATGCATG | 91 | 124 |
| 407937 | 1 | 15237 | 15256 | GAAGGGAGAAATACATTTGG | 59 | 125 |
| 407938 | 1 | 15245 | 15264 | CACCCAGCGAAGGGAGAAAT | 60 | 126 |
| 407939 | 1 | 15255 | 15274 | TGCAGCCCGGCACCCAGCGA | 93 | 127 |
| 407940 | 1 | 15288 | 15307 | TGTGAAGCTGGGAAGCAGGT | 62 | 128 |
| 407941 | 1 | 15305 | 15324 | GAGACGCAGCCGTTTATTGT | 65 | 129 |
| 407942 | 1 | 15320 | 15339 | CACAGGTGTGCGGAGGAGAC | 41 | 130 |
| 407943 | 1 | 15328 | 15347 | GCAGGCACCACAGGTGTGCG | 34 | 131 |
| 407944 | 1 | 15338 | 15357 | CCAGTGGGTGGCAGGCACCA | 59 | 132 |
| 407945 | 1 | 15351 | 15370 | AATCATGGGCAACCCAGTGG | 56 | 133 |
| 407946 | 1 | 15361 | 15380 | TCCAAAAATGAATCATGGGC | 5 | 134 |
| 407947 | 1 | 15393 | 15412 | AAAGAGCATCTCAGAGGATG | 26 | 135 |
| 407948 | 1 | 15403 | 15422 | TTGTGAAAGAAAAGAGCATC | 8 | 136 |
| 407949 | 1 | 15418 | 15437 | CAGTGATGTTGAAAATTGTG | 9 | 137 |
| 407950 | 1 | 15441 | 15460 | AGCTTCCATGTGAGGGTTCA | 58 | 138 |
| 407951 | 1 | 15464 | 15483 | AACAGCTTTTGTTTTTAAAA | 0 | 139 |
| 407952 | 1 | 15498 | 15517 | GGATCCTGGGAGCTACAGCC | 41 | 140 |
| 407953 | 1 | 15513 | 15532 | ACATCCAATTCCACAGGATC | 20 | 141 |
| 407954 | 1 | 15523 | 15542 | CAGGGAGAGAACATCCAATT | 39 | 142 |
| 407955 | 1 | 15551 | 15570 | TGTGAAATATCATTGACAAG | 3 | 143 |
| 407956 | 1 | 15609 | 15628 | AACTTGCATTTAGTGATGCG | 19 | 144 |
| 407957 | 1 | 15617 | 15636 | GGCCTGGGAACTTGCATTTA | 52 | 145 |
| 407958 | 1 | 15754 | 15773 | GCCGTGCTTGCCGGAGTCTG | 58 | 146 |
| 407959 | 1 | 15783 | 15802 | GCAGGGATGGCATCGAGTCC | 0 | 147 |
| 407960 | 1 | 15800 | 15819 | GTGCCCAGGACGGCCCTGCA | 53 | 148 |
| 407961 | 1 | 15899 | 15918 | AAGTAGATGATGCCTGAGTG | 34 | 149 |
| 407962 | 1 | 15944 | 15963 | TTGGAAGCAGCCCACGGCTG | 41 | 150 |
| 407963 | 1 | 15957 | 15976 | AGAGCAGGGTGCCTTGGAAG | 33 | 151 |
| 407604 | 2 | 297 | 316 | CACACTGGTCCCCATCACTG | 34 | 152 |
| 407620 | 2 | 652 | 671 | AGGACCTGCCATGGACACTC | 79 | 153 |
| 407621 | 2 | 657 | 676 | ACAACAGGACCTGCCATGGA | 62 | 154 |
| 407622 | 2 | 663 | 682 | TCACCAACAACAGGACCTGC | 55 | 155 |
| 407627 | 2 | 773 | 792 | GCCCAGCACCGCGATCAGGT | 79 | 156 |
| 407888 | 2 | 102 | 121 | CGAAGACTGCAGCCAGGCAG | 34 | 157 |
| 407889 | 2 | 112 | 131 | TCCTGGGTTACGAAGACTGC | 54 | 158 |
| 407662 | 3 | 50 | 69 | TCCTGCAGCCAGGCAGCCCT | 86 | 159 |

Certain gapmers from Table 1 are 100% homologous to the rhesus monkey genomic sequence (nucleotides 691000 to 706000 of GENBANK Accession No. NW_{—}00104507.1; incorporated herein as SEQ ID NO: 162) or the rhesus monkey mRNA sequence (GENBANK Accession No. 3360_{—}061_B; incorporated herein as SEQ ID NO: 163). Shown in Table 2 are the chimeric antisense oligonucleotides from Table 1, which are homologous with rhesus monkey. Gapmers are arranged by human target start site.

TABLE 2

Human/rhesus monkey cross-reactive chimeric antisense
oligonucleotides having 5-10-5 MOE wings and deoxy gap

| | | | | | | | | |
|---|---|---|---|---|---|---|---|---|
| | | | | | | Rhesus | Rhesus | |
| | Human | Human | Human | | Rhesus | monkey | monkey | |
| | Target | Target | Target | | Target | Target | Target | |
| ISIS | SEQ ID | Start | Stop | | SEQ | Start | Stop | SEQ |
| No. | NO | Site | Site | Sequence (5′ to 3′) | ID No. | Site | Site | ID No. |

| | | | | | | | | |
|---|---|---|---|---|---|---|---|---|
| 407597 | 1 | 1114 | 1133 | TGTTGACATTCCCCATGGGA | 160 | 537 | 556 | 9 |
| 407598 | 1 | 1147 | 1166 | CCATGATGAAATCTCTGCAG | 160 | 570 | 589 | 10 |
| 407599 | 1 | 1156 | 1175 | CCTGGGAGACCATGATGAAA | 160 | 579 | 598 | 11 |
| 407886 | 1 | 1173 | 1192 | GCAGAGGAGCCTGAGGGCCT | 160 | 596 | 615 | 76 |
| 407600 | 1 | 1190 | 1209 | TGAAGCCCAAGCAGAAGGCA | 160 | 613 | 632 | 12 |
| 407601 | 1 | 1196 | 1215 | CAGCCCTGAAGCCCAAGCAG | 160 | 619 | 638 | 13 |
| 407887 | 1 | 1201 | 1220 | CCAGGCAGCCCTGAAGCCCA | 160 | 624 | 643 | 77 |
| 407602 | 1 | 1207 | 1226 | CTGCAGCCAGGCAGCCCTGA | 160 | 630 | 649 | 14 |
| 407594 | 1 | 1208 | 1227 | CCTGCAGCCAGGCAGCCCTG | 160 | 631 | 650 | 6 |
| 407595 | 1 | 2169 | 2188 | TCCTGAGGCCTTAGCGACCC | 160 | 1628 | 1647 | 7 |
| 407596 | 1 | 2206 | 2225 | GAGGCCCCGGCTTCCACGGC | 160 | 1666 | 1685 | 8 |
| 407664 | 1 | 5219 | 5238 | CAGGGCGAGGCAACCCCGTG | 160 | 3001 | 3020 | 70 |
| 407666 | 1 | 8877 | 8896 | TCCCCAGGACATCTGGAACT | 160 | 6444 | 6463 | 72 |
| 407603 | 1 | 9073 | 9092 | TAAGAAATCCAGAACAGCTT | 160 | 6637 | 6656 | 15 |
| 407663 | 1 | 9112 | 9131 | TTGCAGGCAGGACTGGTGGT | 160 | 6676 | 6695 | 69 |
| 407605 | 1 | 9169 | 9188 | TTGAGGCACACTGGTCCCCA | 160 | 6736 | 6755 | 16 |
| 407891 | 1 | 9176 | 9195 | CATGGACTTGAGGCACACTG | 160 | 6743 | 6762 | 79 |
| 407892 | 1 | 9187 | 9206 | CCCCATTCTGGCATGGACTT | 160 | 6754 | 6773 | 80 |
| 407606 | 1 | 9204 | 9223 | CTGGTCCTTGCAGGAGCCCC | 160 | 6771 | 6790 | 17 |
| 407607 | 1 | 9209 | 9228 | TGGAGCTGGTCCTTGCAGGA | 160 | 6776 | 6795 | 18 |
| 407608 | 1 | 9217 | 9236 | TATAGGACTGGAGCTGGTCC | 160 | 6784 | 6803 | 19 |
| 407609 | 1 | 9226 | 9245 | AGAAGCAGATATAGGACTGG | 160 | 6793 | 6812 | 20 |
| 407610 | 1 | 9234 | 9253 | AGGGAGGCAGAAGCAGATAT | 160 | 6801 | 6820 | 21 |
| 407893 | 1 | 9242 | 9261 | TCGAAGGCAGGGAGGCAGAA | 160 | 6809 | 6828 | 81 |
| 407894 | 1 | 9252 | 9271 | GTTCCGGCCCTCGAAGGCAG | 160 | 6819 | 6838 | 82 |
| 407611 | 1 | 9259 | 9278 | TCTCACAGTTCCGGCCCTCG | 160 | 6826 | 6845 | 22 |
| 407612 | 1 | 10982 | 11001 | AGATCAGCTGGTCATCCTTG | 160 | 8679 | 8698 | 23 |
| 407895 | 1 | 10987 | 11006 | CACACAGATCAGCTGGTCAT | 160 | 8684 | 8703 | 83 |
| 407613 | 1 | 11010 | 11029 | TGCTCACAGCCGCCGTTCTC | 160 | 8707 | 8726 | 24 |
| 407614 | 1 | 11018 | 11037 | TGCAGTACTGCTCACAGCCG | 160 | 8715 | 8734 | 25 |
| 407896 | 1 | 11029 | 11048 | CGTGTGGTCACTGCAGTACT | 160 | 8726 | 8745 | 84 |
| 407897 | 1 | 11039 | 11058 | GCTTGGTGCCCGTGTGGTCA | 160 | 8736 | 8755 | 85 |
| 407898 | 1 | 11075 | 11094 | CCAGCAGAGAGTACCCCTCG | 160 | 8772 | 8791 | 86 |
| 407615 | 1 | 11088 | 11107 | GACACCCCGTCTGCCAGCAG | 160 | 8785 | 8804 | 26 |
| 407616 | 1 | 11093 | 11112 | TGCAGGACACCCCGTCTGCC | 160 | 8790 | 8809 | 27 |
| 407899 | 1 | 11098 | 11117 | GGGTGTGCAGGACACCCCGT | 160 | 8795 | 8814 | 158 |
| 407617 | 1 | 12084 | 12103 | TCCACATGGATATTCAACTG | 160 | 9808 | 9827 | 28 |
| 407618 | 1 | 12091 | 12110 | GTATTTTTCCACATGGATAT | 160 | 9815 | 9834 | 29 |
| 407619 | 1 | 12098 | 12117 | AGAATAGGTATTTTTCCACA | 160 | 9822 | 9841 | 30 |
| 407900 | 1 | 12141 | 12160 | CCCCACAATTCGGCCTTGGG | 160 | 9865 | 9884 | 88 |
| 407668 | 1 | 12187 | 12206 | GAAGCCTTACCTGCCATGGA | 160 | 9911 | 9930 | 74 |
| 407669 | 1 | 12387 | 12406 | TAGACCCTCAGTGAGTGTCG | 160 | 10116 | 10135 | 75 |
| 407623 | 1 | 12795 | 12814 | TCCATTCACCAACAACAGGA | 160 | 10524 | 10543 | 31 |
| 407901 | 1 | 12829 | 12848 | TGGTGTTGATCAGGGTCCCC | 160 | 10558 | 10577 | 89 |
| 407902 | 1 | 12837 | 12856 | CACCCAGATGGTGTTGATCA | 160 | 10566 | 10585 | 90 |
| 407624 | 1 | 12842 | 12861 | GAGACCACCCAGATGGTGTT | 160 | 10571 | 10590 | 32 |
| 407903 | 1 | 12847 | 12866 | CCGCGGAGACCACCCAGATG | 160 | 10576 | 10595 | 91 |
| 407904 | 1 | 12858 | 12877 | GAAACAGTGGGCCGCGGAGA | 160 | 10587 | 10606 | 92 |
| 407625 | 1 | 12863 | 12882 | TTGTCGAAACAGTGGGCCGC | 160 | 10592 | 10611 | 33 |
| 407626 | 1 | 12871 | 12890 | TCTTGATTTTGTCGAAACAG | 160 | 10600 | 10619 | 34 |
| 407905 | 1 | 12876 | 12895 | CCAGTTCTTGATTTTGTCGA | 160 | 10605 | 10624 | 93 |
| 407628 | 1 | 13777 | 13796 | TGATGACCTGCGCCACCCGC | 160 | 11499 | 11518 | 35 |
| 407906 | 1 | 13847 | 13866 | ACCACGGGCTGGTGCAGGCG | 160 | 11569 | 11588 | 94 |
| 407629 | 1 | 13856 | 13875 | TCAGTGAGGACCACGGGCTG | 160 | 11578 | 11597 | 36 |
| 407630 | 1 | 13863 | 13882 | CACATGGTCAGTGAGGACCA | 160 | 11585 | 11604 | 37 |
| 407631 | 1 | 13869 | 13888 | GGGCACCACATGGTCAGTGA | 160 | 11591 | 11610 | 38 |
| 407632 | 1 | 13888 | 13907 | TCCGTTCGGGCAGGCAGAGG | 160 | 11610 | 11629 | 39 |
| 407907 | 1 | 13928 | 13947 | AATGAGAAGCGCACGAAGGC | 160 | 11650 | 11669 | 95 |
| 407908 | 1 | 13943 | 13962 | CCCCAGCCGCTGACCAATGA | 160 | 11665 | 11684 | 96 |
| 403090 | 1 | 14017 | 14036 | AGTCCTGGGTCATCAGCCGG | 160 | 11739 | 11758 | 4 |
| 407633 | 1 | 14023 | 14042 | GCAGGCAGTCCTGGGTCATC | 160 | 11745 | 11764 | 40 |
| 407634 | 1 | 14032 | 14051 | GTGACTGCTGCAGGCAGTCC | 160 | 11754 | 11773 | 41 |
| 407909 | 1 | 14093 | 14112 | CCATCCGAGTAGCCGGCACA | 160 | 11815 | 11834 | 97 |
| 407910 | 1 | 14104 | 14123 | AGTCCTTGCTGCCATCCGAG | 160 | 11826 | 11845 | 98 |
| 407911 | 1 | 14115 | 14134 | CCCCTTGCAGGAGTCCTTGC | 160 | 11837 | 11856 | 99 |
| 407912 | 1 | 14149 | 14168 | CCCGGTAGTGGGTGGCATGT | 160 | 11871 | 11890 | 100 |
| 407913 | 1 | 14172 | 14191 | GATGCCCGTCAGGTACCACG | 160 | 11894 | 11913 | 101 |
| 407914 | 1 | 14181 | 14200 | CCAGCTGACGATGCCCGTCA | 160 | 11903 | 11922 | 102 |
| 407635 | 1 | 14186 | 14205 | TGGCCCCAGCTGACGATGCC | 160 | 11908 | 11927 | 42 |
| 407636 | 1 | 14193 | 14212 | GCAGCCCTGGCCCCAGCTGA | 160 | 11915 | 11934 | 43 |
| 407915 | 1 | 14198 | 14217 | GTTGCGCAGCCCTGGCCCCA | 160 | 11920 | 11939 | 103 |
| 407916 | 1 | 14208 | 14227 | GTGGCCCACGGTTGCGCAGC | 160 | 11930 | 11949 | 104 |
| 407917 | 1 | 14218 | 14237 | ACACCCCAAAGTGGCCCACG | 160 | 11940 | 11959 | 105 |
| 407918 | 1 | 14226 | 14245 | CCTGGTGTACACCCCAAAGT | 160 | 11948 | 11967 | 106 |
| 403093 | 1 | 14231 | 14250 | GAGACCCTGGTGTACACCCC | 160 | 11953 | 11972 | 5 |
| 407637 | 1 | 14238 | 14257 | GTACTGGGAGACCCTGGTGT | 160 | 11960 | 11979 | 44 |
| 407919 | 1 | 14243 | 14262 | TCGATGTACTGGGAGACCCT | 160 | 11965 | 11984 | 107 |
| 407638 | 1 | 14248 | 14267 | GCCACTCGATGTACTGGGAG | 160 | 11970 | 11989 | 45 |
| 407639 | 1 | 14254 | 14273 | TTTGCAGCCACTCGATGTAC | 160 | 11976 | 11995 | 46 |
| 407640 | 1 | 14263 | 14282 | GCATGAGCTTTTGCAGCCAC | 160 | 11985 | 12004 | 47 |
| 407920 | 1 | 14268 | 14287 | TGAGCGCATGAGCTTTTGCA | 160 | 11990 | 12009 | 108 |
| 407922 | 1 | 14354 | 14373 | TCGACGCAGCCTTGGCTTTC | 160 | 12076 | 12095 | 110 |
| 407923 | 1 | 14363 | 14382 | CAGGACAGTTCGACGCAGCC | 160 | 12085 | 12104 | 111 |
| 407924 | 1 | 14373 | 14392 | GATTTGGTGCCAGGACAGTT | 160 | 12095 | 12114 | 112 |
| 407925 | 1 | 14383 | 14402 | GAATATATGGGATTTGGTGC | 160 | 12105 | 12124 | 113 |
| 407927 | 1 | 14664 | 14683 | AGGTAAGGAGGCTCAGCTGG | 160 | 12384 | 12403 | 115 |
| 407928 | 1 | 14677 | 14696 | CTTGGCTGAAGGGAGGTAAG | 160 | 12397 | 12416 | 116 |
| 407641 | 1 | 14707 | 14726 | CCTGAGGCCAGCAGATCACG | 160 | 12427 | 12446 | 48 |
| 407642 | 1 | 14713 | 14732 | CAGCAGCCTGAGGCCAGCAG | 160 | 12433 | 12452 | 49 |
| 407929 | 1 | 14719 | 14738 | GCAGAGCAGCAGCCTGAGGC | 160 | 12397 | 12416 | 117 |
| 407930 | 1 | 14727 | 14746 | CAATGAAGGCAGAGCAGCAG | 160 | 12447 | 12466 | 118 |
| 407643 | 1 | 15098 | 15117 | CACACATGGAGTCAGCATCG | 160 | 12815 | 12834 | 50 |
| 407644 | 1 | 15106 | 15125 | GAGGACAGCACACATGGAGT | 160 | 12823 | 12842 | 51 |
| 407931 | 1 | 15111 | 15130 | CTTCAGAGGACAGCACACAT | 160 | 12828 | 12847 | 119 |
| 407645 | 1 | 15128 | 15147 | GAGAGCTAAACAACCGCCTT | 160 | 12845 | 12864 | 52 |
| 407932 | 1 | 15141 | 15160 | GAACCAGAAAAGTGAGAGCT | 160 | 12858 | 12847 | 120 |
| 407933 | 1 | 15154 | 15173 | TGATAATGGATAAGAACCAG | 160 | 12871 | 12890 | 121 |
| 407934 | 1 | 15166 | 15185 | CTGAAGTGAAGATGATAATG | 160 | 12883 | 12902 | 122 |
| 407646 | 1 | 15185 | 15204 | GGTGATGCTTCTGAATTGTC | 160 | 12902 | 12921 | 53 |
| 407935 | 1 | 15191 | 15210 | ATGCATGGTGATGCTTCTGA | 160 | 12908 | 12927 | 123 |
| 407936 | 1 | 15204 | 15223 | GGCATTCGCCACCATGCATG | 160 | 12921 | 12940 | 124 |
| 407940 | 1 | 15288 | 15307 | TGTGAAGCTGGGAAGCAGGT | 160 | 12985 | 13004 | 128 |
| 407647 | 1 | 15300 | 15319 | GCAGCCGTTTATTGTGAAGC | 160 | 12997 | 13016 | 54 |
| 407941 | 1 | 15305 | 15324 | GAGACGCAGCCGTTTATTGT | 160 | 13002 | 13021 | 129 |
| 407942 | 1 | 15320 | 15339 | CACAGGTGTGCGGAGGAGAC | 160 | 13017 | 13036 | 130 |
| 407943 | 1 | 15328 | 15347 | GCAGGCACCACAGGTGTGCG | 160 | 13025 | 13044 | 131 |
| 407944 | 1 | 15338 | 15357 | CCAGTGGGTGGCAGGCACCA | 160 | 13035 | 13054 | 132 |
| 407648 | 1 | 15388 | 15407 | GCATCTCAGAGGATGAGCAC | 160 | 13085 | 13104 | 136 |
| 407947 | 1 | 15393 | 15412 | AAAGAGCATCTCAGAGGATG | 160 | 13090 | 13109 | 54 |
| 407948 | 1 | 15403 | 15422 | TTGTGAAAGAAAAGAGCATC | 160 | 13100 | 13119 | 55 |
| 407949 | 1 | 15418 | 15437 | CAGTGATGTTGAAAATTGTG | 160 | 13115 | 13134 | 57 |
| 407649 | 1 | 15430 | 15449 | GAGGGTTCATTTCAGTGATG | 160 | 13127 | 13146 | 56 |
| 407650 | 1 | 15436 | 15455 | CCATGTGAGGGTTCATTTCA | 160 | 13133 | 13152 | 57 |
| 407950 | 1 | 15441 | 15460 | AGCTTCCATGTGAGGGTTCA | 160 | 13138 | 13157 | 138 |
| 407951 | 1 | 15464 | 15483 | AACAGCTTTTGTTTTTAAAA | 160 | 13163 | 13182 | 139 |
| 407651 | 1 | 15482 | 15501 | AGCCTCAAACATCTATCAAA | 160 | 13181 | 13200 | 58 |
| 407652 | 1 | 15492 | 15511 | TGGGAGCTACAGCCTCAAAC | 160 | 13191 | 13210 | 59 |
| 407952 | 1 | 15498 | 15517 | GGATCCTGGGAGCTACAGCC | 160 | 13197 | 13216 | 140 |
| 407953 | 1 | 15513 | 15532 | ACATCCAATTCCACAGGATC | 160 | 13212 | 13231 | 141 |
| 407954 | 1 | 15523 | 15542 | CAGGGAGAGAACATCCAATT | 160 | 13222 | 13241 | 142 |
| 407653 | 1 | 15546 | 15565 | AATATCATTGACAAGGGCTG | 160 | 13245 | 13264 | 60 |
| 407955 | 1 | 15551 | 15570 | TGTGAAATATCATTGACAAG | 160 | 13250 | 13269 | 143 |
| 407957 | 1 | 15617 | 15636 | GGCCTGGGAACTTGCATTTA | 160 | 13312 | 13331 | 145 |
| 407654 | 1 | 15630 | 15649 | CCACTGCAGCCAGGGCCTGG | 160 | 13325 | 13344 | 61 |
| 407655 | 1 | 15653 | 15672 | AGAGTGCAGCTTGCCAGGTC | 160 | 13348 | 13367 | 62 |
| 407656 | 1 | 15658 | 15677 | CAGCAAGAGTGCAGCTTGCC | 160 | 13353 | 13372 | 63 |
| 407657 | 1 | 15664 | 15683 | GGGACTCAGCAAGAGTGCAG | 160 | 13359 | 13378 | 64 |
| 407658 | 1 | 15749 | 15768 | GCTTGCCGGAGTCTGAGTGG | 160 | 13444 | 13463 | 65 |
| 407958 | 1 | 15754 | 15773 | GCCGTGCTTGCCGGAGTCTG | 160 | 13449 | 13468 | 146 |
| 407659 | 1 | 15778 | 15797 | GATGGCATCGAGTCCACTCT | 160 | 13473 | 13492 | 66 |
| 407959 | 1 | 15783 | 15802 | GCAGGGATGGCATCGAGTCC | 160 | 13478 | 13497 | 147 |
| 407960 | 1 | 15800 | 15819 | GTGCCCAGGACGGCCCTGCA | 160 | 13495 | 13514 | 148 |
| 407961 | 1 | 15899 | 15918 | AAGTAGATGATGCCTGAGTG | 160 | 13564 | 13583 | 149 |
| 407660 | 1 | 15905 | 15924 | GCTCTGAAGTAGATGATGCC | 160 | 13570 | 13589 | 67 |
| 407962 | 1 | 15944 | 15963 | TTGGAAGCAGCCCACGGCTG | 160 | 13609 | 13628 | 150 |
| 407963 | 1 | 15957 | 15976 | AGAGCAGGGTGCCTTGGAAG | 160 | 13622 | 13641 | 151 |
| 407661 | 1 | 15963 | 15982 | TTTACAAGAGCAGGGTGCCT | 160 | 13628 | 13647 | 68 |
| 407604 | 2 | 297 | 316 | CACACTGGTCCCCATCACTG | 160 | 6730 | 6749 | 152 |
| 407620 | 2 | 652 | 671 | AGGACCTGCCATGGACACTC | 160 | 9906 | 9925 | 153 |
| 407621 | 2 | 657 | 676 | ACAACAGGACCTGCCATGGA | 161 | 723 | 742 | 154 |
| 407622 | 2 | 663 | 682 | TCACCAACAACAGGACCTGC | 160 | 10519 | 10538 | 155 |
| 407627 | 2 | 773 | 792 | GCCCAGCACCGCGATCAGGT | 160 | 10629 | 10648 | 156 |
| 407662 | 3 | 50 | 69 | TCCTGCAGCCAGGCAGCCCT | 160 | 632 | 651 | 159 |

### Example 2

### Dose-Dependent Antisense Inhibition of Human Factor 7 in HepB3 Cells

Several antisense oligonucleotides from Example 1 (see Table 1) exhibiting at least 80% in vitro inhibition of human Factor 7 were tested at various doses in HepB3 cells. Cells were plated at a density of 4,000 cells per well and treated with lipofectin reagent with 3.125 nM, 6.25 nM, 12.5 nM, 25 nM, 50 nM, and 100 nM concentrations of antisense oligonucleotide, as indicated in Table 3. After a treatment period of approximately 16 hours, RNA was isolated from the cells and Factor 7 mRNA levels were measured by real-time RT-PCR, as described herein. Human Factor 7 primer probe set RTS 2927 (forward sequence: GGGACCCTGATCAACACCAT, incorporated herein as SEQ ID NO: 164; reverse sequence: CCAGTTCTTGATTTTGTCGAAACA, incorporated herein as SEQ ID NO: 165; probe sequence: TGGGTGGTCTCCGCGGCCX, incorporated herein as SEQ ID NO: 166) was used to measure mRNA levels. Factor 7 mRNA levels were adjusted according to total RNA content as measured by RIBOGREEN®. Results are presented as percent inhibition of Factor 7, relative to untreated control cells. As illustrated in Table 3, Factor 7 mRNA levels were reduced in a dose-dependent manner.

TABLE 3
Dose-dependent antisense inhibition of human Factor 7 in HepB3 cells

| | | | 12.5 | 25.0 | 50.0 | 100.0 | SEQ ID |
|---|---|---|---|---|---|---|---|
| ISIS No. | 3.125 nM | 6.25 nM | nM | nM | nM | nM | No. |
| 407641 | 53 | 53 | 71 | 84 | 88 | 89 | 48 |
| 407606 | 20 | 0 | 49 | 55 | 83 | 84 | 17 |
| 407594 | 10 | 34 | 63 | 77 | 84 | 80 | 6 |
| 407662 | 0 | 35 | 58 | 74 | 81 | 83 | 166 |
| 407643 | 16 | 59 | 68 | 76 | 92 | 95 | 50 |
| 407935 | 57 | 76 | 78 | 89 | 90 | 89 | 123 |
| 407939 | 62 | 79 | 83 | 91 | 92 | 92 | 127 |
| 407900 | 52 | 58 | 80 | 87 | 94 | 91 | 88 |
| 407936 | 45 | 77 | 79 | 86 | 91 | 90 | 124 |
| 407910 | 31 | 44 | 69 | 68 | 82 | 89 | 98 |

### Example 3

### Antisense Inhibition of Human Factor 7 in HepB3 Cells

Antisense oligonucleotides targeted to a Factor 7 nucleic acid were designed and tested for their effects on Factor 7 mRNA in vitro. Certain antisense oligonucleotides from Table 3 were also retested for their effects on Factor 7 mRNA in vitro. Cultured HepB3 cells at a density of 4,000 cells per well were transfected using lipofectin reagent with 50 nM antisense oligonucleotide. After a treatment period of approximately 24 hours, RNA was isolated from the cells and Factor 7 mRNA levels were measured by real-time RT-PCR, as described herein. Human Factor 7 primer probe set RTS 2927 was used to measure mRNA levels. Factor 7 mRNA levels were adjusted according to total RNA content as measured by RIBOGREEN®. Results are presented as percent inhibition of Factor 7, relative to untreated control.

The chimeric antisense oligonucleotides in Table 4 were designed as 5-10-5 MOE gapmers. The gapmers are 20 nucleotides in length, wherein the central gap segment is comprised of ten 2′-deoxynucleotides and is flanked on both sides (in the 5′ and 3′ directions) by wings comprising five nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. The first seven listed gapmers in Table 4 are from Table 3 and are designated by an asterisk (*). “Human Target start site” indicates the 5′-most nucleotide to which the gapmer is targeted in the specified human gene sequence. “Human Target stop site” indicates the 3′-most nucleotide to which the gapmer is targeted in the specified human gene sequence. Each gapmer listed in Table 4 is targeted to SEQ ID NO: 1 (nucleotides 1255000 to 1273000 of GENBANK® Accession No. NT_{—}027140.6), SEQ ID NO: 2 (GENBANK® Accession No. NM_{—}019616.2), or SEQ ID NO: 167 (GENBANK® Accession No. NM_{—}000131.3).

TABLE 4

Inhibition of human Factor 7 mRNA levels by chimeric antisense
oligonucleotides having 5-10-5 MOE wings and deoxy gap targeted
to SEQ ID NO: 1, SEQ ID NO: 2, and SEQ ID NO: 167

| | | | | | | |
|---|---|---|---|---|---|---|
| | Human | Human | Human | | | |
| | Target | Target | Target | | | SEQ |
| | SEQ ID | Start | Stop | | % | ID |
| ISIS No. | NO | Site | Site | Sequence (5′ to 3′) | Inhibition | No. |

| | | | | | | |
|---|---|---|---|---|---|---|
| *407606 | 1 | 9204 | 9223 | CTGGTCCTTGCAGGAGCCCC | 57 | 17 |
| *407900 | 1 | 12141 | 12160 | CCCCACAATTCGGCCTTGGG | 78 | 88 |
| *407910 | 1 | 14104 | 14123 | AGTCCTTGCTGCCATCCGAG | 72 | 98 |
| *407641 | 1 | 14707 | 14726 | CCTGAGGCCAGCAGATCACG | 73 | 48 |
| *407643 | 1 | 15098 | 15117 | CACACATGGAGTCAGCATCG | 80 | 50 |
| *407935 | 1 | 15191 | 15210 | ATGCATGGTGATGCTTCTGA | 72 | 123 |
| *407939 | 1 | 15255 | 15274 | TGCAGCCCGGCACCCAGCGA | 76 | 127 |
| 416492 | 1 | 616 | 635 | GGATCATTCTGGCCCTGAGC | 13 | 168 |
| 416493 | 1 | 738 | 757 | TCTTGGGTGTGGATGTAAAT | 0 | 169 |
| 416494 | 1 | 803 | 822 | CAGATTTAAACTGCAGATGA | 0 | 170 |
| 416495 | 1 | 838 | 857 | TCTAGAATTCCAAACCCCTA | 0 | 171 |
| 416496 | 1 | 855 | 874 | CAACACTTCAAATACGATCT | 0 | 172 |
| 416497 | 1 | 883 | 902 | CGTGCAGGTGTTAAGGTGTG | 0 | 173 |
| 416498 | 1 | 994 | 1013 | GGCCAGTGGCCATGCATCCC | 5 | 174 |
| 416499 | 1 | 1011 | 1030 | GAGAGCTGCACCTGGCCGGC | 7 | 175 |
| 416500 | 1 | 1026 | 1045 | CTGAACACCCCAGCTGAGAG | 1 | 176 |
| 416424 | 1 | 1151 | 1170 | GAGACCATGATGAAATCTCT | 27 | 177 |
| 416425 | 1 | 1182 | 1201 | AAGCAGAAGGCAGAGGAGCC | 13 | 178 |
| 416426 | 1 | 1187 | 1206 | AGCCCAAGCAGAAGGCAGAG | 34 | 179 |
| 416427 | 1 | 1193 | 1212 | CCCTGAAGCCCAAGCAGAAG | 5 | 180 |
| 416501 | 1 | 1251 | 1270 | CTGCCCTTCCACCAAGTTTA | 18 | 181 |
| 416502 | 1 | 1437 | 1456 | GTTCTTTGAAAAATAATCCC | 22 | 182 |
| 416423 | 1 | 2179 | 2198 | GTGTTTCTCCTCCTGAGGCC | 51 | 183 |
| 416503 | 1 | 2311 | 2330 | TAGCCACCCCGCGGGCTGGC | 0 | 184 |
| 416504 | 1 | 2484 | 2503 | TCAGAAAAGCTCTCAAGAAC | 0 | 185 |
| 416505 | 1 | 2495 | 2514 | GCAGATTTGCATCAGAAAAG | 45 | 186 |
| 416506 | 1 | 4766 | 4785 | CTTTAAAATCAGTTTCACAC | 18 | 187 |
| 416507 | 1 | 4847 | 4866 | GGTTACTGAGCGCGGAAGAA | 73 | 188 |
| 416508 | 1 | 4873 | 4892 | CGAGTTCTGCAGGAGCGGCC | 67 | 189 |
| 416509 | 1 | 4880 | 4899 | AGGAGCCCGAGTTCTGCAGG | 56 | 190 |
| 416510 | 1 | 4916 | 4935 | GACGAGGCCTCAGGTGGACG | 30 | 191 |
| 416511 | 1 | 4926 | 4945 | TTGCTGGGAGGACGAGGCCT | 35 | 192 |
| 416512 | 1 | 4934 | 4953 | GACGACCTTTGCTGGGAGGA | 43 | 193 |
| 416429 | 1 | 6022 | 6041 | ACGCCGTGGGCTTCCTCCTG | 47 | 194 |
| 416430 | 1 | 6066 | 6085 | GCAGCTCCTCCAGGAACGCG | 52 | 195 |
| 416431 | 1 | 6079 | 6098 | AGGGAGCCCGGCCGCAGCTC | 25 | 196 |
| 416432 | 1 | 6108 | 6127 | AGCACTGCTCCTCCTTGCAC | 44 | 197 |
| 416513 | 1 | 6399 | 6418 | CTGATGTGAAAACCGGCATG | 37 | 198 |
| 416514 | 1 | 6406 | 6425 | GTATTTTCTGATGTGAAAAC | 8 | 199 |
| 416515 | 1 | 8547 | 8566 | TAGGCATGACCATCCTCAAT | 39 | 200 |
| 416516 | 1 | 8599 | 8618 | GTGAGAATACAACAGATGAG | 24 | 201 |
| 416517 | 1 | 8708 | 8727 | GGGTGCAGTAGCAGATGCAA | 23 | 202 |
| 416518 | 1 | 8855 | 8874 | GGGTGACCACACATTTCCTG | 55 | 203 |
| 416434 | 1 | 9076 | 9095 | CTGTAAGAAATCCAGAACAG | 0 | 204 |
| 416491 | 1 | 9120 | 9139 | GAGAAGGGTTGCAGGCAGGA | 2 | 205 |
| 416438 | 1 | 9194 | 9213 | CAGGAGCCCCCATTCTGGCA | 63 | 206 |
| 416439 | 1 | 9201 | 9220 | GTCCTTGCAGGAGCCCCCAT | 56 | 207 |
| 416440 | 1 | 9213 | 9232 | GGACTGGAGCTGGTCCTTGC | 44 | 208 |
| 416441 | 1 | 9220 | 9239 | AGATATAGGACTGGAGCTGG | 32 | 209 |
| 416442 | 1 | 9223 | 9242 | AGCAGATATAGGACTGGAGC | 57 | 210 |
| 416443 | 1 | 9231 | 9250 | GAGGCAGAAGCAGATATAGG | 17 | 211 |
| 416444 | 1 | 9255 | 9274 | ACAGTTCCGGCCCTCGAAGG | 24 | 212 |
| 416519 | 1 | 9290 | 9309 | AAATATGGGACCCAAAGTGG | 9 | 213 |
| 416520 | 1 | 9298 | 9317 | CCCTCTGCAAATATGGGACC | 14 | 214 |
| 416521 | 1 | 9362 | 9381 | CACCCCACCAGGTTGTGCAC | 36 | 215 |
| 416522 | 1 | 9515 | 9534 | GGCTGAGAATTGCCCAGGGC | 25 | 216 |
| 416523 | 1 | 9522 | 9541 | TCTCGAGGGCTGAGAATTGC | 18 | 217 |
| 416524 | 1 | 9665 | 9684 | TAATTTAATATTCAGATGGT | 0 | 218 |
| 416525 | 1 | 9725 | 9744 | TATGAGTCCTTCTAGTGAAT | 5 | 219 |
| 416526 | 1 | 9848 | 9867 | TGTCCACATGACCCCACAGG | 19 | 220 |
| 416527 | 1 | 9912 | 9931 | GAGCTTCCCAAGTTGGCAGT | 49 | 221 |
| 416528 | 1 | 9942 | 9961 | TGATAAAACCTCTGGACACC | 10 | 222 |
| 416529 | 1 | 9999 | 10018 | GGGCTGAGACTGAGGTCAGC | 18 | 223 |
| 416530 | 1 | 10166 | 10185 | AGGGTAGCCTTTGCCTTGGC | 35 | 224 |
| 416531 | 1 | 10317 | 10336 | AGATGACCAGCAGGAAGCCT | 19 | 225 |
| 416532 | 1 | 10323 | 10342 | GGACCCAGATGACCAGCAGG | 12 | 226 |
| 416533 | 1 | 10330 | 10349 | GCATTCTGGACCCAGATGAC | 41 | 227 |
| 416534 | 1 | 10377 | 10396 | ATGCACACCAGGGCTGCTGG | 32 | 228 |
| 416535 | 1 | 10382 | 10401 | GCAGGATGCACACCAGGGCT | 47 | 229 |
| 416536 | 1 | 10398 | 10417 | CGGGAAGGCCTGCCCTGCAG | 23 | 230 |
| 416537 | 1 | 10677 | 10696 | TGACCACTCTTCCGAGCAGC | 55 | 231 |
| 416538 | 1 | 10807 | 10826 | CGTGGACTGATCCAAAGGAC | 46 | 232 |
| 416539 | 1 | 10837 | 10856 | GACAGAGCCTGAGCTTGGCA | 53 | 233 |
| 416445 | 1 | 11013 | 11032 | TACTGCTCACAGCCGCCGTT | 57 | 234 |
| 416446 | 1 | 11024 | 11043 | GGTCACTGCAGTACTGCTCA | 64 | 235 |
| 416540 | 1 | 11143 | 11162 | GGACTGGTGTCATCTGGGAC | 38 | 236 |
| 416541 | 1 | 11259 | 11278 | CCACCCTTGGTGCCCAGATC | 53 | 237 |
| 416542 | 1 | 11297 | 11316 | CAGGGTGCCCATCCTAGTCA | 56 | 238 |
| 416543 | 1 | 11395 | 11414 | TCCTGCGAGTGGGAGTTGGA | 0 | 239 |
| 416544 | 1 | 11499 | 11518 | ATCCCATTTTCCCAGGAGCC | 58 | 240 |
| 416545 | 1 | 11505 | 11524 | AGAAACATCCCATTTTCCCA | 8 | 241 |
| 416546 | 1 | 11519 | 11538 | CCAGGCTGGTTTGGAGAAAC | 21 | 242 |
| 416547 | 1 | 11729 | 11748 | ATGAAATTCTACCTAAAGAT | 0 | 243 |
| 416548 | 1 | 11735 | 11754 | AGGTGAATGAAATTCTACCT | 29 | 244 |
| 416549 | 1 | 11838 | 11857 | GACAATGGTCAGGGCTGGTT | 68 | 245 |
| 416550 | 1 | 11852 | 11871 | TGGCTGGCTGAGGAGACAAT | 55 | 246 |
| 416551 | 1 | 12000 | 12019 | CAGAAACACCCATCCTCTGA | 19 | 247 |
| 416448 | 1 | 12088 | 12107 | TTTTTCCACATGGATATTCA | 34 | 248 |
| 416449 | 1 | 12094 | 12113 | TAGGTATTTTTCCACATGGA | 68 | 249 |
| 416450 | 1 | 12122 | 12141 | GGTTTGCTGGCATTTCTTTT | 49 | 250 |
| 416451 | 1 | 12175 | 12194 | GCCATGGACACTCCCCTTTG | 75 | 251 |
| 416552 | 1 | 12398 | 12417 | TCTGCACAGGGTAGACCCTC | 46 | 252 |
| 416553 | 1 | 12403 | 12422 | GGTTCTCTGCACAGGGTAGA | 56 | 253 |
| 416554 | 1 | 12467 | 12486 | AAAGATCCCACCTCAAAGAG | 7 | 254 |
| 416555 | 1 | 12478 | 12497 | AAAGATCAGGCAAAGATCCC | 6 | 255 |
| 416556 | 1 | 12508 | 12527 | ATAGCTTTGATCCAATGCTC | 53 | 256 |
| 416557 | 1 | 12639 | 12658 | TCCCAGGCAAAGCTGCTCAG | 56 | 257 |
| 416453 | 1 | 12867 | 12886 | GATTTTGTCGAAACAGTGGG | 45 | 258 |
| 416558 | 1 | 13159 | 13178 | TGACAGCACGAAGCCCAGAG | 19 | 259 |
| 416559 | 1 | 13638 | 13657 | GCCATTTCTAGGTCTGCAGG | 25 | 260 |
| 416455 | 1 | 13760 | 13779 | CGCCGGCTCTGCTCATCCCC | 72 | 261 |
| 416456 | 1 | 13770 | 13789 | CTGCGCCACCCGCCGGCTCT | 58 | 262 |
| 416457 | 1 | 13780 | 13799 | GGATGATGACCTGCGCCACC | 48 | 263 |
| 416458 | 1 | 13831 | 13850 | GGCGGAGCAGCGCGATGTCG | 29 | 264 |
| 416459 | 1 | 13859 | 13878 | TGGTCAGTGAGGACCACGGG | 23 | 265 |
| 416460 | 1 | 13866 | 13885 | CACCACATGGTCAGTGAGGA | 49 | 266 |
| 416461 | 1 | 13923 | 13942 | GAAGCGCACGAAGGCCAGCG | 43 | 267 |
| 416462 | 1 | 14020 | 14039 | GGCAGTCCTGGGTCATCAGC | 60 | 268 |
| 416463 | 1 | 14027 | 14046 | TGCTGCAGGCAGTCCTGGGT | 39 | 269 |
| 416464 | 1 | 14072 | 14091 | AACATGTACTCCGTGATATT | 53 | 270 |
| 416465 | 1 | 14122 | 14141 | CACTGTCCCCCTTGCAGGAG | 51 | 271 |
| 416466 | 1 | 14132 | 14151 | TGTGGGCCTCCACTGTCCCC | 57 | 272 |
| 416467 | 1 | 14189 | 14208 | CCCTGGCCCCAGCTGACGAT | 55 | 273 |
| 416468 | 1 | 14234 | 14253 | TGGGAGACCCTGGTGTACAC | 46 | 274 |
| 416469 | 1 | 14251 | 14270 | GCAGCCACTCGATGTACTGG | 55 | 275 |
| 416470 | 1 | 14257 | 14276 | GCTTTTGCAGCCACTCGATG | 39 | 276 |
| 416471 | 1 | 14260 | 14279 | TGAGCTTTTGCAGCCACTCG | 62 | 277 |
| 416472 | 1 | 14348 | 14367 | CAGCCTTGGCTTTCTCTCCA | 76 | 278 |
| 416473 | 1 | 14613 | 14632 | CCCTGCCCCTCTGTCCAGCG | 61 | 279 |
| 416474 | 1 | 14642 | 14661 | TGTCTGCCTCCAGGACAACC | 40 | 280 |
| 416475 | 1 | 14653 | 14672 | CTCAGCTGGGCTGTCTGCCT | 70 | 281 |
| 416476 | 1 | 14686 | 14705 | GCAGGTGGGCTTGGCTGAAG | 58 | 282 |
| 416477 | 1 | 14710 | 14729 | CAGCCTGAGGCCAGCAGATC | 74 | 283 |
| 416478 | 1 | 14735 | 14754 | GTCTCCAGCAATGAAGGCAG | 44 | 284 |
| 416479 | 1 | 15103 | 15122 | GACAGCACACATGGAGTCAG | 56 | 285 |
| 416480 | 1 | 15132 | 15151 | AAGTGAGAGCTAAACAACCG | 25 | 286 |
| 416481 | 1 | 15157 | 15176 | AGATGATAATGGATAAGAAC | 15 | 287 |
| 416482 | 1 | 15188 | 15207 | CATGGTGATGCTTCTGAATT | 64 | 288 |
| 416483 | 1 | 15433 | 15452 | TGTGAGGGTTCATTTCAGTG | 22 | 289 |
| 416484 | 1 | 15485 | 15504 | TACAGCCTCAAACATCTATC | 0 | 290 |
| 416485 | 1 | 15489 | 15508 | GAGCTACAGCCTCAAACATC | 7 | 291 |
| 416486 | 1 | 15540 | 15559 | ATTGACAAGGGCTGTGGCAG | 12 | 292 |
| 416487 | 1 | 15571 | 15590 | GCAGGTGCTCCCAGGGTCTC | 39 | 293 |
| 416488 | 1 | 15639 | 15658 | CAGGTCCTCCCACTGCAGCC | 41 | 294 |
| 416489 | 1 | 15650 | 15669 | GTGCAGCTTGCCAGGTCCTC | 38 | 295 |
| 416490 | 1 | 15661 | 15680 | ACTCAGCAAGAGTGCAGCTT | 33 | 296 |
| 416560 | 1 | 15973 | 15992 | TAAAACTTTATTTACAAGAG | 0 | 297 |
| 416561 | 1 | 15985 | 16004 | GGTGTGTTCCCATAAAACTT | 16 | 298 |
| 416562 | 1 | 16185 | 16204 | AAAGCAGAGCCAGCTCTGAC | 12 | 299 |
| 416563 | 1 | 16596 | 16615 | GCCTGCATTTCCCATTGGCA | 0 | 300 |
| 416564 | 1 | 16738 | 16757 | GCCACTCACAGAAAGCTGGA | 0 | 301 |
| 416565 | 1 | 16872 | 16891 | CAGGATGCTCATGGCAGACA | 0 | 302 |
| 416566 | 1 | 16911 | 16930 | GTTTGTATGGAGAGACCAAT | 0 | 303 |
| 416567 | 1 | 16977 | 16996 | TGGTGGCACCAGATGTCTGA | 2 | 304 |
| 416568 | 1 | 17112 | 17131 | CATTGTGCCTGGCACACAGG | 14 | 305 |
| 416569 | 1 | 17136 | 17155 | GCCTGGTGTGCACACATTGT | 12 | 306 |
| 416428 | 2 | 110 | 129 | CTGGGTTACGAAGACTGCAG | 11 | 307 |
| 416422 | 167 | 107 | 126 | AGCGACCCCGCCTGCAGCCA | 8 | 308 |
| 416433 | 167 | 341 | 360 | AGAAATCCAGAACAGCTTCG | 0 | 309 |
| 416435 | 167 | 353 | 372 | CCCATCACTGTAAGAAATCC | 3 | 310 |
| 416436 | 167 | 360 | 379 | ACTGGTCCCCATCACTGTAA | 27 | 311 |
| 416437 | 167 | 366 | 385 | AGGCACACTGGTCCCCATCA | 52 | 312 |
| 416447 | 167 | 616 | 635 | CATGGATATTCAACTGTGGG | 17 | 313 |
| 416452 | 167 | 726 | 745 | CCAACAACAGGACCTGCCAT | 19 | 314 |
| 416454 | 167 | 846 | 865 | CGTGCTCGCCCAGCACCGCG | 56 | 315 |

Certain gapmers from Table 4 are 100% homologous to the rhesus monkey genomic sequence (nucleotides 691000 to 706000 of GENBANK Accession No. NW_{—}00104507.1; incorporated herein as SEQ ID NO: 162) or the rhesus monkey mRNA sequence (GENBANK Accession No. 3360_{—}061_B; incorporated herein as SEQ ID NO: 163). Shown in Table 5 are the chimeric antisense oligonucleotides from Table 4, which are homologous with rhesus monkey.

TABLE 5

Human/rhesus monkey cross-reactive chimeric antisense
oligonucleotides having 5-10-5 MOE wings and deoxy gap

| | | | | | | | | |
|---|---|---|---|---|---|---|---|---|
| | Human | Human | Human | | Rhesus | Rhesus | Rhesus | |
| | Target | Target | Target | | Target | Monkey | Monkey | SEQ |
| | SEQ | Start | Stop | | SEQ ID | Target | Target | ID |
| ISIS No. | ID No. | Site | Site | Sequence (5′ to 3′) | No. | Start Site | Stop Site | No. |

| | | | | | | | | |
|---|---|---|---|---|---|---|---|---|
| 407606 | 1 | 9204 | 9223 | CTGGTCCTTGCAGGAGCCCC | 162 | 6771 | 6790 | 17 |
| 407900 | 1 | 12141 | 12160 | CCCCACAATTCGGCCTTGGG | 162 | 9865 | 9884 | 88 |
| 407910 | 1 | 14104 | 14123 | AGTCCTTGCTGCCATCCGAG | 162 | 11826 | 11845 | 98 |
| 407641 | 1 | 14707 | 14726 | CCTGAGGCCAGCAGATCACG | 162 | 12427 | 12446 | 48 |
| 407643 | 1 | 15098 | 15117 | CACACATGGAGTCAGCATCG | 162 | 12815 | 12834 | 50 |
| 407935 | 1 | 15191 | 15210 | ATGCATGGTGATGCTTCTGA | 162 | 12908 | 12927 | 123 |
| 416492 | 1 | 616 | 635 | GGATCATTCTGGCCCTGAGC | 162 | 24 | 43 | 168 |
| 416493 | 1 | 738 | 757 | TCTTGGGTGTGGATGTAAAT | 162 | 149 | 168 | 169 |
| 416494 | 1 | 803 | 822 | CAGATTTAAACTGCAGATGA | 162 | 214 | 233 | 170 |
| 416495 | 1 | 838 | 857 | TCTAGAATTCCAAACCCCTA | 162 | 259 | 278 | 171 |
| 416496 | 1 | 855 | 874 | CAACACTTCAAATACGATCT | 162 | 276 | 295 | 172 |
| 416497 | 1 | 883 | 902 | CGTGCAGGTGTTAAGGTGTG | 162 | 306 | 325 | 173 |
| 416498 | 1 | 994 | 1013 | GGCCAGTGGCCATGCATCCC | 162 | 417 | 436 | 174 |
| 416499 | 1 | 1011 | 1030 | GAGAGCTGCACCTGGCCGGC | 162 | 434 | 453 | 175 |
| 416500 | 1 | 1026 | 1045 | CTGAACACCCCAGCTGAGAG | 162 | 449 | 468 | 176 |
| 416424 | 1 | 1151 | 1170 | GAGACCATGATGAAATCTCT | 162 | 574 | 593 | 177 |
| 416425 | 1 | 1182 | 1201 | AAGCAGAAGGCAGAGGAGCC | 162 | 605 | 624 | 178 |
| 416426 | 1 | 1187 | 1206 | AGCCCAAGCAGAAGGCAGAG | 162 | 610 | 629 | 179 |
| 416427 | 1 | 1193 | 1212 | CCCTGAAGCCCAAGCAGAAG | 162 | 616 | 635 | 180 |
| 416501 | 1 | 1251 | 1270 | CTGCCCTTCCACCAAGTTTA | 162 | 674 | 693 | 181 |
| 416502 | 1 | 1437 | 1456 | GTTCTTTGAAAAATAATCCC | 162 | 858 | 877 | 182 |
| 416423 | 1 | 2179 | 2198 | GTGTTTCTCCTCCTGAGGCC | 162 | 1638 | 1657 | 183 |
| 416503 | 1 | 2311 | 2330 | TAGCCACCCCGCGGGCTGGC | 162 | 1771 | 1790 | 184 |
| 416504 | 1 | 2484 | 2503 | TCAGAAAAGCTCTCAAGAAC | 162 | 1944 | 1963 | 185 |
| 416505 | 1 | 2495 | 2514 | GCAGATTTGCATCAGAAAAG | 162 | 1955 | 1974 | 186 |
| 416506 | 1 | 4766 | 4785 | CTTTAAAATCAGTTTCACAC | 162 | 2556 | 2575 | 187 |
| 416507 | 1 | 4847 | 4866 | GGTTACTGAGCGCGGAAGAA | 162 | 2637 | 2656 | 188 |
| 416508 | 1 | 4873 | 4892 | CGAGTTCTGCAGGAGCGGCC | 162 | 2663 | 2682 | 189 |
| 416509 | 1 | 4880 | 4899 | AGGAGCCCGAGTTCTGCAGG | 162 | 2670 | 2689 | 190 |
| 416510 | 1 | 4916 | 4935 | GACGAGGCCTCAGGTGGACG | 162 | 2706 | 2725 | 191 |
| 416511 | 1 | 4926 | 4945 | TTGCTGGGAGGACGAGGCCT | 162 | 2716 | 2735 | 192 |
| 416512 | 1 | 4934 | 4953 | GACGACCTTTGCTGGGAGGA | 162 | 2724 | 2743 | 193 |
| 416513 | 1 | 6399 | 6418 | CTGATGTGAAAACCGGCATG | 162 | 4144 | 4163 | 198 |
| 416514 | 1 | 6406 | 6425 | GTATTTTCTGATGTGAAAAC | 162 | 4151 | 4170 | 199 |
| 416515 | 1 | 8547 | 8566 | TAGGCATGACCATCCTCAAT | 162 | 6104 | 6123 | 200 |
| 416516 | 1 | 8599 | 8618 | GTGAGAATACAACAGATGAG | 162 | 6156 | 6175 | 201 |
| 416517 | 1 | 8708 | 8727 | GGGTGCAGTAGCAGATGCAA | 162 | 6265 | 6284 | 202 |
| 416518 | 1 | 8855 | 8874 | GGGTGACCACACATTTCCTG | 162 | 6422 | 6441 | 203 |
| 416434 | 1 | 9076 | 9095 | CTGTAAGAAATCCAGAACAG | 162 | 6640 | 6659 | 204 |
| 416491 | 1 | 9120 | 9139 | GAGAAGGGTTGCAGGCAGGA | 162 | 6684 | 6703 | 205 |
| 416438 | 1 | 9194 | 9213 | CAGGAGCCCCCATTCTGGCA | 162 | 6761 | 6780 | 206 |
| 416439 | 1 | 9201 | 9220 | GTCCTTGCAGGAGCCCCCAT | 162 | 6768 | 6787 | 207 |
| 416440 | 1 | 9213 | 9232 | GGACTGGAGCTGGTCCTTGC | 162 | 6780 | 6799 | 208 |
| 416441 | 1 | 9220 | 9239 | AGATATAGGACTGGAGCTGG | 162 | 6787 | 6806 | 209 |
| 416442 | 1 | 9223 | 9242 | AGCAGATATAGGACTGGAGC | 162 | 6790 | 6809 | 210 |
| 416443 | 1 | 9231 | 9250 | GAGGCAGAAGCAGATATAGG | 162 | 6798 | 6817 | 211 |
| 416444 | 1 | 9255 | 9274 | ACAGTTCCGGCCCTCGAAGG | 162 | 6822 | 6841 | 212 |
| 416519 | 1 | 9290 | 9309 | AAATATGGGACCCAAAGTGG | 162 | 6857 | 6876 | 213 |
| 416520 | 1 | 9298 | 9317 | CCCTCTGCAAATATGGGACC | 162 | 6865 | 6884 | 214 |
| 416521 | 1 | 9362 | 9381 | CACCCCACCAGGTTGTGCAC | 162 | 6906 | 6925 | 215 |
| 416522 | 1 | 9515 | 9534 | GGCTGAGAATTGCCCAGGGC | 162 | 7059 | 7078 | 216 |
| 416523 | 1 | 9522 | 9541 | TCTCGAGGGCTGAGAATTGC | 162 | 7066 | 7085 | 217 |
| 416524 | 1 | 9665 | 9684 | TAATTTAATATTCAGATGGT | 162 | 7239 | 7258 | 218 |
| 416525 | 1 | 9725 | 9744 | TATGAGTCCTTCTAGTGAAT | 162 | 7299 | 7318 | 219 |
| 416526 | 1 | 9848 | 9867 | TGTCCACATGACCCCACAGG | 162 | 7422 | 7441 | 220 |
| 416527 | 1 | 9912 | 9931 | GAGCTTCCCAAGTTGGCAGT | 162 | 7479 | 7498 | 221 |
| 416528 | 1 | 9942 | 9961 | TGATAAAACCTCTGGACACC | 162 | 7509 | 7528 | 222 |
| 416529 | 1 | 9999 | 10018 | GGGCTGAGACTGAGGTCAGC | 162 | 7566 | 7585 | 223 |
| 416530 | 1 | 10166 | 10185 | AGGGTAGCCTTTGCCTTGGC | 162 | 7852 | 7871 | 224 |
| 416531 | 1 | 10317 | 10336 | AGATGACCAGCAGGAAGCCT | 162 | 8006 | 8025 | 225 |
| 416532 | 1 | 10323 | 10342 | GGACCCAGATGACCAGCAGG | 162 | 8012 | 8031 | 226 |
| 416533 | 1 | 10330 | 10349 | GCATTCTGGACCCAGATGAC | 162 | 8019 | 8038 | 227 |
| 416534 | 1 | 10377 | 10396 | ATGCACACCAGGGCTGCTGG | 162 | 8066 | 8085 | 228 |
| 416535 | 1 | 10382 | 10401 | GCAGGATGCACACCAGGGCT | 162 | 8071 | 8090 | 229 |
| 416536 | 1 | 10398 | 10417 | CGGGAAGGCCTGCCCTGCAG | 162 | 8087 | 8106 | 230 |
| 416537 | 1 | 10677 | 10696 | TGACCACTCTTCCGAGCAGC | 162 | 8376 | 8395 | 231 |
| 416538 | 1 | 10807 | 10826 | CGTGGACTGATCCAAAGGAC | 162 | 8505 | 8524 | 232 |
| 416539 | 1 | 10837 | 10856 | GACAGAGCCTGAGCTTGGCA | 162 | 8535 | 8554 | 233 |
| 416445 | 1 | 11013 | 11032 | TACTGCTCACAGCCGCCGTT | 162 | 8710 | 8729 | 234 |
| 416446 | 1 | 11024 | 11043 | GGTCACTGCAGTACTGCTCA | 162 | 8721 | 8740 | 235 |
| 416540 | 1 | 11143 | 11162 | GGACTGGTGTCATCTGGGAC | 162 | 8840 | 8859 | 236 |
| 416541 | 1 | 11259 | 11278 | CCACCCTTGGTGCCCAGATC | 162 | 8953 | 8972 | 237 |
| 416542 | 1 | 11297 | 11316 | CAGGGTGCCCATCCTAGTCA | 162 | 8991 | 9010 | 238 |
| 416543 | 1 | 11395 | 11414 | TCCTGCGAGTGGGAGTTGGA | 162 | 9089 | 9108 | 239 |
| 416544 | 1 | 11499 | 11518 | ATCCCATTTTCCCAGGAGCC | 162 | 9193 | 9212 | 240 |
| 416545 | 1 | 11505 | 11524 | AGAAACATCCCATTTTCCCA | 162 | 9199 | 9218 | 241 |
| 416546 | 1 | 11519 | 11538 | CCAGGCTGGTTTGGAGAAAC | 162 | 9213 | 9232 | 242 |
| 416547 | 1 | 11729 | 11748 | ATGAAATTCTACCTAAAGAT | 162 | 9434 | 9453 | 243 |
| 416548 | 1 | 11735 | 11754 | AGGTGAATGAAATTCTACCT | 162 | 9440 | 9459 | 244 |
| 416549 | 1 | 11838 | 11857 | GACAATGGTCAGGGCTGGTT | 162 | 9543 | 9562 | 245 |
| 416550 | 1 | 11852 | 11871 | TGGCTGGCTGAGGAGACAAT | 162 | 9557 | 9576 | 246 |
| 416551 | 1 | 12000 | 12019 | CAGAAACACCCATCCTCTGA | 162 | 9724 | 9743 | 247 |
| 416448 | 1 | 12088 | 12107 | TTTTTCCACATGGATATTCA | 162 | 9812 | 9831 | 248 |
| 416449 | 1 | 12094 | 12113 | TAGGTATTTTTCCACATGGA | 162 | 9818 | 9837 | 249 |
| 416450 | 1 | 12122 | 12141 | GGTTTGCTGGCATTTCTTTT | 162 | 9846 | 9865 | 250 |
| 416451 | 1 | 12175 | 12194 | GCCATGGACACTCCCCTTTG | 162 | 9899 | 9918 | 251 |
| 416552 | 1 | 12398 | 12417 | TCTGCACAGGGTAGACCCTC | 162 | 10127 | 10146 | 252 |
| 416553 | 1 | 12403 | 12422 | GGTTCTCTGCACAGGGTAGA | 162 | 10132 | 10151 | 253 |
| 416554 | 1 | 12467 | 12486 | AAAGATCCCACCTCAAAGAG | 162 | 10196 | 10215 | 254 |
| 416555 | 1 | 12478 | 12497 | AAAGATCAGGCAAAGATCCC | 162 | 10207 | 10226 | 255 |
| 416556 | 1 | 12508 | 12527 | ATAGCTTTGATCCAATGCTC | 162 | 10237 | 10256 | 256 |
| 416557 | 1 | 12639 | 12658 | TCCCAGGCAAAGCTGCTCAG | 162 | 10368 | 10387 | 257 |
| 416453 | 1 | 12867 | 12886 | GATTTTGTCGAAACAGTGGG | 162 | 10596 | 10615 | 258 |
| 416558 | 1 | 13159 | 13178 | TGACAGCACGAAGCCCAGAG | 162 | 10880 | 10899 | 259 |
| 416559 | 1 | 13638 | 13657 | GCCATTTCTAGGTCTGCAGG | 162 | 11360 | 11379 | 260 |
| 416455 | 1 | 13760 | 13779 | CGCCGGCTCTGCTCATCCCC | 162 | 11482 | 11501 | 261 |
| 416456 | 1 | 13770 | 13789 | CTGCGCCACCCGCCGGCTCT | 162 | 11492 | 11511 | 262 |
| 416457 | 1 | 13780 | 13799 | GGATGATGACCTGCGCCACC | 162 | 11502 | 11521 | 263 |
| 416458 | 1 | 13831 | 13850 | GGCGGAGCAGCGCGATGTCG | 162 | 11553 | 11572 | 264 |
| 416459 | 1 | 13859 | 13878 | TGGTCAGTGAGGACCACGGG | 162 | 11581 | 11600 | 265 |
| 416460 | 1 | 13866 | 13885 | CACCACATGGTCAGTGAGGA | 162 | 11588 | 11607 | 266 |
| 416461 | 1 | 13923 | 13942 | GAAGCGCACGAAGGCCAGCG | 162 | 11645 | 11664 | 267 |
| 416462 | 1 | 14020 | 14039 | GGCAGTCCTGGGTCATCAGC | 162 | 11742 | 11761 | 268 |
| 416463 | 1 | 14027 | 14046 | TGCTGCAGGCAGTCCTGGGT | 162 | 11749 | 11768 | 269 |
| 416464 | 1 | 14072 | 14091 | AACATGTACTCCGTGATATT | 162 | 11794 | 11813 | 270 |
| 416465 | 1 | 14122 | 14141 | CACTGTCCCCCTTGCAGGAG | 162 | 11844 | 11863 | 271 |
| 416466 | 1 | 14132 | 14151 | TGTGGGCCTCCACTGTCCCC | 162 | 11854 | 11873 | 272 |
| 416467 | 1 | 14189 | 14208 | CCCTGGCCCCAGCTGACGAT | 162 | 11911 | 11930 | 273 |
| 416468 | 1 | 14234 | 14253 | TGGGAGACCCTGGTGTACAC | 162 | 11956 | 11975 | 274 |
| 416469 | 1 | 14251 | 14270 | GCAGCCACTCGATGTACTGG | 162 | 11973 | 11992 | 275 |
| 416470 | 1 | 14257 | 14276 | GCTTTTGCAGCCACTCGATG | 162 | 11979 | 11998 | 276 |
| 416471 | 1 | 14260 | 14279 | TGAGCTTTTGCAGCCACTCG | 162 | 11982 | 12001 | 277 |
| 416472 | 1 | 14348 | 14367 | CAGCCTTGGCTTTCTCTCCA | 162 | 12070 | 12089 | 278 |
| 416473 | 1 | 14613 | 14632 | CCCTGCCCCTCTGTCCAGCG | 162 | 12333 | 125352 | 279 |
| 416474 | 1 | 14642 | 14661 | TGTCTGCCTCCAGGACAACC | 162 | 12362 | 12381 | 280 |
| 416475 | 1 | 14653 | 14672 | CTCAGCTGGGCTGTCTGCCT | 162 | 12373 | 12392 | 281 |
| 416476 | 1 | 14686 | 14705 | GCAGGTGGGCTTGGCTGAAG | 162 | 12406 | 12425 | 282 |
| 416477 | 1 | 14710 | 14729 | CAGCCTGAGGCCAGCAGATC | 162 | 12430 | 12449 | 283 |
| 416478 | 1 | 14735 | 14754 | GTCTCCAGCAATGAAGGCAG | 162 | 12455 | 12474 | 284 |
| 416479 | 1 | 15103 | 15122 | GACAGCACACATGGAGTCAG | 162 | 12820 | 12839 | 285 |
| 416480 | 1 | 15132 | 15151 | AAGTGAGAGCTAAACAACCG | 162 | 12849 | 12868 | 286 |
| 416481 | 1 | 15157 | 15176 | AGATGATAATGGATAAGAAC | 162 | 12874 | 12893 | 287 |
| 416482 | 1 | 15188 | 15207 | CATGGTGATGCTTCTGAATT | 162 | 12905 | 12924 | 288 |
| 416483 | 1 | 15433 | 15452 | TGTGAGGGTTCATTTCAGTG | 162 | 13130 | 13149 | 289 |
| 416484 | 1 | 15485 | 15504 | TACAGCCTCAAACATCTATC | 162 | 13184 | 13203 | 290 |
| 416485 | 1 | 15489 | 15508 | GAGCTACAGCCTCAAACATC | 162 | 13188 | 13207 | 291 |
| 416486 | 1 | 15540 | 15559 | ATTGACAAGGGCTGTGGCAG | 162 | 13239 | 13258 | 292 |
| 416487 | 1 | 15571 | 15590 | GCAGGTGCTCCCAGGGTCTC | 162 | 13270 | 13289 | 293 |
| 416488 | 1 | 15639 | 15658 | CAGGTCCTCCCACTGCAGCC | 162 | 13334 | 13353 | 294 |
| 416489 | 1 | 15650 | 15669 | GTGCAGCTTGCCAGGTCCTC | 162 | 13345 | 13364 | 295 |
| 416490 | 1 | 15661 | 15680 | ACTCAGCAAGAGTGCAGCTT | 162 | 13356 | 13375 | 296 |
| 416560 | 1 | 15973 | 15992 | TAAAACTTTATTTACAAGAG | 162 | 13638 | 13657 | 297 |
| 416561 | 1 | 15985 | 16004 | GGTGTGTTCCCATAAAACTT | 162 | 13650 | 13669 | 298 |
| 416562 | 1 | 16185 | 16204 | AAAGCAGAGCCAGCTCTGAC | 162 | 13849 | 13868 | 299 |
| 416563 | 1 | 16596 | 16615 | GCCTGCATTTCCCATTGGCA | 162 | 13931 | 13950 | 300 |
| 416564 | 1 | 16738 | 16757 | GCCACTCACAGAAAGCTGGA | 162 | 14071 | 14090 | 301 |
| 416565 | 1 | 16872 | 16891 | CAGGATGCTCATGGCAGACA | 162 | 14205 | 14224 | 302 |
| 416566 | 1 | 16911 | 16930 | GTTTGTATGGAGAGACCAAT | 162 | 14244 | 14263 | 303 |
| 416567 | 1 | 16977 | 16996 | TGGTGGCACCAGATGTCTGA | 162 | 14310 | 14329 | 304 |
| 416568 | 1 | 17112 | 17131 | CATTGTGCCTGGCACACAGG | 162 | 14445 | 14464 | 305 |
| 416569 | 1 | 17136 | 17155 | GCCTGGTGTGCACACATTGT | 162 | 14469 | 14488 | 306 |
| 416422 | 167 | 107 | 126 | AGCGACCCCGCCTGCAGCCA | 163 | 107 | 126 | 308 |
| 416433 | 167 | 341 | 360 | AGAAATCCAGAACAGCTTCG | 163 | 341 | 360 | 309 |
| 416435 | 167 | 353 | 372 | CCCATCACTGTAAGAAATCC | 163 | 353 | 372 | 310 |
| 416436 | 167 | 360 | 379 | ACTGGTCCCCATCACTGTAA | 163 | 360 | 379 | 311 |
| 416437 | 167 | 366 | 385 | AGGCACACTGGTCCCCATCA | 163 | 366 | 385 | 312 |
| 416447 | 167 | 616 | 635 | CATGGATATTCAACTGTGGG | 163 | 616 | 635 | 313 |
| 416452 | 167 | 726 | 745 | CCAACAACAGGACCTGCCAT | 163 | 726 | 745 | 314 |
| 416454 | 167 | 846 | 865 | CGTGCTCGCCCAGCACCGCG | 163 | 846 | 865 | 315 |

### Example 4

### Antisense Inhibition of Human Factor 7 in HepB3 Cells

Antisense oligonucleotides targeted to a Factor 7 nucleic acid were designed and tested for their effects on Factor 7 mRNA in vitro. Cultured HepB3 cells at a density of 4,000 cells per well were transfected using lipofectin reagent with 50 nM antisense oligonucleotide. After a treatment period of approximately 24 hours, RNA was isolated from the cells and Factor 7 mRNA levels were measured by real-time RT-PCR, as described herein. Human Factor 7 primer probe set RTS 2927 was used to measure mRNA levels. Factor 7 mRNA levels were adjusted according to total RNA content as measured by RIBOGREEN®. Results are presented as percent inhibition of Factor 7, relative to untreated control.

The chimeric antisense oligonucleotides in Table 6 were designed as 5-10-5 MOE, 3-14-3 MOE, and 2-13-5 MOE gapmers. The 5-10-5 gapmers are 20 nucleotides in length, wherein the central gap segment is comprised of ten 2′-deoxynucleotides and is flanked on both sides (in the 5′ and 3′ directions) by wings comprising five nucleotides each. The 3-14-3 gapmers are 20 nucleotides in length, wherein the central gap segment is comprised of fourteen 2′-deoxynucleotides and is flanked on both sides (in the 5′ and 3′ directions) by wings comprising three nucleotides each. The 2-13-5 gapmers are 20 nucleotides in length, wherein the central gap segment is comprised of thirteen 2′-deoxynucleotides. The central gap is flanked on the 5′ end with a wing comprising two nucleotides and on the 3′ end with a wing comprising five nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 6 is targeted to SEQ ID NO: 1 (nucleotides 1255000 to 1273000 of GENBANK Accession No. NT_{—}027140.6).

TABLE 6

Inhibition of human Factor 7 mRNA levels by chimeric antisense
oligonucleotides targeted to SEQ ID NO: 1

| | | | | | | |
|---|---|---|---|---|---|---|
| | Target | Target | | | | SEQ |
| ISIS | Start | Stop | | | % | ID |
| No. | Site | Site | Sequence (5′ to 3′) | Motif | Inhibition | No. |

| | | | | | | |
|---|---|---|---|---|---|---|
| 422117 | 4834 | 4853 | GGAAGAAGGGACGGAAGGTG | 5-10-5 | 1 | 316 |
| 422186 | 4834 | 4853 | GGAAGAAGGGACGGAAGGTG | 3-14-3 | 14 | 316 |
| 422263 | 4834 | 4853 | GGAAGAAGGGACGGAAGGTG | 2-13-5 | 19 | 316 |
| 422118 | 4835 | 4854 | CGGAAGAAGGGACGGAAGGT | 5-10-5 | 15 | 317 |
| 422187 | 4835 | 4854 | CGGAAGAAGGGACGGAAGGT | 3-14-3 | 28 | 317 |
| 422264 | 4835 | 4854 | CGGAAGAAGGGACGGAAGGT | 2-13-5 | 24 | 317 |
| 422119 | 4836 | 4855 | GCGGAAGAAGGGACGGAAGG | 5-10-5 | 39 | 318 |
| 422188 | 4836 | 4855 | GCGGAAGAAGGGACGGAAGG | 3-14-3 | 37 | 318 |
| 422265 | 4836 | 4855 | GCGGAAGAAGGGACGGAAGG | 2-13-5 | 34 | 318 |
| 422120 | 4837 | 4856 | CGCGGAAGAAGGGACGGAAG | 5-10-5 | 40 | 319 |
| 422189 | 4837 | 4856 | CGCGGAAGAAGGGACGGAAG | 3-14-3 | 0 | 319 |
| 422266 | 4837 | 4856 | CGCGGAAGAAGGGACGGAAG | 2-13-5 | 13 | 319 |
| 422121 | 4838 | 4857 | GCGCGGAAGAAGGGACGGAA | 5-10-5 | 43 | 320 |
| 422190 | 4838 | 4857 | GCGCGGAAGAAGGGACGGAA | 3-14-3 | 12 | 320 |
| 422267 | 4838 | 4857 | GCGCGGAAGAAGGGACGGAA | 2-13-5 | 38 | 320 |
| 422122 | 4839 | 4858 | AGCGCGGAAGAAGGGACGGA | 5-10-5 | 24 | 321 |
| 422191 | 4839 | 4858 | AGCGCGGAAGAAGGGACGGA | 3-14-3 | 27 | 321 |
| 422268 | 4839 | 4858 | AGCGCGGAAGAAGGGACGGA | 2-13-5 | 63 | 321 |
| 422123 | 4840 | 4859 | GAGCGCGGAAGAAGGGACGG | 5-10-5 | 16 | 322 |
| 422192 | 4840 | 4859 | GAGCGCGGAAGAAGGGACGG | 3-14-3 | 27 | 322 |
| 422269 | 4840 | 4859 | GAGCGCGGAAGAAGGGACGG | 2-13-5 | 27 | 322 |
| 422124 | 4841 | 4860 | TGAGCGCGGAAGAAGGGACG | 5-10-5 | 0 | 323 |
| 422193 | 4841 | 4860 | TGAGCGCGGAAGAAGGGACG | 3-14-3 | 6 | 323 |
| 422270 | 4841 | 4860 | TGAGCGCGGAAGAAGGGACG | 2-13-5 | 15 | 323 |
| 422125 | 4842 | 4861 | CTGAGCGCGGAAGAAGGGAC | 5-10-5 | 8 | 324 |
| 422194 | 4842 | 4861 | CTGAGCGCGGAAGAAGGGAC | 3-14-3 | 11 | 324 |
| 422271 | 4842 | 4861 | CTGAGCGCGGAAGAAGGGAC | 2-13-5 | 32 | 324 |
| 422126 | 4843 | 4862 | ACTGAGCGCGGAAGAAGGGA | 5-10-5 | 22 | 325 |
| 422195 | 4843 | 4862 | ACTGAGCGCGGAAGAAGGGA | 3-14-3 | 37 | 325 |
| 422272 | 4843 | 4862 | ACTGAGCGCGGAAGAAGGGA | 2-13-5 | 12 | 325 |
| 422127 | 4844 | 4863 | TACTGAGCGCGGAAGAAGGG | 5-10-5 | 17 | 326 |
| 422196 | 4844 | 4863 | TACTGAGCGCGGAAGAAGGG | 3-14-3 | 2 | 326 |
| 422273 | 4844 | 4863 | TACTGAGCGCGGAAGAAGGG | 2-13-5 | 0 | 326 |
| 422128 | 4845 | 4864 | TTACTGAGCGCGGAAGAAGG | 5-10-5 | 27 | 327 |
| 422197 | 4845 | 4864 | TTACTGAGCGCGGAAGAAGG | 3-14-3 | 26 | 327 |
| 422274 | 4845 | 4864 | TTACTGAGCGCGGAAGAAGG | 2-13-5 | 24 | 327 |
| 422129 | 4846 | 4865 | GTTACTGAGCGCGGAAGAAG | 5-10-5 | 45 | 328 |
| 422198 | 4846 | 4865 | GTTACTGAGCGCGGAAGAAG | 3-14-3 | 50 | 328 |
| 422275 | 4846 | 4865 | GTTACTGAGCGCGGAAGAAG | 2-13-5 | 42 | 328 |
| 422199 | 4847 | 4866 | GGTTACTGAGCGCGGAAGAA | 3-14-3 | 65 | 188 |
| 422276 | 4847 | 4866 | GGTTACTGAGCGCGGAAGAA | 2-13-5 | 66 | 188 |

### Example 5

### Dose-Dependent Antisense Inhibition of Human Coagulation Factor 7 in HepB3 Cells

Gapmers (from Tables 1 through 6, above) exhibiting in vitro inhibition of Factor 7 were selected and tested at various doses in HepB3 cells. Cells were plated at a density of 4,000 cells per well and transfected using lipofectin reagent with 6.25 nM, 12.5 nM, 25.0 nM, 50.0 nM, and 100.0 nM concentrations of antisense oligonucleotide, as indicated in Table 7. After a treatment period of approximately 16 hours, RNA was isolated from the cells and Factor 7 mRNA levels were measured by real-time RT-PCR, as described herein. Human Factor 7 primer probe set RTS 2927 was used to measure mRNA levels. Factor 7 mRNA levels were adjusted according to total RNA content as measured by RIBOGREEN®. Results are presented as percent inhibition of Factor 7, relative to untreated control cells. As illustrated in Table 7, Factor 7 mRNA levels were reduced in a dose-dependent manner.

TABLE 7
Dose-dependent antisense inhibition of human Factor 7 in HepB3 cells

| ISIS | | | | | 100.0 | SEQ ID |
|---|---|---|---|---|---|---|
| No. | 6.25 nM | 12.5 nM | 25.0 nM | 50.0 nM | nM | No. |
| 407643 | 36 | 49 | 70 | 83 | 93 | 50 |
| 407900 | 18 | 36 | 61 | 82 | 91 | 88 |
| 407935 | 38 | 53 | 70 | 82 | 86 | 123 |
| 407939 | 37 | 57 | 77 | 84 | 87 | 127 |
| 416438 | 11 | 33 | 56 | 75 | 82 | 206 |
| 416446 | 25 | 24 | 50 | 69 | 64 | 235 |
| 416449 | 19 | 33 | 45 | 65 | 85 | 249 |
| 416455 | 28 | 44 | 64 | 78 | 87 | 261 |
| 416472 | 16 | 44 | 64 | 80 | 88 | 278 |
| 416477 | 21 | 46 | 64 | 78 | 87 | 283 |
| 416507 | 30 | 53 | 70 | 77 | 72 | 188 |
| 416508 | 42 | 51 | 71 | 79 | 89 | 189 |
| 416549 | 38 | 44 | 63 | 73 | 78 | 245 |

### Example 6

### Antisense Inhibition of Human Factor 7 in HepB3 Cells by Oligonucleotides Designed by Microwalk

Additional gapmers were designed based on the gapmers presented in Table 7. These gapmers were designed by creating gapmers shifted slightly upstream and downstream (i.e. “microwalk”) of the original gapmers from Table 7. Gapmers were also created with various motifs, e.g. 5-10-5 MOE, 3-14-3 MOE, and 2-13-5 MOE. These gapmers were tested in vitro. Cultured HepB3 cells at a density of 4,000 cells per well were transfected using lipofectin reagent with 50 nM antisense oligonucleotide. After a treatment period of approximately 24 hours, RNA was isolated from the cells and Factor 7 mRNA levels were measured by real-time RT-PCR. Factor 7 mRNA levels were adjusted according to total RNA content, as measured by RIBOGREEN®. Results are presented as percent inhibition of Factor 7, relative to untreated control cells.

The in vitro inhibition data for the gapmers designed by microwalk was then compared with the in vitro inhibition data for the gapmers from Table 7, as indicated in Tables 8, 9, 10, 11, 12, and 13. The oligonucleotides are displayed according to the region on the human gene sequence to which they map.

The chimeric antisense oligonucleotides in Table 8 were designed as 5-10-5 MOE, 3-14-3 MOE, and 2-13-5 MOE gapmers. The first listed gapmer in Table 8 is the original gapmer (see Table 7) from which the remaining gapmers were designed via microwalk and is designated by an asterisk (*). The 5-10-5 gapmers are 20 nucleotides in length, wherein the central gap segment is comprised of ten 2′-deoxynucleotides and is flanked on both sides (in the 5′ and 3′ directions) by wings comprising five nucleotides each. The 3-14-3 gapmers are 20 nucleotides in length, wherein the central gap segment is comprised of fourteen 2′-deoxynucleotides and is flanked on both sides (in the 5′ and 3′ directions) by wings comprising three nucleotides each. The 2-13-5 gapmers are 20 nucleotides in length, wherein the central gap segment is comprised of thirteen 2′-deoxynucleotides. The central gap is flanked on the 5′ end with a wing comprising two nucleotides and on the 3′ end with a wing comprising five nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 8 is targeted to SEQ ID NO: 1 (nucleotides 1255000 to 1273000 of GENBANK Accession No. NT_{—}027140.6).

As shown in Table 8, all of the 5-10-5 MOE gapmers, 3-14-3 MOE gapmers, and 2-13-5 MOE gapmers targeted to the target region beginning at target start site 4868 and ending at the target stop site 4899 (i.e. nucleobases 4868-4899) of SEQ ID NO: 1 inhibited Factor 7 mRNA by at least 48%.

Certain gapmers within the target region (i.e. nucleobases 4868-4899) inhibited Factor 7 mRNA expression by at least 40%, for example, ISIS numbers 416508, 422138, 422213, 422290, 422139, 422214, 422291, 422140, 422215, 422292, 422141, 422216, 422293, 422142, 422217, 422294, 422218, 422295, 422143, 422219, 422296, 422144, 422220, 422297, 422145, 422221, 422298, 422146, 422222, 422299, 422147, 422223, 422300, 422148, 422224, 422301, 416509, 422225, and 422302.

Certain gapmers within the target region (i.e. nucleobases 4868-4899) inhibited Factor 7 mRNA expression by at least 50%, for example, ISIS numbers 416508, 422138, 422213, 422290, 422139, 422214, 422291, 422140, 422215, 422292, 422141, 422216, 422293, 422142, 422217, 422294, 422218, 422295, 422143, 422219, 422296, 422144, 422220, 422297, 422145, 422221, 422298, 422146, 422222, 422299, 422147, 422300, 422148, 422224, 422301, 416509, 422225, and 422302.

Certain gapmers within the target region (i.e. nucleobases 4868-4899) inhibited Factor 7 mRNA expression by at least 60%, for example, ISIS numbers 416508, 422138, 422213, 422139, 422140, 422215, 422292, 422141, 422216, 422293, 422142, 422217, 422294, 422218, 422295, 422143, 422219, 422296, 422297, 422298, 422299, 422147, 422300, 422224, 422301, 416509, and 422302.

Certain gapmers within the target region (i.e. nucleobases 4868-4899) inhibited Factor 7 mRNA expression by at least 70%, for example, ISIS numbers 422138, 422140, 422215, 422292, 422142, 422217, 422294, 422218, 422295, 422143, and 422296.

TABLE 8

Inhibition of human Factor 7 mRNA levels by chimeric
antisense oligonucleotides targeted to nucleobases
4868 to 4899 of SEQ ID NO: 1

| | | | | | | |
|---|---|---|---|---|---|---|
| | Target | Target | | | | |
| | Start | Stop | | | % | SEQ ID |
| ISIS No. | Site | Site | Sequence (5′ to 3′) | Motif | Inhibition | NO |

| | | | | | | |
|---|---|---|---|---|---|---|
| *416508 | 4873 | 4892 | CGAGTTCTGCAGGAGCGGCC | 5-10-5 | 67 | 189 |
| 422138 | 4868 | 4887 | TCTGCAGGAGCGGCCTAAAT | 5-10-5 | 70 | 329 |
| 422213 | 4868 | 4887 | TCTGCAGGAGCGGCCTAAAT | 3-14-3 | 67 | 329 |
| 422290 | 4868 | 4887 | TCTGCAGGAGCGGCCTAAAT | 2-13-5 | 50 | 329 |
| 422139 | 4869 | 4888 | TTCTGCAGGAGCGGCCTAAA | 5-10-5 | 66 | 330 |
| 422214 | 4869 | 4888 | TTCTGCAGGAGCGGCCTAAA | 3-14-3 | 60 | 330 |
| 422291 | 4869 | 4888 | TTCTGCAGGAGCGGCCTAAA | 2-13-5 | 53 | 330 |
| 422140 | 4870 | 4889 | GTTCTGCAGGAGCGGCCTAA | 5-10-5 | 74 | 331 |
| 422215 | 4870 | 4889 | GTTCTGCAGGAGCGGCCTAA | 3-14-3 | 73 | 331 |
| 422292 | 4870 | 4889 | GTTCTGCAGGAGCGGCCTAA | 2-13-5 | 75 | 331 |
| 422141 | 4871 | 4890 | AGTTCTGCAGGAGCGGCCTA | 5-10-5 | 64 | 332 |
| 422216 | 4871 | 4890 | AGTTCTGCAGGAGCGGCCTA | 3-14-3 | 68 | 332 |
| 422293 | 4871 | 4890 | AGTTCTGCAGGAGCGGCCTA | 2-13-5 | 69 | 332 |
| 422142 | 4872 | 4891 | GAGTTCTGCAGGAGCGGCCT | 5-10-5 | 73 | 333 |
| 422217 | 4872 | 4891 | GAGTTCTGCAGGAGCGGCCT | 3-14-3 | 75 | 333 |
| 422294 | 4872 | 4891 | GAGTTCTGCAGGAGCGGCCT | 2-13-5 | 78 | 333 |
| 422218 | 4873 | 4892 | CGAGTTCTGCAGGAGCGGCC | 3-1-4-3 | 70 | 189 |
| 422295 | 4873 | 4892 | CGAGTTCTGCAGGAGCGGCC | 2-13-5 | 74 | 189 |
| 422143 | 4874 | 4893 | CCGAGTTCTGCAGGAGCGGC | 5-10-5 | 70 | 334 |
| 422219 | 4874 | 4893 | CCGAGTTCTGCAGGAGCGGC | 3-14-3 | 65 | 334 |
| 422296 | 4874 | 4893 | CCGAGTTCTGCAGGAGCGGC | 2-13-5 | 74 | 334 |
| 422144 | 4875 | 4894 | CCCGAGTTCTGCAGGAGCGG | 5-10-5 | 58 | 335 |
| 422220 | 4875 | 4894 | CCCGAGTTCTGCAGGAGCGG | 3-1-4-3 | 59 | 335 |
| 422297 | 4875 | 4894 | CCCGAGTTCTGCAGGAGCGG | 2-13-5 | 63 | 335 |
| 422145 | 4876 | 4895 | GCCCGAGTTCTGCAGGAGCG | 5-10-5 | 57 | 336 |
| 422221 | 4876 | 4895 | GCCCGAGTTCTGCAGGAGCG | 3-1-4-3 | 59 | 336 |
| 422298 | 4876 | 4895 | GCCCGAGTTCTGCAGGAGCG | 2-1-3-5 | 62 | 336 |
| 422146 | 4877 | 4896 | AGCCCGAGTTCTGCAGGAGC | 5-10-5 | 58 | 337 |
| 422222 | 4877 | 4896 | AGCCCGAGTTCTGCAGGAGC | 3-14-3 | 55 | 337 |
| 422299 | 4877 | 4896 | AGCCCGAGTTCTGCAGGAGC | 2-13-5 | 64 | 337 |
| 422147 | 4878 | 4897 | GAGCCCGAGTTCTGCAGGAG | 5-10-5 | 64 | 338 |
| 422223 | 4878 | 4897 | GAGCCCGAGTTCTGCAGGAG | 3-1-4-3 | 48 | 338 |
| 422300 | 4878 | 4897 | GAGCCCGAGTTCTGCAGGAG | 2-13-5 | 65 | 338 |
| 422148 | 4879 | 4898 | GGAGCCCGAGTTCTGCAGGA | 5-10-5 | 57 | 339 |
| 422224 | 4879 | 4898 | GGAGCCCGAGTTCTGCAGGA | 3-14-3 | 62 | 339 |
| 422301 | 4879 | 4898 | GGAGCCCGAGTTCTGCAGGA | 2-13-5 | 67 | 339 |
| 416509 | 4880 | 4899 | AGGAGCCCGAGTTCTGCAGG | 5-10-5 | 60 | 190 |
| 422225 | 4880 | 4899 | AGGAGCCCGAGTTCTGCAGG | 3-14-3 | 56 | 190 |
| 422302 | 4880 | 4899 | AGGAGCCCGAGTTCTGCAGG | 2-13-5 | 67 | 190 |

The chimeric antisense oligonucleotides in Table 9 were designed as 5-10-5 MOE, 3-14-3 MOE, and 2-13-5 MOE gapmers. The first listed gapmer in Table 9 is the original gapmer (see Table 7) from which the remaining gapmers were designed via microwalk and is designated by an asterisk (*). The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 7 is targeted to SEQ ID NO: 1 (nucleotides 1255000 to 1273000 of GENBANK Accession No. NT_{—}027140.6).

As shown in Table 9, most of the 5-10-5 MOE gapmers, 3-14-3 MOE gapmers, and 2-13-5 MOE gapmers targeted to the target region beginning at target start site 11830 and ending at the target stop site 11869 (i.e. nucleobases 11830-11869) of SEQ ID NO: 1 inhibited Factor 7 mRNA by at least 40%.

Certain gapmers within the target region (i.e. nucleobases 11830-11869) inhibited Factor 7 mRNA expression by at least 20%, for example, ISIS numbers 416549, 422154, 422231, 422089, 422155, 422232, 422090, 422156, 422233, 422091, 422157, 422234, 422092, 422158, 422235, 422093, 422159, 422236, 422094, 422160, 422237, 422095, 422161, 422238, 422162, 422239, 422096, 422163, 422240, 422097, 422164, 422241, 422098, 422165, 422242, 422099, 422166, 422243, 422100, 422167, 422244, 422101, 422168, 422245, 422102, 422169, 422246, 422103, 422170, 422247, 422104, 422171, 422248, 422105, 422172, 422249, 422106, 422173, 422250, 422107, 422174, and 422251.

Certain gapmers within the target region (i.e. nucleobases 11830-11869) inhibited Factor 7 mRNA expression by at least 30%, for example, ISIS numbers 416549, 422154, 422155, 422232, 422090, 422156, 422233, 422091, 422157, 422234, 422092, 422158, 422235, 422093, 422159, 422236, 422094, 422160, 422237, 422095, 422161, 422238, 422162, 422239, 422096, 422163, 422240, 422097, 422164, 422241, 422098, 422165, 422242, 422099, 422166, 422243, 422100, 422167, 422244, 422101, 422168, 422102, 422169, 422246, 422103, 422247, 422104, 422171, 422248, 422105, 422172, 422249, 422106, 422173, 422250, 422107, 422174, and 422251.

Certain gapmers within the target region (i.e. nucleobases 11830-11869) inhibited Factor 7 mRNA expression by at least 40%, for example, ISIS numbers 416549, 422232, 422090, 422233, 422091, 422157, 422234, 422158, 422235, 422093, 422159, 422236, 422094, 422160, 422237, 422095, 422161, 422238, 422162, 422239, 422096, 422163, 422240, 422097, 422164, 422241, 422098, 422165, 422242, 422099, 422166, 422243, 422100, 422167, 422244, 422101, 422102, 422169, 422246, 422104, 422171, 422248, 422105, 422249, 422173, 422250, and 422174.

Certain gapmers within the target region (i.e. nucleobases 11830-11869) inhibited Factor 7 mRNA expression by at least 50%, for example, ISIS numbers 416549, 422234, 422235, 422237, 422095, 422161, 422238, 422162, 422239, 422096, 422163, 422240, 422097, 422164, 422241, 422098, 422165, 422242, 422166, 422243, 422100, 422167, 422244, 422102, 422169, 422104, 422171, 422248, 422105, 422249, 422173, 422250, and 422174.

Certain gapmers within the target region (i.e. nucleobases 11830-11869) inhibited Factor 7 mRNA expression by at least 60%, for example, ISIS numbers 416549, 422234, 422095, 422238, 422239, 422096, 422240, 422164, 422241, 422242, 422166, 422243, 422102, 422171, 422248, and 422105.

Certain gapmers within the target region (i.e. nucleobases 11830-11869) inhibited Factor 7 mRNA expression by at least 70%, for example, ISIS number 422096.

TABLE 9

Inhibition of human Factor 7 mRNA levels by chimeric antisense
oligonucleotides targeted to nucleobases
11830 to 11869 of SEQ ID NO: 1

| | | | | | | |
|---|---|---|---|---|---|---|
| | Target | Target | | | | |
| | Start | Stop | | | % | SEQ |
| Oligo ID | Site | Site | Sequence (5′ to 3′) | Motif | Inhibition | ID NO |

| | | | | | | |
|---|---|---|---|---|---|---|
| *416549 | 11838 | 11857 | GACAATGGTCAGGGCTGGTT | 5-10-5 | 69 | 245 |
| 422088 | 11830 | 11849 | TCAGGGCTGGTTTTGGAGGA | 5-10-5 | 8 | 340 |
| 422154 | 11830 | 11849 | TCAGGGCTGGTTTTGGAGGA | 3-14-3 | 31 | 340 |
| 422231 | 11830 | 11849 | TCAGGGCTGGTTTTGGAGGA | 2-13-5 | 22 | 340 |
| 422089 | 11831 | 11850 | GTCAGGGCTGGTTTTGGAGG | 5-10-5 | 22 | 341 |
| 422155 | 11831 | 11850 | GTCAGGGCTGGTTTTGGAGG | 3-1-4-3 | 34 | 341 |
| 422232 | 11831 | 11850 | GTCAGGGCTGGTTTTGGAGG | 2-13-5 | 41 | 341 |
| 422090 | 11832 | 11851 | GGTCAGGGCTGGTTTTGGAG | 5-10-5 | 42 | 342 |
| 422156 | 11832 | 11851 | GGTCAGGGCTGGTTTTGGAG | 3-1-4-3 | 38 | 342 |
| 422233 | 11832 | 11851 | GGTCAGGGCTGGTTTTGGAG | 2-13-5 | 46 | 342 |
| 422091 | 11833 | 11852 | TGGTCAGGGCTGGTTTTGGA | 5-10-5 | 42 | 343 |
| 422157 | 11833 | 11852 | TGGTCAGGGCTGGTTTTGGA | 3-14-3 | 49 | 343 |
| 422234 | 11833 | 11852 | TGGTCAGGGCTGGTTTTGGA | 2-13-5 | 62 | 343 |
| 422092 | 11834 | 11853 | ATGGTCAGGGCTGGTTTTGG | 5-10-5 | 36 | 344 |
| 422158 | 11834 | 11853 | ATGGTCAGGGCTGGTTTTGG | 3-14-3 | 49 | 344 |
| 422235 | 11834 | 11853 | ATGGTCAGGGCTGGTTTTGG | 2-13-5 | 50 | 344 |
| 422093 | 11835 | 11854 | AATGGTCAGGGCTGGTTTTG | 5-10-5 | 42 | 345 |
| 422159 | 11835 | 11854 | AATGGTCAGGGCTGGTTTTG | 3-14-3 | 45 | 345 |
| 422236 | 11835 | 11854 | AATGGTCAGGGCTGGTTTTG | 2-1-3-5 | 44 | 345 |
| 422094 | 11836 | 11855 | CAATGGTCAGGGCTGGTTTT | 5-10-5 | 48 | 346 |
| 422160 | 11836 | 11855 | CAATGGTCAGGGCTGGTTTT | 3-14-3 | 42 | 346 |
| 422237 | 11836 | 11855 | CAATGGTCAGGGCTGGTTTT | 2-13-5 | 50 | 346 |
| 422095 | 11837 | 11856 | ACAATGGTCAGGGCTGGTTT | 5-10-5 | 60 | 347 |
| 422161 | 11837 | 11856 | ACAATGGTCAGGGCTGGTTT | 3-14-3 | 53 | 347 |
| 422238 | 11837 | 11856 | ACAATGGTCAGGGCTGGTTT | 2-13-5 | 64 | 347 |
| 422162 | 11838 | 11857 | GACAATGGTCAGGGCTGGTT | 3-14-3 | 59 | 245 |
| 422239 | 11838 | 11857 | GACAATGGTCAGGGCTGGTT | 2-13-5 | 67 | 245 |
| 422096 | 11839 | 11858 | AGACAATGGTCAGGGCTGGT | 5-10-5 | 76 | 348 |
| 422163 | 11839 | 11858 | AGACAATGGTCAGGGCTGGT | 3-14-3 | 56 | 348 |
| 422240 | 11839 | 11858 | AGACAATGGTCAGGGCTGGT | 2-13-5 | 66 | 348 |
| 422097 | 11840 | 11859 | GAGACAATGGTCAGGGCTGG | 5-10-5 | 59 | 349 |
| 422164 | 11840 | 11859 | GAGACAATGGTCAGGGCTGG | 3-14-3 | 64 | 349 |
| 422241 | 11840 | 11859 | GAGACAATGGTCAGGGCTGG | 2-13-5 | 61 | 349 |
| 422098 | 11841 | 11860 | GGAGACAATGGTCAGGGCTG | 5-10-5 | 53 | 350 |
| 422165 | 11841 | 11860 | GGAGACAATGGTCAGGGCTG | 3-14-3 | 57 | 350 |
| 422242 | 11841 | 11860 | GGAGACAATGGTCAGGGCTG | 2-13-5 | 64 | 350 |
| 422099 | 11842 | 11861 | AGGAGACAATGGTCAGGGCT | 5-10-5 | 48 | 351 |
| 422166 | 11842 | 11861 | AGGAGACAATGGTCAGGGCT | 3-14-3 | 63 | 351 |
| 422243 | 11842 | 11861 | AGGAGACAATGGTCAGGGCT | 2-13-5 | 62 | 351 |
| 422100 | 11843 | 11862 | GAGGAGACAATGGTCAGGGC | 5-10-5 | 59 | 352 |
| 422167 | 11843 | 11862 | GAGGAGACAATGGTCAGGGC | 3-14-3 | 53 | 352 |
| 422244 | 11843 | 11862 | GAGGAGACAATGGTCAGGGC | 2-13-5 | 55 | 352 |
| 422101 | 11844 | 11863 | TGAGGAGACAATGGTCAGGG | 5-10-5 | 42 | 353 |
| 422168 | 11844 | 11863 | TGAGGAGACAATGGTCAGGG | 3-1-4-3 | 30 | 353 |
| 422245 | 11844 | 11863 | TGAGGAGACAATGGTCAGGG | 2-13-5 | 24 | 353 |
| 422102 | 11845 | 11864 | CTGAGGAGACAATGGTCAGG | 5-10-5 | 62 | 354 |
| 422169 | 11845 | 11864 | CTGAGGAGACAATGGTCAGG | 3-14-3 | 56 | 354 |
| 422246 | 11845 | 11864 | CTGAGGAGACAATGGTCAGG | 2-13-5 | 46 | 354 |
| 422103 | 11846 | 11865 | GCTGAGGAGACAATGGTCAG | 5-10-5 | 38 | 355 |
| 422170 | 11846 | 11865 | GCTGAGGAGACAATGGTCAG | 3-14-3 | 28 | 355 |
| 422247 | 11846 | 11865 | GCTGAGGAGACAATGGTCAG | 2-13-5 | 36 | 355 |
| 422104 | 11847 | 11866 | GGCTGAGGAGACAATGGTCA | 5-10-5 | 59 | 356 |
| 422171 | 11847 | 11866 | GGCTGAGGAGACAATGGTCA | 3-14-3 | 61 | 356 |
| 422248 | 11847 | 11866 | GGCTGAGGAGACAATGGTCA | 2-13-5 | 60 | 356 |
| 422105 | 11848 | 11867 | TGGCTGAGGAGACAATGGTC | 5-10-5 | 60 | 357 |
| 422172 | 11848 | 11867 | TGGCTGAGGAGACAATGGTC | 3-14-3 | 39 | 357 |
| 422249 | 11848 | 11867 | TGGCTGAGGAGACAATGGTC | 2-13-5 | 52 | 357 |
| 422106 | 11849 | 11868 | CTGGCTGAGGAGACAATGGT | 5-10-5 | 32 | 358 |
| 422173 | 11849 | 11868 | CTGGCTGAGGAGACAATGGT | 3-14-3 | 51 | 358 |
| 422250 | 11849 | 11868 | CTGGCTGAGGAGACAATGGT | 2-1-3-5 | 54 | 358 |
| 422107 | 11850 | 11869 | GCTGGCTGAGGAGACAATGG | 5-10-5 | 36 | 359 |
| 422174 | 11850 | 11869 | GCTGGCTGAGGAGACAATGG | 3-14-3 | 55 | 359 |
| 422251 | 11850 | 11869 | GCTGGCTGAGGAGACAATGG | 2-13-5 | 36 | 359 |

The chimeric antisense oligonucleotides in Table 10 were designed as 5-10-5 MOE, 3-14-3 MOE, and 2-13-5 MOE gapmers. The first listed gapmer in Table 10 is the original gapmer (see Table 7) from which the remaining gapmers were designed via microwalk and is designated by an asterisk (*). The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 10 is targeted to SEQ ID NO: 1 (nucleotides 1255000 to 1273000 of GENBANK Accession No. NT_{—}027140.6).

As shown in Table 10, most of the 5-10-5 MOE gapmers, 3-14-3 MOE gapmers, and 2-13-5 MOE gapmers targeted to the target region beginning at target start site 13760 and ending at the target stop site 13789 (i.e. nucleobases 13760-13789) of SEQ ID NO: 1 inhibited Factor 7 mRNA by at least 30%.

Certain gapmers within the target region (i.e. nucleobases 13760-13789) inhibited Factor 7 mRNA expression by at least 20%, for example, ISIS numbers 416455, 422175, 422252, 422108, 422176, 422253, 422109, 422177, 422254, 422110, 422178, 422255, 422111, 422179, 422256, 422112, 422180, 422257, 422113, 422181, 422258, 422114, 422259, 422115, 422183, 422260, 422116, 422184, 422261, 416456, and 422185.

Certain gapmers within the target region (i.e. nucleobases 13760-13789) inhibited Factor 7 mRNA expression by at least 30%, for example, ISIS numbers 416455, 422175, 422252, 422108, 422176, 422253, 422109, 422177, 422254, 422110, 422178, 422255, 422111, 422179, 422112, 422180, 422257, 422113, 422181, 422258, 422114, 422259, 422115, 422183, 422260, 422116, 422184, 422261, 416456, and 422185.

Certain gapmers within the target region (i.e. nucleobases 13760-13789) inhibited Factor 7 mRNA expression by at least 40%, for example, ISIS numbers 416455, 422175, 422252, 422108, 422176, 422253, 422109, 422177, 422254, 422110, 422179, 422112, 422180, 422257, 422113, 422181, 422258, 422114, 422259, 422115, 422183, 422260, 422116, 422184, 422261, 416456, and 422185.

Certain gapmers within the target region (i.e. nucleobases 13760-13789) inhibited Factor 7 mRNA expression by at least 50%, for example, ISIS numbers 416455, 422175, 422252, 422108, 422176, 422253, 422109, 422177, 422110, 422112, 422180, 422257, 422113, 422181, 422258, 422114, 422259, 422115, 422183, 422260, 422116, 422184, 422261, and 416456.

Certain gapmers within the target region (i.e. nucleobases 13760-13789) inhibited Factor 7 mRNA expression by at least 60%, for example, ISIS numbers 422175, 422252, 422108, 422253, 422109, 422177, 422112, 422257, 422113, 422181, 422258, 422259, 422115, 422183, and 422261.

Certain gapmers within the target region (i.e. nucleobases 13760-13789) inhibited Factor 7 mRNA expression by at least 70%, for example, ISIS numbers 422252, 422177, 422183, and 422261.

TABLE 10

Inhibition of human Factor 7 mRNA levels by chimeric antisense
oligonucleotides targeted to nucleobases
13760 to 13789 of SEQ ID NO: 1

| | | | | | | |
|---|---|---|---|---|---|---|
| | Target | Target | | | | |
| | Start | Stop | | | % | SEQ ID |
| ISIS No. | Site | Site | Sequence (5′ to 3′) | Motif | Inhibition | NO |

| | | | | | | |
|---|---|---|---|---|---|---|
| *416455 | 13760 | 13779 | CGCCGGCTCTGCTCATCCCC | 5-10-5 | 51 | 261 |
| 422175 | 13760 | 13779 | CGCCGGCTCTGCTCATCCCC | 3-14-3 | 69 | 261 |
| 422252 | 13760 | 13779 | CGCCGGCTCTGCTCATCCCC | 2-13-5 | 70 | 261 |
| 422108 | 13761 | 13780 | CCGCCGGCTCTGCTCATCCC | 5-10-5 | 69 | 360 |
| 422176 | 13761 | 13780 | CCGCCGGCTCTGCTCATCCC | 3-14-3 | 52 | 360 |
| 422253 | 13761 | 13780 | CCGCCGGCTCTGCTCATCCC | 2-13-5 | 61 | 360 |
| 422109 | 13762 | 13781 | CCCGCCGGCTCTGCTCATCC | 5-10-5 | 68 | 361 |
| 422177 | 13762 | 13781 | CCCGCCGGCTCTGCTCATCC | 3-14-3 | 74 | 361 |
| 422254 | 13762 | 13781 | CCCGCCGGCTCTGCTCATCC | 2-13-5 | 42 | 361 |
| 422110 | 13763 | 13782 | ACCCGCCGGCTCTGCTCATC | 5-10-5 | 53 | 362 |
| 422178 | 13763 | 13782 | ACCCGCCGGCTCTGCTCATC | 3-14-3 | 37 | 362 |
| 422255 | 13763 | 13782 | ACCCGCCGGCTCTGCTCATC | 2-13-5 | 30 | 362 |
| 422111 | 13764 | 13783 | CACCCGCCGGCTCTGCTCAT | 5-10-5 | 37 | 363 |
| 422179 | 13764 | 13783 | CACCCGCCGGCTCTGCTCAT | 3-14-3 | 43 | 363 |
| 422256 | 13764 | 13783 | CACCCGCCGGCTCTGCTCAT | 2-13-5 | 29 | 363 |
| 422112 | 13765 | 13784 | CCACCCGCCGGCTCTGCTCA | 5-10-5 | 61 | 364 |
| 422180 | 13765 | 13784 | CCACCCGCCGGCTCTGCTCA | 3-14-3 | 54 | 364 |
| 422257 | 13765 | 13784 | CCACCCGCCGGCTCTGCTCA | 2-13-5 | 65 | 364 |
| 422113 | 13766 | 13785 | GCCACCCGCCGGCTCTGCTC | 5-10-5 | 68 | 365 |
| 422181 | 13766 | 13785 | GCCACCCGCCGGCTCTGCTC | 3-14-3 | 66 | 365 |
| 422258 | 13766 | 13785 | GCCACCCGCCGGCTCTGCTC | 2-13-5 | 66 | 365 |
| 422114 | 13767 | 13786 | CGCCACCCGCCGGCTCTGCT | 5-10-5 | 50 | 366 |
| 422182 | 13767 | 13786 | CGCCACCCGCCGGCTCTGCT | 3-14-3 | 11 | 366 |
| 422259 | 13767 | 13786 | CGCCACCCGCCGGCTCTGCT | 2-13-5 | 61 | 366 |
| 422115 | 13768 | 13787 | GCGCCACCCGCCGGCTCTGC | 5-10-5 | 66 | 367 |
| 422183 | 13768 | 13787 | GCGCCACCCGCCGGCTCTGC | 3-14-3 | 79 | 367 |
| 422260 | 13768 | 13787 | GCGCCACCCGCCGGCTCTGC | 2-13-5 | 56 | 367 |
| 422116 | 13769 | 13788 | TGCGCCACCCGCCGGCTCTG | 5-10-5 | 52 | 368 |
| 422184 | 13769 | 13788 | TGCGCCACCCGCCGGCTCTG | 3-14-3 | 55 | 368 |
| 422261 | 13769 | 13788 | TGCGCCACCCGCCGGCTCTG | 2-13-5 | 47 | 368 |
| 416456 | 13770 | 13789 | CTGCGCCACCCGCCGGCTCT | 5-10-5 | 50 | 262 |
| 422185 | 13770 | 13789 | CTGCGCCACCCGCCGGCTCT | 3-14-3 | 48 | 262 |
| 422262 | 13770 | 13789 | CTGCGCCACCCGCCGGCTCT | 2-13-5 | 0 | 262 |

The chimeric antisense oligonucleotides in Table 11 were designed as 5-10-5 MOE, 3-14-3 MOE, and 2-13-5 MOE gapmers. The first listed gapmer in Table 11 is the original gapmer (see Table 7) from which the remaining gapmers were designed via microwalk and is designated by an asterisk (*). The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 11 is targeted to SEQ ID NO: 1 (nucleotides 1255000 to 1273000 of GENBANK Accession No. NT_{—}027140.6).

As shown in Table 11, all of the 5-10-5 MOE gapmers, 3-14-3 MOE gapmers, and 2-13-5 MOE gapmers targeted to the target region beginning at target start site 14707 and ending at the target stop site 14732 (i.e. nucleobases 14707-14732) of SEQ ID NO: 1 inhibited Factor 7 mRNA by at least 48%.

Certain gapmers within the target region (i.e. nucleobases 14707-14732) inhibited Factor 7 mRNA expression by at least 40%, for example, ISIS numbers 416477, 407641, 422200, 422277, 422130, 422201, 422278, 422131, 422202, 422279, 422203, 422280, 422132, 422204, 422281, 422133, 422205, 422282, 407642, 422206, and 422283.

Certain gapmers within the target region (i.e. nucleobases 14707-14732) inhibited Factor 7 mRNA expression by at least 50%, for example, ISIS numbers 416477, 407641, 422200, 422277, 422130, 422201, 422278, 422131, 422279, 422203, 422280, 422132, 422204, 422281, 422133, 422205, 407642, 422206, and 422283.

Certain gapmers within the target region (i.e. nucleobases 14707-14732) inhibited Factor 7 mRNA expression by at least 60%, for example, ISIS numbers 416477, 407641, 422130, 422201, 422278, 422131, 422204, 422133, 422205, 407642, and 422206.

Certain gapmers within the target region (i.e. nucleobases 14707-14732) inhibited Factor 7 mRNA expression by at least 70%, for example, ISIS numbers 416477, 422130, 422201, and 422204.

TABLE 11

Inhibition of human Factor 7 mRNA levels by chimeric antisense
oligonucleotides targeted to nucleobases
14707 to 14732 of SEQ ID NO: 1

| | | | | | | |
|---|---|---|---|---|---|---|
| | Target | Target | | | | SEQ |
| | Start | Stop | | | % | ID |
| ISIS No. | Site | Site | Sequence (5′ to 3′) | Motif | Inhibition | NO |

| | | | | | | |
|---|---|---|---|---|---|---|
| *416477 | 14710 | 14729 | CAGCCTGAGGCCAGCAGATC | 5-10-5 | 71 | 283 |
| 407641 | 14707 | 14726 | CCTGAGGCCAGCAGATCACG | 5-10-5 | 68 | 48 |
| 422200 | 14707 | 14726 | CCTGAGGCCAGCAGATCACG | 3-14-3 | 58 | 48 |
| 422277 | 14707 | 14726 | CCTGAGGCCAGCAGATCACG | 2-13-5 | 56 | 48 |
| 422130 | 14708 | 14727 | GCCTGAGGCCAGCAGATCAC | 5-10-5 | 79 | 369 |
| 422201 | 14708 | 14727 | GCCTGAGGCCAGCAGATCAC | 3-14-3 | 71 | 369 |
| 422278 | 14708 | 14727 | GCCTGAGGCCAGCAGATCAC | 2-13-5 | 64 | 369 |
| 422131 | 14709 | 14728 | AGCCTGAGGCCAGCAGATCA | 5-10-5 | 68 | 370 |
| 422202 | 14709 | 14728 | AGCCTGAGGCCAGCAGATCA | 3-14-3 | 49 | 370 |
| 422279 | 14709 | 14728 | AGCCTGAGGCCAGCAGATCA | 2-13-5 | 56 | 370 |
| 422203 | 14710 | 14729 | CAGCCTGAGGCCAGCAGATC | 3-14-3 | 52 | 283 |
| 422280 | 14710 | 14729 | CAGCCTGAGGCCAGCAGATC | 2-13-5 | 52 | 283 |
| 422132 | 14711 | 14730 | GCAGCCTGAGGCCAGCAGAT | 5-10-5 | 54 | 371 |
| 422204 | 14711 | 14730 | GCAGCCTGAGGCCAGCAGAT | 3-14-3 | 72 | 371 |
| 422281 | 14711 | 14730 | GCAGCCTGAGGCCAGCAGAT | 2-13-5 | 57 | 371 |
| 422133 | 14712 | 14731 | AGCAGCCTGAGGCCAGCAGA | 5-10-5 | 65 | 372 |
| 422205 | 14712 | 14731 | AGCAGCCTGAGGCCAGCAGA | 3-14-3 | 63 | 372 |
| 422282 | 14712 | 14731 | AGCAGCCTGAGGCCAGCAGA | 2-13-5 | 48 | 372 |
| 407642 | 14713 | 14732 | CAGCAGCCTGAGGCCAGCAG | 5-10-5 | 63 | 49 |
| 422206 | 14713 | 14732 | CAGCAGCCTGAGGCCAGCAG | 3-14-3 | 65 | 49 |
| 422283 | 14713 | 14732 | CAGCAGCCTGAGGCCAGCAG | 2-13-5 | 50 | 49 |

The chimeric antisense oligonucleotides in Table 12 were designed as 5-10-5 MOE, 3-14-3 MOE, and 2-13-5 MOE gapmers. The first listed gapmer in Table 12 is the original gapmer (see Table 7) from which the remaining gapmers were designed via microwalk and is designated by an asterisk (*). The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 10 is targeted to SEQ ID NO: 1 (nucleotides 1255000 to 1273000 of GENBANK Accession No. NT_{—}027140.6).

As shown in Table 12, all of the 5-10-5 MOE gapmers, 3-14-3 MOE gapmers, and 2-13-5 MOE gapmers targeted to the target region beginning at target start site 15098 and ending at the target stop site 15122 (i.e. nucleobases 15098-15122) of SEQ ID NO: 1 inhibited Factor 7 mRNA by at least 25%.

Certain gapmers within the target region (i.e. nucleobases 15098-15122) inhibited Factor 7 mRNA expression by at least 20%, for example, ISIS numbers 407643, 422207, 422284, 422134, 422208, 422285, 422135, 422209, 422286, 422136, 422210, 422287, 422137, 422211, 422288, 416479, 422212, and 422289.

Certain gapmers within the target region (i.e. nucleobases 15098-15122) inhibited Factor 7 mRNA expression by at least 30%, for example, ISIS numbers 407643, 422207, 422284, 422134, 422208, 422285, 422135, 422209, 422286, 422136, 422287, 422137, 422211, 422288, 416479, 422212, and 422289.

Certain gapmers within the target region (i.e. nucleobases 15098-15122) inhibited Factor 7 mRNA expression by at least 40%, for example, ISIS numbers 407643, 422207, 422284, 422134, 422208, 422135, 422209, 422286, 422136, 422287, 422137, 422211, 422288, and 416479.

Certain gapmers within the target region (i.e. nucleobases 15098-15122) inhibited Factor 7 mRNA expression by at least 50%, for example, ISIS numbers 407643, 422134, 422208, 422135, 422286, and 422136.

Certain gapmers within the target region (i.e. nucleobases 15098-15122) inhibited Factor 7 mRNA expression by at least 60%, for example, ISIS numbers 407643 and 422134.

TABLE 12

Inhibition of human Factor 7 mRNA levels by chimeric antisense
oligonucleotides targeted to nucleobases
15098 to 15122 of SEQ ID NO: 1

| | | | | | | |
|---|---|---|---|---|---|---|
| | Target | Target | | | | |
| | Start | Stop | | | % | SEQ ID |
| Oligo ID | Site | Site | Sequence (5′ to 3′) | Motif | Inhibition | NO |

| | | | | | | |
|---|---|---|---|---|---|---|
| *407643 | 15098 | 15117 | CACACATGGAGTCAGCATCG | 5-10-5 | 69 | 50 |
| 422207 | 15098 | 15117 | CACACATGGAGTCAGCATCG | 3-14-3 | 41 | 50 |
| 422284 | 15098 | 15117 | CACACATGGAGTCAGCATCG | 2-13-5 | 43 | 50 |
| 422134 | 15099 | 15118 | GCACACATGGAGTCAGCATC | 5-10-5 | 67 | 373 |
| 422208 | 15099 | 15118 | GCACACATGGAGTCAGCATC | 3-14-3 | 56 | 373 |
| 422285 | 15099 | 15118 | GCACACATGGAGTCAGCATC | 2-13-5 | 39 | 373 |
| 422135 | 15100 | 15119 | AGCACACATGGAGTCAGCAT | 5-10-5 | 53 | 374 |
| 422209 | 15100 | 15119 | AGCACACATGGAGTCAGCAT | 3-14-3 | 47 | 374 |
| 422286 | 15100 | 15119 | AGCACACATGGAGTCAGCAT | 2-13-5 | 53 | 374 |
| 422136 | 15101 | 15120 | CAGCACACATGGAGTCAGCA | 5-10-5 | 57 | 375 |
| 422210 | 15101 | 15120 | CAGCACACATGGAGTCAGCA | 3-14-3 | 25 | 375 |
| 422287 | 15101 | 15120 | CAGCACACATGGAGTCAGCA | 2-13-5 | 41 | 375 |
| 422137 | 15102 | 15121 | ACAGCACACATGGAGTCAGC | 5-10-5 | 45 | 376 |
| 422211 | 15102 | 15121 | ACAGCACACATGGAGTCAGC | 3-14-3 | 42 | 376 |
| 422288 | 15102 | 15121 | ACAGCACACATGGAGTCAGC | 2-13-5 | 44 | 376 |
| 416479 | 15103 | 15122 | GACAGCACACATGGAGTCAG | 5-10-5 | 47 | 285 |
| 422212 | 15103 | 15122 | GACAGCACACATGGAGTCAG | 3-14-3 | 30 | 285 |
| 422289 | 15103 | 15122 | GACAGCACACATGGAGTCAG | 2-13-5 | 34 | 285 |

The chimeric antisense oligonucleotides in Table 13 were designed as 5-10-5 MOE, 3-14-3 MOE, and 2-13-5 MOE gapmers. The first listed gapmer in Table 13 is the original gapmer (see Table 7) from which the remaining gapmers were designed via microwalk and is designated by an asterisk (*). The 5-10-5 gapmers are 20 nucleotides in length, wherein the central gap segment is comprised of 10 2′-deoxynucleotides and is flanked on both sides (in the 5′ and 3′ directions) by wings comprising 5 nucleotides each. The 3-14-3 gapmers are 20 nucleotides in length, wherein the central gap segment is comprised of 14 2′-deoxynucleotides and is flanked on both sides (in the 5′ and 3′ directions) by wings comprising 3 nucleotides each. The 2-13-5 gapmers are 20 nucleotides in length, wherein the central gap segment is comprised of 13 2′-deoxynucleotides. The central gap is flanked on the 5′ end with a wing comprising 2 nucleotides and on the 3′ end with a wing comprising 5 nucleotides. For each of the motifs (5-10-5, 3-14-3, and 2-13-5), each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines. “Target start site” indicates the 5′-most nucleotide to which the gapmer is targeted. “Target stop site” indicates the 3′-most nucleotide to which the gapmer is targeted. Each gapmer listed in Table 11 is targeted to SEQ ID NO: 1 (nucleotides 1255000 to 1273000 of GENBANK Accession No. NT_{—}027140.6).

As shown in Table 13, all of the 5-10-5 MOE gapmers, 3-14-3 MOE gapmers, and 2-13-5 MOE gapmers targeted to the target region beginning at target start site 15188 and ending at the target stop site 15211 (i.e. nucleobases 15188-15211) of SEQ ID NO: 1 inhibited Factor 7 mRNA by at least 41%.

Certain gapmers within the target region (i.e. nucleobases 15188-15211) inhibited Factor 7 mRNA expression by at least 40%, for example, ISIS numbers 407935, 416482, 422149, 422226, 422085, 422150, 422227, 422086, 422151, 422228, 422152, 422229, 422087, 422153, and 422230.

Certain gapmers within the target region (i.e. nucleobases 15188-15211) inhibited Factor 7 mRNA expression by at least 50%, for example, ISIS numbers 407935, 416482, 422149, 422085, 422150, 422227, 422086, 422151, 422228, 422152, 422229, 422087, 422153, and 422230.

Certain gapmers within the target region (i.e. nucleobases 15188-15211) inhibited Factor 7 mRNA expression by at least 60%, for example, ISIS numbers 407935, 416482, 422149, 422085, 422150, 422227, 422086, 422151, 422228, 422152, 422229, 422087, 422153, and 422230.

Certain gapmers within the target region (i.e. nucleobases 15188-15211) inhibited Factor 7 mRNA expression by at least 70%, for example, ISIS numbers 407935, 422085, 422150, 422086, 422228, 422152, 422229, and 422087.

Certain gapmers within the target region (i.e. nucleobases 15188-15211) inhibited Factor 7 mRNA expression by at least 80%, for example, ISIS numbers 422086 and 422087.

TABLE 13

Inhibition of human Factor 7 mRNA levels by chimeric
antisense oligonucleotides targeted to nucleobases
15188 to 15211 of SEQ ID NO: 1

| | | | | | | |
|---|---|---|---|---|---|---|
| | Target | Target | | | | |
| | Start | Stop | | | % | SEQ |
| Oligo ID | Site | Site | Sequence (5′ to 3′) | Motif | Inhibition | ID NO |

| | | | | | | |
|---|---|---|---|---|---|---|
| *407935 | 15191 | 15210 | ATGCATGGTGATGCTTCTGA | 5-10-5 | 79 | 123 |
| 416482 | 15188 | 15207 | CATGGTGATGCTTCTGAATT | 5-10-5 | 64 | 288 |
| 422149 | 15188 | 15207 | CATGGTGATGCTTCTGAATT | 3-14-3 | 61 | 288 |
| 422226 | 15188 | 15207 | CATGGTGATGCTTCTGAATT | 2-13-5 | 41 | 288 |
| 422085 | 15189 | 15208 | GCATGGTGATGCTTCTGAAT | 5-10-5 | 70 | 377 |
| 422150 | 15189 | 15208 | GCATGGTGATGCTTCTGAAT | 3-14-3 | 74 | 377 |
| 422227 | 15189 | 15208 | GCATGGTGATGCTTCTGAAT | 2-13-5 | 67 | 377 |
| 422086 | 15190 | 15209 | TGCATGGTGATGCTTCTGAA | 5-10-5 | 81 | 378 |
| 422151 | 15190 | 15209 | TGCATGGTGATGCTTCTGAA | 3-14-3 | 68 | 378 |
| 422228 | 15190 | 15209 | TGCATGGTGATGCTTCTGAA | 2-13-5 | 73 | 378 |
| 422152 | 15191 | 15210 | ATGCATGGTGATGCTTCTGA | 3-14-3 | 74 | 123 |
| 422229 | 15191 | 15210 | ATGCATGGTGATGCTTCTGA | 2-13-5 | 71 | 123 |
| 422087 | 15192 | 15211 | CATGCATGGTGATGCTTCTG | 5-10-5 | 83 | 379 |
| 422153 | 15192 | 15211 | CATGCATGGTGATGCTTCTG | 3-14-3 | 67 | 379 |
| 422230 | 15192 | 15211 | CATGCATGGTGATGCTTCTG | 2-13-5 | 65 | 379 |

### Example 7

### Dose Response Antisense Inhibition of Human Factor 7 in HepB3 Cells

Gapmers from Examples 5 and 6 (see Tables 7, 8, 9, 10, 11, 12, and 13), exhibiting in vitro inhibition of human Factor 7, were tested at various doses in HepB3 cells. Cells were plated at a density of 4,000 cells per well and transfected using lipofectin reagent with 3.125 nM, 6.25 nM, 12.5 nM, 25 nM, 50 nM, and 100 nM concentrations of antisense oligonucleotide, as specified in Table 14. After a treatment period of approximately 16 hours, RNA was isolated from the cells and Factor 7 mRNA levels were measured by quantitative real-time PCR. Human Factor 7 primer probe set RTS 2927 was used to measure mRNA levels. Factor 7 mRNA levels were adjusted according to total RNA content, as measured by RIBOGREEN®. Results are presented as percent inhibition of Factor 7, relative to untreated control cells. As illustrated in Table 14, Factor 7 mRNA levels were reduced in a dose-dependent manner.

TABLE 14
Dose-dependent antisense inhibition of human Factor 7 in HepB3 cells via
transfection of oligonucleotides with lipofectin

| ISIS | 3.125 | | | | | 100 | SEQ ID |
|---|---|---|---|---|---|---|---|
| No. | nM | 6.25 M | 12.5 nM | 25 nM | 50 nM | nM | NO |
| 407935 | 19 | 39 | 58 | 70 | 85 | 87 | 123 |
| 407939 | 17 | 35 | 57 | 75 | 80 | 83 | 127 |
| 422086 | 8 | 26 | 46 | 65 | 83 | 90 | 378 |
| 422087 | 14 | 24 | 51 | 73 | 83 | 90 | 379 |
| 422096 | 17 | 23 | 43 | 60 | 68 | 82 | 348 |
| 422150 | 9 | 23 | 38 | 59 | 79 | 87 | 377 |
| 422152 | 18 | 29 | 47 | 67 | 83 | 89 | 123 |
| 422177 | 12 | 24 | 44 | 64 | 81 | 88 | 361 |
| 422183 | 28 | 37 | 56 | 78 | 84 | 88 | 367 |
| 422228 | 17 | 25 | 43 | 60 | 78 | 90 | 378 |
| 422229 | 21 | 32 | 53 | 72 | 86 | 92 | 123 |
| 422130 | 4 | 15 | 34 | 59 | 77 | 85 | 369 |
| 422140 | 18 | 35 | 49 | 64 | 74 | 74 | 331 |
| 422142 | 16 | 34 | 51 | 66 | 72 | 68 | 333 |
| 422215 | 13 | 30 | 54 | 65 | 84 | 82 | 331 |
| 422217 | 8 | 18 | 45 | 63 | 79 | 83 | 333 |
| 422252 | 18 | 17 | 45 | 61 | 77 | 87 | 261 |
| 422292 | 13 | 21 | 44 | 63 | 81 | 83 | 331 |
| 422294 | 9 | 21 | 32 | 61 | 77 | 84 | 333 |
| 422295 | 17 | 22 | 44 | 64 | 80 | 85 | 189 |
| 422296 | 21 | 27 | 39 | 67 | 78 | 82 | 334 |

The gapmers were also transfected via electroporation and their dose-dependent inhibition of human Factor 7 mRNA was measured. Cells were plated at a density of 20,000 cells per well and transfected via electroporation with 3.125 μM, 6.25 μM, 12.5 μM, 25 μM, 50 μM, and 100 μM concentrations of antisense oligonucleotide, as specified in Table 15. After a treatment period of approximately 16 hours, RNA was isolated from the cells and Factor 7 mRNA levels were measured by quantitative real-time PCR. Human Factor 7 primer probe set RTS 2927 was used to measure mRNA levels. Factor 7 mRNA levels were adjusted according to total RNA content, as measured by RIBOGREEN®. Results are presented as percent inhibition of Factor 7, relative to untreated control cells. As illustrated in Table 15, Factor 7 mRNA levels were reduced in a dose-dependent manner.

TABLE 15
Dose-dependent antisense inhibition of human Factor 7 in HepB3 cells via
transfection of oligonucleotides with electroporation

| | 3.125 | | | | 50 | 100 | SEQ ID |
|---|---|---|---|---|---|---|---|
| ISIS No. | μM | 6.25 μM | 12.5 μM | 25 μM | μM | μM | No. |
| 407935 | 37 | 47 | 67 | 73 | 80 | 87 | 123 |
| 407939 | 18 | 28 | 42 | 56 | 68 | 82 | 127 |
| 422086 | 20 | 34 | 44 | 63 | 75 | 84 | 378 |
| 422087 | 22 | 26 | 48 | 57 | 72 | 80 | 379 |
| 422096 | 26 | 30 | 46 | 50 | 62 | 85 | 348 |
| 422150 | 17 | 20 | 40 | 54 | 68 | 83 | 377 |
| 422152 | 14 | 22 | 32 | 44 | 65 | 76 | 123 |
| 422177 | 3 | 10 | 29 | 21 | 31 | 63 | 361 |
| 422183 | 16 | 28 | 37 | 44 | 59 | 71 | 367 |
| 422228 | 18 | 33 | 40 | 47 | 71 | 83 | 378 |
| 422229 | 21 | 30 | 33 | 44 | 65 | 77 | 123 |
| 422130 | 28 | 29 | 59 | 61 | 73 | 86 | 369 |
| 422140 | 29 | 34 | 51 | 64 | 69 | 77 | 331 |
| 422142 | 26 | 47 | 63 | 67 | 77 | 78 | 333 |
| 422215 | 11 | 9 | 31 | 50 | 68 | 84 | 331 |
| 422217 | 15 | 19 | 29 | 55 | 61 | 83 | 333 |
| 422252 | 23 | 12 | 26 | 38 | 58 | 80 | 261 |
| 422292 | 36 | 25 | 47 | 59 | 75 | 86 | 331 |
| 422295 | 10 | 8 | 37 | 58 | 68 | 80 | 189 |
| 422296 | 62 | 40 | 56 | 56 | 82 | 84 | 334 |

### Example 8

### Selection and Confirmation of Effective Dose-Dependent Antisense Inhibition of Human Factor 7 in HepB3 Cells

Gapmers exhibiting in vitro inhibition of human Factor 7 in Example 7 were selected and tested at various doses in HepB3 cells. Cells were plated at a density of 20,000 cells per well and transfected via electroporation with 3.125 μM, 6.25 μM, 12.5 μM, 25 μM, 50 μM, and 100 μM concentrations of antisense oligonucleotide, as specified in Table 16. After a treatment period of approximately 16 hours, RNA was isolated from the cells and human Factor 7 mRNA levels were measured by quantitative real-time PCR. Human Factor 7 primer probe set RTS 2927 was used to measure mRNA levels. Factor 7 mRNA levels were adjusted according to total RNA content, as measured by RIBOGREEN®. Results are presented as percent inhibition of Factor 7, relative to untreated control cells. As illustrated in Table 16, Factor 7 mRNA levels were reduced in a dose-dependent manner.

TABLE 16
Dose-dependent antisense inhibition of human Factor 7 in HepB3 cells via
transfection of oligonucleotides with electroporation

| | 3.125 | 6.25 | | | | 100 | SEQ ID |
|---|---|---|---|---|---|---|---|
| ISIS No. | μM | μM | 12.5 μM | 25 μM | 50 μM | μM | NO: |
| 407935 | 63 | 79 | 84 | 90 | 90 | 91 | 123 |
| 407939 | 35 | 60 | 60 | 73 | 81 | 83 | 127 |
| 422086 | 25 | 48 | 74 | 82 | 86 | 90 | 378 |
| 422087 | 19 | 38 | 66 | 75 | 80 | 87 | 379 |
| 422130 | 14 | 24 | 61 | 68 | 82 | 88 | 369 |
| 422142 | 34 | 47 | 62 | 67 | 65 | 65 | 333 |
| 422150 | 0 | 31 | 53 | 67 | 77 | 86 | 377 |
| 422183 | 2 | 3 | 24 | 50 | 64 | 71 | 367 |
| 422229 | 30 | 45 | 67 | 79 | 90 | 92 | 123 |
| 422292 | 31 | 40 | 68 | 75 | 82 | 83 | 331 |
| 422296 | 47 | 44 | 70 | 78 | 80 | 82 | 334 |

### Example 9

### Antisense Inhibition of Human Factor 7 with Short (14-Mer) Oligonucleotides

Short antisense oligonucleotides (shortmers) were designed to target a Factor 7 nucleic acid. The shortmers in Table 17 were designed as 2-10-2 MOE gapmers. The gapmers are 14 nucleotides in length, wherein the central gap segment is comprised of 10 2′-deoxynucleotides and is flanked on both sides (in the 5′ and 3′ directions) by wings comprising 2 nucleotides each. Each nucleotide in the 5′ wing segment and each nucleotide in the 3′ wing segment has a 2′-MOE modification. The internucleoside linkages throughout each gapmer are phosphorothioate (P═S) linkages. All cytidine residues throughout each gapmer are 5-methylcytidines.

Shortmers were evaluated for their ability to reduce human Factor 7 mRNA in HepB3 cells and compared with one 5-10-5 chimeric oligonucleotide from Table 16, ISIS 407939. HepB3 cells at a density of 20,000 cells per well in a 96-well plate were transfected using electroporation with 1,000 nM of antisense oligonucleotide. After a treatment period of approximately 24 hours, RNA was isolated from the cells and Factor 7 mRNA levels were measured by real-time RT-PCR, as described herein. Factor 7 mRNA levels were adjusted according to total RNA content as measured by RIBOGREEN®. Results are presented in Table 17 as percent inhibition of Factor 7 mRNA, relative to untreated control cells. ISIS 407939 is the first oligonucleotide in Table 18 to which the shortmers were compared, and is marked by an asterisk (*).

Each gapmer listed in Table 17 is targeted to human gene sequences, SEQ ID NO: 1 (nucleotides 1255000 to 1273000 of GENBANK Accession No. NT_{—}027140.6) or SEQ ID NO: 2 (GENBANK Accession No. NM_{—}0196162). “Target start site” indicates the 5′-most nucleotide to which the gapmer is targeted in the human gene sequence. “Target stop site” indicates the 3′-most nucleotide to which the gapmer is targeted in the human gene sequence.

TABLE 17

Inhibition of human Factor 7 mRNA levels by chimeric
antisense oligonucleotides having 2-10-2 MOE wings
and deoxy gap targeted to SEQ ID NO: 1 or SEQ ID NO: 2

| | | | | | | |
|---|---|---|---|---|---|---|
| | Human | Human | Human | | | |
| | Target | Target | Target | | | |
| | SEQ ID | Start | Stop | | % | SEQ ID |
| ISIS No. | NO: | Site | Site | Sequence (5′ to 3′) | inhibition | NO |

| | | | | | | |
|---|---|---|---|---|---|---|
| *407939 | 1 | 15255 | 15274 | TGCAGCCCGGCACCCAGCGA | 81 | 127 |
| 435758 | 1 | 610 | 623 | CCCTGAGCTGGGCA | 4 | 380 |
| 435759 | 1 | 657 | 670 | GAGCCACAGAGCCT | 11 | 381 |
| 435760 | 1 | 744 | 757 | TCTTGGGTGTGGAT | 17 | 382 |
| 435761 | 1 | 796 | 809 | CAGATGAAAACTTG | 5 | 383 |
| 435762 | 1 | 885 | 898 | CAGGTGTTAAGGTG | 0 | 384 |
| 435763 | 1 | 945 | 958 | AGGAGAAAGGTCAG | 2 | 385 |
| 435764 | 1 | 1018 | 1031 | TGAGAGCTGCACCT | 8 | 386 |
| 435765 | 1 | 1092 | 1105 | CAAAGTTCTCTGCC | 1 | 387 |
| 435634 | 1 | 1147 | 1160 | TGAAATCTCTGCAG | 5 | 388 |
| 435635 | 1 | 1152 | 1165 | CATGATGAAATCTC | 0 | 389 |
| 435636 | 1 | 1157 | 1170 | GAGACCATGATGAA | 13 | 390 |
| 435637 | 1 | 1196 | 1209 | TGAAGCCCAAGCAG | 11 | 391 |
| 435638 | 1 | 1201 | 1214 | AGCCCTGAAGCCCA | 55 | 392 |
| 435766 | 1 | 1217 | 1230 | GCACCTGCAGCCAG | 51 | 393 |
| 435767 | 1 | 1248 | 1261 | CACCAAGTTTATGG | 0 | 394 |
| 435768 | 1 | 1384 | 1397 | GCCTGGATGCTGGT | 75 | 395 |
| 435769 | 1 | 1442 | 1455 | TTCTTTGAAAAATA | 0 | 396 |
| 435770 | 1 | 1529 | 1542 | CTCTGAGAGGCCCC | 81 | 397 |
| 435771 | 1 | 2130 | 2143 | GTCATAAGGCCATG | 13 | 398 |
| 435772 | 1 | 2233 | 2246 | GCAGTCCCTGCTCA | 42 | 399 |
| 435773 | 1 | 2348 | 2361 | CAGGGAACACCCTC | 57 | 400 |
| 435774 | 1 | 2390 | 2403 | GTCCCAGGGAAGGC | 17 | 401 |
| 435775 | 1 | 2439 | 2452 | TCTGGAAGGAACAG | 25 | 402 |
| 435777 | 1 | 4713 | 4726 | CTGAGGATGCAGGC | 53 | 403 |
| 435778 | 1 | 4817 | 4830 | GCTACTGGGCCACG | 49 | 404 |
| 435779 | 1 | 4847 | 4860 | TGAGCGCGGAAGAA | 50 | 405 |
| 435780 | 1 | 4945 | 4958 | GGAGGGACGACCTT | 28 | 406 |
| 435781 | 1 | 5124 | 5137 | ACCACGCGGTGGTG | 0 | 407 |
| 435782 | 1 | 5209 | 5222 | CGTGCGCTCAGCTC | 36 | 408 |
| 435783 | 1 | 6396 | 6409 | AAACCGGCATGCGC | 49 | 409 |
| 435784 | 1 | 6433 | 6446 | CCTAAGTGTGCTTT | 17 | 410 |
| 435785 | 1 | 6540 | 6553 | AGCTTCTCACCACG | 46 | 411 |
| 435787 | 1 | 8481 | 8494 | GAGTGTGAGGCTTC | 34 | 412 |
| 435789 | 1 | 8518 | 8531 | CATGTTAGCCTTTG | 3 | 413 |
| 435790 | 1 | 8548 | 8561 | ATGACCATCCTCAA | 1 | 414 |
| 435791 | 1 | 8590 | 8603 | ATGAGAAATGCTTT | 0 | 415 |
| 435792 | 1 | 8651 | 8664 | TGCTGAAGAAACTG | 9 | 416 |
| 435793 | 1 | 8711 | 8724 | TGCAGTAGCAGATG | 27 | 417 |
| 435794 | 1 | 8776 | 8789 | GAGGTACACAGGCT | 26 | 418 |
| 435795 | 1 | 8828 | 8841 | ACACCCGAGGTTCA | 66 | 419 |
| 435796 | 1 | 8858 | 8871 | TGACCACACATTTC | 30 | 420 |
| 435797 | 1 | 8895 | 8908 | AATCTTGGCCCCTC | 16 | 421 |
| 435798 | 1 | 8933 | 8946 | AGCCAGACTTTCCT | 54 | 422 |
| 435639 | 1 | 9072 | 9085 | TCCAGAACAGCTTC | 31 | 423 |
| 435640 | 1 | 9081 | 9094 | TGTAAGAAATCCAG | 2 | 424 |
| 435799 | 1 | 9165 | 9178 | CTGGTCCCCATCTG | 33 | 425 |
| 435642 | 1 | 9168 | 9181 | ACACTGGTCCCCAT | 60 | 426 |
| 435478 | 1 | 9173 | 9186 | GAGGCACACTGGTC | 43 | 427 |
| 435643 | 1 | 9204 | 9217 | CTTGCAGGAGCCCC | 51 | 428 |
| 435644 | 1 | 9209 | 9222 | TGGTCCTTGCAGGA | 30 | 429 |
| 435645 | 1 | 9214 | 9227 | GGAGCTGGTCCTTG | 44 | 430 |
| 435646 | 1 | 9221 | 9234 | TAGGACTGGAGCTG | 13 | 431 |
| 435647 | 1 | 9226 | 9239 | AGATATAGGACTGG | 0 | 432 |
| 435648 | 1 | 9231 | 9244 | GAAGCAGATATAGG | 0 | 433 |
| 435649 | 1 | 9236 | 9249 | AGGCAGAAGCAGAT | 21 | 434 |
| 435650 | 1 | 9255 | 9268 | CCGGCCCTCGAAGG | 6 | 435 |
| 435651 | 1 | 9261 | 9274 | ACAGTTCCGGCCCT | 71 | 436 |
| 435800 | 1 | 9289 | 9302 | GGACCCAAAGTGGG | 0 | 437 |
| 435801 | 1 | 9339 | 9352 | CGGTTGGCCAGGCC | 38 | 438 |
| 435802 | 1 | 9420 | 9433 | GTCACCTAGACCAA | 61 | 439 |
| 435803 | 1 | 9517 | 9530 | GAGAATTGCCCAGG | 0 | 440 |
| 435804 | 1 | 9549 | 9562 | CAGGGACTCTCAGC | 26 | 441 |
| 435805 | 1 | 9716 | 9729 | TGAATTACTGAACC | 0 | 442 |
| 435806 | 1 | 9846 | 9859 | TGACCCCACAGGGA | 7 | 443 |
| 435807 | 1 | 9902 | 9915 | CAGTACTTCCCACG | 35 | 444 |
| 435808 | 1 | 9939 | 9952 | CTCTGGACACCGGG | 66 | 445 |
| 435809 | 1 | 10062 | 10075 | CTGACAATGTGATG | 5 | 446 |
| 435810 | 1 | 10114 | 10127 | CTGAGGGAATGTTG | 1 | 447 |
| 435811 | 1 | 10203 | 10216 | GTGGTCAAGGATGA | 27 | 448 |
| 435812 | 1 | 10355 | 10368 | GCTGAGCAGAGATC | 35 | 449 |
| 435813 | 1 | 10385 | 10398 | GGATGCACACCAGG | 55 | 450 |
| 435814 | 1 | 10584 | 10597 | TGACGAGAGGACCA | 31 | 451 |
| 435815 | 1 | 10677 | 10690 | CTCTTCCGAGCAGC | 59 | 452 |
| 435816 | 1 | 10803 | 10816 | TCCAAAGGACAAGG | 4 | 453 |
| 435817 | 1 | 10835 | 10848 | CTGAGCTTGGCACC | 39 | 454 |
| 435818 | 1 | 10978 | 10991 | GTCATCCTTGTCTG | 43 | 455 |
| 435652 | 1 | 10981 | 10994 | CTGGTCATCCTTGT | 48 | 456 |
| 435653 | 1 | 10986 | 10999 | ATCAGCTGGTCATC | 32 | 457 |
| 435654 | 1 | 11005 | 11018 | GCCGTTCTCGTTCA | 71 | 458 |
| 435655 | 1 | 11011 | 11024 | ACAGCCGCCGTTCT | 49 | 459 |
| 435656 | 1 | 11018 | 11031 | ACTGCTCACAGCCG | 58 | 460 |
| 435657 | 1 | 11023 | 11036 | GCAGTACTGCTCAC | 69 | 461 |
| 435658 | 1 | 11028 | 11041 | TCACTGCAGTACTG | 60 | 462 |
| 435659 | 1 | 11070 | 11083 | TACCCCTCGTGGCA | 18 | 463 |
| 435660 | 1 | 11088 | 11101 | CCGTCTGCCAGCAG | 58 | 464 |
| 435661 | 1 | 11095 | 11108 | GGACACCCCGTCTG | 18 | 465 |
| 435819 | 1 | 11181 | 11194 | TTTGTCCAGTAAGA | 1 | 466 |
| 435820 | 1 | 11294 | 11307 | CATCCTAGTCACTG | 19 | 467 |
| 435821 | 1 | 11454 | 11467 | AGTCAGTACAGACA | 0 | 468 |
| 435822 | 1 | 11494 | 11507 | CCAGGAGCCTTTAC | 37 | 469 |
| 435823 | 1 | 11622 | 11635 | TGAGTCCCAGGCTG | 41 | 470 |
| 435824 | 1 | 11724 | 11737 | CCTAAAGATGAATC | 21 | 471 |
| 435776 | 1 | 11732 | 11745 | GGGACACTCACACT | 51 | 472 |
| 435825 | 1 | 11830 | 11843 | CTGGTTTTGGAGGA | 0 | 473 |
| 435826 | 1 | 11861 | 11874 | AAGTGGCTGGCTGA | 27 | 474 |
| 435827 | 1 | 12057 | 12070 | TCAGAAAGATGCAG | 5 | 475 |
| 435662 | 1 | 12084 | 12097 | TGGATATTCAACTG | 19 | 476 |
| 435663 | 1 | 12089 | 12102 | CCACATGGATATTC | 50 | 477 |
| 435664 | 1 | 12094 | 12107 | TTTTTCCACATGGA | 4 | 478 |
| 435665 | 1 | 12099 | 12112 | AGGTATTTTTCCAC | 0 | 479 |
| 435666 | 1 | 12128 | 12141 | GGTTTGCTGGCATT | 71 | 480 |
| 435667 | 1 | 12141 | 12154 | AATTCGGCCTTGGG | 16 | 481 |
| 435479 | 1 | 12173 | 12186 | CACTCCCCTTTGGG | 9 | 482 |
| 435668 | 1 | 12182 | 12195 | TGCCATGGACACTC | 75 | 483 |
| 435828 | 1 | 12277 | 12290 | GACCTGCCCATTTT | 0 | 484 |
| 435829 | 1 | 12396 | 12409 | GGGTAGACCCTCAG | 18 | 485 |
| 435830 | 1 | 12493 | 12506 | TGATTTTGCAAAGA | 0 | 486 |
| 435831 | 1 | 12523 | 12536 | GATCTTCACATAGC | 51 | 487 |
| 435832 | 1 | 12632 | 12645 | TGCTCAGACCTGGC | 70 | 488 |
| 435671 | 1 | 12796 | 12809 | TCACCAACAACAGG | 28 | 489 |
| 435672 | 1 | 12842 | 12855 | ACCCAGATGGTGTT | 12 | 490 |
| 435673 | 1 | 12847 | 12860 | AGACCACCCAGATG | 34 | 491 |
| 435674 | 1 | 12863 | 12876 | AAACAGTGGGCCGC | 34 | 492 |
| 435675 | 1 | 12868 | 12881 | TGTCGAAACAGTGG | 24 | 493 |
| 435676 | 1 | 12873 | 12886 | GATTTTGTCGAAAC | 11 | 494 |
| 435833 | 1 | 13133 | 13146 | AGACACTTGAGAGC | 24 | 495 |
| 435834 | 1 | 13165 | 13178 | TGACAGCACGAAGC | 20 | 496 |
| 435835 | 1 | 13264 | 13277 | TTATTTGGGCATGG | 7 | 497 |
| 435836 | 1 | 13637 | 13650 | CTAGGTCTGCAGGG | 41 | 498 |
| 435837 | 1 | 13725 | 13738 | CTCGCCTGGAAGGA | 37 | 499 |
| 435678 | 1 | 13733 | 13746 | AGGTCGTGCTCGCC | 67 | 500 |
| 435679 | 1 | 13738 | 13751 | CGCTGAGGTCGTGC | 62 | 501 |
| 435680 | 1 | 13743 | 13756 | GTGCTCGCTGAGGT | 52 | 502 |
| 435681 | 1 | 13781 | 13794 | ATGACCTGCGCCAC | 40 | 503 |
| 435682 | 1 | 13786 | 13799 | GGATGATGACCTGC | 36 | 504 |
| 435683 | 1 | 13823 | 13836 | ATGTCGTGGTTGGT | 3 | 505 |
| 435684 | 1 | 13832 | 13845 | AGCAGCGCGATGTC | 56 | 506 |
| 435685 | 1 | 13856 | 13869 | AGGACCACGGGCTG | 40 | 507 |
| 435686 | 1 | 13861 | 13874 | CAGTGAGGACCACG | 31 | 508 |
| 435687 | 1 | 13866 | 13879 | ATGGTCAGTGAGGA | 7 | 509 |
| 435688 | 1 | 13871 | 13884 | ACCACATGGTCAGT | 0 | 510 |
| 435689 | 1 | 13890 | 13903 | TTCGGGCAGGCAGA | 23 | 511 |
| 435690 | 1 | 13895 | 13908 | GTCCGTTCGGGCAG | 55 | 512 |
| 435691 | 1 | 13946 | 13959 | CAGCCGCTGACCAA | 25 | 513 |
| 435692 | 1 | 13964 | 13977 | CGGTCCAGCAGCTG | 32 | 514 |
| 435480 | 1 | 14016 | 14029 | GGTCATCAGCCGGG | 53 | 515 |
| 435481 | 1 | 14017 | 14030 | GGGTCATCAGCCGG | 50 | 516 |
| 435482 | 1 | 14018 | 14031 | TGGGTCATCAGCCG | 62 | 517 |
| 435483 | 1 | 14019 | 14032 | CTGGGTCATCAGCC | 82 | 518 |
| 435484 | 1 | 14020 | 14033 | CCTGGGTCATCAGC | 45 | 519 |
| 435485 | 1 | 14021 | 14034 | TCCTGGGTCATCAG | 34 | 520 |
| 435486 | 1 | 14022 | 14035 | GTCCTGGGTCATCA | 64 | 521 |
| 435487 | 1 | 14023 | 14036 | AGTCCTGGGTCATC | 36 | 522 |
| 435488 | 1 | 14024 | 14037 | CAGTCCTGGGTCAT | 17 | 523 |
| 435693 | 1 | 14026 | 14039 | GGCAGTCCTGGGTC | 43 | 524 |
| 435694 | 1 | 14031 | 14044 | CTGCAGGCAGTCCT | 39 | 525 |
| 435695 | 1 | 14036 | 14049 | GACTGCTGCAGGCA | 48 | 526 |
| 435696 | 1 | 14081 | 14094 | CAGAACATGTACTC | 10 | 527 |
| 435697 | 1 | 14092 | 14105 | AGTAGCCGGCACAG | 53 | 528 |
| 435698 | 1 | 14119 | 14132 | CCTTGCAGGAGTCC | 61 | 529 |
| 435551 | 1 | 14144 | 14157 | GTGGCATGTGGGCC | | 530 |
| 435699 | 1 | 14190 | 14203 | GCCCCAGCTGACGA | 48 | 531 |
| 435489 | 1 | 14231 | 14244 | CTGGTGTACACCCC | 72 | 532 |
| 435490 | 1 | 14232 | 14245 | CCTGGTGTACACCC | 49 | 533 |
| 435491 | 1 | 14233 | 14246 | CCCTGGTGTACACC | 61 | 534 |
| 435492 | 1 | 14234 | 14247 | ACCCTGGTGTACAC | 45 | 535 |
| 435493 | 1 | 14235 | 14248 | GACCCTGGTGTACA | 58 | 536 |
| 435494 | 1 | 14236 | 14249 | AGACCCTGGTGTAC | 47 | 537 |
| 435495 | 1 | 14237 | 14250 | GAGACCCTGGTGTA | 27 | 538 |
| 435496 | 1 | 14238 | 14251 | GGAGACCCTGGTGT | 47 | 539 |
| 435497 | 1 | 14239 | 14252 | GGGAGACCCTGGTG | 27 | 540 |
| 435498 | 1 | 14240 | 14253 | TGGGAGACCCTGGT | 37 | 541 |
| 435499 | 1 | 14241 | 14254 | CTGGGAGACCCTGG | 59 | 542 |
| 435500 | 1 | 14242 | 14255 | ACTGGGAGACCCTG | 49 | 543 |
| 435501 | 1 | 14243 | 14256 | TACTGGGAGACCCT | 51 | 544 |
| 435502 | 1 | 14244 | 14257 | GTACTGGGAGACCC | 69 | 545 |
| 435503 | 1 | 14245 | 14258 | TGTACTGGGAGACC | 17 | 546 |
| 435504 | 1 | 14246 | 14259 | ATGTACTGGGAGAC | 8 | 547 |
| 435714 | 1 | 14251 | 14264 | ACTCGATGTACTGG | 3 | 548 |
| 435715 | 1 | 14256 | 14269 | CAGCCACTCGATGT | 35 | 549 |
| 435716 | 1 | 14261 | 14274 | TTTTGCAGCCACTC | 22 | 550 |
| 435717 | 1 | 14266 | 14279 | TGAGCTTTTGCAGC | 0 | 551 |
| 435718 | 1 | 14303 | 14316 | GCTCGCAGGAGGAC | 26 | 552 |
| 435719 | 1 | 14354 | 14367 | CAGCCTTGGCTTTC | 50 | 553 |
| 435720 | 1 | 14662 | 14675 | AGGCTCAGCTGGGC | 16 | 554 |
| 435721 | 1 | 14667 | 14680 | TAAGGAGGCTCAGC | 12 | 555 |
| 435722 | 1 | 14687 | 14700 | TGGGCTTGGCTGAA | 50 | 556 |
| 435723 | 1 | 14707 | 14720 | GCCAGCAGATCACG | 68 | 557 |
| 435724 | 1 | 14712 | 14725 | CTGAGGCCAGCAGA | 52 | 558 |
| 435725 | 1 | 14717 | 14730 | GCAGCCTGAGGCCA | 79 | 559 |
| 435726 | 1 | 14734 | 14747 | GCAATGAAGGCAGA | 26 | 560 |
| 435727 | 1 | 15098 | 15111 | TGGAGTCAGCATCG | 73 | 561 |
| 435728 | 1 | 15103 | 15116 | ACACATGGAGTCAG | 16 | 562 |
| 435729 | 1 | 15108 | 15121 | ACAGCACACATGGA | 30 | 563 |
| 435730 | 1 | 15128 | 15141 | TAAACAACCGCCTT | 49 | 564 |
| 435731 | 1 | 15136 | 15149 | GTGAGAGCTAAACA | 11 | 565 |
| 435732 | 1 | 15141 | 15154 | GAAAAGTGAGAGCT | 0 | 566 |
| 435733 | 1 | 15181 | 15194 | CTGAATTGTCTGAA | 5 | 567 |
| 435734 | 1 | 15187 | 15200 | ATGCTTCTGAATTG | 16 | 568 |
| 435735 | 1 | 15192 | 15205 | TGGTGATGCTTCTG | 81 | 569 |
| 435736 | 1 | 15197 | 15210 | ATGCATGGTGATGC | 50 | 570 |
| 435737 | 1 | 15262 | 15275 | GTGCAGCCCGGCAC | 70 | 571 |
| 435738 | 1 | 15388 | 15401 | CAGAGGATGAGCAC | 16 | 572 |
| 435739 | 1 | 15393 | 15406 | CATCTCAGAGGATG | 5 | 573 |
| 435740 | 1 | 15430 | 15443 | TCATTTCAGTGATG | 0 | 574 |
| 435741 | 1 | 15435 | 15448 | AGGGTTCATTTCAG | 6 | 575 |
| 435742 | 1 | 15440 | 15453 | ATGTGAGGGTTCAT | 11 | 576 |
| 435743 | 1 | 15487 | 15500 | GCCTCAAACATCTA | 40 | 577 |
| 435744 | 1 | 15492 | 15505 | CTACAGCCTCAAAC | 19 | 578 |
| 435745 | 1 | 15497 | 15510 | GGGAGCTACAGCCT | 0 | 579 |
| 435746 | 1 | 15546 | 15559 | ATTGACAAGGGCTG | 32 | 580 |
| 435747 | 1 | 15551 | 15564 | ATATCATTGACAAG | 0 | 581 |
| 435748 | 1 | 15569 | 15582 | TCCCAGGGTCTCTG | 18 | 582 |
| 435749 | 1 | 15630 | 15643 | CAGCCAGGGCCTGG | 29 | 583 |
| 435750 | 1 | 15635 | 15648 | CACTGCAGCCAGGG | 14 | 584 |
| 435751 | 1 | 15650 | 15663 | CTTGCCAGGTCCTC | 28 | 585 |
| 435752 | 1 | 15655 | 15668 | TGCAGCTTGCCAGG | 12 | 586 |
| 435753 | 1 | 15660 | 15673 | AAGAGTGCAGCTTG | 9 | 587 |
| 435754 | 1 | 15665 | 15678 | TCAGCAAGAGTGCA | 16 | 588 |
| 435755 | 1 | 15670 | 15683 | GGGACTCAGCAAGA | 0 | 589 |
| 435756 | 1 | 15805 | 15818 | TGCCCAGGACGGCC | 20 | 590 |
| 435757 | 1 | 15895 | 15908 | TGCCTGAGTGCCCC | 51 | 591 |
| 435838 | 1 | 16009 | 16022 | TCCATGGACACTAA | 29 | 592 |
| 435839 | 1 | 16051 | 16064 | GGTCAGCTGGTCTG | 14 | 593 |
| 435840 | 1 | 16163 | 16176 | GGGCCCGCCACTGG | 0 | 594 |
| 435841 | 1 | 16205 | 16218 | CTCGGACAAAAGAG | 1 | 595 |
| 435842 | 1 | 16596 | 16609 | ATTTCCCATTGGCA | 0 | 596 |
| 435843 | 1 | 16730 | 16743 | GCTGGAGCTGAGCC | 0 | 597 |
| 435844 | 1 | 16773 | 16786 | TGGCCAGTGGCCTC | 30 | 598 |
| 435845 | 1 | 16872 | 16885 | GCTCATGGCAGACA | 21 | 599 |
| 435846 | 1 | 16910 | 16923 | TGGAGAGACCAATG | 7 | 600 |
| 435847 | 1 | 17139 | 17152 | TGGTGTGCACACAT | 36 | 601 |
| 435848 | 1 | 17207 | 17220 | TGGCCACTGCCTCA | 32 | 602 |
| 435849 | 1 | 17250 | 17263 | TGCCGTAGTGGCCG | 31 | 603 |
| 435850 | 1 | 17280 | 17293 | CTTGGCCAGTGTGG | 15 | 604 |
| 435851 | 1 | 17588 | 17601 | ACAGGCCAGGCTGG | 0 | 605 |
| 435786 | 1 | 48751 | 48764 | AGGTGACCCGTGAG | 68 | 606 |
| 435788 | 1 | 127793 | 127806 | CCTGTGAGTGTGAG | 20 | 607 |
| 435641 | 2 | 297 | 310 | GGTCCCCATCACTG | 51 | 608 |
| 435669 | 2 | 657 | 670 | GGACCTGCCATGGA | 36 | 609 |
| 435670 | 2 | 662 | 675 | CAACAGGACCTGCC | 47 | 610 |
| 435677 | 2 | 785 | 798 | GTGCTCGCCCAGCA | 60 | 611 |

### Example 10

### Antisense Inhibition of Murine Factor 7 In Vitro

Chimeric antisense oligonucleotides were designed as 5-10-5 MOE wings and deoxy gap were designed to target murine Factor 7 (nucleotides 10024000 to 10037000 of GENBANK Accession No. NT_{—}039455.6; incorporated herein as SEQ ID NO: 160). The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in each wing segment has a 2′-MOE modification. The internucleoside linkages throughout each gapmer are phosphorothioate (P═S) linkages. All cytidine residues throughout each gapmer are 5′ methylcytidines. The antisense oligonucleotides were evaluated for their ability to reduce murine Factor 7 mRNA in primary mouse hepatocytes. The antisense oligonucleotides were evaluated for their ability to reduce Factor 7 mRNA in primary mouse hepatocytes.

Primary mouse hepatocytes were treated with 6.25 nM, 12.5 nM, 25 nM, 50 nM, 100 nM, and 200 nM of antisense oligonucleotides for a period of approximately 24 hours. RNA was isolated from the cells and Factor 7 mRNA levels were measured by quantitative real-time PCR, as described herein. Murine Factor 7 primer probe set RTS 2855 (forward sequence AATGAGGAACAGTGCTCCTTTGA, SEQ ID NO: 612; reverse sequence TGTAAACAATCCAGAACTGCTTGGT, SEQ ID NO: 613; probe sequence CCCGGGAGATCTTCAAGAGCCCX, SEQ ID NO: 614) was used to measure mRNA levels. Factor 7 mRNA levels were adjusted according to total RNA content as measured by RIBOGREEN®. Certain murine antisense oligonucleotides reduced Factor 7 mRNA levels in a dose-dependent manner.

### Example 11

### Antisense Inhibition of Murine Factor 7 In Vivo

Four antisense oligonucleotides showing significant dose-dependent inhibition from the in vitro study (see Example 10) were evaluated for their ability to reduce Factor 7 mRNA in vivo. The antisense oligonucleotides are targeted to murine Factor 7 mRNA (nucleotides 10024000 to 10037000 of GENBANK Accession No. NT_{—}039455.6; SEQ ID NO: 160). Target start sites for the four of the antisense oligonucleotides are as follows: 11326, 11336, 11613, and 11766.

### Treatment

BALB/c mice were treated with ISIS 403102 (CCATAGAACAGCTTCACAGG, target site 11336, incorporated herein as SEQ ID NO: 161). BALB/c mice were injected subcutaneously with 5 mg/kg, 10 mg/kg, 25 mg/kg, or 50 mg/kg of ISIS 403102 twice a week for 3 weeks. A control group of mice was injected subcutaneously with PBS twice a week for 3 weeks. After the treatment period, whole liver was collected for RNA and protein analysis, and plasma was collected for clotting analysis (PT/aPTT).

### RNA Analysis

RNA was extracted from liver tissue for real-time RT-PCR analysis of Factor 7. As shown in Table 18, ISIS 403102 achieved a dose-dependent reduction of murine Factor 7 over the PBS control. Results are presented as percent inhibition of Factor 7, relative to the control.

TABLE 18
Dose-dependent antisense inhibition of
murine Factor 7 mRNA in BALB/c mice

| mg/kg | % inhibition |
|---|---|
| 5 | 64 |
| 10 | 84 |
| 25 | 96 |
| 50 | 99 |

### Protein Analysis

Plasma Factor 7 protein was measured using a Factor 7 chromogenic assay (Hyphen BioMed). As shown in Table 19, ISIS 403102 achieved a dose-dependent reduction of murine Factor 7 protein over the PBS control. Results are presented as percent inhibition of Factor 7, relative to control.

TABLE 19
Dose-dependent antisense inhibition of murine Factor 7 protein in
BALB/c mice

| mg/kg | % inhibition |
|---|---|
| 5 | 71 |
| 10 | 88 |
| 25 | 99 |
| 50 | 99 |

### Clotting Analysis

Prothrombin Time (PT) and Activated Partial Thromboplastin Time (aPTT) were measured using platelet poor plasma (PPP) from mice treated with ISIS 403102. PT and aPTT values provided in Table 20 are reported as International Normalized Ratio (INR) values. INR values for PT and aPTT were determined by dividing the PT or aPTT value for the experimental group by the PT or aPTT for the PBS treated group. This ratio was then raised to the power of the International Sensitivity Index (ISI) of the tissue factor used. As shown in Table 20, PT was significantly prolonged in mice treated with ISIS 403102 compared to the control. aPTT was slightly prolonged in mice treated with ISIS 403102. These data suggest that ISIS 403102 has a greater affect on the extrinsic pathway of blood coagulation than the intrinsic pathway of blood coagulation.

TABLE 20
Effect of ISIS 403102 on PT and aPTT in BALB/c mice

| mg/kg | PT INR | aPTT INR |
|---|---|---|
| 5 | 1.09 | 1.05 |
| 10 | 1.33 | 1.04 |
| 25 | 2.33 | 1.09 |
| 50 | 4.37 | 1.16 |

### Example 12

### Single Dose Pharmacokinetic Assay of ISIS 403102

### Treatment

The half-life and duration of action of ISIS 403102 in mice was evaluated. A group of 27 BALB/c mice was injected with 50 mg/kg of ISIS 403102. Three mice from the group were sacrificed at days 1, 2, 3, 4, 6, 8, 12, 24, and 56 after the single dose of ISIS 403102 was administered. A control group of 3 mice was injected with a single dose of PBS, and mice in this group were sacrificed on day 1. Mice in all groups were sacrificed by cervical dislocation following anesthesia with 150 mg/kg ketamine mixed with 10 mg/kg xylazine administered by intraperitoneal injection. Liver was harvested for RNA analysis and plasma was collected for clotting analysis (PT and aPTT).

### RNA Analysis

RNA was extracted from liver tissue for real-time RT-PCR analysis of Factor 7. Results are presented as percent inhibition of Factor 7, relative to PBS control. As shown in Table 21, treatment with ISIS 403102 resulted in 92% inhibition of Factor 7 mRNA by day 4 after which the effect of ISIS 403102 gradually decreased and was reduced to 11% by day 24. By day 56, Factor 7 mRNA levels in ISIS 403102 treated mice are equal to that of the PBS control. Results are presented as percent inhibition of Factor 7, relative to control. These data show that the peak effect of a single dose of 50 mg/kg of ISIS 403102 occurs on about day 4 and duration of action lasts for at least 24 days.

TABLE 21
Antisense inhibition of murine Factor 7 mRNA in BALB/c mice in
a single dose pharmacokinetic study

| Days | % Inhibition |
|---|---|
| 1 | 61 |
| 2 | 87 |
| 3 | 92 |
| 4 | 92 |
| 6 | 86 |
| 8 | 80 |
| 12 | 72 |
| 24 | 11 |
| 56 | 0 |

PT and aPTT Assay

Prothrombin Time (PT) and Activated Partial Thromboplastin Time (aPTT) were measured using platelet poor plasma (PPP) from mice treated with ISIS 403102. PT and aPTT values provided in Table 22 are reported as International Normalized Ratio (INR) values. INR values for PT and aPTT were determined by dividing the PT or aPTT value for the experimental group (i.e. 50 mg/kg treatment with ISIS 403102) by the PT or aPTT for the PBS treated group. This ratio was then raised to the power of the International Sensitivity Index (ISI) of the tissue factor used.

As shown in Table 22, PT increased from 1.11 on day 1 to 1.97 on day 4. PT decreased gradually after day 4 until PT reached 1.10 on day 56. aPTT increased from 1.00 to 1.24 on day 4. aPTT decreased gradually after day 4 until aPTT reached 0.97 on day 56. Consistent with the mRNA expression data (above), these data show that the peak effect of a single dose of 50 mg/kg of ISIS 403102 occurs on about day 4 and duration of action lasts at least 24 days.

TABLE 22
PT and aPTT analysis in BALB/c mice in a single dose
pharmacokinetic study

| Days | PT INR | aPTT INR |
|---|---|---|
| 1 | 1.11 | 1.00 |
| 2 | 1.64 | 1.05 |
| 3 | 1.81 | 1.24 |
| 4 | 1.97 | 1.15 |
| 6 | 1.59 | 1.23 |
| 8 | 1.55 | 1.18 |
| 12 | 1.30 | 1.18 |
| 24 | 1.12 | 1.02 |
| 56 | 1.10 | 0.97 |

### Example 13

### Multiple Dose Pharmacokinetic Assay of ISIS 403102

### Treatment

The duration of action of multiple doses of ISIS 403102 on antisense inhibition of murine Factor 7 and clotting time was evaluated. In a first group of mice, 25 mg/kg of ISIS 403102 was injected subcutaneously as a single dose. Mice from the first group were sacrificed on days 1 and 3 after the single dose. In a second group of mice, 25 mg/kg of ISIS 403102 was administered subcutaneously twice a week for 1 week. Mice from the second group were sacrificed on day 3 after the last dose of ISIS 403102 was administered. In a third group of mice, 25 mg/kg of ISIS 403102 was administered subcutaneously twice a week for 2 weeks. Mice from the third group were sacrificed on day 3 after the last dose of ISIS 403102 was administered. In a fourth group of mice, 25 mg/kg of ISIS 403102 was administered subcutaneously twice a week for 3 weeks. Mice from the fourth group were sacrificed on days 2, 7, 14, 28, 42, and 56 after the last dose of ISIS 403102 was administered. A control group of 3 mice was injected with PBS in a single dose. Mice from the control group were sacrificed 1 day later. Mice in all groups were sacrificed by cervical dislocation following anesthesia with 150 mg/kg ketamine mixed with 10 mg/kg xylazine administered by intraperitoneal injection. Liver was harvested for RNA analysis and plasma was collected for clotting analysis (PT and aPTT) for mice in all groups.

### RNA Analysis

RNA was extracted from liver tissue for quantitative RT-PCR analysis of Factor 7. Results are presented as percent inhibition of Factor 7, relative to PBS control. As shown in Table 23, a single dose treatment of ISIS 403102 resulted in inhibition of Factor 7 as early as day 1. Inhibition increased through day 3 in the single dose treatment group. Two doses of ISIS 403102 resulted in increased inhibition on day 3 as compared to one dose of ISIS 403102. Inhibition increased through day 3 in the 2 dose treatment group. Four doses of ISIS 403102 resulted in increased inhibition in comparison to the 2 dose treatment group on day 3.

Six doses of ISIS 403102 resulted in increased inhibition on day 7 as compared to 6 doses of ISIS 403102 on day 2. In mice treated with 6 doses of ISIS 403102, Factor 7 inhibition declined progressively on days 14, 28, 42, and 56.

TABLE 23
Dose-dependent reduction of Factor 7 mRNA in a multiple
dose pharmacokinetic study

| No. of | Days after | % |
|---|---|---|
| doses | last dose | inhibition |
| 1 | 1 | 44 |
| 1 | 3 | 74 |
| 2 | 3 | 86 |
| 4 | 3 | 91 |
| 6 | 2 | 83 |
| 6 | 7 | 88 |
| 6 | 14 | 64 |
| 6 | 28 | 33 |
| 6 | 42 | 5 |
| 6 | 56 | 11 |

PT and aPTT Assay

Prothrombin Time (PT) and Activated Partial Thromboplastin Time (aPTT) were measured using platelet poor plasma (PPP) from mice treated with ISIS 403102. PT and aPTT values provided in Table 24 are reported as International Normalized Ratio (INR) values. INR values for PT and aPTT were determined by dividing the PT or aPTT value for the experimental group (i.e. 50 mg/kg treatment with ISIS 403102) by the PT or aPTT for the PBS treated group. This ratio was then raised to the power of the International Sensitivity Index (ISI) of the tissue factor used.

As shown in Table 24, PT was increased on day 3 in mice treated with a single dose of ISIS 403102 in comparison to mice treated with a single dose of ISIS 403102 on day 1. On day 3, PT increased in mice treated with 2 doses of ISIS 403102 over mice treated with a single dose of ISIS 403102. PT increased in mice treated with 4 doses of ISIS 403102 over those mice treated with 2 doses of ISIS 403102 on day 3. PT decreased in mice receiving 6 doses of ISIS 403102 from day 7 to day 56.

aPTT was slightly increased on day 3 in mice treated with a single dose of ISIS 403102 in comparison to mice treated with a single dose of ISIS 403102 on day 3. On day 3, aPTT increased in mice treated with 2 doses of ISIS 403102 over mice treated with a single dose of ISIS 403102. aPTT increased in mice treated with 4 doses of ISIS 403102 over those mice treated with 2 doses of ISIS 403102 on day 3. aPTT decreased in mice receiving 6 doses of ISIS 403102 from day 7 to day 56.

TABLE 24
PT and aPTT analysis in BALB/c mice in a multiple dose
pharmacokinetic study

| No. of | Days after | | |
|---|---|---|---|
| doses | last dose | PT INR | aPTT INR |
| 1 | 1 | 1.04 | 1.08 |
| 1 | 3 | 1.20 | 1.10 |
| 2 | 3 | 1.43 | 1.29 |
| 4 | 3 | 2.13 | 1.53 |
| 6 | 2 | 1.64 | 1.62 |
| 6 | 7 | 2.08 | 1.67 |
| 6 | 14 | 1.37 | 1.25 |
| 6 | 28 | 0.96 | 0.90 |
| 6 | 42 | 1.00 | 1.00 |
| 6 | 56 | 0.99 | 0.98 |

### Example 14

### In Vivo Effect of Antisense Inhibition of Factor 7 with ISIS 403102 in the FeCl₃ Induced Venous Thrombosis (VT) Model

### Treatment

Three groups of BALB/c mice were injected with 25 mg/kg, 37.5 mg/kg, or 50 mg/kg of ISIS 403102, administered subcutaneously twice a week for 3 weeks. Two control groups of BALB/c mice were treated with PBS, administered subcutaneously twice a week for 3 weeks. Thrombus formation was induced with FeCl₃ in half of the mice while the rest of the mice were assayed for tail bleeding. Two days after receiving the last dose of ISIS 403102 or PBS, mice were anesthetized with 150 mg/kg ketamine mixed with 10 mg/kg xylazine administered by intraperitoneal injection. Thrombus formation was induced with FeCl₃ in all groups of the VT mice except the first control group.

In mice undergoing FeCl₃ treatment, thrombus formation was induced by applying a piece of filter paper (2×4 min) pre-saturated with 10% FeCl₃ solution directly on the vena cava. After 3 minutes of exposure, the filter paper was removed. Thirty minutes after the filter paper application, a fixed length of the vein containing the thrombus was dissected out for platelet analysis. Liver was collected for RNA analysis.

### RNA Analysis

RNA was extracted from liver tissue for real-time RT-PCR analysis of Factor 7. Results are presented as percent inhibition of Factor 7, relative to PBS control. As shown in Table 25, treatment with ISIS 403102 resulted in significant dose-dependent reduction of Factor 7 mRNA in comparison to the PBS control. These data show that antisense oligonucleotides can be used to inhibit expression of Factor 7.

TABLE 25
Dose-dependent reduction of Factor 7 mRNA in the FeCl₃ induced
venous thrombosis model

| | % |
|---|---|
| mg/kg | inhibition |
| 25 | 24 |
| 37.5 | 74 |
| 50 | 69 |

### Quantification of Platelet Composition

Real-time RT-PCR quantification of platelet factor-4 (PF-4) was used to quantify platelets in the vena cava as a measure of thrombus formation. Results are presented as a percentage of PF-4 in ISIS 403102, as compared to the two PBS-treated control groups. As shown in Table 26, treatment with ISIS 403102 resulted in a reduction of PF-4 in comparison to the PBS control. Therefore, antisense oligonucleotides are useful for inhibiting thrombus and clot formation.

TABLE 26
Analysis of thrombus formation by real-time RT-PCR quantification
of PF-4 in the FeCl₃ induced venous thrombosis model

| | mg/kg | PF-4 |
|---|---|---|
| PBS − FeCl₃ | | 0 |
| PBS + FeCl₃ | | 100 |
| ISIS 403102 | 25 | 34 |
| | 37.5 | 24 |
| | 50 | 24 |

### Tail Bleeding Assay

Mice not receiving treatment with FeCl₃ solution were evaluated in a tail bleeding chamber. Mice were placed into the bleeding chamber two days after the final treatment of ISIS 403102 or PBS. Mice were anesthetized in the chamber with isofluorane and a small piece of tail (approximately 4 mm from the tip) was cut with sterile scissors. The cut tail was immediately placed in a 15 mL Falcon tube filled with approximately 10 mL of 0.9% NaCl buffer solution warmed to 37° C. The blood was collected over the course of 40 minutes. The saline filled tubes were weighed both before and after bleeding. The results are provided in Table 27.

Treatment with 25 mg/kg and 37.5 mg/kg ISIS 403102 caused a slight decrease in the amount of bleeding in comparison to PBS treated mice. Bleeding was the same in mice treated with 50 mg/kg ISIS 403102 and mice treated with PBS. These data suggest that treatment with ISIS 403102 does not increase hemorrhagic potential.

TABLE 27
Tail bleeding assay

| | Mg/kg | Blood (g) |
|---|---|---|
| PBS | | 0.10 |
| 403102 | 25 | 0.06 |
| | 37.5 | 0.06 |
| | 50 | 0.10 |

### Example 15

### In Vivo Antisense Inhibition of Murine Factor 7 by ISIS 403102 Compared to Warfarin

### Treatment

ISIS 403102 and warfarin (Coumadin®) were evaluated in BALB/c mice. Four groups of BALB/c mice were treated with 5 mg/kg, 10 mg/kg, 20 mg/kg, or 40 mg/kg of ISIS 403102, administered subcutaneously twice a week for 3 weeks. Two days after receiving the last dose of ISIS 403102, mice were anesthetized with 150 mg/kg ketamine mixed with 10 mg/kg xylazine administered by intraperitoneal injection. A fifth group of BALB/c mice was treated with 3 mg/kg of warfarin, administered intraperioneally daily for 6 days. Four hours after the last dose of warfarin, mice were sacrificed. A control group of BALB/c mice were treated with PBS, administered subcutaneously twice a week for 3 weeks. Two days after the last dose of PBS, mice were anesthetized with 150 mg/kg ketamine mixed with 10 mg/kg xylazine administered by intraperitoneal injection. Thrombus formation was induced with FeCl₃ in groups of mice except the first control group.

In mice undergoing FeCl₃ treatment, thrombus formation was induced by applying a piece of filter paper (2×4 mm) pre-saturated with 10% FeCl₃ solution directly on the vena cava. After 3 minutes of exposure, the filter paper was removed. Thirty minutes after the filter paper application, a fixed length of the vein containing the thrombus was dissected out for platelet analysis. Liver was collected for RNA analysis.

### RNA Analysis

RNA was extracted from liver tissue for real-time RT-PCR analysis of Factor 7. Results are presented as percent inhibition of Factor 7, relative to PBS control. As shown in Table 28, treatment with ISIS 403102 resulted in significant dose-dependent reduction of Factor 7 mRNA in comparison to the PBS control. Conversely, treatment with warfarin did not result in significant reduction of Factor 7 as compared to the PBS control.

TABLE 28
Dose-dependent reduction of Factor 7 mRNA in the FeCl₃ induced
venous thrombosis model

| | | % |
|---|---|---|
| | mg/kg | inhibition |
| PBS − FeCl₃ | | 0 |
| PBS + FeCl₃ | | 0 |
| Warfarin | 3 | 10 |
| ISIS 403102 | 5 | 59 |
| | 10 | 84 |
| | 20 | 95 |
| | 40 | 99 |

### Example 16

### Effect of Dose-Dependent Antisense Inhibition of Murine Factor 7 on the FeCl₃ Induced Venous Thrombosis (VT) Model Compared to Warfarin

### Treatment

ISIS 403102 and warfarin (Coumadin®) were evaluated in BALB/c mice. Four groups of BALB/c mice were treated with 5 mg/kg, 10 mg/kg, 20 mg/kg, or 40 mg/kg of ISIS 403102, administered subcutaneously twice a week for 3 weeks. Two days after receiving the last dose of ISIS 403102, mice were anesthetized with 150 mg/kg ketamine mixed with 10 mg/kg xylazine administered by intraperitoneal injection. Six additional groups of BALB/c mice was treated with 0.5 mg/kg, 1 mg/kg, 2 mg·kg, 3 mg/kg, 4 mg/kg, or 5 mg/kg of warfarin, administered intraperioneally daily for 6 days. Four hours after the last dose of warfarin, mice were sacrificed. A control group of BALB/c mice were treated with PBS, administered subcutaneously twice a week for 3 weeks. Two days after the last dose of PBS, mice were anesthetized with 150 mg/kg ketamine mixed with 10 mg/kg xylazine administered by intraperitoneal injection. Thrombus formation was induced with FeCl₃ in groups of mice except the first control group.

In mice undergoing FeCl₃ treatment, thrombus formation was induced by applying a piece of filter paper (2×4 mm) pre-saturated with 10% FeCl₃ solution directly on the vena cava. After 3 minutes of exposure, the filter paper was removed. Thirty minutes after the filter paper application, a fixed length of the vein containing the thrombus was dissected out for platelet analysis. Liver was collected for RNA analysis.

### Quantification of Platelet Composition

Real-time RT-PCR quantification of platelet factor-4 (PF-4) was used to quantify platelets in the vena cava as a measure of thrombus formation. Results are presented as a percentage of PF-4 in ISIS 403102 or warfarin treated mice, as compared to the two PBS-treated control groups. As shown in Table 29, treatment with ISIS 403102 resulted in a dose-dependent reduction of PF-4 in comparison to the PBS control for dosages of 5 mg/kg and higher. Treatment with warfarin resulted in a reduction of PF-4 in comparison to the PBS control at a dose of 1 mg/kg and higher. Therefore, ISIS antisense oligonucleotides are useful for inhibiting thrombus and clot formation.

TABLE 29
Analysis of thrombus formation by real-time RT-PCR quantification of
PF-4 in the FeCl₃ induced venous thrombosis model

| | mg/kg | PF-4 |
|---|---|---|
| PBS − FeCl₃ | | 0 |
| PBS + FeCl₃ | | 100 |
| Warfarin | 0.5 | 165 |
| | 1 | 63 |
| | 2 | 47 |
| | 3 | 35 |
| | 4 | 22 |
| | 5 | 0 |
| ISIS 403102 | 1 | 120 |
| | 3 | 112 |
| | 5 | 69 |
| | 10 | 22 |
| | 20 | 41 |
| | 40 | 38 |

### Example 17

### Effect of Antisense Inhibition of Murine Factor 7 in a Tail Bleeding Assay Compared to Warfarin

### Treatment

Tail-bleeding was measured to observe whether treatment with ISIS 403102 or warfarin causes internal hemorrhage in mice. ISIS 403102 and warfarin (Coumadin®) were evaluated in the tail bleeding assay. Six groups of BALB/c mice were treated with 1.25 mg/kg, 2.5 mg/kg, 5 mg/kg, 10 mg/kg, 20 mg/kg, or 40 mg/kg of ISIS 403102, administered subcutaneously twice a week for 3 weeks. An additional 6 groups of BALB/c mice were treated with 0.5 mg/kg, 1 mg/kg, 2 mg/kg, 3 mg/kg, 4 mg/kg, and 5 mg/kg of warfarin, administered intraperioneally daily for 6 days. A separate control group of BALB/c mice was treated with PBS, administered subcutaneously twice a week for 3 weeks.

### Tail-Bleeding Assay

Two days after the final treatment of ISIS 403102, warfarin, or PBS, mice were placed in a tail bleeding chamber. Mice were anesthetized in the chamber with isofluorane and a small piece of tail (approximately 4 mm from the tip) was cut with sterile scissors. The cut tail was immediately placed in a 15 mL Falcon tube filled with approximately 10 mL of 0.9% NaCl buffer solution warmed to 37° C. The blood was collected over the course of 40 minutes. The saline filled tubes were weighed both before and after bleeding. The results are provided in Table 30.

Treatment with ISIS 403102 did not significantly affect bleeding as compared to PBS control mice. However, warfarin did increase bleeding in mice as compared to the PBS control mice. Increased doses of warfarin correlated positively with increased blood loss. These data suggest that the hemorrhagic potential of ISIS 403102 is low, especially in comparison to warfarin.

TABLE 30
Tail bleeding assay in the FeCl₃ induced venous thrombosis model

| | Dose in | |
|---|---|---|
| Treatment | mg/kg | Blood (g) |
| PBS | 0 | 0.06 |
| Warfarin | 0.5 | 0.16 |
| | 1 | 0.28 |
| | 2 | 0.58 |
| | 3 | 0.78 |
| | 4 | 0.66 |
| | 5 | 0.93 |
| ISIS 404071 | 1.25 | 0.03 |
| | 2.5 | 0.03 |
| | 5 | 0.03 |
| | 10 | 0.09 |
| | 20 | 0.09 |
| | 40 | 0.09 |

### Example 18

### Effect of Antisense Inhibition of Murine Factor 7 in the Tail Bleeding Assay Compared to Apixaban

### Treatment

ISIS 403102 and Apixaban were evaluated in BALB/c mice. In a first group of BALB/c mice, 40 mg/kg of ISIS 403102 was administered subcutaneously twice a week for 3 weeks. An additional 3 groups of BALB/c mice were treated with 5 mg/kg and 10 mg/kg of Apixaban, administered in a single intraperitoneal dose, and 10 mg/kg of Apixaban administered as a single subcutaneous dose. A control group of BALB/c mice was treated with PBS, administered subcutaneously twice a week for 3 weeks.

### Tail-Bleeding Assay

Two days after the final treatment of ISIS 403102 or PBS, mice were placed in a tail bleeding chamber. Mice from the groups treated with Apixaban were analyzed 30 minutes after the single dose. Mice were anesthetized in the chamber with isofluorane and a small piece of tail (approximately 4 mm from the tip) was cut with sterile scissors. The cut tail was immediately placed in a 15 mL Falcon tube filled with approximately 10 mL of 0.9% NaCl buffer solution warmed to 37° C. The blood was collected over the course of 40 minutes. The saline filled tubes were weighed before and after bleeding. The results are provided in Table 31.

Mice treated with ISIS 403102 had less bleeding than PBS treated mice. Mice treated with 5 mg/kg of apixaban by intraperitoneal injection had the same amount of bleeding as PBS treated mice. Mice treated with 10 mg/kg of apixaban by intraperitoneal injection had increased bleeding as compared to the PBS treated mice. Mice treated with 10 mg/kg of apixaban by subcutaneous injection had less bleeding than PBS mice. These data suggest that the hemorrhagic potential of ISIS 403102 is low.

TABLE 31
Tail bleeding assay in BALB/c mice

| | mg/kg | Blood (g) |
|---|---|---|
| PBS | | 0.22 |
| ISIS 403102 | 40 (s.c.) | 0.10 |
| Apixaban | 5 (i.p.) | 0.22 |
| | 10 (i.p.) | 0.58 |
| | 10 (s.c.) | 0.04 |

### Example 19

### In Vivo Effect of Antisense Inhibition of Murine Factor 7 on Cancer Metastasis

The effect of inhibition of Factor 7 with ISIS 403102 on the formation of tissue factor-Factor 7 complex and its role in extravasation of cancer cells during metastasis will be evaluated. Two groups of severe combined immunodeficiency (SCID) mice will be treated with ISIS 403102, injected at a dose of 20 mg/kg twice a week for 3 weeks. A control group of mice will be injected with PBS twice a week for 3 weeks. Two days after the last dose of ISIS 403102 or PBS, one of the ISIS 403102 treated groups and the control group will be injected intravenously with 50×10⁶ MDA-MB-231 breast carcinoma cells.

Two weeks after the injection with MDA-MB-231 breast carcinoma cells, mice will be sacrificed. The lungs will be harvested and real-time RT-PCR analysis of human GAPDH mRNA levels performed. The results will be normalized with mouse cyclophilin A mRNA levels. Human GAPDH levels will in the group treated with ISIS 403102 and MDA-MB-231 breast carcinoma cells group will be compared to human GAPDH levels in the other two groups of mice. This experiment is designed to assess the effect of inhibition of Factor 7 on the development of metastasis in the lungs.

### Example 20

### In Vivo Effect of Antisense Inhibition of Murine Factor 7 on Liver Fibrosis

The effect of inhibition of Factor 7 with ISIS 403102 on experimental liver fibrosis will be evaluated in the carbon tetrachloride liver injury model.

### Treatment

In a first group of BALB/c mice, 20 mg/kg ISIS 403102 will be injected subcutaneously twice a week for 8 weeks. In a second group of mice, PBS will be injected subcutaneously twice a week for 8 weeks. Two weeks after the first treatment with ISIS 403102 or PBS, both groups of mice will be dosed intraperitoneally with 5 μl of carbon tetrachloride (CCl₄) dissolved in 95 μl of mineral oil twice a week for 5 weeks. A third group of mice will be injected with 100 μl mineral oil alone. Mice will be sacrificed by cervical dislocation following anesthesia with isofluorane. Liver tissue will be harvested from all mice. Real-time RT-PCR will be used to determine the expression of fibrosis related genes, including, collagen type 1, α-smooth muscle actin, matrix metalloproteinase (MMP) 3, TGF-β, Timp1 and Timp2 (MMP inhibitors). The levels in the experimental group will be compared to the levels in the control mice to assess the effect of inhibition of Factor 7 on the development of liver fibrosis.

### Example 21

### In Vivo Effect of Antisense Inhibition of Murine Factor 7 on Collagen-Induced Arthritis

The effect of inhibition of Factor 7 with ISIS 403102 on the formation of tissue factor-Factor 7 complex and its role in fibrin accumulation in the joints leading to joint inflammation and rheumatoid arthritis will be evaluated in a collagen-induced arthritis model.

### Treatment

In a first group of DBA/1J mice, 20 mg/kg of ISIS 403102 will be injected subcutaneously twice a week for 8 weeks. Two groups of mice will be injected with PBS twice a week for 8 weeks. Two weeks after the first treatment of ISIS 403102, type II bovine collagen (Chondrex) will be mixed with complete Freund's adjuvant, homogenized on ice and the emulsion, containing 100 μg of collagen, will be injected subcutaneously in the experimental group and the first control group. A booster injection containing 100 μg collagen type II in incomplete Freund's adjuvant will be injected subcutaneously 7 days after the first collagen injection in both these groups.

Mice in all groups will be examined each day from day 18 after the first collagen injection for the visual appearance of arthritis in peripheral joints. The clinical severity of arthritis will be scored as follows: 1 point for each swollen digit except the thumb (maximum, 4), 1 point for the tarsal or carpal joint, and 1 point for the metatarsal or metacarpal joint with a maximum score of 6 for a hindpaw and 5 for a forepaw. Each paw will be graded individually, the cumulative clinical arthritic score per mouse reaching a maximum of 22 points. Arthritis in the experimental groups will be compared to the control group to assess the effect of inhibition of Factor 7 on the development of arthritis in the joints.

Six weeks after the initial injection of collagen, the maximal level of arthritis will be induced. After mice are anesthetized with isofluorane and plasma is collected, the mice will be sacrificed by cervical dislocation. Livers will be harvested for RNA analysis of Factor 7 mRNA. Plasma collected from all three groups will be analyzed for clotting time (PT and aPTT). The measurement of thrombin-antithrombin (TAT) complexes in the plasma will also be performed by ELISA. The results in the experimental groups will be compared to the control group to assess the effect of inhibition of Factor 7 on the clotting time and formation of TAT complexes.

### Example 22

### In Vivo Effect of Antisense Inhibition of Murine Factor 7 in Combination with Plavix in the FeCl₃ Induced Venous Thrombosis (VT) Model

### Treatment

The combination of ISIS 403102 and Plavix was evaluated in the FeCl₃ induced VT mouse model. Four groups of eight BALB/c mice, weighing approximately 25 g each, were treated with 6.25 mg/kg, 12.50 mg/kg, 25.00 mg/kg, or 50.00 mg/kg of Plavix. Mice were given two doses of Plavix on day one and one dose of Plavix on day two, two hours before surgery.

An additional four groups of eight BALB/c mice, weighing approximately 25 g each, were treated with 20 mg/kg of ISIS 403102, administered subcutaneously twice a week for three weeks. After the last dose of ISIS 403102, mice were treated with 6.25 mg/kg, 12.50 mg/kg, 25.00 mg/kg, or 50.00 mg/kg of Plavix. Two doses of Plavix were administered to the mice on day one and one dose of Plavix was administered on day two, two hours before surgery.

Two control groups of eight BALB/c mice, weighing approximately 25 g each, were not treated with ISIS 403102 or Plavix. An additional two control groups of eight BALB/c mice, weighing approximately 25 g each, were treated with 20 mg/kg of ISIS 403102, administered subcutaneously twice a week for three weeks, but were not treated with Plavix. Thrombus formation was induced with FeCl₃ in all of the mice except the first and third control groups. All mice were anesthetized with 150 mg/kg of ketamine mixed with 10 mg/kg of xylazine administered by intraperitoneal injection.

In mice undergoing FeCl₃ treatment, thrombus formation was induced by applying a piece of filter paper (2×4 mm) pre-saturated with 10% FeCl₃ solution directly on the inferior vena cava. After 3 minutes of exposure, the filter paper was removed. Thirty minutes after the filter paper application, a fixed length of the vein containing the thrombus was dissected out for platelet analysis.

### Quantification of Platelet Composition

Real-time PCR quantification of PF-4 was used to quantify platelets in the vena cava as a measure of thrombus formation. As shown in Table 32, treatment with Plavix resulted in a reduction of PF-4 in comparison to the PBS control. Treatment with Plavix in combination with ISIS 403102 resulted in a higher reduction of PF-4 in comparison to Plavix alone. Therefore, the combination of anti-platelet therapy with Factor 7 ASO increases antithrombotic activity. Data is presented as percent of PF-4 mRNA as compared to the PBS+ FeCl₃ control,

TABLE 32

| | ISIS 403102 | Plavix | |
|---|---|---|---|
| Treatment | mg/kg | mg/kg | PF-4 |
| PBS − FeCl₃ | 0 | 0 | 0 |
| PBS + FeCl₃ | 0 | 0 | 100 |
| Plavix only | 0 | 6.25 | 61 |
| | 0 | 12.50 | 24 |
| | 0 | 25.00 | 13 |
| | 0 | 50.00 | 6 |
| ISIS 403102 − FeCl₃ | 20 | 0 | 2 |
| ISIS 403102 + FeCl₃ | 20 | 0 | 39 |
| Plavix (+ISIS 403102) | 20 | 6.25 | 32 |
| | 20 | 12.50 | 9 |
| | 20 | 25.00 | 11 |
| | 20 | 50.00 | 4 |

### Example 23

### In Vivo Effect of Antisense Inhibition of Murine Factor 7 in Combination with Plavix on Bleeding

### Treatment

Tail-bleeding was measured to observe whether treatment with ISIS 403102 in combination with Plavix causes an increase in bleeding tendency. ISIS 403102 was administered subcutaneously at a dosage of 20 mg/kg twice a week for 3 weeks to 5 groups of eight BALB/c mice. After the last dose of ISIS 403102, mice were treated with 0 mg/kg, 6.25 mg/kg, 12.50 mg/kg, 25.00 mg/kg, or 50.00 mg/kg of Plavix. Two doses of Plavix were administered to the mice on day one and one dose of Plavix was administered on day two, two hours before bleeding.

An additional 5 groups of eight BABL/c mice were treated similarly, except they did not receive ISIS 403102 injections.

### Tail-Bleeding Assay

Two hours after receiving their final treatment, mice were placed in a tail bleeding chamber. Mice were anesthetized in the chamber with isoflurane and a small piece of tail (approximately 4 mm from the tip) was cut with sterile scissors. The cut tail was immediately placed in a 15 mL Falcon tube filled with approximately 10 mL of 0.9% NaCl buffer solution warmed to 37° C. The blood was collected for the course of 40 minutes. The saline filled tubes were weighed both before and after bleeding.

Taken with the results of Example 22, these data show that the combination of anti-platelet therapy with Factor 7 ASO increases antithrombotic activity without increased bleeding risk.

TABLE 33

| | ISIS 403102 | Plavix | |
|---|---|---|---|
| Treatment | mg/kg | mg/kg | Blood (g) |
| No treatment | 0 | 0 | 0.007 |
| Plavix only | 0 | 6.25 mg/kg | 0.142 |
| | 0 | 12.50 mg/kg | 0.157 |
| | 0 | 25.00 mg/kg | 0.516 |
| | 0 | 50.00 mg/kg | 0.465 |
| ISIS 404071 only | 20 mg/kg | 0 | 0.003 |
| Plavix (+ISIS 404071) | 20 mg/kg | 6.25 mg/kg | 0.116 |
| | 20 mg/kg | 12.50 mg/kg | 0.213 |
| | 20 mg/kg | 25.00 mg/kg | 0.520 |
| | 20 mg/kg | 50.00 mg/kg | 0.478 |

1-74. (canceled)

75. A method of increasing antithrombotic activity of in a human comprising:
co-administering to the human an amount of a compound comprising a modified oligonucleotide consisting of 12 to 30 linked nucleosides wherein the modified oligonucleotide has a nucleobase sequence that is at least 90% complementary to a human Factor 7 nucleobase sequence, and an amount of an antiplatelet agent;
wherein the amount of the modified oligonucleotide compound and the amount of antiplatelet agent are selected such that co-administration results in antithrombotic activity that is greater than administering the same amount of the modified oligonucleotide compound alone, and greater than administering the same amount of the antiplatelet agent alone, and wherein the risk of bleeding is not increased.

76. The method of claim 75, wherein the antiplatelet agent is clopidogrel.

77. The method of claim 76, wherein the compound consists of a single-stranded modified oligonucleotide.

78. The method of claim 77, wherein the nucleobase sequence of the modified oligonucleotide is at least 95% complementary to a human Factor 7 nucleobase sequence.

79. The method of claim 77, wherein the nucleobase sequence of the modified oligonucleotide is 100% complementary to a human Factor 7 nucleobase sequence.

80. The method of claim 77, wherein at least one internucleoside linkage of the modified oligonucleotide is a modified internucleoside linkage.

81. The method of claim 80, wherein each internucleoside linkage of the modified oligonucleotide is a phosphorothioate internucleoside linkage.

82. The method of claim 77, wherein at least one nucleoside of the modified oligonucleotide comprises a modified sugar.

83. The method of claim 82, wherein at least one modified sugar is a bicyclic sugar.

84. The method of claim 82, wherein at least one modified sugar comprises a 2′-O-methoxyethyl or a 4′-(CH₂)ₙ—O-2′ bridge, wherein n is 1 or 2.

85. The method of claim 77, wherein at least one nucleoside of the modified oligonucleotide comprises a modified nucleobase.

86. The method of claim 85, wherein the modified nucleobase is a 5-methylcytosine.

87. The method of claim 76, wherein the modified oligonucleotide comprises:
a gap segment consisting of linked deoxynucleosides;
a 5′ wing segment consisting of linked nucleosides;
a 3′ wing segment consisting of linked nucleosides;
wherein the gap segment is positioned between the 5′ wing segment and the 3′ wing segment and wherein each nucleoside of each wing segment comprises a modified sugar.

88. The method of claim 87, wherein the modified oligonucleotide comprises:
a gap segment consisting of ten linked deoxynucleosides;
a 5′ wing segment consisting of five linked nucleosides;
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar; wherein each cytosine in said modified oligonucleotide is a 5-methylcytosine, and wherein each internucleoside linkage of said modified oligonucleotide is a phosphorothioate linkage.

89. The method of claim 88, wherein the modified oligonucleotide consists of 20 linked nucleosides.

90. The method of claim 89, wherein the modified oligonucleotide consists of a single-stranded modified oligonucleotide.

91. The method of claim 90, wherein the nucleobase sequence of the modified oligonucleotide is at least 95% complementary to a human Factor 7 nucleobase sequence.

92. The method of claim 90, wherein the nucleobase sequence of the modified oligonucleotide is 100% complementary to a human Factor 7 nucleobase sequence.

93. The method of claim 90, wherein the nucleobase sequence of the modified oligonucleotide is 100% complementary to a nucleobase sequence selected from the group consisting of SEQ ID NOs: 1, 2, 3 and 167.
